SEQUENCE LISTING (89 sequences)

SEQ ID NO: 1 - DNA, 17 bp, Ralstonia eutropha
tttcaggagattcaaga

SEQ ID NO: 2 - DNA, 13042 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccaatattaaaaaaataagagttaccatttaaggtaactcttatttttattagtcggacgcgcgctccggcaccgggagattcacccactccttgtagaaggcctcgatgtccggctcgaagtcgtggatcacgggataccacggggtaggcgcttcgacgtcgtgcatggtgccgcacgatgggcagtagtattcgcgatacacctgccactgggtgtcgggggccatcagcttcggatacacctctgtcatcgcctcctcggtgtctcgcacgtagatcgcggcatgcagcttccagttctcgcggtaatcacagaactcgtgaccgcagtcgcacttgactacccacttcttgctcttgaccgactgcacgataaacatgtgcgggctgagcggtagcacgatgcggtcaggaaagttcaccttggcctgcaaggctgacaggtactgcgcgaaccggctctcgtccttgggcatcgagagcatgcggaaggtggtctcccaatcgagggaaccatcgacgaggtgggcgacctgttcattggtataggttgacattcttgaatctcctgaaattactcttcgacctgaacgacggtggtgacatcgggcagcagggagaggtccatgcggtgcttggcgccgtaggatggcacgcccagctcttcttcacgcacctgccagtcggcgggcaggctccagaattccttgaactcccgcgtgaacttctcagacagggcaaagctggtggcgaacatgtgctgcacctggacggacgcgtgcttgttgagaatgcgttcgcgctcttccttcatccattcccgcgtcggcagcgcccgggccaggcgttccttgcgaacttcggcacggcgtgcttgggtcttgacggcatccacggtaaagacgcccttgtcgtcttgggtgaagacagccccatataccttctgcgcgtaggccggcagcaggaacttctggttcaggtccgcttcgatggcttgcgggtcccggtcgagagggtcgccaaagcccgggccaccgcgcaggtagttcaggtacagatcatggttggcgtagcagtcctccgtcgtgatgcactgcttgtcgcgcttgacctcggcggtggcgtcgatgtggcgttcgaagtcggggttactcggatcgatgtcgccgcccagcggcagggagtcgccgatggcgatgcgctggtcgaggccggtcttgtgcgcctcgaagcgatagccggtagcggagggatagccgcccatcatgccccagtcgctgttcatgaagccgttgcccatgaagaacatggtccagtcctgggcgttccacaccatgcgcagcgtctcgaacccgcaaccgccgcggtacttgccgtagccgccggaattggccttgacgttgcggccaaggtagagcagcggctcagccatctcccagatttcgatgtcgcccatatcgccttccggattccagatggcggcagcgtggttgagaccatcctttacggcgcaggcgccggtgccacaggaactggcctcgaagctgttgaccgcatggatctctccgtcctggttgatgccgccgccctgcagccagttggaggtgttggcgttgccggcgttgacctcttccaggtagccgcggctgaagtaggactgcgacaggccgcgccacagcgccgcccagccggagaccaggaagtgccaggcataggcatggccggtgcggcgatcatcggggttgcaccacgtgcccttgggcaagcggaactcggtagcgaaatacgccccgtcgttgatacgctgggtcggcacaagggtctggcacatcatcacccagattccggaagtgaaggccacctggtgggcgttgaaggtatgccagccccagcggctcgcgccctcaaagtcgaggcgccactttccgtccgacttgatggtcatctccaccggcgaatgcatgatggagtcgagcttggcgaaggcgttggagacctgcacgtcgtcatgcttgtagggcacgtcgacaaaggaaaccttgcggtacttgccaggcagggtcatcgccttgatgcggctctgcagtccgcggcggccttcttcgatgacctcataggcgaacttctcgtaggcctcgaggccgtcggcacgaatcacttcttcgaccagttcgcggatcatgtggcagccagcgatccgggttttctcatcgaggatccagtatttcggcgtgcgcaccgagcgctgcgactcgtgcagccagtcgcgcagcggctcgtcgttcacgccggtcttgcggcaggtgatcatgtagccgtcgccaaagcgctgcgtctgcccggttgacatcgagcccggcgttaccgagccggtgtcgatgacgtgggtgacgccgcctacccagccgatcagcttcccctcccagaagatcggcacgatggtggcgatgtcgcaggggtgcacgttgccgatggagcagtcgttgttggtgaacatgtcaccgggattgatgccggggttggcctcccagttgttctcgatcatgtacttgatcgccgcccccatggtgccgacgtggatgatgatgccggtcgaggtcagcacgcagtcacccacagcgttgtagagcgtgaagcagagttcgccttcctgctcgacgatggggctggcggcaattttcttcgcggtttcgcgggcatgcaccaagccgccgcgcagcttggagaacagcttctcatagccgatcgggtcgctgtcgcggaactccagtttgtcgaggccgttgtagtggccggacgcctgagtgcgagcgatgatcgcgtcgcggagctccttgagggtcttgccgttctgcagcagattgcccaggcccacttccttgttgctcatcatattcatatcttgaatctcctgaaattactcttcgacctgaacgacggtggtgacatcgggcagcagggagaggtccatgcggtgcttggcgccgtaggatggcacgcccagctcttcttcacgcacctgccagtcggcgggcaggctccagaattccttgaactcccgcgtgaacttctcagacagggcaaagctggtggcgaacatgtgctgcacctggacggacgcgtgcttgttgagaatgcgttcgcgctcttccttcatccattcccgcgtcggcagcgcccgggccaggcgttccttgcgaacttcggcacggcgtgcttgggtcttgacggcatccacggtaaagacgcccttgtcgtcttgggtgaagacagccccatataccttctgcgcgtaggccggcagcaggaacttctggttcaggtccgcttcgatggcttgcgggtcccggtcgagagggtcgccaaagcccgggccaccgcgcaggtagttcaggtacagatcatggttggcgtagcagtcctccgtcgtgatgcactgcttgtcgcgcttgacctcggcggtggcgtcgatgtggcgttcgaagtcggggttactcggatcgatgtcgccgcccagcggcagggagtcgccgatggcgatgcgctggtcgaggccggtcttgtgcgcctcgaagcgatagccggtagcggagggatagccgcccatcatgccccagtcgctgttcatgaagccgttgcccatgaagaacatggtccagtcctgggcgttccacaccatgcgcagcgtctcgaacccgcaaccgccgcggtacttgccgtagccgccggaattggccttgacgttgcggccaaggtagagcagcggctcagccatctcccagatttcgatgtcgcccatatcgccttccggattccagatggcggcagcgtggttgagaccatcctttacggcgcaggcgccggtgccacaggaactggcctcgaagctgttgaccgcatggatctctccgtcctggttgatgccgccgccctgcagccagttggaggtgttggcgttgccggcgttgacctcttccaggtagccgcggctgaagtaggactgcgacaggccgcgccacagcgccgcccagccggagaccaggaagtgccaggcataggcatggccggtgcggcgatcatcggggttgcaccacgtgcccttgggcaagcggaactcggtagcgaaatacgccccgtcgttgatacgctgggtcggcacaagggtctggcacatcatcacccagattccggaagtgaaggccacctggtgggcgttgaaggtatgccagccccagcggctcgcgccctcaaagtcgaggcgccactttccgtccgacttgatggtcatctccaccggcgaatgcatgatggagtcgagcttggcgaaggcgttggagacctgcacgtcgtcatgcttgtagggcacgtcgacaaaggaaaccttgcggtacttgccaggcagggtcatcgccttgatgcggctctgcagtccgcggcggccttcttcgatgacctcataggcgaacttctcgtaggcctcgaggccgtcggcacgaatcacttcttcgaccagttcgcggatcatgtggcagccagcgatccgggttttctcatcgaggatccagtatttcggcgtgcgcaccgagcgctgcgactcgtgcagccagtcgcgcagcggctcgtcgttcacgccggtcttgcggcaggtgatcatgtagccgtcgccaaagcgctgcgtctgcccggttgacatcgagcccggcgttaccgagccggtgtcgatgacgtgggtgacgccgcctacccagccgatcagcttcccctcccagaagatcggcacgatggtggcgatgtcgcaggggtgcacgttgccgatggagcagtcgttgttggtgaacatgtcaccgggattgatgccggggttggcctcccagttgttctcgatcatgtacttgatcgccgcccccatggtgccgacgtggatgatgatgccggtcgaggtcagcacgcagtcacccacagcgttgtagagcgtgaagcagagttcgccttcctgctcgacgatggggctggcggcaattttcttcgcggtttcgcgggcatgcaccaagccgccgcgcagcttggagaacagcttctcatagccgatcgggtcgctgtcgcggaactccagtttgtcgaggccgttgtagtggccggacgcctgagtgcgagcgatgatcgcgtcgcggagctccttgagggtcttgccgttctgcagcagattgcccaggcccacttccttgttgctcatcatattcattcttgaatctcctgaaatgtgaaattgttatccgctcacaattccacacaacatacgagccggaagcataaagtgtaaagcctggggaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaactttctagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 3 - DNA, 13050 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccaatattaaaaaaataagagttaccatttaaggtaactcttatttttattagtcggacgcgcgctccggcaccgggagattcacccactccttgtagaaggcctcgatgtccggctcgaagtcgtggatcacgggataccacggggtaggcgcttcgacgtcgtgcatggtgccgcacgatgggcagtagtattcgcgatacacctgccactgggtgtcgggggccatcagcttcggatacacctctgtcatcgcctcctcggtgtctcgcacgtagatcgcggcatgcagcttccagttctcgcggtaatcacagaactcgtgaccgcagtcgcacttgactacccacttcttgctcttgaccgactgcacgataaacatgtgcgggctgagcggtagcacgatgcggtcaggaaagttcaccttggcctgcaaggctgacaggtactgcgcgaaccggctctcgtccttgggcatcgagagcatgcggaaggtggtctcccaatcgagggaaccatcgacgaggtgggcgacctgttcattggtataggttgacattcttgaatctcctgaaattactcttcgacctgaacgacggtggtgacatcgggcagcagggagaggtccatgcggtgcttggcgccgtaggatggcacgcccagctcttcttcacgcacctgccagtcggcgggcaggctccagaattccttgaactcccgcgtgaacttctcagacagggcaaagctggtggcgaacatgtgctgcacctggacggacgcgtgcttgttgagaatgcgttcgcgctcttccttcatccattcccgcgtcggcagcgcccgggccaggcgttccttgcgaacttcggcacggcgtgcttgggtcttgacggcatccacggtaaagacgcccttgtcgtcttgggtgaagacagccccatataccttctgcgcgtaggccggcagcaggaacttctggttcaggtccgcttcgatggcttgcgggtcccggtcgagagggtcgccaaagcccgggccaccgcgcaggtagttcaggtacagatcatggttggcgtagcagtcctccgtcgtgatgcactgcttgtcgcgcttgacctcggcggtggcgtcgatgtggcgttcgaagtcggggttactcggatcgatgtcgccgcccagcggcagggagtcgccgatggcgatgcgctggtcgaggccggtcttgtgcgcctcgaagcgatagccggtagcggagggatagccgcccatcatgccccagtcgctgttcatgaagccgttgcccatgaagaacatggtccagtcctgggcgttccacaccatgcgcagcgtctcgaacccgcaaccgccgcggtacttgccgtagccgccggaattggccttgacgttgcggccaaggtagagcagcggctcagccatctcccagatttcgatgtcgcccatatcgccttccggattccagatggcggcagcgtggttgagaccatcctttacggcgcaggcgccggtgccacaggaactggcctcgaagctgttgaccgcatggatctctccgtcctggttgatgccgccgccctgcagccagttggaggtgttggcgttgccggcgttgacctcttccaggtagccgcggctgaagtaggactgcgacaggccgcgccacagcgccgcccagccggagaccaggaagtgccaggcataggcatggccggtgcggcgatcatcggggttgcaccacgtgcccttgggcaagcggaactcggtagcgaaatacgccccgtcgttgatacgctgggtcggcacaagggtctggcacatcatcacccagattccggaagtgaaggccacctggtgggcgttgaaggtatgccagccccagcggctcgcgccctcaaagtcgaggcgccactttccgtccgacttgatggtcatctccaccggcgaatgcatgatggagtcgagcttggcgaaggcgttggagacctgcacgtcgtcatgcttgtagggcacgtcgacaaaggaaaccttgcggtacttgccaggcagggtcatcgccttgatgcggctctgcagtccgcggcggccttcttcgatgacctcataggcgaacttctcgtaggcctcgaggccgtcggcacgaatcacttcttcgaccagttcgcggatcatgtggcagccagcgatccgggttttctcatcgaggatccagtatttcggcgtgcgcaccgagcgctgcgactcgtgcagccagtcgcgcagcggctcgtcgttcacgccggtcttgcggcaggtgatcatgtagccgtcgccaaagcgctgcgtctgcccggttgacatcgagcccggcgttaccgagccggtgtcgatgacgtgggtgacgccgcctacccagccgatcagcttcccctcccagaagatcggcacgatggtggcgatgtcgcaggggtgcacgttgccgatggagcagtcgttgttggtgaacatgtcaccgggattgatgccggggttggcctcccagttgttctcgatcatgtacttgatcgccgcccccatggtgccgacgtggatgatgatgccggtcgaggtcagcacgcagtcacccacagcgttgtagagcgtgaagcagagttcgccttcctgctcgacgatggggctggcggcaattttcttcgcggtttcgcgggcatgcaccaagccgccgcgcagcttggagaacagcttctcatagccgatcgggtcgctgtcgcggaactccagtttgtcgaggccgttgtagtggccggacgcctgagtgcgagcgatgatcgcgtcgcggagctccttgagggtcttgccgttctgcagcagattgcccaggcccacttccttgttgctcatcatattcatatcttgaatctcctgaaattactcttcgacctgaacgacggtggtgacatcgggcagcagggagaggtccatgcggtgcttggcgccgtaggatggcacgcccagctcttcttcacgcacctgccagtcggcgggcaggctccagaattccttgaactcccgcgtgaacttctcagacagggcaaagctggtggcgaacatgtgctgcacctggacggacgcgtgcttgttgagaatgcgttcgcgctcttccttcatccattcccgcgtcggcagcgcccgggccaggcgttccttgcgaacttcggcacggcgtgcttgggtcttgacggcatccacggtaaagacgcccttgtcgtcttgggtgaagacagccccatataccttctgcgcgtaggccggcagcaggaacttctggttcaggtccgcttcgatggcttgcgggtcccggtcgagagggtcgccaaagcccgggccaccgcgcaggtagttcaggtacagatcatggttggcgtagcagtcctccgtcgtgatgcactgcttgtcgcgcttgacctcggcggtggcgtcgatgtggcgttcgaagtcggggttactcggatcgatgtcgccgcccagcggcagggagtcgccgatggcgatgcgctggtcgaggccggtcttgtgcgcctcgaagcgatagccggtagcggagggatagccgcccatcatgccccagtcgctgttcatgaagccgttgcccatgaagaacatggtccagtcctgggcgttccacaccatgcgcagcgtctcgaacccgcaaccgccgcggtacttgccgtagccgccggaattggccttgacgttgcggccaaggtagagcagcggctcagccatctcccagatttcgatgtcgcccatatcgccttccggattccagatggcggcagcgtggttgagaccatcctttacggcgcaggcgccggtgccacaggaactggcctcgaagctgttgaccgcatggatctctccgtcctggttgatgccgccgccctgcagccagttggaggtgttggcgttgccggcgttgacctcttccaggtagccgcggctgaagtaggactgcgacaggccgcgccacagcgccgcccagccggagaccaggaagtgccaggcataggcatggccggtgcggcgatcatcggggttgcaccacgtgcccttgggcaagcggaactcggtagcgaaatacgccccgtcgttgatacgctgggtcggcacaagggtctggcacatcatcacccagattccggaagtgaaggccacctggtgggcgttgaaggtatgccagccccagcggctcgcgccctcaaagtcgaggcgccactttccgtccgacttgatggtcatctccaccggcgaatgcatgatggagtcgagcttggcgaaggcgttggagacctgcacgtcgtcatgcttgtagggcacgtcgacaaaggaaaccttgcggtacttgccaggcagggtcatcgccttgatgcggctctgcagtccgcggcggccttcttcgatgacctcataggcgaacttctcgtaggcctcgaggccgtcggcacgaatcacttcttcgaccagttcgcggatcatgtggcagccagcgatccgggttttctcatcgaggatccagtatttcggcgtgcgcaccgagcgctgcgactcgtgcagccagtcgcgcagcggctcgtcgttcacgccggtcttgcggcaggtgatcatgtagccgtcgccaaagcgctgcgtctgcccggttgacatcgagcccggcgttaccgagccggtgtcgatgacgtgggtgacgccgcctacccagccgatcagcttcccctcccagaagatcggcacgatggtggcgatgtcgcaggggtgcacgttgccgatggagcagtcgttgttggtgaacatgtcaccgggattgatgccggggttggcctcccagttgttctcgatcatgtacttgatcgccgcccccatggtgccgacgtggatgatgatgccggtcgaggtcagcacgcagtcacccacagcgttgtagagcgtgaagcagagttcgccttcctgctcgacgatggggctggcggcaattttcttcgcggtttcgcgggcatgcaccaagccgccgcgcagcttggagaacagcttctcatagccgatcgggtcgctgtcgcggaactccagtttgtcgaggccgttgtagtggccggacgcctgagtgcgagcgatgatcgcgtcgcggagctccttgagggtcttgccgttctgcagcagattgcccaggcccacttccttgttgctcatcatattcattcttgaatctcctgaaatgtgaaattgttatccgctcacaattccacacaacatacgagccggaagcataaagtgtaaagcctggggaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaattccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 4 - DNA, 13020 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccaatattaaaaaaataagagttaccatttaaggtaactcttatttttattagtcggacgcgcgctccggcaccgggagattcacccactccttgtagaaggcctcgatgtccggctcgaagtcgtggatcacgggataccacggggtaggcgcttcgacgtcgtgcatggtgccgcacgatgggcagtagtattcgcgatacacctgccactgggtgtcgggggccatcagcttcggatacacctctgtcatcgcctcctcggtgtctcgcacgtagatcgcggcatgcagcttccagttctcgcggtaatcacagaactcgtgaccgcagtcgcacttgactacccacttcttgctcttgaccgactgcacgataaacatgtgcgggctgagcggtagcacgatgcggtcaggaaagttcaccttggcctgcaaggctgacaggtactgcgcgaaccggctctcgtccttgggcatcgagagcatgcggaaggtggtctcccaatcgagggaaccatcgacgaggtgggcgacctgttcattggtataggttgacattcttgaatctcctgaaattactcttcgacctgaacgacggtggtgacatcgggcagcagggagaggtccatgcggtgcttggcgccgtaggatggcacgcccagctcttcttcacgcacctgccagtcggcgggcaggctccagaattccttgaactcccgcgtgaacttctcagacagggcaaagctggtggcgaacatgtgctgcacctggacggacgcgtgcttgttgagaatgcgttcgcgctcttccttcatccattcccgcgtcggcagcgcccgggccaggcgttccttgcgaacttcggcacggcgtgcttgggtcttgacggcatccacggtaaagacgcccttgtcgtcttgggtgaagacagccccatataccttctgcgcgtaggccggcagcaggaacttctggttcaggtccgcttcgatggcttgcgggtcccggtcgagagggtcgccaaagcccgggccaccgcgcaggtagttcaggtacagatcatggttggcgtagcagtcctccgtcgtgatgcactgcttgtcgcgcttgacctcggcggtggcgtcgatgtggcgttcgaagtcggggttactcggatcgatgtcgccgcccagcggcagggagtcgccgatggcgatgcgctggtcgaggccggtcttgtgcgcctcgaagcgatagccggtagcggagggatagccgcccatcatgccccagtcgctgttcatgaagccgttgcccatgaagaacatggtccagtcctgggcgttccacaccatgcgcagcgtctcgaacccgcaaccgccgcggtacttgccgtagccgccggaattggccttgacgttgcggccaaggtagagcagcggctcagccatctcccagatttcgatgtcgcccatatcgccttccggattccagatggcggcagcgtggttgagaccatcctttacggcgcaggcgccggtgccacaggaactggcctcgaagctgttgaccgcatggatctctccgtcctggttgatgccgccgccctgcagccagttggaggtgttggcgttgccggcgttgacctcttccaggtagccgcggctgaagtaggactgcgacaggccgcgccacagcgccgcccagccggagaccaggaagtgccaggcataggcatggccggtgcggcgatcatcggggttgcaccacgtgcccttgggcaagcggaactcggtagcgaaatacgccccgtcgttgatacgctgggtcggcacaagggtctggcacatcatcacccagattccggaagtgaaggccacctggtgggcgttgaaggtatgccagccccagcggctcgcgccctcaaagtcgaggcgccactttccgtccgacttgatggtcatctccaccggcgaatgcatgatggagtcgagcttggcgaaggcgttggagacctgcacgtcgtcatgcttgtagggcacgtcgacaaaggaaaccttgcggtacttgccaggcagggtcatcgccttgatgcggctctgcagtccgcggcggccttcttcgatgacctcataggcgaacttctcgtaggcctcgaggccgtcggcacgaatcacttcttcgaccagttcgcggatcatgtggcagccagcgatccgggttttctcatcgaggatccagtatttcggcgtgcgcaccgagcgctgcgactcgtgcagccagtcgcgcagcggctcgtcgttcacgccggtcttgcggcaggtgatcatgtagccgtcgccaaagcgctgcgtctgcccggttgacatcgagcccggcgttaccgagccggtgtcgatgacgtgggtgacgccgcctacccagccgatcagcttcccctcccagaagatcggcacgatggtggcgatgtcgcaggggtgcacgttgccgatggagcagtcgttgttggtgaacatgtcaccgggattgatgccggggttggcctcccagttgttctcgatcatgtacttgatcgccgcccccatggtgccgacgtggatgatgatgccggtcgaggtcagcacgcagtcacccacagcgttgtagagcgtgaagcagagttcgccttcctgctcgacgatggggctggcggcaattttcttcgcggtttcgcgggcatgcaccaagccgccgcgcagcttggagaacagcttctcatagccgatcgggtcgctgtcgcggaactccagtttgtcgaggccgttgtagtggccggacgcctgagtgcgagcgatgatcgcgtcgcggagctccttgagggtcttgccgttctgcagcagattgcccaggcccacttccttgttgctcatcatattcatatcttgaatctcctgaaattactcttcgacctgaacgacggtggtgacatcgggcagcagggagaggtccatgcggtgcttggcgccgtaggatggcacgcccagctcttcttcacgcacctgccagtcggcgggcaggctccagaattccttgaactcccgcgtgaacttctcagacagggcaaagctggtggcgaacatgtgctgcacctggacggacgcgtgcttgttgagaatgcgttcgcgctcttccttcatccattcccgcgtcggcagcgcccgggccaggcgttccttgcgaacttcggcacggcgtgcttgggtcttgacggcatccacggtaaagacgcccttgtcgtcttgggtgaagacagccccatataccttctgcgcgtaggccggcagcaggaacttctggttcaggtccgcttcgatggcttgcgggtcccggtcgagagggtcgccaaagcccgggccaccgcgcaggtagttcaggtacagatcatggttggcgtagcagtcctccgtcgtgatgcactgcttgtcgcgcttgacctcggcggtggcgtcgatgtggcgttcgaagtcggggttactcggatcgatgtcgccgcccagcggcagggagtcgccgatggcgatgcgctggtcgaggccggtcttgtgcgcctcgaagcgatagccggtagcggagggatagccgcccatcatgccccagtcgctgttcatgaagccgttgcccatgaagaacatggtccagtcctgggcgttccacaccatgcgcagcgtctcgaacccgcaaccgccgcggtacttgccgtagccgccggaattggccttgacgttgcggccaaggtagagcagcggctcagccatctcccagatttcgatgtcgcccatatcgccttccggattccagatggcggcagcgtggttgagaccatcctttacggcgcaggcgccggtgccacaggaactggcctcgaagctgttgaccgcatggatctctccgtcctggttgatgccgccgccctgcagccagttggaggtgttggcgttgccggcgttgacctcttccaggtagccgcggctgaagtaggactgcgacaggccgcgccacagcgccgcccagccggagaccaggaagtgccaggcataggcatggccggtgcggcgatcatcggggttgcaccacgtgcccttgggcaagcggaactcggtagcgaaatacgccccgtcgttgatacgctgggtcggcacaagggtctggcacatcatcacccagattccggaagtgaaggccacctggtgggcgttgaaggtatgccagccccagcggctcgcgccctcaaagtcgaggcgccactttccgtccgacttgatggtcatctccaccggcgaatgcatgatggagtcgagcttggcgaaggcgttggagacctgcacgtcgtcatgcttgtagggcacgtcgacaaaggaaaccttgcggtacttgccaggcagggtcatcgccttgatgcggctctgcagtccgcggcggccttcttcgatgacctcataggcgaacttctcgtaggcctcgaggccgtcggcacgaatcacttcttcgaccagttcgcggatcatgtggcagccagcgatccgggttttctcatcgaggatccagtatttcggcgtgcgcaccgagcgctgcgactcgtgcagccagtcgcgcagcggctcgtcgttcacgccggtcttgcggcaggtgatcatgtagccgtcgccaaagcgctgcgtctgcccggttgacatcgagcccggcgttaccgagccggtgtcgatgacgtgggtgacgccgcctacccagccgatcagcttcccctcccagaagatcggcacgatggtggcgatgtcgcaggggtgcacgttgccgatggagcagtcgttgttggtgaacatgtcaccgggattgatgccggggttggcctcccagttgttctcgatcatgtacttgatcgccgcccccatggtgccgacgtggatgatgatgccggtcgaggtcagcacgcagtcacccacagcgttgtagagcgtgaagcagagttcgccttcctgctcgacgatggggctggcggcaattttcttcgcggtttcgcgggcatgcaccaagccgccgcgcagcttggagaacagcttctcatagccgatcgggtcgctgtcgcggaactccagtttgtcgaggccgttgtagtggccggacgcctgagtgcgagcgatgatcgcgtcgcggagctccttgagggtcttgccgttctgcagcagattgcccaggcccacttccttgttgctcatcatattcattcttgaatctcctgaaatgtgaaattgttatccgctcacaattccacacaacatacgagccggaagcataaagtgtaaagcctggggaagctttcgatgttcaagaaaacacccgataactttcgctatcgggtgtttttattgattagttgaggcgttcgatcaccatggcgatgccctggcccccgccgatgcacagggtggccaggcccagcgtcttatcccgggcctgcatggcgtgcagcagggtcaccaggatgcgcgcgcccgaggcgccgatcggatggcccagggcgatcgcgccgccgttcacattcaccttctccgagtcgaagcccaggttcttgcccaccgccaggaactgggcggcgaaggcctcgttggcctcgatcaggtcgatgtccgccagttgcaggcccgccagctgcagggccttctgggtggccggcaccgggcccatgcccatcagggccggcggcaccccgcccgaggcgtacgacttgatgcgggccagcggggtcagccccgcggccagcgcggccgactcctccatgatcaccagcgccgcggcgccgtcgttgatgcccgaggcgttgcccgcggtcacggtgccggctttgtcgaaggccgggcgcagggcccccagggcctcggcggtcgagttggccttcgggaactcgtcctgcgagaacacgaaggtcttcttgcgggtcaccacgttcaccggcacgatctcggccgtgaaggcgcccgactcgatggcggcggcggccttgcgctgcgagtgcagggccagctcgtcctgcatctcgcgggtgatgccgtactccttggccacgttctcggcggtgatgcccatgtggtagccgtgggtggcgcacatgaggccgtcgcggaggatcacgtcgtacacctggccgtcccccagccgatagcccgagcgggccttggcgtccagcaggtacggggccagcgacatgttctccatgccgccggccacgatcgattgggcttgcccggcttggatggcttgggcggcgagggcgaccgacttcaggccggagccgcacaccttgttcacggtgaagccgcacacggtctcggcgaggcccgacttcaggagggcctgccgggccgggttctggcccagcccggcttgcagcacgttgcccatgatcacctcgtccacgtgttgcgagtcgatcttggcgcgctcgatggccgccttgatcacggtggcccccaggtcgatggccgaggtcgaggcgagcgagccgttgaacgagccgatcgcggtgcggacggccgacacgatcacgcagttcttcattttatattccttctgtttgtaggggtgccgtcacaggtcgccgcgctgggtgttcaggtcggcggccacctcgaagcgggcctcggtcttggcgcgcacggtcgcgaggtcgcagccgtcggcgatctcggtcagccacatcttgccgtcgatgaagcggaacacggccagttcggtcaccagcatgtgcacggcgtgctgggccgtcagcggcatggtgcagcggcgcaggatcttggccgagccatccttggcgcagtgctccatcgcaatgatcaccttgcgcgagccggtcacgaggtccatggccccccccatgcccgggaccattttgcccggcacgacccagttggccaggttggcctcctcgtccacctggagcccgccgaggacgcaggcgtcgatgtgccccccccggatcagggcgaacgacatggccgagtcgaacatcgcggcgcccggcagcaccccgcacggctggccccccgcgttcaccaggtccgggtgggcggtggtcaccgggcccaggcccaggaagccgttctccgactgcagggtgatgtggatgccctccggcagatagttggccaccatggtcggcaggccgatgcccaggttcacgatgtcgccgtcgcgcagttcctgcgcgacgcggcgggcgatgcgctgcttggcgtccattacttcgactcttgggagacgatgatatggtcgatcaccgcgccgggggtcacgatatgatccggttgcagctccccggtttcgacgagttcgtccggttccacgagggtgatatccgcggccagcgcaatcagcgggttgaagttccgggcggacagctggtacgtcaggttgcccagggtgtcgcagcgatgggcgcgaatcagggccagatcggcgcgcagcgggcgttccagcagccaggtcttcccgtccagggtcagcgtctgcttgccttcctcgaccacggtgcccacgccggtcggcgtcaggaacccgccgaggcccgccccgccgcaccggatttgctcaatgagggtcccctgcgggaccagcaccacatccatctcgcccgagatcatccggcggccggtctccgggttcgtgccgatgtggctggcgatcactttgcggacgcggccgttgacaatcagggggccaatgccggtgtccacgaaggcggtgtcgttggcaatgagcgtcagatcgcgcacccccgactccaggagggcttcgaccagccgcgacggcgtcccgatgcccatgaagccccccaccatgatggtcatgccatcgcggaaaaagccggtggcgtcctgcagcgtcatgagcttggtcttcattttttatccctcttgcatacggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 5 - DNA, 10031 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagttagtcggacgcgcgctccggcaccgggagattcacccactccttgtagaaggcctcgatgtccggctcgaagtcgtggatcacgggataccacggggtaggcgcttcgacgtcgtgcatggtgccgcacgatgggcagtagtattcgcgatacacctgccactgggtgtcgggggccatcagcttcggatacacctctgtcatcgcctcctcggtgtctcgcacgtagatcgcggcatgcagcttccagttctcgcggtaatcacagaactcgtgaccgcagtcgcacttgactacccacttcttgctcttgaccgactgcacgataaacatgtgcgggctgagcggtagcacgatgcggtcaggaaagttcaccttggcctgcaaggctgacaggtactgcgcgaaccggctctcgtccttgggcatcgagagcatgcggaaggtggtctcccaatcgagggaaccatcgacgaggtgggcgacctgttcattggtataggttgacattcttgaatctcctgaaattactcttcgacctgaacgacggtggtgacatcgggcagcagggagaggtccatgcggtgcttggcgccgtaggatggcacgcccagctcttcttcacgcacctgccagtcggcgggcaggctccagaattccttgaactcccgcgtgaacttctcagacagggcaaagctggtggcgaacatgtgctgcacctggacggacgcgtgcttgttgagaatgcgttcgcgctcttccttcatccattcccgcgtcggcagcgcccgggccaggcgttccttgcgaacttcggcacggcgtgcttgggtcttgacggcatccacggtaaagacgcccttgtcgtcttgggtgaagacagccccatataccttctgcgcgtaggccggcagcaggaacttctggttcaggtccgcttcgatggcttgcgggtcccggtcgagagggtcgccaaagcccgggccaccgcgcaggtagttcaggtacagatcatggttggcgtagcagtcctccgtcgtgatgcactgcttgtcgcgcttgacctcggcggtggcgtcgatgtggcgttcgaagtcggggttactcggatcgatgtcgccgcccagcggcagggagtcgccgatggcgatgcgctggtcgaggccggtcttgtgcgcctcgaagcgatagccggtagcggagggatagccgcccatcatgccccagtcgctgttcatgaagccgttgcccatgaagaacatggtccagtcctgggcgttccacaccatgcgcagcgtctcgaacccgcaaccgccgcggtacttgccgtagccgccggaattggccttgacgttgcggccaaggtagagcagcggctcagccatctcccagatttcgatgtcgcccatatcgccttccggattccagatggcggcagcgtggttgagaccatcctttacggcgcaggcgccggtgccacaggaactggcctcgaagctgttgaccgcatggatctctccgtcctggttgatgccgccgccctgcagccagttggaggtgttggcgttgccggcgttgacctcttccaggtagccgcggctgaagtaggactgcgacaggccgcgccacagcgccgcccagccggagaccaggaagtgccaggcataggcatggccggtgcggcgatcatcggggttgcaccacgtgcccttgggcaagcggaactcggtagcgaaatacgccccgtcgttgatacgctgggtcggcacaagggtctggcacatcatcacccagattccggaagtgaaggccacctggtgggcgttgaaggtatgccagccccagcggctcgcgccctcaaagtcgaggcgccactttccgtccgacttgatggtcatctccaccggcgaatgcatgatggagtcgagcttggcgaaggcgttggagacctgcacgtcgtcatgcttgtagggcacgtcgacaaaggaaaccttgcggtacttgccaggcagggtcatcgccttgatgcggctctgcagtccgcggcggccttcttcgatgacctcataggcgaacttctcgtaggcctcgaggccgtcggcacgaatcacttcttcgaccagttcgcggatcatgtggcagccagcgatccgggttttctcatcgaggatccagtatttcggcgtgcgcaccgagcgctgcgactcgtgcagccagtcgcgcagcggctcgtcgttcacgccggtcttgcggcaggtgatcatgtagccgtcgccaaagcgctgcgtctgcccggttgacatcgagcccggcgttaccgagccggtgtcgatgacgtgggtgacgccgcctacccagccgatcagcttcccctcccagaagatcggcacgatggtggcgatgtcgcaggggtgcacgttgccgatggagcagtcgttgttggtgaacatgtcaccgggattgatgccggggttggcctcccagttgttctcgatcatgtacttgatcgccgcccccatggtgccgacgtggatgatgatgccggtcgaggtcagcacgcagtcacccacagcgttgtagagcgtgaagcagagttcgccttcctgctcgacgatggggctggcggcaattttcttcgcggtttcgcgggcatgcaccaagccgccgcgcagcttggagaacagcttctcatagccgatcgggtcgctgtcgcggaactccagtttgtcgaggccgttgtagtggccggacgcctgagtgcgagcgatgatcgcgtcgcggagctccttgagggtcttgccgttctgcagcagattgcccaggcccacttccttgttgctcatcatattcatatcttgaatctcctgaaattactcttcgacctgaacgacggtggtgacatcgggcagcagggagaggtccatgcggtgcttggcgccgtaggatggcacgcccagctcttcttcacgcacctgccagtcggcgggcaggctccagaattccttgaactcccgcgtgaacttctcagacagggcaaagctggtggcgaacatgtgctgcacctggacggacgcgtgcttgttgagaatgcgttcgcgctcttccttcatccattcccgcgtcggcagcgcccgggccaggcgttccttgcgaacttcggcacggcgtgcttgggtcttgacggcatccacggtaaagacgcccttgtcgtcttgggtgaagacagccccatataccttctgcgcgtaggccggcagcaggaacttctggttcaggtccgcttcgatggcttgcgggtcccggtcgagagggtcgccaaagcccgggccaccgcgcaggtagttcaggtacagatcatggttggcgtagcagtcctccgtcgtgatgcactgcttgtcgcgcttgacctcggcggtggcgtcgatgtggcgttcgaagtcggggttactcggatcgatgtcgccgcccagcggcagggagtcgccgatggcgatgcgctggtcgaggccggtcttgtgcgcctcgaagcgatagccggtagcggagggatagccgcccatcatgccccagtcgctgttcatgaagccgttgcccatgaagaacatggtccagtcctgggcgttccacaccatgcgcagcgtctcgaacccgcaaccgccgcggtacttgccgtagccgccggaattggccttgacgttgcggccaaggtagagcagcggctcagccatctcccagatttcgatgtcgcccatatcgccttccggattccagatggcggcagcgtggttgagaccatcctttacggcgcaggcgccggtgccacaggaactggcctcgaagctgttgaccgcatggatctctccgtcctggttgatgccgccgccctgcagccagttggaggtgttggcgttgccggcgttgacctcttccaggtagccgcggctgaagtaggactgcgacaggccgcgccacagcgccgcccagccggagaccaggaagtgccaggcataggcatggccggtgcggcgatcatcggggttgcaccacgtgcccttgggcaagcggaactcggtagcgaaatacgccccgtcgttgatacgctgggtcggcacaagggtctggcacatcatcacccagattccggaagtgaaggccacctggtgggcgttgaaggtatgccagccccagcggctcgcgccctcaaagtcgaggcgccactttccgtccgacttgatggtcatctccaccggcgaatgcatgatggagtcgagcttggcgaaggcgttggagacctgcacgtcgtcatgcttgtagggcacgtcgacaaaggaaaccttgcggtacttgccaggcagggtcatcgccttgatgcggctctgcagtccgcggcggccttcttcgatgacctcataggcgaacttctcgtaggcctcgaggccgtcggcacgaatcacttcttcgaccagttcgcggatcatgtggcagccagcgatccgggttttctcatcgaggatccagtatttcggcgtgcgcaccgagcgctgcgactcgtgcagccagtcgcgcagcggctcgtcgttcacgccggtcttgcggcaggtgatcatgtagccgtcgccaaagcgctgcgtctgcccggttgacatcgagcccggcgttaccgagccggtgtcgatgacgtgggtgacgccgcctacccagccgatcagcttcccctcccagaagatcggcacgatggtggcgatgtcgcaggggtgcacgttgccgatggagcagtcgttgttggtgaacatgtcaccgggattgatgccggggttggcctcccagttgttctcgatcatgtacttgatcgccgcccccatggtgccgacgtggatgatgatgccggtcgaggtcagcacgcagtcacccacagcgttgtagagcgtgaagcagagttcgccttcctgctcgacgatggggctggcggcaattttcttcgcggtttcgcgggcatgcaccaagccgccgcgcagcttggagaacagcttctcatagccgatcgggtcgctgtcgcggaactccagtttgtcgaggccgttgtagtggccggacgcctgagtgcgagcgatgatcgcgtcgcggagctccttgagggtcttgccgttctgcagcagattgcccaggcccacttccttgttgctcatcatattcattcttgaatctcctgaaactttctagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 6 - DNA, 5330 bp, Artificial Sequence
  vector
ccccaggctttacactttatgcttccggctcgtatgttgtgtggaattgtgagcggataacaatttcacatttcaggagattcaagaatgaatatgatgagcaacaaggaagtgggcctgggcaatctgctgcagaacggcaagaccctcaaggagctccgcgacgcgatcatcgctcgcactcaggcgtccggccactacaacggcctcgacaaactggagttccgcgacagcgacccgatcggctatgagaagctgttctccaagctgcgcggcggcttggtgcatgcccgcgaaaccgcgaagaaaattgccgccagccccatcgtcgagcaggaaggcgaactctgcttcacgctctacaacgctgtgggtgactgcgtgctgacctcgaccggcatcatcatccacgtcggcaccatgggggcggcgatcaagtacatgatcgagaacaactgggaggccaaccccggcatcaatcccggtgacatgttcaccaacaacgactgctccatcggcaacgtgcacccctgcgacatcgccaccatcgtgccgatcttctgggaggggaagctgatcggctgggtaggcggcgtcacccacgtcatcgacaccggctcggtaacgccgggctcgatgtcaaccgggcagacgcagcgctttggcgacggctacatgatcacctgccgcaagaccggcgtgaacgacgagccgctgcgcgactggctgcacgagtcgcagcgctcggtgcgcacgccgaaatactggatcctcgatgagaaaacccggatcgctggctgccacatgatccgcgaactggtcgaagaagtgattcgtgccgacggcctcgaggcctacgagaagttcgcctatgaggtcatcgaagaaggccgccgcggactgcagagccgcatcaaggcgatgaccctgcctggcaagtaccgcaaggtttcctttgtcgacgtgccctacaagcatgacgacgtgcaggtctccaacgccttcgccaagctcgactccatcatgcattcgccggtggagatgaccatcaagtcggacggaaagtggcgcctcgactttgagggcgcgagccgctggggctggcataccttcaacgcccaccaggtggccttcacttccggaatctgggtgatgatgtgccagacccttgtgccgacccagcgtatcaacgacggggcgtatttcgctaccgagttccgcttgcccaagggcacgtggtgcaaccccgatgatcgccgcaccggccatgcctatgcctggcacttcctggtctccggctgggcggcgctgtggcgcggcctgtcgcagtcctacttcagccgcggctacctggaagaggtcaacgccggcaacgccaacacctccaactggctgcagggcggcggcatcaaccaggacggagagatccatgcggtcaacagcttcgaggccagttcctgtggcaccggcgcctgcgccgtaaaggatggtctcaaccacgctgccgccatctggaatccggaaggcgatatgggcgacatcgaaatctgggagatggctgagccgctgctctaccttggccgcaacgtcaaggccaattccggcggctacggcaagtaccgcggcggttgcgggttcgagacgctgcgcatggtgtggaacgcccaggactggaccatgttcttcatgggcaacggcttcatgaacagcgactggggcatgatgggcggctatccctccgctaccggctatcgcttcgaggcgcacaagaccggcctcgaccagcgcatcgccatcggcgactccctgccgctgggcggcgacatcgatccgagtaaccccgacttcgaacgccacatcgacgccaccgccgaggtcaagcgcgacaagcagtgcatcacgacggaggactgctacgccaaccatgatctgtacctgaactacctgcgcggtggcccgggctttggcgaccctctcgaccgggacccgcaagccatcgaagcggacctgaaccagaagttcctgctgccggcctacgcgcagaaggtatatggggctgtcttcacccaagacgacaagggcgtctttaccgtggatgccgtcaagacccaagcacgccgtgccgaagttcgcaaggaacgcctggcccgggcgctgccgacgcgggaatggatgaaggaagagcgcgaacgcattctcaacaagcacgcgtccgtccaggtgcagcacatgttcgccaccagctttgccctgtctgagaagttcacgcgggagttcaaggaattctggagcctgcccgccgactggcaggtgcgtgaagaagagctgggcgtgccatcctacggcgccaagcaccgcatggacctctccctgctgcccgatgtcaccaccgtcgttcaggtcgaagagtaatttcaggagattcaagatatgaatatgatgagcaacaaggaagtgggcctgggcaatctgctgcagaacggcaagaccctcaaggagctccgcgacgcgatcatcgctcgcactcaggcgtccggccactacaacggcctcgacaaactggagttccgcgacagcgacccgatcggctatgagaagctgttctccaagctgcgcggcggcttggtgcatgcccgcgaaaccgcgaagaaaattgccgccagccccatcgtcgagcaggaaggcgaactctgcttcacgctctacaacgctgtgggtgactgcgtgctgacctcgaccggcatcatcatccacgtcggcaccatgggggcggcgatcaagtacatgatcgagaacaactgggaggccaaccccggcatcaatcccggtgacatgttcaccaacaacgactgctccatcggcaacgtgcacccctgcgacatcgccaccatcgtgccgatcttctgggaggggaagctgatcggctgggtaggcggcgtcacccacgtcatcgacaccggctcggtaacgccgggctcgatgtcaaccgggcagacgcagcgctttggcgacggctacatgatcacctgccgcaagaccggcgtgaacgacgagccgctgcgcgactggctgcacgagtcgcagcgctcggtgcgcacgccgaaatactggatcctcgatgagaaaacccggatcgctggctgccacatgatccgcgaactggtcgaagaagtgattcgtgccgacggcctcgaggcctacgagaagttcgcctatgaggtcatcgaagaaggccgccgcggactgcagagccgcatcaaggcgatgaccctgcctggcaagtaccgcaaggtttcctttgtcgacgtgccctacaagcatgacgacgtgcaggtctccaacgccttcgccaagctcgactccatcatgcattcgccggtggagatgaccatcaagtcggacggaaagtggcgcctcgactttgagggcgcgagccgctggggctggcataccttcaacgcccaccaggtggccttcacttccggaatctgggtgatgatgtgccagacccttgtgccgacccagcgtatcaacgacggggcgtatttcgctaccgagttccgcttgcccaagggcacgtggtgcaaccccgatgatcgccgcaccggccatgcctatgcctggcacttcctggtctccggctgggcggcgctgtggcgcggcctgtcgcagtcctacttcagccgcggctacctggaagaggtcaacgccggcaacgccaacacctccaactggctgcagggcggcggcatcaaccaggacggagagatccatgcggtcaacagcttcgaggccagttcctgtggcaccggcgcctgcgccgtaaaggatggtctcaaccacgctgccgccatctggaatccggaaggcgatatgggcgacatcgaaatctgggagatggctgagccgctgctctaccttggccgcaacgtcaaggccaattccggcggctacggcaagtaccgcggcggttgcgggttcgagacgctgcgcatggtgtggaacgcccaggactggaccatgttcttcatgggcaacggcttcatgaacagcgactggggcatgatgggcggctatccctccgctaccggctatcgcttcgaggcgcacaagaccggcctcgaccagcgcatcgccatcggcgactccctgccgctgggcggcgacatcgatccgagtaaccccgacttcgaacgccacatcgacgccaccgccgaggtcaagcgcgacaagcagtgcatcacgacggaggactgctacgccaaccatgatctgtacctgaactacctgcgcggtggcccgggctttggcgaccctctcgaccgggacccgcaagccatcgaagcggacctgaaccagaagttcctgctgccggcctacgcgcagaaggtatatggggctgtcttcacccaagacgacaagggcgtctttaccgtggatgccgtcaagacccaagcacgccgtgccgaagttcgcaaggaacgcctggcccgggcgctgccgacgcgggaatggatgaaggaagagcgcgaacgcattctcaacaagcacgcgtccgtccaggtgcagcacatgttcgccaccagctttgccctgtctgagaagttcacgcgggagttcaaggaattctggagcctgcccgccgactggcaggtgcgtgaagaagagctgggcgtgccatcctacggcgccaagcaccgcatggacctctccctgctgcccgatgtcaccaccgtcgttcaggtcgaagagtaatttcaggagattcaagaatgtcaacctataccaatgaacaggtcgcccacctcgtcgatggttccctcgattgggagaccaccttccgcatgctctcgatgcccaaggacgagagccggttcgcgcagtacctgtcagccttgcaggccaaggtgaactttcctgaccgcatcgtgctaccgctcagcccgcacatgtttatcgtgcagtcggtcaagagcaagaagtgggtagtcaagtgcgactgcggtcacgagttctgtgattaccgcgagaactggaagctgcatgccgcgatctacgtgcgagacaccgaggaggcgatgacagaggtgtatccgaagctgatggcccccgacacccagtggcaggtgtatcgcgaatactactgcccatcgtgcggcaccatgcacgacgtcgaagcgcctaccccgtggtatcccgtgatccacgacttcgagccggacatcgaggccttctacaaggagtgggtgaatctcccggtgccggagcgcgcgtccgactaataaaaataagagttaccttaaatggtaactcttatttttttaatattg

SEQ ID NO: 7 - DNA, 5329 bp, Artificial Sequence
  expression construct
ccccaggctttacactttatgcttccggctcgtatgttgtgtggaattgtgagcggataacaatttcacatttcaggagattcaagaatgaatatgatgagcaacaaggaagtgggcctgggcaatctgctgcagaacggcaagaccctcaaggagctccgcgacgcgatcatcgctcgcactcaggcgtccggccactacaacggcctcgacaaactggagttccgcgacagcgacccgatcggctatgagaagctgttctccaagctgcgcggcggcttggtgcatgcccgcgaaaccgcgaagaaaattgccgccagccccatcgtcgagcaggaaggcgaactctgcttcacgctctacaacgctgtgggtgactgcgtgctgacctcgaccggcatcatcatccacgtcggcaccatgggggcggcgatcaagtacatgatcgagaacaactgggaggccaaccccggcatcaatcccggtgacatgttcaccaacaacgactgctccatcggcaacgtgcacccctgcgacatcgccaccatcgtgccgatcttctgggaggggaagctgatcggctgggtaggcggcgtcacccacgtcatcgacaccggctcggtaacgccgggctcgatgtcaaccgggcagacgcagcgctttggcgacggctacatgatcacctgccgcaagaccggcgtgaacgacgagccgctgcgcgactggctgcacgagtcgcagcgctcggtgcgcacgccgaaatactggatcctcgatgagaaaacccggatcgctggctgccacatgatccgcgaactggtcgaagaagtgattcgtgccgacggcctcgaggcctacgagaagttcgcctatgaggtcatcgaagaaggccgccgcggactgcagagccgcatcaaggcgatgaccctgcctggcaagtaccgcaaggtttcctttgtcgacgtgccctacaagcatgacgacgtgcaggtctccaacgccttcgccaagctcgactccatcatgcattcgccggtggagatgaccatcaagtcggacggaaagtggcgcctcgactttgagggcgcgagccgctggggctggcataccttcaacgcccaccaggtggccttcacttccggaatctgggtgatgatgtgccagacccttgtgccgacccagcgtatcaacgacggggcgtatttcgctaccgagttccgcttgcccaagggcacgtggtgcaaccccgatgatcgccgcaccggccatgcctatgcctggcacttcctggtctccggctgggcggcgctgtggcgcggcctgtcgcagtcctacttcagccgcggctacctggaagaggtcaacgccggcaacgccaacacctccaactggctgcagggcggcggcatcaaccaggacggagagatccatgcggtcaacagcttcgaggccagttcctgtggcaccggcgcctgcgccgtaaaggatggtctcaaccacgctgccgccatctggaatccggaaggcgatatgggcgacatcgaaatctgggagatggctgagccgctgctctaccttggccgcaacgtcaaggccaattccggcggctacggcaagtaccgcggcggttgcgggttcgagacgctgcgcatggtgtggaacgcccaggactggaccatgttcttcatgggcaacggcttcatgaacagcgactggggcatgatgggcggctatccctccgctaccggctatcgcttcgaggcgcacaagaccggcctcgaccagcgcatcgccatcggcgactccctgccgctgggcggcgacatcgatccgagtaaccccgacttcgaacgccacatcgacgccaccgccgaggtcaagcgcgacaagcagtgcatcacgacggaggactgctacgccaaccatgatctgtacctgaactacctgcgcggtggcccgggctttggcgaccctctcgaccgggacccgcaagccatcgaagcggacctgaaccagaagttcctgctgccggcctacgcgcagaaggtatatggggctgtcttcacccaagacgacaagggcgtctttaccgtggatgccgtcaagacccaagcacgccgtgccgaagttcgcaaggaacgcctggcccgggcgctgccgacgcgggaatggatgaaggaagagcgcgaacgcattctcaacaagcacgcgtccgtccaggtgcagcacatgttcgccaccagctttgccctgtctgagaagttcacgcgggagttcaaggaattctggagcctgcccgccgactggcaggtgcgtgaagaagagctgggcgtgccatcctacggcgccaagcaccgcatggacctctccctgctgcccgatgtcaccaccgtcgttcaggtcgaagagtaatttcaggagattcaagatatgaatatgatgagcaacaaggaagtgggcctgggcaatctgctgcagaacggcaagaccctcaaggagctccgcgacgcgatcatcgctcgcactcaggcgtccggccactacaacggcctcgacaaactggagttccgcgacagcgacccgatcggctatgagaagctgttctccaagctgcgcggcggcttggtgcatgcccgcgaaaccgcgaagaaaattgccgccagccccatcgtcgagcaggaaggcgaactctgcttcacgctctacaacgctgtgggtgactgcgtgctgacctcgaccggcatcatcatccacgtcggcaccatgggggcggcgatcaagtacatgatcgagaacaactgggaggccaaccccggcatcaatcccggtgacatgttcaccaacaacgactgctccatcggcaacgtgcacccctgcgacatcgccaccatcgtgccgatcttctgggaggggaagctgatcggctgggtaggcggcgtcacccacgtcatcgacaccggctcggtaacgccgggctcgatgtcaaccgggcagacgcagcgctttggcgacggctacatgatcacctgccgcaagaccggcgtgaacgacgagccgctgcgcgactggctgcacgagtcgcagcgctcggtgcgcacgccgaaatactggatcctcgatgagaaaacccggatcgctggctgccacatgatccgcgaactggtcgaagaagtgattcgtgccgacggcctcgaggcctacgagaagttcgcctatgaggtcatcgaagaaggccgccgcggactgcagagccgcatcaaggcgatgaccctgcctggcaagtaccgcaaggtttcctttgtcgacgtgccctacaagcatgacgacgtgcaggtctccaacgccttcgccaagctcgactccatcatgcattcgccggtggagatgaccatcaagtcggacggaaagtggcgcctcgactttgagggcgcgagccgctggggctggcataccttcaacgcccaccaggtggccttcacttccggaatctgggtgatgatgtgccagacccttgtgccgacccagcgtatcaacgacggggcgtatttcgctaccgagttccgcttgcccaagggcacgtggtgcaaccccgatgatcgccgcaccggccatgcctatgcctggcacttcctggtctccggctgggcggcgctgtggcgcggcctgtcgcagtcctacttcagccgcggctacctggaagaggtcaacgccggcaacgccaacacctccaactggctgcagggcggcggcatcaaccaggacggagagatccatgcggtcaacagcttcgaggccagttcctgtggcaccggcgcctgcgccgtaaaggatggtctcaaccacgctgccgccatctggaatccggaaggcgatatgggcgacatcgaaatctgggagatggctgagccgctgctctaccttggccgcaacgtcaaggccaattccggcggctacggcaagtaccgcggcggttgcgggttcgagacgctgcgcatggtgtggaacgcccaggactggaccatgttcttcatgggcaacggcttcatgaacagcgactggggcatgatgggcggctatccctccgctaccggctatcgcttcgaggcgcacaagaccggcctcgaccagcgcatcgccatcggcgactccctgccgctgggcggcgacatcgatccgagtaaccccgacttcgaacgccacatcgacgccaccgccgaggtcaagcgcgacaagcagtgcatcacgacggaggactgctacgccaaccatgatctgtacctgaactacctgcgcggtggcccgggctttggcgaccctctcgaccgggacccgcaagccatcgaagcggacctgaaccagaagttcctgctgccggcctacgcgcagaaggtatatggggctgtcttcacccaagacgacaagggcgtctttaccgtggatgccgtcaagacccaagcacgccgtgccgaagttcgcaaggaacgcctggcccgggcgctgccgacgcgggaatggatgaaggaagagcgcgaacgcattctcaacaagcacgcgtccgtccaggtgcagcacatgttcgccaccagctttgccctgtctgagaagttcacgcgggagttcaaggaattctggagcctgcccgccgactggcaggtgcgtgaagaagagctgggcgtgccatcctacggcgccaagcaccgcatggacctctccctgctgcccgatgtcaccaccgtcgttcaggtcgaagagtaatttcaggagattcaagaatgtcaacctataccaatgaacaggtcgcccacctcgtcgatggttccctcgattgggagaccaccttccgcatgctctcgatgcccaaggacgagagccggttcgcgcagtacctgtcagccttgcaggccaaggtgaactttcctgaccgcatcgtgctaccgctcagcccgcacatgtttatcgtgcagtcggtcaagagcaagaagtgggtagtcaagtgcgactgcggtcacgagttctgtgattaccgcgagaactggaagctgcatgccgcgatctacgtgcgagacaccgaggaggcgatgacagaggtgtatccgaagctgatggcccccgacacccagtggcaggtgtatcgcgaatactactgcccatcgtgcggcaccatgcacgacgtcgaagcgcctaccccgtggtatcccgtgatccacgacttcgagccggacatcgaggccttctacaaggagtgggtgaatctcccggtgccggagcgcgcgtccgactaaaaaaataagagttaccttaaatggtaactcttatttttttaatattg

SEQ ID NO: 8 - DNA, 2145 bp, Ralstonia eutropha
atgcaaccgcagtccaccgtacaggtgatgggcatcgatgctggtggcacgatgaccgacacattcttcgtgcgctcggatggccgtttcgtggtcggcaaggcgcaaagcaatcccggcgatgaatcccttgccatctataactcgtccgaagatgcgctcgcacactgggaccggacagtggatgacgtttatccggaactggtgacctgcgtctattccggtacggccatgctcaatcgcatcctgatgcgcaaaggccttgatgttggcctgatttgcaatcgaggcttcgagcagatccattcgatggggcgcgcgctgcagagctacttgggttatgccctggaagaccgcattcacttgaatacgcatcgctacgatgagcccctggtgcccgtttcacgcacccgtggtgtcaccgagcgcaccgacgtgcaagggaaggtagtcatcccgctgcgggaagacgaagtccgccagtccacccgcgaactggtggaggccggctcgcaagccatcgttatttgcctgctgcaatcccacaagaacgaatcgagcgagcaacgcgcccgggacatcgtgcgtgaggaactgaagaggctgaaggcggatattcccgtcttcgcctcggtggactactacccgtcgcgcaaggaaagccaccggatgaacaccaccatccttgaggcttacggcgcagagccttcgcgccagacgctgaagaaggttagcgaccgcttcaggaagcacggggccaagttcgatctgcgcgtcatggctacgcacggcggcaccattagctggaaggccaaggaactcgcgcgcacgattgtttccggcccgattggtggcgtgatcggctcgaaactgctcggggaggccttgggcgacgagaacattgcatgctccgatattggcggcaccagcttcgacgtggcgttgatcaccaagggcaacttcgctatcaagtccgatccggacatggctcgcctcgtgctctctctaccgctagtcgccatggactcggtcggcgcgggtgccggcagcttcgtgcgtctggatccctacagcaagtccatcaagcttggcccggactctgctggctatcgcgtcggcacctgctggccggaaagcggtctcgacaccgtttcggtttccgactgccacgtggtgctgggctacctgaatccggacaactttctcggtggcgccatcaagctcgatgtggaacgcgcccgccagcatatcaaggcgcagatcgccgatccgctcggcctgtcggtggaagatgccgcggccggcgtcatcgagttgctggacctgacgctgtccgaatacctgcgcgccaatatcagcgccaagggctacaacccggcggagttcacgtgcttctcctatggtggcgccggccccgtgcatacctacggttatacggagggggtaggtttcaaggacgtggtcgtgcccgcctgggcggccggcttctcggccttcggctgtgcctgcgccgacttcgaatatcgctatgacaagtcggtcgacgtcggtgtcgcacaactcgcgccggacggcgataaggctgccgcctgcaagaccttgcaggaagcctggtccgaattggccatcaaggttatcgacgagtttgtcctcaacggctacaagcccgaagacgtgctgctgatccccggctacaagatgcagtacatgggccaactgaacgaccttgaaatcgtatcgccggtgaccagtgctgccaccgcagccgactgggacaagatcgtcgaggccttcgagaccacctatggccgggtgtacgccagttccgcgcgctcgccggaactgggcttctcgatcaccggcgcgatcctgcgcggcaccgtcgttacccagaagccggtactgccggaagacccggatgcgggcccattgccgcccaaggaagcctatctcggcacccgccccttctatcgccacaagaagtgggtggatgccgcactgtggaagatggaggcgctcaaggccggcaaccacatcgtgggccccgccatcatcgaatccgatgccaccaccttcgtcgtgccggatggtttcgagacaacggtcgacaagcatcgcctgttccacctcaaggaagtgaagtaa

SEQ ID NO: 9 - DNA, 2325 bp, Ralstonia eutropha
atgaatatgatgagcaacaaggaagtgggcctgggcaatctgctgcagaacggcaagaccctcaaggagctccgcgacgcgatcatcgctcgcactcaggcgtccggccactacaacggcctcgacaaactggagttccgcgacagcgacccgatcggctatgagaagctgttctccaagctgcgcggcggcttggtgcatgcccgcgaaaccgcgaagaaaattgccgccagccccatcgtcgagcaggaaggcgaactctgcttcacgctctacaacgctgtgggtgactgcgtgctgacctcgaccggcatcatcatccacgtcggcaccatgggggcggcgatcaagtacatgatcgagaacaactgggaggccaaccccggcatcaatcccggtgacatgttcaccaacaacgactgctccatcggcaacgtgcacccctgcgacatcgccaccatcgtgccgatcttctgggaggggaagctgatcggctgggtaggcggcgtcacccacgtcatcgacaccggctcggtaacgccgggctcgatgtcaaccgggcagacgcagcgctttggcgacggctacatgatcacctgccgcaagaccggcgtgaacgacgagccgctgcgcgactggctgcacgagtcgcagcgctcggtgcgcacgccgaaatactggatcctcgatgagaaaacccggatcgctggctgccacatgatccgcgaactggtcgaagaagtgattcgtgccgacggcctcgaggcctacgagaagttcgcctatgaggtcatcgaagaaggccgccgcggactgcagagccgcatcaaggcgatgaccctgcctggcaagtaccgcaaggtttcctttgtcgacgtgccctacaagcatgacgacgtgcaggtctccaacgccttcgccaagctcgactccatcatgcattcgccggtggagatgaccatcaagtcggacggaaagtggcgcctcgactttgagggcgcgagccgctggggctggcataccttcaacgcccaccaggtggccttcacttccggaatctgggtgatgatgtgccagacccttgtgccgacccagcgtatcaacgacggggcgtatttcgctaccgagttccgcttgcccaagggcacgtggtgcaaccccgatgatcgccgcaccggccatgcctatgcctggcacttcctggtctccggctgggcggcgctgtggcgcggcctgtcgcagtcctacttcagccgcggctacctggaagaggtcaacgccggcaacgccaacacctccaactggctgcagggcggcggcatcaaccaggacggagagatccatgcggtcaacagcttcgaggccagttcctgtggcaccggcgcctgcgccgtaaaggatggtctcaaccacgctgccgccatctggaatccggaaggcgatatgggcgacatcgaaatctgggagatggctgagccgctgctctaccttggccgcaacgtcaaggccaattccggcggctacggcaagtaccgcggcggttgcgggttcgagacgctgcgcatggtgtggaacgcccaggactggaccatgttcttcatgggcaacggcttcatgaacagcgactggggcatgatgggcggctatccctccgctaccggctatcgcttcgaggcgcacaagaccggcctcgaccagcgcatcgccatcggcgactccctgccgctgggcggcgacatcgatccgagtaaccccgacttcgaacgccacatcgacgccaccgccgaggtcaagcgcgacaagcagtgcatcacgacggaggactgctacgccaaccatgatctgtacctgaactacctgcgcggtggcccgggctttggcgaccctctcgaccgggacccgcaagccatcgaagcggacctgaaccagaagttcctgctgccggcctacgcgcagaaggtatatggggctgtcttcacccaagacgacaagggcgtctttaccgtggatgccgtcaagacccaagcacgccgtgccgaagttcgcaaggaacgcctggcccgggcgctgccgacgcgggaatggatgaaggaagagcgcgaacgcattctcaacaagcacgcgtccgtccaggtgcagcacatgttcgccaccagctttgccctgtctgagaagttcacgcgggagttcaaggaattctggagcctgcccgccgactggcaggtgcgtgaagaagagctgggcgtgccatcctacggcgccaagcaccgcatggacctctccctgctgcccgatgtcaccaccgtcgttcaggtcgaagagtaa

SEQ ID NO: 10 - DNA, 5144 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 11 - protein, 351 aa, Clostridium beijerinckii
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

SEQ ID NO: 12 - DNA, 1301 bp, Artificial Sequence
  expression cassette
aacccgcatcacacccgcgtcttgaaatgcccctaccccgtccctataattagcactcgtcaggggtgagtgctaacagcctcctgcgacacctgaacatttctgacggccgtcgcctcggtagccggccgatttgtgatcaaaaactcactttttcaggagattcaagaatgaagggcttcgccatgctgggcatcaacaaactgggctggatcgagaaggagcgcccggtggccggctcgtatgacgccatcgtgcgcccgctcgccgtgtcgccgtgtacgtcggacatccacaccgtgtttgagggcgcgctgggggaccgcaagaatatgatcctggggcacgaggccgtgggcgaggtggtggaggtggggtcggaggtgaaggactttaagcccggcgaccgggtgatcgtgccgtgcaccacccccgactggcgctcgctggaggtccaggccgggttccagcagcattcgaacggcatgctcgccgggtggaagttctcgaacttcaaggacggcgtgttcggggagtactttcatgtgaacgacgccgacatgaacctggccatcctgccgaaagatatgccgctggaaaacgcggtgatgatcaccgatatgatgaccacggggttccacggggccgagctggcggatatccagatgggcagctcggtcgtcgtgatcggcatcggggcggtggggctcatgggcatcgcgggcgcgaagctgcggggggcgggccgcatcatcggcgtgggctcgcggccgatctgcgtggaggcggccaagttctacggcgccaccgacatcctgaactacaagaacggccatattgtggaccaggtcatgaagctgaccaacggcaagggcgtggaccgcgtcatcatggccgggggggggtccgaaacgctgtcgcaggccgtctcgatggtgaagccgggcgggatcatttcgaacatcaactaccacggctcgggcgacgcgctgctcatcccccgcgtggaatggggctgcggcatggcccataagaccattaagggcggcctgtgccccggcgggcggctgcgcgcggagatgctgcgcgacatggtggtgtataatcgcgtcgacctgtcgaaactggtgacgcacgtgtaccatggcttcgaccacatcgaggaggccctcctgctgatgaaagacaagccgaaggacctgatcaaggccgtcgtcatcctgtaagtggcctgacagtcgccggaaagaaaaagggcggccccgcggggccgccctttttgcttatcgaggcagcctgct

SEQ ID NO: 13 - protein, 220 aa, Escherichia coli
MKTKLMTLQDATGFFRDGMTIMVGGFMGIGTPSRLVEALLESGVRDLTLIANDTAFVDTGIGPLIVNGRVRKVIASHIGTNPETGRRMISGEMDVVLVPQGTLIEQIRCGGAGLGGFLTPTGVGTVVEEGKQTLTLDGKTWLLERPLRADLALIRAHRCDTLGNLTYQLSARNFNPLIALAADITLVEPDELVETGELQPDHIVTPGAVIDHIIVSQESK

SEQ ID NO: 14 - protein, 216 aa, Escherichia coli
MDAKQRIARRVAQELRDGDIVNLGIGLPTMVANYLPEGIHITLQSENGFLGLGPVTTAHPDLVNAGGQPCGVLPGAAMFDSAMSFALIRGGHIDACVLGGLQVDEEANLANWVVPGKMVPGMGGAMDLVTGSRKVIIAMEHCAKDGSAKILRRCTMPLTAQHAVHMLVTELAVFRFIDGKMWLTEIADGCDLATVRAKTEARFEVAADLNTQRGDL

SEQ ID NO: 15 - protein, 394 aa, Escherichia blattae
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 16 - DNA, 2612 bp, Artificial Sequence
  codonoptimized gen
ggtaccgtatgcaagagggataaaaaatgaagaccaagctcatgacgctgcaggacgccaccggctttttccgcgatggcatgaccatcatggtggggggcttcatgggcatcgggacgccgtcgcggctggtcgaagccctcctggagtcgggggtgcgcgatctgacgctcattgccaacgacaccgccttcgtggacaccggcattggccccctgattgtcaacggccgcgtccgcaaagtgatcgccagccacatcggcacgaacccggagaccggccgccggatgatctcgggcgagatggatgtggtgctggtcccgcaggggaccctcattgagcaaatccggtgcggcggggcgggcctcggcgggttcctgacgccgaccggcgtgggcaccgtggtcgaggaaggcaagcagacgctgaccctggacgggaagacctggctgctggaacgcccgctgcgcgccgatctggccctgattcgcgcccatcgctgcgacaccctgggcaacctgacgtaccagctgtccgcccggaacttcaacccgctgattgcgctggccgcggatatcaccctcgtggaaccggacgaactcgtcgaaaccggggagctgcaaccggatcatatcgtgacccccggcgcggtgatcgaccatatcatcgtctcccaagagtcgaagtaatggacgccaagcagcgcatcgcccgccgcgtcgcgcaggaactgcgcgacggcgacatcgtgaacctgggcatcggcctgccgaccatggtggccaactatctgccggagggcatccacatcaccctgcagtcggagaacggcttcctgggcctgggcccggtgaccaccgcccacccggacctggtgaacgcggggggccagccgtgcggggtgctgccgggcgccgcgatgttcgactcggccatgtcgttcgccctgatccggggggggcacatcgacgcctgcgtcctcggcgggctccaggtggacgaggaggccaacctggccaactgggtcgtgccgggcaaaatggtcccgggcatggggggggccatggacctcgtgaccggctcgcgcaaggtgatcattgcgatggagcactgcgccaaggatggctcggccaagatcctgcgccgctgcaccatgccgctgacggcccagcacgccgtgcacatgctggtgaccgaactggccgtgttccgcttcatcgacggcaagatgtggctgaccgagatcgccgacggctgcgacctcgcgaccgtgcgcgccaagaccgaggcccgcttcgaggtggccgccgacctgaacacccagcgcggcgacctgtgacggcacccctacaaacagaaggaatataaaatgaagaactgcgtgatcgtgtcggccgtccgcaccgcgatcggctcgttcaacggctcgctcgcctcgacctcggccatcgacctgggggccaccgtgatcaaggcggccatcgagcgcgccaagatcgactcgcaacacgtggacgaggtgatcatgggcaacgtgctgcaagccgggctgggccagaacccggcccggcaggccctcctgaagtcgggcctcgccgagaccgtgtgcggcttcaccgtgaacaaggtgtgcggctccggcctgaagtcggtcgccctcgccgcccaagccatccaagccgggcaagcccaatcgatcgtggccggcggcatggagaacatgtcgctggccccgtacctgctggacgccaaggcccgctcgggctatcggctgggggacggccaggtgtacgacgtgatcctccgcgacggcctcatgtgcgccacccacggctaccacatgggcatcaccgccgagaacgtggccaaggagtacggcatcacccgcgagatgcaggacgagctggccctgcactcgcagcgcaaggccgccgccgccatcgagtcgggcgccttcacggccgagatcgtgccggtgaacgtggtgacccgcaagaagaccttcgtgttctcgcaggacgagttcccgaaggccaactcgaccgccgaggccctgggggccctgcgcccggccttcgacaaagccggcaccgtgaccgcgggcaacgcctcgggcatcaacgacggcgccgcggcgctggtgatcatggaggagtcggccgcgctggccgcggggctgaccccgctggcccgcatcaagtcgtacgcctcgggcggggtgccgccggccctgatgggcatgggcccggtgccggccacccagaaggccctgcagctggcgggcctgcaactggcggacatcgacctgatcgaggccaacgaggccttcgccgcccagttcctggcggtgggcaagaacctgggcttcgactcggagaaggtgaatgtgaacggcggcgcgatcgccctgggccatccgatcggcgcctcgggcgcgcgcatcctggtgaccctgctgcacgccatgcaggcccgggataagacgctgggcctggccaccctgtgcatcggcgggggccagggcatcgccatggtgatcgaacgcctcaactaatcaataaaaacacccgatagcgaaagttatcgggtgttttcttgaacatcgaaagctt

SEQ ID NO: 17 - protein, 393 aa, Escherichia coli
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 18 - protein, 218 aa, Clostridium acetobutylicum
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 19 - protein, 221 aa, Escherichia coli
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 20 - DNA, 2642 bp, Artificial Sequence
  codonoptimized gen
ggtacctttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaatttaaaaggagggattaaaatgaactcgaagatcatccgctttgagaacctccgctcgttcttcaaggacggcatgacgattatgattgggggctttctgaactgcggcaccccgaccaagctgattgattttctggtgaacctgaacatcaagaatctcaccattatctccaacgatacctgctacccgaacaccggcatcggcaagctcatctcgaacaaccaggtgaagaaactgatcgcctcgtacattggctcgaatccggacacggggaaaaaactgttcaacaatgagctggaagtggaactgtcgccgcagggcacgctggtggagcgcattcgcgcggggggctcgggcctcgggggggtcctgaccaagaccggcctggggaccctcatcgaaaaaggcaaaaagaagatctccatcaacgggaccgaatacctcctggagctgccgctgaccgccgacgtggccctgattaaaggctcgattgtcgacgaagccggcaacaccttctacaagggcaccaccaagaacttcaacccgtacatggcgatggccgccaaaaccgtgatcgtggaagccgaaaacctggtctcctgcgagaagctggagaaggaaaaggccatgacgccgggcgtgctcatcaactacatcgtgaaggagcccgcctaaaatgattaatgacaaaaacctggccaaggagattattgcgaagcgcgtggcccgcgaactgaaaaacgggcagctggtcaatctgggcgtgggcctgcccacgatggtggccgactacatcccgaagaatttcaagatcaccttccagtcggaaaatggcattgtgggcatgggggccagcccgaaaatcaatgaggcggacaaggacgtggtgaatgcggggggcgactataccaccgtgctgccggacggcacctttttcgactcgtcggtgtcgttctcgctcattcggggcgggcatgtggatgtcacggtgctgggcgcgctgcaggtggacgagaagggcaatatcgccaactggatcgtgccgggcaagatgctgtcggggatgggcggcgccatggatctggtgaacggggccaagaaggtcatcattgccatgcgccacaccaacaagggccaaccgaagatcctgaagaagtgcaccctgccgctgaccgccaaatcgcaggcgaacctgattgtgaccgaactgggggtgatcgaagtgattaacgacggcctcctcctcaccgaaatcaacaagaacaccaccattgacgaaattcgctcgctgacggccgccgacctgctcatcagcaatgagctgcgcccgatggccgtgtagaaagaaatactatgaaacaatattaaaaaaataagagttaccatttaaggtaactcttatttttattacttaagataataagctt

SEQ ID NO: 21 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 22 - DNA, 2634 bp, Artificial Sequence
  codonoptimized gen
ggtacctttcaggagattcaagaatgaaggaggtggtgattgcctcggccgtgcgcaccgcgatcggctcgtacggcaagagcctgaaagacgtgcccgccgtggacctgggcgccaccgccattaaggaggccgtgaagaaggccggcatcaagccggaggacgtgaacgaggtgatcctgggcaacgtgctgcaggccgggctcggccagaacccggcccgccaggcgagcttcaaagccggcctcccggtggagatccccgccatgaccatcaacaaggtgtgcggctcgggcctgcgcaccgtgtcgctggcggcccagatcatcaaggccggcgacgccgacgtgatcattgccggcggcatggaaaacatgtcgcgcgccccgtacctggccaacaacgcccgctggggctaccgcatggggaacgccaagttcgtggacgagatgatcaccgacggcctgtgggacgccttcaacgactaccacatgggcatcaccgccgagaacatcgccgagcgctggaacatctcgcgcgaggagcaggacgagttcgccctggcctcgcaaaagaaggcggaggaagccatcaagtcgggccagttcaaggacgagatcgtgccggtggtgattaagggccgcaagggcgagaccgtggtggacacggacgagcacccgcgcttcgggtcgacgatcgagggcctggccaagctgaagccggccttcaagaaggacggcaccgtgaccgcgggcaacgcctcgggcctgaacgactgcgcggccgtgctggtgatcatgtcggccgaaaaggccaaggagctgggcgtgaaaccgctggcgaagatcgtgtcgtatggctcggccggcgtggacccggccatcatgggctacggcccgttctacgccaccaaggcggcgatcgagaaagccggctggacggtcgacgagctggacctgatcgagtcgaacgaggcctttgccgcccagtcgctggccgtcgccaaggacctgaagttcgacatgaacaaagtgaacgtgaatggcggcgccatcgccctggggcacccgatcggcgcctcgggcgcccgcatcctggtgaccctggtgcacgcgatgcagaagcgcgacgccaagaaggggctggcgaccctgtgcatcggcggcggccagggcaccgcgatcctgctggaaaagtgctagaaagtttaaaaggagggattaaaatgaactcgaagatcatccgctttgagaacctccgctcgttcttcaaggacggcatgacgattatgattgggggctttctgaactgcggcaccccgaccaagctgattgattttctggtgaacctgaacatcaagaatctcaccattatctccaacgatacctgctacccgaacaccggcatcggcaagctcatctcgaacaaccaggtgaagaaactgatcgcctcgtacattggctcgaatccggacacggggaaaaaactgttcaacaatgagctggaagtggaactgtcgccgcagggcacgctggtggagcgcattcgcgcggggggctcgggcctcgggggggtcctgaccaagaccggcctggggaccctcatcgaaaaaggcaaaaagaagatctccatcaacgggaccgaatacctcctggagctgccgctgaccgccgacgtggccctgattaaaggctcgattgtcgacgaagccggcaacaccttctacaagggcaccaccaagaacttcaacccgtacatggcgatggccgccaaaaccgtgatcgtggaagccgaaaacctggtctcctgcgagaagctggagaaggaaaaggccatgacgccgggcgtgctcatcaactacatcgtgaaggagcccgcctaaaatgattaatgacaaaaacctggccaaggagattattgcgaagcgcgtggcccgcgaactgaaaaacgggcagctggtcaatctgggcgtgggcctgcccacgatggtggccgactacatcccgaagaatttcaagatcaccttccagtcggaaaatggcattgtgggcatgggggccagcccgaaaatcaatgaggcggacaaggacgtggtgaatgcggggggcgactataccaccgtgctgccggacggcacctttttcgactcgtcggtgtcgttctcgctcattcggggcgggcatgtggatgtcacggtgctgggcgcgctgcaggtggacgagaagggcaatatcgccaactggatcgtgccgggcaagatgctgtcggggatgggcggcgccatggatctggtgaacggggccaagaaggtcatcattgccatgcgccacaccaacaagggccaaccgaagatcctgaagaagtgcaccctgccgctgaccgccaaatcgcaggcgaacctgattgtgaccgaactgggggtgatcgaagtgattaacgacggcctcctcctcaccgaaatcaacaagaacaccaccattgacgaaattcgctcgctgacggccgccgacctgctcatcagcaatgagctgcgcccgatggccgtgtagaaagaaatactatgaaacaatattaaaaaaataagagttaccatttaaggtaactcttatttttattacttaagataataagctt

SEQ ID NO: 23 - protein, 136 aa, Haemophilus influenzae
MLGNCFSFPVRVYYEDTDAGGVVYHARYLHFFERARTEYLRILNFTQQTLLEEQQLAFVVKSLAIDYCVAAKLDDLLMVETEVSEVKGATILFEQRLMRNTLMLSKATVKVACVDLGKMKPVALPKEVKAAFNHLK

SEQ ID NO: 24 - protein, 244 aa, Clostridium acetobutylicum
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 25 - DNA, 2612 bp, Artificial Sequence; identical to SEQ ID NO: 16
  codonoptimized gen

SEQ ID NO: 26 - DNA, 70 bp, Escherichia coli
ccccaggctttacactttatgcttccggctcgtatgttgtgtggaattgtgagcggataacaatttcaca

SEQ ID NO: 27 - DNA, 882 bp, Artificial Sequence
  codonoptimized gen
aagcttccccaggctttacactttatgcttccggctcgtatgttgtgtggaattgtgagcggataacaatttcacatttcaggagattcaagaatgctcaaggacgaagtcatcaaacaaatcagcaccccgctgacgtcccccgcctttccgcgggggccctacaagttccacaaccgcgagtacttcaacatcgtgtaccgcaccgacatggatgccctccgcaaggtcgtcccggaaccgctggagatcgatgagcccctggtgcgcttcgagattatggcgatgcatgacacctccggcctgggctgctataccgaaagcggccaggcgattccggtctcgtttaatggcgtgaagggggattacctgcacatgatgtacctggacaacgaaccggccattgccgtgggccgcgagctgtccgcctaccccaagaagctgggctatccgaagctgtttgtggattcggacacgctggtcggcaccctcgactacggcaaactgcgcgtcgccaccgccaccatgggctacaagcacaaggcgctggacgcgaatgaagcgaaggaccagatttgccgccccaactacatgctgaaaatcatccccaattacgacggctccccgcgcatttgcgaactgatcaacgcgaagatcaccgacgtgaccgtgcatgaagcctggacgggccccacgcgcctgcagctcttcgaccatgcgatggcgcccctgaatgacctgccggtgaaagaaatcgtgtcgtcctcgcatatcctcgccgatattatcctgccccgggcggaggtcatctacgactacctgaagtaataaaaataagagttaccttaaatggtaactcttatttttttaatattgggatcc

SEQ ID NO: 28 - DNA, 7714 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagctttcgatgttcaagaaaacacccgataactttcgctatcgggtgtttttattgattagttgaggcgttcgatcaccatggcgatgccctggcccccgccgatgcacagggtggccaggcccagcgtcttatcccgggcctgcatggcgtgcagcagggtcaccaggatgcgcgcgcccgaggcgccgatcggatggcccagggcgatcgcgccgccgttcacattcaccttctccgagtcgaagcccaggttcttgcccaccgccaggaactgggcggcgaaggcctcgttggcctcgatcaggtcgatgtccgccagttgcaggcccgccagctgcagggccttctgggtggccggcaccgggcccatgcccatcagggccggcggcaccccgcccgaggcgtacgacttgatgcgggccagcggggtcagccccgcggccagcgcggccgactcctccatgatcaccagcgccgcggcgccgtcgttgatgcccgaggcgttgcccgcggtcacggtgccggctttgtcgaaggccgggcgcagggcccccagggcctcggcggtcgagttggccttcgggaactcgtcctgcgagaacacgaaggtcttcttgcgggtcaccacgttcaccggcacgatctcggccgtgaaggcgcccgactcgatggcggcggcggccttgcgctgcgagtgcagggccagctcgtcctgcatctcgcgggtgatgccgtactccttggccacgttctcggcggtgatgcccatgtggtagccgtgggtggcgcacatgaggccgtcgcggaggatcacgtcgtacacctggccgtcccccagccgatagcccgagcgggccttggcgtccagcaggtacggggccagcgacatgttctccatgccgccggccacgatcgattgggcttgcccggcttggatggcttgggcggcgagggcgaccgacttcaggccggagccgcacaccttgttcacggtgaagccgcacacggtctcggcgaggcccgacttcaggagggcctgccgggccgggttctggcccagcccggcttgcagcacgttgcccatgatcacctcgtccacgtgttgcgagtcgatcttggcgcgctcgatggccgccttgatcacggtggcccccaggtcgatggccgaggtcgaggcgagcgagccgttgaacgagccgatcgcggtgcggacggccgacacgatcacgcagttcttcattttatattccttctgtttgtaggggtgccgtcacaggtcgccgcgctgggtgttcaggtcggcggccacctcgaagcgggcctcggtcttggcgcgcacggtcgcgaggtcgcagccgtcggcgatctcggtcagccacatcttgccgtcgatgaagcggaacacggccagttcggtcaccagcatgtgcacggcgtgctgggccgtcagcggcatggtgcagcggcgcaggatcttggccgagccatccttggcgcagtgctccatcgcaatgatcaccttgcgcgagccggtcacgaggtccatggccccccccatgcccgggaccattttgcccggcacgacccagttggccaggttggcctcctcgtccacctggagcccgccgaggacgcaggcgtcgatgtgccccccccggatcagggcgaacgacatggccgagtcgaacatcgcggcgcccggcagcaccccgcacggctggccccccgcgttcaccaggtccgggtgggcggtggtcaccgggcccaggcccaggaagccgttctccgactgcagggtgatgtggatgccctccggcagatagttggccaccatggtcggcaggccgatgcccaggttcacgatgtcgccgtcgcgcagttcctgcgcgacgcggcgggcgatgcgctgcttggcgtccattacttcgactcttgggagacgatgatatggtcgatcaccgcgccgggggtcacgatatgatccggttgcagctccccggtttcgacgagttcgtccggttccacgagggtgatatccgcggccagcgcaatcagcgggttgaagttccgggcggacagctggtacgtcaggttgcccagggtgtcgcagcgatgggcgcgaatcagggccagatcggcgcgcagcgggcgttccagcagccaggtcttcccgtccagggtcagcgtctgcttgccttcctcgaccacggtgcccacgccggtcggcgtcaggaacccgccgaggcccgccccgccgcaccggatttgctcaatgagggtcccctgcgggaccagcaccacatccatctcgcccgagatcatccggcggccggtctccgggttcgtgccgatgtggctggcgatcactttgcggacgcggccgttgacaatcagggggccaatgccggtgtccacgaaggcggtgtcgttggcaatgagcgtcagatcgcgcacccccgactccaggagggcttcgaccagccgcgacggcgtcccgatgcccatgaagccccccaccatgatggtcatgccatcgcggaaaaagccggtggcgtcctgcagcgtcatgagcttggtcttcattttttatccctcttgcatacggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 29 - DNA, 7744 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaattccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 30 - DNA, 7736 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaactttctagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 31 - DNA, 8560 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccaatattaaaaaaataagagttaccatttaaggtaactcttatttttattacttcaggtagtcgtagatgacctccgcccggggcaggataatatcggcgaggatatgcgaggacgacacgatttctttcaccggcaggtcattcaggggcgccatcgcatggtcgaagagctgcaggcgcgtggggcccgtccaggcttcatgcacggtcacgtcggtgatcttcgcgttgatcagttcgcaaatgcgcggggagccgtcgtaattggggatgattttcagcatgtagttggggcggcaaatctggtccttcgcttcattcgcgtccagcgccttgtgcttgtagcccatggtggcggtggcgacgcgcagtttgccgtagtcgagggtgccgaccagcgtgtccgaatccacaaacagcttcggatagcccagcttcttggggtaggcggacagctcgcggcccacggcaatggccggttcgttgtccaggtacatcatgtgcaggtaatcccccttcacgccattaaacgagaccggaatcgcctggccgctttcggtatagcagcccaggccggaggtgtcatgcatcgccataatctcgaagcgcaccaggggctcatcgatctccagcggttccgggacgaccttgcggagggcatccatgtcggtgcggtacacgatgttgaagtactcgcggttgtggaacttgtagggcccccgcggaaaggcgggggacgtcagcggggtgctgatttgtttgatgacttcgtccttgagcattcttgaatctcctgaaatgtgaaattgttatccgctcacaattccacacaacatacgagccggaagcataaagtgtaaagcctggggaagctttcgatgttcaagaaaacacccgataactttcgctatcgggtgtttttattgattagttgaggcgttcgatcaccatggcgatgccctggcccccgccgatgcacagggtggccaggcccagcgtcttatcccgggcctgcatggcgtgcagcagggtcaccaggatgcgcgcgcccgaggcgccgatcggatggcccagggcgatcgcgccgccgttcacattcaccttctccgagtcgaagcccaggttcttgcccaccgccaggaactgggcggcgaaggcctcgttggcctcgatcaggtcgatgtccgccagttgcaggcccgccagctgcagggccttctgggtggccggcaccgggcccatgcccatcagggccggcggcaccccgcccgaggcgtacgacttgatgcgggccagcggggtcagccccgcggccagcgcggccgactcctccatgatcaccagcgccgcggcgccgtcgttgatgcccgaggcgttgcccgcggtcacggtgccggctttgtcgaaggccgggcgcagggcccccagggcctcggcggtcgagttggccttcgggaactcgtcctgcgagaacacgaaggtcttcttgcgggtcaccacgttcaccggcacgatctcggccgtgaaggcgcccgactcgatggcggcggcggccttgcgctgcgagtgcagggccagctcgtcctgcatctcgcgggtgatgccgtactccttggccacgttctcggcggtgatgcccatgtggtagccgtgggtggcgcacatgaggccgtcgcggaggatcacgtcgtacacctggccgtcccccagccgatagcccgagcgggccttggcgtccagcaggtacggggccagcgacatgttctccatgccgccggccacgatcgattgggcttgcccggcttggatggcttgggcggcgagggcgaccgacttcaggccggagccgcacaccttgttcacggtgaagccgcacacggtctcggcgaggcccgacttcaggagggcctgccgggccgggttctggcccagcccggcttgcagcacgttgcccatgatcacctcgtccacgtgttgcgagtcgatcttggcgcgctcgatggccgccttgatcacggtggcccccaggtcgatggccgaggtcgaggcgagcgagccgttgaacgagccgatcgcggtgcggacggccgacacgatcacgcagttcttcattttatattccttctgtttgtaggggtgccgtcacaggtcgccgcgctgggtgttcaggtcggcggccacctcgaagcgggcctcggtcttggcgcgcacggtcgcgaggtcgcagccgtcggcgatctcggtcagccacatcttgccgtcgatgaagcggaacacggccagttcggtcaccagcatgtgcacggcgtgctgggccgtcagcggcatggtgcagcggcgcaggatcttggccgagccatccttggcgcagtgctccatcgcaatgatcaccttgcgcgagccggtcacgaggtccatggccccccccatgcccgggaccattttgcccggcacgacccagttggccaggttggcctcctcgtccacctggagcccgccgaggacgcaggcgtcgatgtgccccccccggatcagggcgaacgacatggccgagtcgaacatcgcggcgcccggcagcaccccgcacggctggccccccgcgttcaccaggtccgggtgggcggtggtcaccgggcccaggcccaggaagccgttctccgactgcagggtgatgtggatgccctccggcagatagttggccaccatggtcggcaggccgatgcccaggttcacgatgtcgccgtcgcgcagttcctgcgcgacgcggcgggcgatgcgctgcttggcgtccattacttcgactcttgggagacgatgatatggtcgatcaccgcgccgggggtcacgatatgatccggttgcagctccccggtttcgacgagttcgtccggttccacgagggtgatatccgcggccagcgcaatcagcgggttgaagttccgggcggacagctggtacgtcaggttgcccagggtgtcgcagcgatgggcgcgaatcagggccagatcggcgcgcagcgggcgttccagcagccaggtcttcccgtccagggtcagcgtctgcttgccttcctcgaccacggtgcccacgccggtcggcgtcaggaacccgccgaggcccgccccgccgcaccggatttgctcaatgagggtcccctgcgggaccagcaccacatccatctcgcccgagatcatccggcggccggtctccgggttcgtgccgatgtggctggcgatcactttgcggacgcggccgttgacaatcagggggccaatgccggtgtccacgaaggcggtgtcgttggcaatgagcgtcagatcgcgcacccccgactccaggagggcttcgaccagccgcgacggcgtcccgatgcccatgaagccccccaccatgatggtcatgccatcgcggaaaaagccggtggcgtcctgcagcgtcatgagcttggtcttcattttttatccctcttgcatacggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 32 - DNA, 8590 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccaatattaaaaaaataagagttaccatttaaggtaactcttatttttattacttcaggtagtcgtagatgacctccgcccggggcaggataatatcggcgaggatatgcgaggacgacacgatttctttcaccggcaggtcattcaggggcgccatcgcatggtcgaagagctgcaggcgcgtggggcccgtccaggcttcatgcacggtcacgtcggtgatcttcgcgttgatcagttcgcaaatgcgcggggagccgtcgtaattggggatgattttcagcatgtagttggggcggcaaatctggtccttcgcttcattcgcgtccagcgccttgtgcttgtagcccatggtggcggtggcgacgcgcagtttgccgtagtcgagggtgccgaccagcgtgtccgaatccacaaacagcttcggatagcccagcttcttggggtaggcggacagctcgcggcccacggcaatggccggttcgttgtccaggtacatcatgtgcaggtaatcccccttcacgccattaaacgagaccggaatcgcctggccgctttcggtatagcagcccaggccggaggtgtcatgcatcgccataatctcgaagcgcaccaggggctcatcgatctccagcggttccgggacgaccttgcggagggcatccatgtcggtgcggtacacgatgttgaagtactcgcggttgtggaacttgtagggcccccgcggaaaggcgggggacgtcagcggggtgctgatttgtttgatgacttcgtccttgagcattcttgaatctcctgaaatgtgaaattgttatccgctcacaattccacacaacatacgagccggaagcataaagtgtaaagcctggggaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaattccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 33 - DNA, 8590 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccaatattaaaaaaataagagttaccatttaaggtaactcttatttttattacttgaggtagtcgtagatgacttcggcgcgcgggaggatgatgtccgccaggatatgcgacgacgacacaatctctttcacggggaggtcgttgagcggcgccatcgcatgatcgaacagctgcaggcgggtggggccggtccacgcttcatgcacggtcacgtcggtaatcttggcgttgatcagttcgcagatgcgcggcgagccgtcataattcggaataatcttcagcatgtagttcggccggcagatttggtccttggcctcattcgcgtcgagcgccttgtgcttgtagcccatggtggccgtcgccacgcggagcttgccatagtccagggtcccgaccagcgtgtccgagtccacgaagagtttcggatagcccagtttcttcgggtaggccgacagctcgcgccccacggcaatcgcgggttcgttgtccagatacatcatgtgcaggtaatcgcccttcacgccgttaaagctcaccggaatggcttggcccgattcggtgtagcagcccaggcccgaggtatcgtgcatcgccatgatctcgaagcgcaccaggggctcgtcgatctccagcggctcgggcaccaccttgcgcagggcgtccatgtcggtgcgatacacgatgttgaagtattcccggttgtggaacttgtagggcccccgcggaaaggcgggcgaggtcagcggggtcgaaatttgcttaatcacctcatctttgagcattcttgaatctcctgaaatgtgaaattgttatccgctcacaattccacacaacatacgagccggaagcataaagtgtaaagcctggggaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaattccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 34 - DNA, 8582 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccaatattaaaaaaataagagttaccatttaaggtaactcttatttttattacttcaggtagtcgtagatgacctccgcccggggcaggataatatcggcgaggatatgcgaggacgacacgatttctttcaccggcaggtcattcaggggcgccatcgcatggtcgaagagctgcaggcgcgtggggcccgtccaggcttcatgcacggtcacgtcggtgatcttcgcgttgatcagttcgcaaatgcgcggggagccgtcgtaattggggatgattttcagcatgtagttggggcggcaaatctggtccttcgcttcattcgcgtccagcgccttgtgcttgtagcccatggtggcggtggcgacgcgcagtttgccgtagtcgagggtgccgaccagcgtgtccgaatccacaaacagcttcggatagcccagcttcttggggtaggcggacagctcgcggcccacggcaatggccggttcgttgtccaggtacatcatgtgcaggtaatcccccttcacgccattaaacgagaccggaatcgcctggccgctttcggtatagcagcccaggccggaggtgtcatgcatcgccataatctcgaagcgcaccaggggctcatcgatctccagcggttccgggacgaccttgcggagggcatccatgtcggtgcggtacacgatgttgaagtactcgcggttgtggaacttgtagggcccccgcggaaaggcgggggacgtcagcggggtgctgatttgtttgatgacttcgtccttgagcattcttgaatctcctgaaatgtgaaattgttatccgctcacaattccacacaacatacgagccggaagcataaagtgtaaagcctggggaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaactttctagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 35 - protein, 246 aa, Ralstonia eutropha
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 36 - protein, 134 aa, Ralstonia eutropha
MSAQSLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALREDKPIATLTTRIFTQGGALAVTGEAVVKLP

SEQ ID NO: 37 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 38 - DNA, 50 bp, Acinetobacter caviae
ggcacgcaggcacaatcagcccggcccctgccgggctgattgttctcccc

SEQ ID NO: 39 - DNA, 5106 bp, Artificial Sequence
  codonoptimized gen
ggtacctttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaaggggttttccggggccgcgcgcggttggcgcggacccggcgacgataacgaagccaatcaaggagtggacatgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctgaaacagaggaggacgccgcatgtcggcccagtcgctggaggtcgggcaaaaagcccggctgtccaagcggtttggcgcggcggaggtggcggcctttgcggcgctctccgaggacttcaacccgctgcacctggaccccgcgtttgccgcgaccaccgcgttcgagcgccccatcgtgcatggcatgctgctcgcctcgctgttctcgggcctcctcggccagcagctccccggcaaggggagcatctacctgggccaatcgctctcgttcaaactgccggtgttcgtgggggacgaagtgaccgccgaggtggaagtcaccgcgctgcgcgaggacaaaccgatcgccacgctgacgacccggatctttacccaaggcggggccctggccgtcaccggggaagccgtggtcaagctgccgtaagcaccggctttcaggagattcaagaatgaaggtgaccaaccagaaggagctgaagcagaagctgaacgagctgcgcgaggcccagaagaagttcgccacctacacccaggagcaggtggacaagatcttcaagcagtgcgccattgccgccgccaaggagcgcatcaacctggccaagctggccgtggaggagaccggcatcggcctggtggaggacaagatcatcaagaaccacttcgccgccgagtacatctacaacaagtacaagaacgagaagacctgcggcattatcgaccacgacgactcgctgggcatcaccaaggtggccgagccgatcggcatcgtggccgccatcgtgccgaccacgaacccgacctcgaccgccatcttcaagtcgctgatctcgctgaagacccgcaacgccatcttcttctcgccgcacccgcgcgccaagaagtcgaccatcgccgccgccaagctgatcctggacgccgccgtgaaggccggggccccgaagaacatcatcggctggattgacgagccgtcgatcgagctgtcgcaggacctgatgtcggaggccgatatcatcctggccaccggcggcccgtcgatggtgaaggccgcctactcgtcgggcaagccggccatcggcgtgggcgcgggcaacaccccggccatcatcgacgagtccgccgacatcgatatggccgtgtcgtcgatcatcctgtcgaagacctacgacaacggcgtgatctgcgcctcggagcagtcgatcctggtgatgaactccatctacgagaaggtgaaggaggagttcgtgaagcgcggctcgtacatcctgaaccagaacgagatcgccaagatcaaggagacgatgttcaagaacggcgccatcaacgccgacatcgtgggcaagtcggcctacatcatcgccaagatggccggcatcgaggtgccgcagaccaccaagatcctcatcggcgaggtgcagtcggtggagaagtcggagctgttctcgcacgagaagctgtcgccggtgctggccatgtacaaggtgaaggacttcgatgaggccctcaagaaggcccagcgcctgatcgagctgggcggctcgggccacacctcgtcgctgtacatcgactcgcagaacaacaaggacaaggtgaaggagttcggcctggccatgaagacctcgcgcaccttcatcaacatgccgtcgtcgcagggcgcctcgggcgacctgtacaacttcgccatcgccccgtcgttcaccctgggctgcgggacctggggcggcaactcggtgtcgcagaacgtggagccgaagcacctgctgaacatcaagtcggtggccgagcgccgcgagaatatgctgtggttcaaggtgccgcagaagatctacttcaagtacggctgcctgcgcttcgccctgaaggagctgaaggacatgaacaagaagcgcgccttcatcgtgaccgacaaggacctgttcaagctgggctacgtgaacaagatcaccaaggtgctggacgagatcgacatcaagtactcgatcttcaccgatatcaagtcggacccgaccattgactcggtgaagaagggcgccaaggagatgctgaacttcgagccggacaccatcatctcgatcggcggcggctcgccgatggacgccgccaaggtgatgcacctgctgtacgagtacccggaggcggagatcgagaacctcgccatcaatttcatggacatccgcaagcgcatctgcaacttcccgaagctgggcaccaaggccatctcggtggccatcccgaccaccgccggcaccggctcggaggccaccccgttcgccgtgatcaccaacgacgagaccggcatgaagtacccgctgacctcgtacgaactgaccccgaacatggccatcattgacaccgagctgatgctgaacatgccgcgcaagctgaccgccgccaccggcattgatgccctggtgcacgccatcgaggcctacgtgtcggtgatggccacggactacaccgacgagctggccctgcgcgccatcaagatgatcttcaagtacctgccgcgcgcctacaagaacggcaccaacgacatcgaggcccgcgagaagatggcccacgcctcgaacatcgccggcatggccttcgccaacgccttcctgggcgtgtgccactcgatggcccacaagctgggcgccatgcaccacgtgccgcacggcatcgcctgcgccgtgctgatcgaggaggtgatcaagtacaacgccaccgactgcccgaccaagcagaccgccttcccgcagtacaagtcgccgaacgccaagcgcaagtacgccgagatcgccgagtacctgaacctgaagggcacctcggacacggagaaggtgaccgccctgatcgaggccatctcgaagctgaagatcgacctgtcgatcccgcagaacatctcggccgccggcatcaacaagaaggatttctacaacaccctggacaagatgtcggagctggccttcgacgaccagtgcaccaccgccaacccgcgctacccgctgatctcggagctgaaggacatctacatcaagtcgttctaaggcacgcaggcacaatcagcccggcccctgccgggctgattgttctccccaagctt

SEQ ID NO: 40 - DNA, 2512 bp, Artificial Sequence
  codonoptimized gen
ggtacctttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaaggggttttccggggccgcgcgcggttggcgcggacccggcgacgataacgaagccaatcaaggagtggacatgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctgaaacagaggaggacgccgcatgtcggcccagtcgctggaggtcgggcaaaaagcccggctgtccaagcggtttggcgcggcggaggtggcggcctttgcggcgctctccgaggacttcaacccgctgcacctggaccccgcgtttgccgcgaccaccgcgttcgagcgccccatcgtgcatggcatgctgctcgcctcgctgttctcgggcctcctcggccagcagctccccggcaaggggagcatctacctgggccaatcgctctcgttcaaactgccggtgttcgtgggggacgaagtgaccgccgaggtggaagtcaccgcgctgcgcgaggacaaaccgatcgccacgctgacgacccggatctttacccaaggcggggccctggccgtcaccggggaagccgtggtcaagctgccgtaagcaccggcggcacgcaggcacaatcagcccggcccctgccgggctgattgttctccccaagctt

SEQ ID NO: 41 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKTNDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 42 - DNA, 66 bp, Clostridium acetobutylicum
actatgaaacaatattaaaaaaataagagttaccatttaaggtaactcttatttttattacttaag

SEQ ID NO: 43 - DNA, 4742 bp, Artificial Sequence
  codonoptimized gen
ggtacctttcaggagattcaagaatgaaggaggtggtgattgcctcggccgtgcgcaccgcgatcggctcgtacggcaagagcctgaaagacgtgcccgccgtggacctgggcgccaccgccattaaggaggccgtgaagaaggccggcatcaagccggaggacgtgaacgaggtgatcctgggcaacgtgctgcaggccgggctcggccagaacccggcccgccaggcgagcttcaaagccggcctcccggtggagatccccgccatgaccatcaacaaggtgtgcggctcgggcctgcgcaccgtgtcgctggcggcccagatcatcaaggccggcgacgccgacgtgatcattgccggcggcatggaaaacatgtcgcgcgccccgtacctggccaacaacgcccgctggggctaccgcatggggaacgccaagttcgtggacgagatgatcaccgacggcctgtgggacgccttcaacgactaccacatgggcatcaccgccgagaacatcgccgagcgctggaacatctcgcgcgaggagcaggacgagttcgccctggcctcgcaaaagaaggcggaggaagccatcaagtcgggccagttcaaggacgagatcgtgccggtggtgattaagggccgcaagggcgagaccgtggtggacacggacgagcacccgcgcttcgggtcgacgatcgagggcctggccaagctgaagccggccttcaagaaggacggcaccgtgaccgcgggcaacgcctcgggcctgaacgactgcgcggccgtgctggtgatcatgtcggccgaaaaggccaaggagctgggcgtgaaaccgctggcgaagatcgtgtcgtatggctcggccggcgtggacccggccatcatgggctacggcccgttctacgccaccaaggcggcgatcgagaaagccggctggacggtcgacgagctggacctgatcgagtcgaacgaggcctttgccgcccagtcgctggccgtcgccaaggacctgaagttcgacatgaacaaagtgaacgtgaatggcggcgccatcgccctggggcacccgatcggcgcctcgggcgcccgcatcctggtgaccctggtgcacgcgatgcagaagcgcgacgccaagaaggggctggcgaccctgtgcatcggcggcggccagggcaccgcgatcctgctggaaaagtgctagtttcaggagattcaagaatgaagaaggtgtgcgtcattggcgcggggacgatggggtccggcatcgcgcaggccttcgccgccaaaggcttcgaggtggtcctccgggacatcaaggatgaattcgtcgatcgcggcctggatttcattaacaagaacctgtcgaaactggtcaaaaaaggcaagatcgaagaggccaccaaggtggagattctcacccgcatcagcgggaccgtggacctgaacatggccgcggactgcgacctcgtgattgaagccgccgtcgagcgcatggacattaagaaacagatctttgcggatctcgacaacatctgcaaaccggagacgattctcgcctccaacaccagctcgctctcgatcacggaggtggcgagcgcgaccaagaccaacgacaaagtgatcggcatgcatttctttaatcccgcccccgtcatgaaactcgtggaggtcattcgcggcattgccacgtcgcaggaaaccttcgacgcggtgaaagagacgtcgattgccattggcaaggaccccgtggaagtcgccgaggcgccggggttcgtcgtcaaccgcattctgatcccgatgatcaatgaagcggtgggcatcctcgccgaagggattgcctcggtcgaggatattgataaggccatgaagctgggggcgaaccacccgatgggcccgctggaactgggggactttatcggcctcgacatttgcctggccatcatggacgtgctgtactcggaaaccggcgattcgaagtatcgcccgcacacgctgctgaaaaagtatgtccgggcgggctggctgggccgcaagagcggcaagggcttttacgactactcgaaataaaaagaaattttcaggagattcaagaatgaaggtgaccaaccagaaggagctgaagcagaagctgaacgagctgcgcgaggcccagaagaagttcgccacctacacccaggagcaggtggacaagatcttcaagcagtgcgccattgccgccgccaaggagcgcatcaacctggccaagctggccgtggaggagaccggcatcggcctggtggaggacaagatcatcaagaaccacttcgccgccgagtacatctacaacaagtacaagaacgagaagacctgcggcattatcgaccacgacgactcgctgggcatcaccaaggtggccgagccgatcggcatcgtggccgccatcgtgccgaccacgaacccgacctcgaccgccatcttcaagtcgctgatctcgctgaagacccgcaacgccatcttcttctcgccgcacccgcgcgccaagaagtcgaccatcgccgccgccaagctgatcctggacgccgccgtgaaggccggggccccgaagaacatcatcggctggattgacgagccgtcgatcgagctgtcgcaggacctgatgtcggaggccgatatcatcctggccaccggcggcccgtcgatggtgaaggccgcctactcgtcgggcaagccggccatcggcgtgggcgcgggcaacaccccggccatcatcgacgagtccgccgacatcgatatggccgtgtcgtcgatcatcctgtcgaagacctacgacaacggcgtgatctgcgcctcggagcagtcgatcctggtgatgaactccatctacgagaaggtgaaggaggagttcgtgaagcgcggctcgtacatcctgaaccagaacgagatcgccaagatcaaggagacgatgttcaagaacggcgccatcaacgccgacatcgtgggcaagtcggcctacatcatcgccaagatggccggcatcgaggtgccgcagaccaccaagatcctcatcggcgaggtgcagtcggtggagaagtcggagctgttctcgcacgagaagctgtcgccggtgctggccatgtacaaggtgaaggacttcgatgaggccctcaagaaggcccagcgcctgatcgagctgggcggctcgggccacacctcgtcgctgtacatcgactcgcagaacaacaaggacaaggtgaaggagttcggcctggccatgaagacctcgcgcaccttcatcaacatgccgtcgtcgcagggcgcctcgggcgacctgtacaacttcgccatcgccccgtcgttcaccctgggctgcgggacctggggcggcaactcggtgtcgcagaacgtggagccgaagcacctgctgaacatcaagtcggtggccgagcgccgcgagaatatgctgtggttcaaggtgccgcagaagatctacttcaagtacggctgcctgcgcttcgccctgaaggagctgaaggacatgaacaagaagcgcgccttcatcgtgaccgacaaggacctgttcaagctgggctacgtgaacaagatcaccaaggtgctggacgagatcgacatcaagtactcgatcttcaccgatatcaagtcggacccgaccattgactcggtgaagaagggcgccaaggagatgctgaacttcgagccggacaccatcatctcgatcggcggcggctcgccgatggacgccgccaaggtgatgcacctgctgtacgagtacccggaggcggagatcgagaacctcgccatcaatttcatggacatccgcaagcgcatctgcaacttcccgaagctgggcaccaaggccatctcggtggccatcccgaccaccgccggcaccggctcggaggccaccccgttcgccgtgatcaccaacgacgagaccggcatgaagtacccgctgacctcgtacgaactgaccccgaacatggccatcattgacaccgagctgatgctgaacatgccgcgcaagctgaccgccgccaccggcattgatgccctggtgcacgccatcgaggcctacgtgtcggtgatggccacggactacaccgacgagctggccctgcgcgccatcaagatgatcttcaagtacctgccgcgcgcctacaagaacggcaccaacgacatcgaggcccgcgagaagatggcccacgcctcgaacatcgccggcatggccttcgccaacgccttcctgggcgtgtgccactcgatggcccacaagctgggcgccatgcaccacgtgccgcacggcatcgcctgcgccgtgctgatcgaggaggtgatcaagtacaacgccaccgactgcccgaccaagcagaccgccttcccgcagtacaagtcgccgaacgccaagcgcaagtacgccgagatcgccgagtacctgaacctgaagggcacctcggacacggagaaggtgaccgccctgatcgaggccatctcgaagctgaagatcgacctgtcgatcccgcagaacatctcggccgccggcatcaacaagaaggatttctacaacaccctggacaagatgtcggagctggccttcgacgaccagtgcaccaccgccaacccgcgctacccgctgatctcggagctgaaggacatctacatcaagtcgttctaaactatgaaacaatattaaaaaaataagagttaccatttaaggtaactcttatttttattacttaagaagctt

SEQ ID NO: 44 - DNA, 2148 bp, Artificial Sequence
  codonoptimized gen
ggtacctttcaggagattcaagaatgaaggaggtggtgattgcctcggccgtgcgcaccgcgatcggctcgtacggcaagagcctgaaagacgtgcccgccgtggacctgggcgccaccgccattaaggaggccgtgaagaaggccggcatcaagccggaggacgtgaacgaggtgatcctgggcaacgtgctgcaggccgggctcggccagaacccggcccgccaggcgagcttcaaagccggcctcccggtggagatccccgccatgaccatcaacaaggtgtgcggctcgggcctgcgcaccgtgtcgctggcggcccagatcatcaaggccggcgacgccgacgtgatcattgccggcggcatggaaaacatgtcgcgcgccccgtacctggccaacaacgcccgctggggctaccgcatggggaacgccaagttcgtggacgagatgatcaccgacggcctgtgggacgccttcaacgactaccacatgggcatcaccgccgagaacatcgccgagcgctggaacatctcgcgcgaggagcaggacgagttcgccctggcctcgcaaaagaaggcggaggaagccatcaagtcgggccagttcaaggacgagatcgtgccggtggtgattaagggccgcaagggcgagaccgtggtggacacggacgagcacccgcgcttcgggtcgacgatcgagggcctggccaagctgaagccggccttcaagaaggacggcaccgtgaccgcgggcaacgcctcgggcctgaacgactgcgcggccgtgctggtgatcatgtcggccgaaaaggccaaggagctgggcgtgaaaccgctggcgaagatcgtgtcgtatggctcggccggcgtggacccggccatcatgggctacggcccgttctacgccaccaaggcggcgatcgagaaagccggctggacggtcgacgagctggacctgatcgagtcgaacgaggcctttgccgcccagtcgctggccgtcgccaaggacctgaagttcgacatgaacaaagtgaacgtgaatggcggcgccatcgccctggggcacccgatcggcgcctcgggcgcccgcatcctggtgaccctggtgcacgcgatgcagaagcgcgacgccaagaaggggctggcgaccctgtgcatcggcggcggccagggcaccgcgatcctgctggaaaagtgctagtttcaggagattcaagaatgaagaaggtgtgcgtcattggcgcggggacgatggggtccggcatcgcgcaggccttcgccgccaaaggcttcgaggtggtcctccgggacatcaaggatgaattcgtcgatcgcggcctggatttcattaacaagaacctgtcgaaactggtcaaaaaaggcaagatcgaagaggccaccaaggtggagattctcacccgcatcagcgggaccgtggacctgaacatggccgcggactgcgacctcgtgattgaagccgccgtcgagcgcatggacattaagaaacagatctttgcggatctcgacaacatctgcaaaccggagacgattctcgcctccaacaccagctcgctctcgatcacggaggtggcgagcgcgaccaagaccaacgacaaagtgatcggcatgcatttctttaatcccgcccccgtcatgaaactcgtggaggtcattcgcggcattgccacgtcgcaggaaaccttcgacgcggtgaaagagacgtcgattgccattggcaaggaccccgtggaagtcgccgaggcgccggggttcgtcgtcaaccgcattctgatcccgatgatcaatgaagcggtgggcatcctcgccgaagggattgcctcggtcgaggatattgataaggccatgaagctgggggcgaaccacccgatgggcccgctggaactgggggactttatcggcctcgacatttgcctggccatcatggacgtgctgtactcggaaaccggcgattcgaagtatcgcccgcacacgctgctgaaaaagtatgtccgggcgggctggctgggccgcaagagcggcaagggcttttacgactactcgaaataaaaagaaatactatgaaacaatattaaaaaaataagagttaccatttaaggtaactcttatttttattacttaagaagctt

SEQ ID NO: 45 - protein, 249 aa, Ralstonia eutropha
MKVLVAVKRVVDYNVKVRVKADGSGVDLANVKMSMNPFDEIAVEEAVRLKEAGVVTEVIAVSCGVTQCQETLRTAMAIGADRGILVESNEDLQPLAVAKLLKALIDKEQPQLVILGKQAIDDDSNQTGQMVAALAGLPQATFASKVVVADGKASVTREVDGGLETLSLKLPAVVTTDLRLNEPRYVTLPNIMKAKKKQLDIVKPEDLGVDVKPRLSTLKVVEPAKRSAGVMVPDVATLVQKLKNEAKVI

SEQ ID NO: 46 - protein, 311 aa, Ralstonia eutropha
MTALVIAEHDNQSIKAATLNTVTAAAQCGGDVHVLVAGANAKAAADAAAKIAGVTKVLLADAPYFGDGLAENVAEQALAIANDYSHILAPATPYGKNILPRVAAKLDVAQISEISKVDAPDTFERPIYAGNAIATVKSEDKIKVITVRGTAFDAAAAEGGSAAVETLPAVADAGVSQFVSREVAKSDRPELTAAKIIVSGGRGVGSGENYTKVLTPLADKLGAALGASRAAVDAGFVPNDYQVGQTGKIVAPQLYIAVGISGAIQHLAGMKDSKVIVAINKDAEAPIFSVADYGLVGDLNTVVPELVAALG

SEQ ID NO: 47 - protein, 594 aa, Ralstonia eutropha
MTYRAPIKDMLFVMNELAGLEAVSKLPGFEEATPETAEAVLDEAAKFNEQVVAPLNRAGDLDPSSWKDGVVTTTPGFKEAFRQFGEGGWQGVLHPQEFGGQGLPKLVATACNEMLNTANLSFALCPLLTDGAIEALLTAGTDEQKATFLPKLISGEWTGTMNLTEPQAGSDLAAVRTRAEPQGDGTYKVFGTKIFITYGEHDMAKNIVHLVLARTPTAPEGVKGISLFIVPKFMVNADGSTGERNDVHCVSIEHKLGIKASPTAVLQFGDHGGAIGTLVGEENRGLEYMFIMMNSARFSVGMQGIAVSERAYQQAVAFARERVQSRPVDGSAREAVTIIHHPDVKRMLMTMRALTEGARAVAYVAAAASDAAHQHPDEAVRQQNQAFYEFLVPVVKGWSTELSIDVTSLGVQVHGGMGFIEETGAAQHYRDARILPIYEGTTAIQANDLIGRKTVRDGGAVARAICAQIAETEAALGNHSCAGFTAVQAQLAKGRAALEDVVAFVVANAKSDPNAVFAGSVPYLKLCGIVFSGWQFGRAMLAADAKRAGDPGFYDAKIATAHFFAEHILSQASALRDAIVSGAAPVNAMTAEQF

SEQ ID NO: 48 - DNA, 75 bp, Ralstonia eutropha
gtggcctgacagtcgccggaaagaaaaagggcggccccgcggggccgccctttttgcttatcgaggcagcctgct

SEQ ID NO: 49 - DNA, 3762 bp, Artificial Sequence
  synthetic gen
aagcttccccaggctttacactttatgcttccggctcgtatgttgtgtggaattgtgagcggataacaatttcacatatcagtggagtgagcgcatgaaagtactcgtcgcagtcaagcgggtggtggattacaacgtcaaggtccgcgtcaaggcggacggcagcggcgtcgatctggccaacgtgaagatgagcatgaaccccttcgacgaaatcgccgtggaagaggccgtgcgcctgaaggaagccggtgttgtcaccgaagtgatcgccgtctcgtgcggtgtcacgcagtgccaggaaaccctgcgcaccgcgatggccatcggtgccgaccgcggcatcctggtggaatcgaacgaagacctgcaaccgctggccgtggccaagctgctgaaggcgctgatcgacaaggaacagccgcaactggtgatcctgggcaagcaggccatcgacgacgactccaaccagaccggccagatggtggccgcgctggctggcctgccgcaagccacgttcgcctccaaggtggtggttgccgacggcaaggcctcggtgacgcgtgaagtcgatggcggcctggaaacgctgtcgctcaagctgccggccgtggtgacgaccgacctgcgcctgaacgagccgcgctacgtgacgctgccgaacatcatgaaggccaagaagaagcagctcgatatcgtcaagccggaagacctcggcgtcgacgtcaagccgcgcctgtcgaccctgaaggtggtcgagcctgccaagcgcagcgcgggtgtgatggtgccggacgttgcgacgctggtgcagaagctgaagaacgaagccaaggttatctgagcgcgggggagaaataacatgactgcactcgtcattgctgaacacgacaatcaatccatcaaggccgccacgctgaacaccgtgactgccgctgcccagtgcggcggcgacgtgcacgtgctggtggctggtgccaacgccaaggccgcggccgatgcggccgccaagatcgccggcgtgaccaaggtcctgctggccgacgccccgtacttcggtgacggcctggccgagaacgtggccgagcaggcgctggccatcgccaacgactactcgcatatcctggctccggccaccccgtacggcaagaacatcctgccgcgcgtggctgccaagctggacgtggcccagatctcggaaatctccaaggtcgacgccccggacacgttcgagcgcccgatctacgccggcaacgccatcgccacggtcaagtcggaagacaagatcaaggtcatcaccgtgcgcggcaccgcctttgacgccgccgccgccgaaggtggctcggctgccgtcgagaccctgccggccgtggccgacgcaggcgtttcgcagtttgtttcgcgcgaagtggccaagagcgaccgtccggaactgaccgccgccaagatcatcgtctcgggtggccgtggcgtgggttcgggcgagaactacaccaaggtgctgacgccgctggccgacaagctgggcgccgcgctgggtgcctcgcgcgccgccgtggacgccggcttcgtgccgaacgactaccaggtcggccagaccggcaagatcgtcgcgccgcagctgtatatcgccgtcggtatctccggcgcgatccagcacctggccggcatgaaggactccaaggtgatcgttgcgatcaacaaggatgccgaagccccgatcttctccgtggccgactacggcctggtaggcgacctgaacaccgtggtgccggagctggtggcagcactgggctgatgccttgccggccgccaggcggccggcactggtagcaagtacgagccgttccgggggtgacccgcgaacggctttttcatacatgttgggagacatcgatgacttaccgtgcgccgatcaaggacatgctgttcgtcatgaacgagctggccgggctagaggccgtgagcaaactgccgggcttcgaggaagccacgccggaaacggccgaggcagtgctggacgaggccgccaagttcaacgagcaggtcgtcgcgccgctgaaccgcgccggcgacctggacccgagcagctggaaggacggcgtggtcaccaccacgcccggctttaaggaggccttccgccagttcggcgagggcggctggcagggcgtgctgcatccgcaggagtttggcggccagggcctgcccaagctggtcgccaccgcctgcaacgagatgctgaacaccgcgaacctgtcgttcgcgctgtgcccgctgctgaccgatggtgccatcgaagcgctgctgacggccggcacggatgagcagaaggccaccttcctgcccaagctgatctcgggcgagtggaccggcaccatgaacctgaccgagccgcaggccggctcggatctggccgcagtgcgcacccgtgccgagccgcagggcgacggcacctacaaagtgttcggcaccaagatcttcatcacctacggcgagcacgacatggcgaagaacatcgtccatctcgtgctggcccgcacgcccaccgcgcccgagggcgtcaagggcatctcgctgttcatcgtgccgaagttcatggtcaacgccgacggcagcaccggtgagcgcaacgacgtgcattgcgtctcgatcgagcacaagctgggcatcaaggcgagccccacggccgtgctgcagttcggcgaccatggcggcgccatcggcacgctggtcggcgaagagaaccgtggcctcgagtacatgttcatcatgatgaactcggcacgcttctcggtcggcatgcagggcatcgccgtatccgaacgcgcctaccagcaggcggtggcctttgcgcgcgagcgcgtgcagagccgcccggtcgatggttcggcgcgcgaggcggtgaccatcatccaccaccctgacgtcaagcgcatgctgatgaccatgcgcgcgctgaccgaaggcgcgcgcgccgtggcctacgtggccgcggccgccagcgacgcggcgcaccagcatcccgacgaagcggtgcgccagcagaaccaggccttctacgagttcctggtgccggtggtgaaaggctggagcaccgagctgtcgatcgacgtcaccagcctgggtgtgcaggtgcacggcggcatgggctttatcgaggagaccggcgcggcgcagcactaccgcgacgcgcgtatcctgccgatctacgaaggcaccacggcaatccaggccaacgacctgatcggccgcaagaccgtgcgcgacggcggcgccgtggcgcgtgcgatctgcgcgcagatcgccgagaccgaggctgcgctgggcaatcacagctgcgccgggttcaccgcggtacaggcgcaactggccaagggtcgcgcggcgctggaagacgtggtggcgtttgtcgtggccaatgccaagtccgacccgaacgccgtctttgccggcagcgtgccttacctgaagctgtgcggcatcgtgttctccggctggcagttcggccgtgcgatgctggccgccgatgcgaagcgggccggggacccgggcttttatgacgccaagatcgccaccgcgcatttctttgcggagcacatcctgtcgcaggcgtcggctttgcgcgatgccatcgtcagtggtgcggcaccggtgaatgcgatgacggccgagcagttctgagtggcctgacagtcgccggaaagaaaaagggcggccccgcggggccgccctttttgcttatcgaggcagcctgctggatcc

SEQ ID NO: 50 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 51 - protein, 252 aa, Clostridium acetobutylicum
MNIVVCLKQVPDTAEVRIDPVKGTLIREGVPSIINPDDKNALEEALVLKDNYGAHVTVISMGPPQAKNALVEALAMGADEAVLLTDRAFGGADTLATSHTIAAGIKKLKYDIVFAGRQAIDGDTAQVGPEIAEHLGIPQVTYVEKVEVDGDTLKIRKAWEDGYEVVEVKTPVLLTAIKELNVPRYMSVEKIFGAFDKEVKMWTADDIDVDKANLGLKGSPTKVKKSSTKEVKGQGEVIDKPVKEAADMLSQN

SEQ ID NO: 52 - protein, 337 aa, Clostridium acetobutylicum
MNKADYKGVWVFAEQRDGELQKVSLELLGKGKEMAEKLGVELTAVLLGHNTEKMSKDLLSHGADKVLAADNELLAHFSTDGYAKVICDLVNERKPEILFIGATFIGRDLGPRIAARLSTGLTADCTSLDIDVENRDLLATRPAFGGNLIATIVCSDHRPQMATVRPGVFFEKLPVNDANVSDDKIEKVAIKLTASDIRTKVSKVVKLAKDIADIGEAKVLVAGGRGVGSKENFEKLEELASLLGGTIAASRAAIEKEWVDKDLQVGQTGKTVRPTLYIACGISGAIQHLAGMQDSDYIIAINKDVEAPIMKVADLAIVGDVNKVVPELIAQVKAANN

SEQ ID NO: 53 - protein, 379 aa, Clostridium acetobutylicum
MDFNLTREQELVRQMVREFAENEVKPIAAEIDETERFPMENVKKMGQYGMMGIPFSKEYGGAGGDVLSYIIAVEELSKVCGTTGVILSAHTSLCASLINEHGTEEQKQKYLVPLAKGEKIGAYGLTEPNAGTDSGAQQTVAVLEGDHYVINGSKIFITNGGVADTFVIFAMTDRTKGTKGISAFIIEKGFKGFSIGKVEQKLGIRASSTTELVFEDMIVPVENMIGKEGKGFPIAMKTLDGGRIGIAAQALGIAEGAFNEARAYMKERKQFGRSLDKFQGLAWMMADMDVAIESARYLVYKAAYLKQAGLPYTVDAARAKLHAANVAMDVTTKAVQLFGGYGYTKDYPVERMMRDAKITEIYEGTSEVQKLVISGKIFR

SEQ ID NO: 54 - DNA, 3941 bp, Artificial Sequence
  synthetic gen
aagcttccccaggctttacactttatgcttccggctcgtatgttgtgtggaattgtgagcggataacaatttcacattttaggaggattagtcatggaactcaacaatgtcatcctggagaaagaaggcaaggtcgcggtggtgaccatcaaccgcccgaaggccctcaacgccctgaactcggacaccctgaaagagatggactacgtgatcggcgaaatcgagaacgactcggaggtgctcgccgtgatcctgacgggcgcgggcgagaagagcttcgtcgccggggcggacatctcggagatgaaagaaatgaataccatcgaggggcgcaagttcggcattctggggaacaaagtgtttcggcggctggagctgctggaaaaaccggtgattgcggccgtgaatgggttcgccctgggcggcgggtgcgaaattgcgatgtcctgcgatatccgcatcgcctcctcgaacgcccgcttcggccagccggaagtgggcctgggcatcaccccgggctttggcggcacccagcggctgtcgcgcctcgtcggcatgggcatggccaagcagctgattttcaccgcgcagaacatcaaggccgatgaggccctgcgcattggcctggtgaacaaggtggtggagccctcggaactgatgaacaccgccaaggagatcgccaacaaaatcgtgagcaatgccccggtggccgtcaaactgtcgaagcaagccattaaccgcggcatgcagtgcgacattgacacggccctcgccttcgagtcggaagcgttcggggaatgcttctcgaccgaggaccagaaagacgccatgaccgccttcattgagaagcgcaagatcgaagggttcaagaaccgctaggaggtaagtttatatggacttcaacctgacgcgcgagcaagagctggtgcgccagatggtccgggagttcgccgagaacgaggtgaagccgattgccgcggagatcgacgagacggagcggttcccgatggagaacgtgaagaagatgggccagtacggcatgatgggcatcccgttctcgaaggagtacggcggggccggcggcgacgtgctgtcgtacatcatcgccgtggaggagctgtcgaaggtgtgcggcaccaccggcgtgatcctgtcggcccacacctcgctgtgcgcctcgctgattaacgagcacggcaccgaggagcagaagcagaagtacctggtgccgctggccaagggggagaagatcggcgcctacggcctgaccgagccgaacgccggcaccgattcgggcgcccagcagaccgtggccgtgctggagggcgaccactacgtgatcaacggctcgaaaatcttcatcaccaacggcggcgtcgccgacaccttcgtcatcttcgccatgaccgaccgcaccaaggggaccaagggcatctcggcctttatcatcgagaagggcttcaagggcttctcgatcggcaaggtggagcaaaagctgggcatccgcgcgtcgtcgaccaccgagctggtgttcgaggacatgatcgtgccggtggagaacatgattggcaaggagggcaagggcttcccgatcgccatgaagaccctggacgggggccgcatcggcatcgcggcccaggccctgggcatcgccgaaggcgcctttaacgaggcccgcgcgtacatgaaggaacgcaagcagttcggccgctcgctggacaagttccagggcctggcctggatgatggccgacatggacgtggccatcgagtcggcccgctacctggtgtacaaggccgcctacctgaagcaggcgggcctgccgtacaccgtggatgccgcccgcgccaagctgcatgccgccaacgtggccatggacgtgaccaccaaggccgtgcagctgttcggcggctatggctacaccaaggactaccccgtggagcgcatgatgcgcgacgccaagatcacggagatctacgagggcacctcggaggtgcagaagctggtgatctcgggcaagatcttccgctaatttaaggaggttaagaggatgaatattgtggtctgcctcaaacaagtccccgacaccgccgaggtccgcattgatcccgtcaaaggcaccctgattcgggagggcgtcccgtccatcattaaccccgacgacaaaaacgcgctcgaggaagccctcgtgctgaaagataactatggggcgcacgtcacggtcatctcgatgggccccccccaagccaagaacgccctggtcgaagcgctggccatgggggccgatgaggcggtgctgctcacggaccgggcgtttgggggcgcggacaccctcgccacctcgcataccattgccgccggcattaagaagctgaaatatgacattgtctttgccggccgccaggccatcgacggcgatacggcgcaggtcgggccggagattgcggaacatctcgggattccccaagtcacctacgtggaaaaagtggaggtggatggggacacgctcaagatccggaaggcctgggaggatgggtacgaggtcgtggaagtcaagaccccggtgctcctgacggccattaaggaactgaacgtcccgcggtacatgtcggtggagaaaatcttcggcgcgttcgacaaggaggtgaaaatgtggaccgcggatgatatcgacgtggataaagccaatctgggcctgaaggggtcgccgaccaaagtgaagaagtcgagcacgaaagaagtgaaagggcagggcgaggtcattgataaaccggtgaaggaagcggccgatatgctctcgcagaactaaaagaagaacacatatttaagttaggagggatttttcaatgaacaaggccgactacaagggcgtgtgggtgtttgcggagcagcgcgacggcgagctgcagaaggtgtcgctggagctgctgggcaagggcaaggagatggccgagaagctgggggtggaactgaccgccgtgctgctgggccacaacaccgagaagatgtcgaaggacctgctgagccacggcgcggataaggtgctcgcggccgacaatgaactcctcgcccacttctcgaccgacggctacgcgaaggtgatctgcgacctcgtgaacgagcgcaagccggagatcctgttcattggcgccacgttcatcgggcgggacctgggcccgcggatcgcggcgcgcctgtcgaccggcctgaccgccgactgcacctcgctggacatcgacgtggagaaccgggacctcctggcgacccgcccggcgttcggcggcaacctgatcgccaccatcgtctgctcggaccaccgcccgcagatggccacggtccgccccggcgtcttcttcgagaagctgccggtgaacgacgccaacgtgtccgacgataagatcgagaaagtcgccatcaagctgaccgcgtccgacatccgcaccaaagtgtcgaaagtggtgaagctggccaaagacatcgcggacatcggcgaggccaaggtgctcgtggccgggggccgcggcgtgggctcgaaggagaacttcgaaaaactggaggagctggcctcgctcctgggcggcaccatcgccgcctcgcgcgcggccatcgagaaagagtgggtggacaaagacctccaggtgggccagaccggcaagaccgtgcgcccgaccctgtacatcgcctgcggcatctcgggggcgatccagcacctggccggcatgcaggactcggactacatcatcgccatcaataaggacgtggaggccccgattatgaaggtggccgacctggcgatcgtgggcgacgtgaacaaagtggtcccggagctgatcgcgcaggtgaaggccgccaacaactgagtggcctgacagtcgccggaaagaaaaagggcggccccgcggggccgccctttttgcttatcgaggcagcctgctggatcc

SEQ ID NO: 55 - DNA, 3145 bp, Artificial Sequence
  synthetic gen
aagcttccccaggctttacactttatgcttccggctcgtatgttgtgtggaattgtgagcggataacaatttcacatagataggaggtaagtttatatggacttcaacctgacccgcgagcaggagctggtgcgccagatggtgcgcgagttcgcggaaaacgaggtgaagcccattgccgcggagatcgacgagaccgagcgcttcccgatggagaacgtgaagaagatgggccagtatggcatgatggggattccgttctcgaaggagtacggcggggcggggggggacgtgctgtcgtacatcatcgccgtggaggagctgtccaaggtgtgcggcaccaccggggtgatcctgtcggcccacacctccctctgcgcctcgctgatcaacgagcacgggaccgaggagcagaaacagaagtacctggtcccgctggccaaaggggagaagatcggcgcgtacggcctgacggaaccgaacgccggcaccgactcgggcgcgcagcagaccgtggccgtcctggaaggcgaccactacgtgatcaacggctccaaaatctttattaccaacggcggcgtggcggacacgttcgtgatcttcgcgatgaccgaccgcaccaagggcaccaaaggcatctcggcctttatcattgagaaagggtttaagggcttctcgatcggcaaggtggagcaaaagctgggcatccgcgcgtcgtcgaccaccgaactggtgttcgaggacatgatcgtgccggtggaaaacatgattggcaaagaggggaaggggttcccgattgcgatgaaaaccctggatggggggcggatcggcattgcggcccaagccctggggatcgcggagggcgccttcaacgaagcccgggcctacatgaaggagcgcaagcagttcggccgctcgctggacaagtttcagggcctggcctggatgatggccgatatggacgtggccattgagtccgcccggtatctggtctataaggccgcctacctgaagcaggcgggcctgccgtacaccgtggacgccgcgcgcgccaagctgcatgccgccaacgtcgccatggacgtgaccaccaaagcggtgcaactgttcggcggctacggctacacgaaggactacccggtcgagcgcatgatgcgcgacgccaagattaccgagatctacgagggcacgtcggaagtgcagaagctggtgattagcggcaagattttccgctaatttaaggaggttaagaggatgaacatcgtggtgtgtctgaagcaggtgccggacaccgccgaggtgcgcatcgatccggtgaaggggaccctgatccgcgagggcgtgccgtcgatcatcaacccggacgacaagaatgcgctggaggaagcgctggtcctgaaagacaactacggggcccatgtgaccgtgatctcgatgggccccccgcaggccaagaacgccctggtggaggccctggccatgggggcggacgaggcggtgctgctgaccgaccgcgccttcgggggggcggataccctggcgacctcgcacaccatcgccgcgggcatcaagaagctgaaatatgatatcgtgtttgccgggcgccaggccattgacggcgacacggcccaggtcgggccggagatcgccgagcatctgggcatcccccaggtgacctacgtggagaaagtcgaggtggatggcgatacgctgaaaatccgcaaggcctgggaggacggctacgaggtcgtggaggtgaagaccccggtcctgctgacggccatcaaggagctgaacgtgccgcgctacatgtcggtggaaaagattttcggcgcgttcgacaaggaggtcaagatgtggaccgcggatgacatcgacgtcgacaaggccaacctggggctgaagggctcgccgaccaaggtgaaaaagtcgtcgaccaaagaagtcaagggccaaggcgaagtgatcgataagcccgtgaaggaggcggccgatatgctgtcgcagaactaaaagaagaacacatatttaagttaggagggatttttcaatgaacaaagccgactacaagggcgtgtgggtgttcgccgagcaacgcgacggcgagctgcagaaggtgtcgctggaactgctggggaagggcaaggaaatggccgagaaactgggcgtggagctgacggccgtgctgctggggcacaacaccgagaagatgtcgaaggacctgctgtcgcacggcgcggacaaggtcctggccgccgacaacgagctgctggcccatttcagcaccgacggctacgccaaggtgatctgtgacctggtgaacgagcgcaagccggagatcctgttcatcggcgccacgttcatcggccgcgacctcggcccgcggatcgccgcccggctgtcgaccggcctgaccgccgactgcacctcgctggacatcgacgtggagaaccgcgatctgctcgcgacccgcccggccttcggcggcaacctgatcgccaccatcgtgtgctcggaccaccgcccgcagatggccacggtgcggcccggcgtgttcttcgagaagctgccggtgaacgacgccaacgtgtcggacgacaagatcgagaaagtggccatcaagctgaccgcctcggacatccgcaccaaggtctcgaaagtggtgaagctcgccaaggatattgccgatatcggcgaggccaaggtgctcgtggccgggggccgcggcgtgggctcgaaggagaactttgagaagctggaggagctggcctccctcctcggcggcaccatcgccgcctcgcgcgccgccatcgaaaaggagtgggtggataaggacctccaagtggggcagaccggcaagaccgtgcgcccgaccctgtacatcgcctgcggcatctcgggcgccatccagcacctggccggcatgcaggactcggactacatcatcgcgatcaacaaggacgtggaagccccgatcatgaaggtggccgacctggccattgtgggcgatgtgaacaaggtggtgcccgagctgatcgcccaagtcaaggccgccaacaactgagtggcctgacagtcgccggaaagaaaaagggcggccccgcggggccgccctttttgcttatcgaggcagcctgctggatcc

SEQ ID NO: 56 - DNA, 10208 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttggggagaacaatcagcccggcaggggccgggctgattgtgcctgcgtgccttagaacgacttgatgtagatgtccttcagctccgagatcagcgggtagcgcgggttggcggtggtgcactggtcgtcgaaggccagctccgacatcttgtccagggtgttgtagaaatccttcttgttgatgccggcggccgagatgttctgcgggatcgacaggtcgatcttcagcttcgagatggcctcgatcagggcggtcaccttctccgtgtccgaggtgcccttcaggttcaggtactcggcgatctcggcgtacttgcgcttggcgttcggcgacttgtactgcgggaaggcggtctgcttggtcgggcagtcggtggcgttgtacttgatcacctcctcgatcagcacggcgcaggcgatgccgtgcggcacgtggtgcatggcgcccagcttgtgggccatcgagtggcacacgcccaggaaggcgttggcgaaggccatgccggcgatgttcgaggcgtgggccatcttctcgcgggcctcgatgtcgttggtgccgttcttgtaggcgcgcggcaggtacttgaagatcatcttgatggcgcgcagggccagctcgtcggtgtagtccgtggccatcaccgacacgtaggcctcgatggcgtgcaccagggcatcaatgccggtggcggcggtcagcttgcgcggcatgttcagcatcagctcggtgtcaatgatggccatgttcggggtcagttcgtacgaggtcagcgggtacttcatgccggtctcgtcgttggtgatcacggcgaacggggtggcctccgagccggtgccggcggtggtcgggatggccaccgagatggccttggtgcccagcttcgggaagttgcagatgcgcttgcggatgtccatgaaattgatggcgaggttctcgatctccgcctccgggtactcgtacagcaggtgcatcaccttggcggcgtccatcggcgagccgccgccgatcgagatgatggtgtccggctcgaagttcagcatctccttggcgcccttcttcaccgagtcaatggtcgggtccgacttgatatcggtgaagatcgagtacttgatgtcgatctcgtccagcaccttggtgatcttgttcacgtagcccagcttgaacaggtccttgtcggtcacgatgaaggcgcgcttcttgttcatgtccttcagctccttcagggcgaagcgcaggcagccgtacttgaagtagatcttctgcggcaccttgaaccacagcatattctcgcggcgctcggccaccgacttgatgttcagcaggtgcttcggctccacgttctgcgacaccgagttgccgccccaggtcccgcagcccagggtgaacgacggggcgatggcgaagttgtacaggtcgcccgaggcgccctgcgacgacggcatgttgatgaaggtgcgcgaggtcttcatggccaggccgaactccttcaccttgtccttgttgttctgcgagtcgatgtacagcgacgaggtgtggcccgagccgcccagctcgatcaggcgctgggccttcttgagggcctcatcgaagtccttcaccttgtacatggccagcaccggcgacagcttctcgtgcgagaacagctccgacttctccaccgactgcacctcgccgatgaggatcttggtggtctgcggcacctcgatgccggccatcttggcgatgatgtaggccgacttgcccacgatgtcggcgttgatggcgccgttcttgaacatcgtctccttgatcttggcgatctcgttctggttcaggatgtacgagccgcgcttcacgaactcctccttcaccttctcgtagatggagttcatcaccaggatcgactgctccgaggcgcagatcacgccgttgtcgtaggtcttcgacaggatgatcgacgacacggccatatcgatgtcggcggactcgtcgatgatggccggggtgttgcccgcgcccacgccgatggccggcttgcccgacgagtaggcggccttcaccatcgacgggccgccggtggccaggatgatatcggcctccgacatcaggtcctgcgacagctcgatcgacggctcgtcaatccagccgatgatgttcttcggggccccggccttcacggcggcgtccaggatcagcttggcggcggcgatggtcgacttcttggcgcgcgggtgcggcgagaagaagatggcgttgcgggtcttcagcgagatcagcgacttgaagatggcggtcgaggtcgggttcgtggtcggcacgatggcggccacgatgccgatcggctcggccaccttggtgatgcccagcgagtcgtcgtggtcgataatgccgcaggtcttctcgttcttgtacttgttgtagatgtactcggcggcgaagtggttcttgatgatcttgtcctccaccaggccgatgccggtctcctccacggccagcttggccaggttgatgcgctccttggcggcggcaatggcgcactgcttgaagatcttgtccacctgctcctgggtgtaggtggcgaacttcttctgggcctcgcgcagctcgttcagcttctgcttcagctccttctggttggtcaccttcattcttgaatctcctgaaagccggtgcttacggcagcttgaccacggcttccccggtgacggccagggccccgccttgggtaaagatccgggtcgtcagcgtggcgatcggtttgtcctcgcgcagcgcggtgacttccacctcggcggtcacttcgtcccccacgaacaccggcagtttgaacgagagcgattggcccaggtagatgctccccttgccggggagctgctggccgaggaggcccgagaacagcgaggcgagcagcatgccatgcacgatggggcgctcgaacgcggtggtcgcggcaaacgcggggtccaggtgcagcgggttgaagtcctcggagagcgccgcaaaggccgccacctccgccgcgccaaaccgcttggacagccgggctttttgcccgacctccagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 57 - DNA, 7614 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttggggagaacaatcagcccggcaggggccgggctgattgtgcctgcgtgccgccggtgcttacggcagcttgaccacggcttccccggtgacggccagggccccgccttgggtaaagatccgggtcgtcagcgtggcgatcggtttgtcctcgcgcagcgcggtgacttccacctcggcggtcacttcgtcccccacgaacaccggcagtttgaacgagagcgattggcccaggtagatgctccccttgccggggagctgctggccgaggaggcccgagaacagcgaggcgagcagcatgccatgcacgatggggcgctcgaacgcggtggtcgcggcaaacgcggggtccaggtgcagcgggttgaagtcctcggagagcgccgcaaaggccgccacctccgccgcgccaaaccgcttggacagccgggctttttgcccgacctccagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 58 - DNA, 9844 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttcttaagtaataaaaataagagttaccttaaatggtaactcttatttttttaatattgtttcatagtttagaacgacttgatgtagatgtccttcagctccgagatcagcgggtagcgcgggttggcggtggtgcactggtcgtcgaaggccagctccgacatcttgtccagggtgttgtagaaatccttcttgttgatgccggcggccgagatgttctgcgggatcgacaggtcgatcttcagcttcgagatggcctcgatcagggcggtcaccttctccgtgtccgaggtgcccttcaggttcaggtactcggcgatctcggcgtacttgcgcttggcgttcggcgacttgtactgcgggaaggcggtctgcttggtcgggcagtcggtggcgttgtacttgatcacctcctcgatcagcacggcgcaggcgatgccgtgcggcacgtggtgcatggcgcccagcttgtgggccatcgagtggcacacgcccaggaaggcgttggcgaaggccatgccggcgatgttcgaggcgtgggccatcttctcgcgggcctcgatgtcgttggtgccgttcttgtaggcgcgcggcaggtacttgaagatcatcttgatggcgcgcagggccagctcgtcggtgtagtccgtggccatcaccgacacgtaggcctcgatggcgtgcaccagggcatcaatgccggtggcggcggtcagcttgcgcggcatgttcagcatcagctcggtgtcaatgatggccatgttcggggtcagttcgtacgaggtcagcgggtacttcatgccggtctcgtcgttggtgatcacggcgaacggggtggcctccgagccggtgccggcggtggtcgggatggccaccgagatggccttggtgcccagcttcgggaagttgcagatgcgcttgcggatgtccatgaaattgatggcgaggttctcgatctccgcctccgggtactcgtacagcaggtgcatcaccttggcggcgtccatcggcgagccgccgccgatcgagatgatggtgtccggctcgaagttcagcatctccttggcgcccttcttcaccgagtcaatggtcgggtccgacttgatatcggtgaagatcgagtacttgatgtcgatctcgtccagcaccttggtgatcttgttcacgtagcccagcttgaacaggtccttgtcggtcacgatgaaggcgcgcttcttgttcatgtccttcagctccttcagggcgaagcgcaggcagccgtacttgaagtagatcttctgcggcaccttgaaccacagcatattctcgcggcgctcggccaccgacttgatgttcagcaggtgcttcggctccacgttctgcgacaccgagttgccgccccaggtcccgcagcccagggtgaacgacggggcgatggcgaagttgtacaggtcgcccgaggcgccctgcgacgacggcatgttgatgaaggtgcgcgaggtcttcatggccaggccgaactccttcaccttgtccttgttgttctgcgagtcgatgtacagcgacgaggtgtggcccgagccgcccagctcgatcaggcgctgggccttcttgagggcctcatcgaagtccttcaccttgtacatggccagcaccggcgacagcttctcgtgcgagaacagctccgacttctccaccgactgcacctcgccgatgaggatcttggtggtctgcggcacctcgatgccggccatcttggcgatgatgtaggccgacttgcccacgatgtcggcgttgatggcgccgttcttgaacatcgtctccttgatcttggcgatctcgttctggttcaggatgtacgagccgcgcttcacgaactcctccttcaccttctcgtagatggagttcatcaccaggatcgactgctccgaggcgcagatcacgccgttgtcgtaggtcttcgacaggatgatcgacgacacggccatatcgatgtcggcggactcgtcgatgatggccggggtgttgcccgcgcccacgccgatggccggcttgcccgacgagtaggcggccttcaccatcgacgggccgccggtggccaggatgatatcggcctccgacatcaggtcctgcgacagctcgatcgacggctcgtcaatccagccgatgatgttcttcggggccccggccttcacggcggcgtccaggatcagcttggcggcggcgatggtcgacttcttggcgcgcgggtgcggcgagaagaagatggcgttgcgggtcttcagcgagatcagcgacttgaagatggcggtcgaggtcgggttcgtggtcggcacgatggcggccacgatgccgatcggctcggccaccttggtgatgcccagcgagtcgtcgtggtcgataatgccgcaggtcttctcgttcttgtacttgttgtagatgtactcggcggcgaagtggttcttgatgatcttgtcctccaccaggccgatgccggtctcctccacggccagcttggccaggttgatgcgctccttggcggcggcaatggcgcactgcttgaagatcttgtccacctgctcctgggtgtaggtggcgaacttcttctgggcctcgcgcagctcgttcagcttctgcttcagctccttctggttggtcaccttcattcttgaatctcctgaaaatttctttttatttcgagtagtcgtaaaagcccttgccgctcttgcggcccagccagcccgcccggacatactttttcagcagcgtgtgcgggcgatacttcgaatcgccggtttccgagtacagcacgtccatgatggccaggcaaatgtcgaggccgataaagtcccccagttccagcgggcccatcgggtggttcgcccccagcttcatggccttatcaatatcctcgaccgaggcaatcccttcggcgaggatgcccaccgcttcattgatcatcgggatcagaatgcggttgacgacgaaccccggcgcctcggcgacttccacggggtccttgccaatggcaatcgacgtctctttcaccgcgtcgaaggtttcctgcgacgtggcaatgccgcgaatgacctccacgagtttcatgacgggggcgggattaaagaaatgcatgccgatcactttgtcgttggtcttggtcgcgctcgccacctccgtgatcgagagcgagctggtgttggaggcgagaatcgtctccggtttgcagatgttgtcgagatccgcaaagatctgtttcttaatgtccatgcgctcgacggcggcttcaatcacgaggtcgcagtccgcggccatgttcaggtccacggtcccgctgatgcgggtgagaatctccaccttggtggcctcttcgatcttgccttttttgaccagtttcgacaggttcttgttaatgaaatccaggccgcgatcgacgaattcatccttgatgtcccggaggaccacctcgaagcctttggcggcgaaggcctgcgcgatgccggaccccatcgtccccgcgccaatgacgcacaccttcttcattcttgaatctcctgaaactagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 59 - DNA, 7250 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttcttaagtaataaaaataagagttaccttaaatggtaactcttatttttttaatattgtttcatagtatttctttttatttcgagtagtcgtaaaagcccttgccgctcttgcggcccagccagcccgcccggacatactttttcagcagcgtgtgcgggcgatacttcgaatcgccggtttccgagtacagcacgtccatgatggccaggcaaatgtcgaggccgataaagtcccccagttccagcgggcccatcgggtggttcgcccccagcttcatggccttatcaatatcctcgaccgaggcaatcccttcggcgaggatgcccaccgcttcattgatcatcgggatcagaatgcggttgacgacgaaccccggcgcctcggcgacttccacggggtccttgccaatggcaatcgacgtctctttcaccgcgtcgaaggtttcctgcgacgtggcaatgccgcgaatgacctccacgagtttcatgacgggggcgggattaaagaaatgcatgccgatcactttgtcgttggtcttggtcgcgctcgccacctccgtgatcgagagcgagctggtgttggaggcgagaatcgtctccggtttgcagatgttgtcgagatccgcaaagatctgtttcttaatgtccatgcgctcgacggcggcttcaatcacgaggtcgcagtccgcggccatgttcaggtccacggtcccgctgatgcgggtgagaatctccaccttggtggcctcttcgatcttgccttttttgaccagtttcgacaggttcttgttaatgaaatccaggccgcgatcgacgaattcatccttgatgtcccggaggaccacctcgaagcctttggcggcgaaggcctgcgcgatgccggaccccatcgtccccgcgccaatgacgcacaccttcttcattcttgaatctcctgaaactagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 60 - DNA, 14113 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccagcaggctgcctcgataagcaaaaagggcggccccgcggggccgccctttttctttccggcgactgtcaggccactcagttgttggcggccttcacctgcgcgatcagctccgggaccactttgttcacgtcgcccacgatcgccaggtcggccaccttcataatcggggcctccacgtccttattgatggcgatgatgtagtccgagtcctgcatgccggccaggtgctggatcgcccccgagatgccgcaggcgatgtacagggtcgggcgcacggtcttgccggtctggcccacctggaggtctttgtccacccactctttctcgatggccgcgcgcgaggcggcgatggtgccgcccaggagcgaggccagctcctccagtttttcgaagttctccttcgagcccacgccgcggcccccggccacgagcaccttggcctcgccgatgtccgcgatgtctttggccagcttcaccactttcgacactttggtgcggatgtcggacgcggtcagcttgatggcgactttctcgatcttatcgtcggacacgttggcgtcgttcaccggcagcttctcgaagaagacgccggggcggaccgtggccatctgcgggcggtggtccgagcagacgatggtggcgatcaggttgccgccgaacgccgggcgggtcgccaggaggtcccggttctccacgtcgatgtccagcgaggtgcagtcggcggtcaggccggtcgacaggcgcgccgcgatccgcgggcccaggtcccgcccgatgaacgtggcgccaatgaacaggatctccggcttgcgctcgttcacgaggtcgcagatcaccttcgcgtagccgtcggtcgagaagtgggcgaggagttcattgtcggccgcgagcaccttatccgcgccgtggctcagcaggtccttcgacatcttctcggtgttgtggcccagcagcacggcggtcagttccacccccagcttctcggccatctccttgcccttgcccagcagctccagcgacaccttctgcagctcgccgtcgcgctgctccgcaaacacccacacgcccttgtagtcggccttgttcattgaaaaatccctcctaacttaaatatgtgttcttcttttagttctgcgagagcatatcggccgcttccttcaccggtttatcaatgacctcgccctgccctttcacttctttcgtgctcgacttcttcactttggtcggcgaccccttcaggcccagattggctttatccacgtcgatatcatccgcggtccacattttcacctccttgtcgaacgcgccgaagattttctccaccgacatgtaccgcgggacgttcagttccttaatggccgtcaggagcaccggggtcttgacttccacgacctcgtacccatcctcccaggccttccggatcttgagcgtgtccccatccacctccactttttccacgtaggtgacttggggaatcccgagatgttccgcaatctccggcccgacctgcgccgtatcgccgtcgatggcctggcggccggcaaagacaatgtcatatttcagcttcttaatgccggcggcaatggtatgcgaggtggcgagggtgtccgcgcccccaaacgcccggtccgtgagcagcaccgcctcatcggcccccatggccagcgcttcgaccagggcgttcttggcttggggggggcccatcgagatgaccgtgacgtgcgccccatagttatctttcagcacgagggcttcctcgagcgcgtttttgtcgtcggggttaatgatggacgggacgccctcccgaatcagggtgcctttgacgggatcaatgcggacctcggcggtgtcggggacttgtttgaggcagaccacaatattcatcctcttaacctccttaaattagcggaagatcttgcccgagatcaccagcttctgcacctccgaggtgccctcgtagatctccgtgatcttggcgtcgcgcatcatgcgctccacggggtagtccttggtgtagccatagccgccgaacagctgcacggccttggtggtcacgtccatggccacgttggcggcatgcagcttggcgcgggcggcatccacggtgtacggcaggcccgcctgcttcaggtaggcggccttgtacaccaggtagcgggccgactcgatggccacgtccatgtcggccatcatccaggccaggccctggaacttgtccagcgagcggccgaactgcttgcgttccttcatgtacgcgcgggcctcgttaaaggcgccttcggcgatgcccagggcctgggccgcgatgccgatgcggcccccgtccagggtcttcatggcgatcgggaagcccttgccctccttgccaatcatgttctccaccggcacgatcatgtcctcgaacaccagctcggtggtcgacgacgcgcggatgcccagcttttgctccaccttgccgatcgagaagcccttgaagcccttctcgatgataaaggccgagatgcccttggtccccttggtgcggtcggtcatggcgaagatgacgaaggtgtcggcgacgccgccgttggtgatgaagattttcgagccgttgatcacgtagtggtcgccctccagcacggccacggtctgctgggcgcccgaatcggtgccggcgttcggctcggtcaggccgtaggcgccgatcttctcccccttggccagcggcaccaggtacttctgcttctgctcctcggtgccgtgctcgttaatcagcgaggcgcacagcgaggtgtgggccgacaggatcacgccggtggtgccgcacaccttcgacagctcctccacggcgatgatgtacgacagcacgtcgccgccggccccgccgtactccttcgagaacgggatgcccatcatgccgtactggcccatcttcttcacgttctccatcgggaaccgctccgtctcgtcgatctccgcggcaatcggcttcacctcgttctcggcgaactcccggaccatctggcgcaccagctcttgctcgcgcgtcaggttgaagtccatataaacttacctcctagcggttcttgaacccttcgatcttgcgcttctcaatgaaggcggtcatggcgtctttctggtcctcggtcgagaagcattccccgaacgcttccgactcgaaggcgagggccgtgtcaatgtcgcactgcatgccgcggttaatggcttgcttcgacagtttgacggccaccggggcattgctcacgattttgttggcgatctccttggcggtgttcatcagttccgagggctccaccaccttgttcaccaggccaatgcgcagggcctcatcggccttgatgttctgcgcggtgaaaatcagctgcttggccatgcccatgccgacgaggcgcgacagccgctgggtgccgccaaagcccggggtgatgcccaggcccacttccggctggccgaagcgggcgttcgaggaggcgatgcggatatcgcaggacatcgcaatttcgcacccgccgcccagggcgaacccattcacggccgcaatcaccggtttttccagcagctccagccgccgaaacactttgttccccagaatgccgaacttgcgcccctcgatggtattcatttctttcatctccgagatgtccgccccggcgacgaagctcttctcgcccgcgcccgtcaggatcacggcgagcacctccgagtcgttctcgatttcgccgatcacgtagtccatctctttcagggtgtccgagttcagggcgttgagggccttcgggcggttgatggtcaccaccgcgaccttgccttctttctccaggatgacattgttgagttccatgactaatcctcctaaaatgtgaaattgttatccgctcacaattccacacaacatacgagccggaagcataaagtgtaaagcctggggaagcttggggagaacaatcagcccggcaggggccgggctgattgtgcctgcgtgccttagaacgacttgatgtagatgtccttcagctccgagatcagcgggtagcgcgggttggcggtggtgcactggtcgtcgaaggccagctccgacatcttgtccagggtgttgtagaaatccttcttgttgatgccggcggccgagatgttctgcgggatcgacaggtcgatcttcagcttcgagatggcctcgatcagggcggtcaccttctccgtgtccgaggtgcccttcaggttcaggtactcggcgatctcggcgtacttgcgcttggcgttcggcgacttgtactgcgggaaggcggtctgcttggtcgggcagtcggtggcgttgtacttgatcacctcctcgatcagcacggcgcaggcgatgccgtgcggcacgtggtgcatggcgcccagcttgtgggccatcgagtggcacacgcccaggaaggcgttggcgaaggccatgccggcgatgttcgaggcgtgggccatcttctcgcgggcctcgatgtcgttggtgccgttcttgtaggcgcgcggcaggtacttgaagatcatcttgatggcgcgcagggccagctcgtcggtgtagtccgtggccatcaccgacacgtaggcctcgatggcgtgcaccagggcatcaatgccggtggcggcggtcagcttgcgcggcatgttcagcatcagctcggtgtcaatgatggccatgttcggggtcagttcgtacgaggtcagcgggtacttcatgccggtctcgtcgttggtgatcacggcgaacggggtggcctccgagccggtgccggcggtggtcgggatggccaccgagatggccttggtgcccagcttcgggaagttgcagatgcgcttgcggatgtccatgaaattgatggcgaggttctcgatctccgcctccgggtactcgtacagcaggtgcatcaccttggcggcgtccatcggcgagccgccgccgatcgagatgatggtgtccggctcgaagttcagcatctccttggcgcccttcttcaccgagtcaatggtcgggtccgacttgatatcggtgaagatcgagtacttgatgtcgatctcgtccagcaccttggtgatcttgttcacgtagcccagcttgaacaggtccttgtcggtcacgatgaaggcgcgcttcttgttcatgtccttcagctccttcagggcgaagcgcaggcagccgtacttgaagtagatcttctgcggcaccttgaaccacagcatattctcgcggcgctcggccaccgacttgatgttcagcaggtgcttcggctccacgttctgcgacaccgagttgccgccccaggtcccgcagcccagggtgaacgacggggcgatggcgaagttgtacaggtcgcccgaggcgccctgcgacgacggcatgttgatgaaggtgcgcgaggtcttcatggccaggccgaactccttcaccttgtccttgttgttctgcgagtcgatgtacagcgacgaggtgtggcccgagccgcccagctcgatcaggcgctgggccttcttgagggcctcatcgaagtccttcaccttgtacatggccagcaccggcgacagcttctcgtgcgagaacagctccgacttctccaccgactgcacctcgccgatgaggatcttggtggtctgcggcacctcgatgccggccatcttggcgatgatgtaggccgacttgcccacgatgtcggcgttgatggcgccgttcttgaacatcgtctccttgatcttggcgatctcgttctggttcaggatgtacgagccgcgcttcacgaactcctccttcaccttctcgtagatggagttcatcaccaggatcgactgctccgaggcgcagatcacgccgttgtcgtaggtcttcgacaggatgatcgacgacacggccatatcgatgtcggcggactcgtcgatgatggccggggtgttgcccgcgcccacgccgatggccggcttgcccgacgagtaggcggccttcaccatcgacgggccgccggtggccaggatgatatcggcctccgacatcaggtcctgcgacagctcgatcgacggctcgtcaatccagccgatgatgttcttcggggccccggccttcacggcggcgtccaggatcagcttggcggcggcgatggtcgacttcttggcgcgcgggtgcggcgagaagaagatggcgttgcgggtcttcagcgagatcagcgacttgaagatggcggtcgaggtcgggttcgtggtcggcacgatggcggccacgatgccgatcggctcggccaccttggtgatgcccagcgagtcgtcgtggtcgataatgccgcaggtcttctcgttcttgtacttgttgtagatgtactcggcggcgaagtggttcttgatgatcttgtcctccaccaggccgatgccggtctcctccacggccagcttggccaggttgatgcgctccttggcggcggcaatggcgcactgcttgaagatcttgtccacctgctcctgggtgtaggtggcgaacttcttctgggcctcgcgcagctcgttcagcttctgcttcagctccttctggttggtcaccttcattcttgaatctcctgaaagccggtgcttacggcagcttgaccacggcttccccggtgacggccagggccccgccttgggtaaagatccgggtcgtcagcgtggcgatcggtttgtcctcgcgcagcgcggtgacttccacctcggcggtcacttcgtcccccacgaacaccggcagtttgaacgagagcgattggcccaggtagatgctccccttgccggggagctgctggccgaggaggcccgagaacagcgaggcgagcagcatgccatgcacgatggggcgctcgaacgcggtggtcgcggcaaacgcggggtccaggtgcagcgggttgaagtcctcggagagcgccgcaaaggccgccacctccgccgcgccaaaccgcttggacagccgggctttttgcccgacctccagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 61 - DNA, 11340 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccagcaggctgcctcgataagcaaaaagggcggccccgcggggccgccctttttctttccggcgactgtcaggccactcagaactgctcggccgtcatcgcattcaccggtgccgcaccactgacgatggcatcgcgcaaagccgacgcctgcgacaggatgtgctccgcaaagaaatgcgcggtggcgatcttggcgtcataaaagcccgggtccccggcccgcttcgcatcggcggccagcatcgcacggccgaactgccagccggagaacacgatgccgcacagcttcaggtaaggcacgctgccggcaaagacggcgttcgggtcggacttggcattggccacgacaaacgccaccacgtcttccagcgccgcgcgacccttggccagttgcgcctgtaccgcggtgaacccggcgcagctgtgattgcccagcgcagcctcggtctcggcgatctgcgcgcagatcgcacgcgccacggcgccgccgtcgcgcacggtcttgcggccgatcaggtcgttggcctggattgccgtggtgccttcgtagatcggcaggatacgcgcgtcgcggtagtgctgcgccgcgccggtctcctcgataaagcccatgccgccgtgcacctgcacacccaggctggtgacgtcgatcgacagctcggtgctccagcctttcaccaccggcaccaggaactcgtagaaggcctggttctgctggcgcaccgcttcgtcgggatgctggtgcgccgcgtcgctggcggccgcggccacgtaggccacggcgcgcgcgccttcggtcagcgcgcgcatggtcatcagcatgcgcttgacgtcagggtggtggatgatggtcaccgcctcgcgcgccgaaccatcgaccgggcggctctgcacgcgctcgcgcgcaaaggccaccgcctgctggtaggcgcgttcggatacggcgatgccctgcatgccgaccgagaagcgtgccgagttcatcatgatgaacatgtactcgaggccacggttctcttcgccgaccagcgtgccgatggcgccgccatggtcgccgaactgcagcacggccgtggggctcgccttgatgcccagcttgtgctcgatcgagacgcaatgcacgtcgttgcgctcaccggtgctgccgtcggcgttgaccatgaacttcggcacgatgaacagcgagatgcccttgacgccctcgggcgcggtgggcgtgcgggccagcacgagatggacgatgttcttcgccatgtcgtgctcgccgtaggtgatgaagatcttggtgccgaacactttgtaggtgccgtcgccctgcggctcggcacgggtgcgcactgcggccagatccgagccggcctgcggctcggtcaggttcatggtgccggtccactcgcccgagatcagcttgggcaggaaggtggccttctgctcatccgtgccggccgtcagcagcgcttcgatggcaccatcggtcagcagcgggcacagcgcgaacgacaggttcgcggtgttcagcatctcgttgcaggcggtggcgaccagcttgggcaggccctggccgccaaactcctgcggatgcagcacgccctgccagccgccctcgccgaactggcggaaggcctccttaaagccgggcgtggtggtgaccacgccgtccttccagctgctcgggtccaggtcgccggcgcggttcagcggcgcgacgacctgctcgttgaacttggcggcctcgtccagcactgcctcggccgtttccggcgtggcttcctcgaagcccggcagtttgctcacggcctctagcccggccagctcgttcatgacgaacagcatgtccttgatcggcgcacggtaagtcatcgatgtctcccaacatgtatgaaaaagccgttcgcgggtcacccccggaacggctcgtacttgctaccagtgccggccgcctggcggccggcaaggcatcagcccagtgctgccaccagctccggcaccacggtgttcaggtcgcctaccaggccgtagtcggccacggagaagatcggggcttcggcatccttgttgatcgcaacgatcaccttggagtccttcatgccggccaggtgctggatcgcgccggagataccgacggcgatatacagctgcggcgcgacgatcttgccggtctggccgacctggtagtcgttcggcacgaagccggcgtccacggcggcgcgcgaggcacccagcgcggcgcccagcttgtcggccagcggcgtcagcaccttggtgtagttctcgcccgaacccacgccacggccacccgagacgatgatcttggcggcggtcagttccggacggtcgctcttggccacttcgcgcgaaacaaactgcgaaacgcctgcgtcggccacggccggcagggtctcgacggcagccgagccaccttcggcggcggcggcgtcaaaggcggtgccgcgcacggtgatgaccttgatcttgtcttccgacttgaccgtggcgatggcgttgccggcgtagatcgggcgctcgaacgtgtccggggcgtcgaccttggagatttccgagatctgggccacgtccagcttggcagccacgcgcggcaggatgttcttgccgtacggggtggccggagccaggatatgcgagtagtcgttggcgatggccagcgcctgctcggccacgttctcggccaggccgtcaccgaagtacggggcgtcggccagcaggaccttggtcacgccggcgatcttggcggccgcatcggccgcggccttggcgttggcaccagccaccagcacgtgcacgtcgccgccgcactgggcagcggcagtcacggtgttcagcgtggcggccttgatggattgattgtcgtgttcagcaatgacgagtgcagtcatgttatttctcccccgcgctcagataaccttggcttcgttcttcagcttctgcaccagcgtcgcaacgtccggcaccatcacacccgcgctgcgcttggcaggctcgaccaccttcagggtcgacaggcgcggcttgacgtcgacgccgaggtcttccggcttgacgatatcgagctgcttcttcttggccttcatgatgttcggcagcgtcacgtagcgcggctcgttcaggcgcaggtcggtcgtcaccacggccggcagcttgagcgacagcgtttccaggccgccatcgacttcacgcgtcaccgaggccttgccgtcggcaaccaccaccttggaggcgaacgtggcttgcggcaggccagccagcgcggccaccatctggccggtctggttggagtcgtcgtcgatggcctgcttgcccaggatcaccagttgcggctgttccttgtcgatcagcgccttcagcagcttggccacggccagcggttgcaggtcttcgttcgattccaccaggatgccgcggtcggcaccgatggccatcgcggtgcgcagggtttcctggcactgcgtgacaccgcacgagacggcgatcacttcggtgacaacaccggcttccttcaggcgcacggcctcttccacggcgatttcgtcgaaggggttcatgctcatcttcacgttggccagatcgacgccgctgccgtccgccttgacgcggaccttgacgttgtaatccaccacccgcttgactgcgacgagtactttcatgcgctcactccactgatatgtgaaattgttatccgctcacaattccacacaacatacgagccggaagcataaagtgtaaagcctggggaagcttggggagaacaatcagcccggcaggggccgggctgattgtgcctgcgtgccgccggtgcttacggcagcttgaccacggcttccccggtgacggccagggccccgccttgggtaaagatccgggtcgtcagcgtggcgatcggtttgtcctcgcgcagcgcggtgacttccacctcggcggtcacttcgtcccccacgaacaccggcagtttgaacgagagcgattggcccaggtagatgctccccttgccggggagctgctggccgaggaggcccgagaacagcgaggcgagcagcatgccatgcacgatggggcgctcgaacgcggtggtcgcggcaaacgcggggtccaggtgcagcgggttgaagtcctcggagagcgccgcaaaggccgccacctccgccgcgccaaaccgcttggacagccgggctttttgcccgacctccagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 62 - DNA, 14113 bp, Artificial Sequence; identical to SEQ ID NO: 60
  vector

SEQ ID NO: 63 - DNA, 11519 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccagcaggctgcctcgataagcaaaaagggcggccccgcggggccgccctttttctttccggcgactgtcaggccactcagttgttggcggccttcacctgcgcgatcagctccgggaccactttgttcacgtcgcccacgatcgccaggtcggccaccttcataatcggggcctccacgtccttattgatggcgatgatgtagtccgagtcctgcatgccggccaggtgctggatcgcccccgagatgccgcaggcgatgtacagggtcgggcgcacggtcttgccggtctggcccacctggaggtctttgtccacccactctttctcgatggccgcgcgcgaggcggcgatggtgccgcccaggagcgaggccagctcctccagtttttcgaagttctccttcgagcccacgccgcggcccccggccacgagcaccttggcctcgccgatgtccgcgatgtctttggccagcttcaccactttcgacactttggtgcggatgtcggacgcggtcagcttgatggcgactttctcgatcttatcgtcggacacgttggcgtcgttcaccggcagcttctcgaagaagacgccggggcggaccgtggccatctgcgggcggtggtccgagcagacgatggtggcgatcaggttgccgccgaacgccgggcgggtcgccaggaggtcccggttctccacgtcgatgtccagcgaggtgcagtcggcggtcaggccggtcgacaggcgcgccgcgatccgcgggcccaggtcccgcccgatgaacgtggcgccaatgaacaggatctccggcttgcgctcgttcacgaggtcgcagatcaccttcgcgtagccgtcggtcgagaagtgggcgaggagttcattgtcggccgcgagcaccttatccgcgccgtggctcagcaggtccttcgacatcttctcggtgttgtggcccagcagcacggcggtcagttccacccccagcttctcggccatctccttgcccttgcccagcagctccagcgacaccttctgcagctcgccgtcgcgctgctccgcaaacacccacacgcccttgtagtcggccttgttcattgaaaaatccctcctaacttaaatatgtgttcttcttttagttctgcgagagcatatcggccgcttccttcaccggtttatcaatgacctcgccctgccctttcacttctttcgtgctcgacttcttcactttggtcggcgaccccttcaggcccagattggctttatccacgtcgatatcatccgcggtccacattttcacctccttgtcgaacgcgccgaagattttctccaccgacatgtaccgcgggacgttcagttccttaatggccgtcaggagcaccggggtcttgacttccacgacctcgtacccatcctcccaggccttccggatcttgagcgtgtccccatccacctccactttttccacgtaggtgacttggggaatcccgagatgttccgcaatctccggcccgacctgcgccgtatcgccgtcgatggcctggcggccggcaaagacaatgtcatatttcagcttcttaatgccggcggcaatggtatgcgaggtggcgagggtgtccgcgcccccaaacgcccggtccgtgagcagcaccgcctcatcggcccccatggccagcgcttcgaccagggcgttcttggcttggggggggcccatcgagatgaccgtgacgtgcgccccatagttatctttcagcacgagggcttcctcgagcgcgtttttgtcgtcggggttaatgatggacgggacgccctcccgaatcagggtgcctttgacgggatcaatgcggacctcggcggtgtcggggacttgtttgaggcagaccacaatattcatcctcttaacctccttaaattagcggaagatcttgcccgagatcaccagcttctgcacctccgaggtgccctcgtagatctccgtgatcttggcgtcgcgcatcatgcgctccacggggtagtccttggtgtagccatagccgccgaacagctgcacggccttggtggtcacgtccatggccacgttggcggcatgcagcttggcgcgggcggcatccacggtgtacggcaggcccgcctgcttcaggtaggcggccttgtacaccaggtagcgggccgactcgatggccacgtccatgtcggccatcatccaggccaggccctggaacttgtccagcgagcggccgaactgcttgcgttccttcatgtacgcgcgggcctcgttaaaggcgccttcggcgatgcccagggcctgggccgcgatgccgatgcggcccccgtccagggtcttcatggcgatcgggaagcccttgccctccttgccaatcatgttctccaccggcacgatcatgtcctcgaacaccagctcggtggtcgacgacgcgcggatgcccagcttttgctccaccttgccgatcgagaagcccttgaagcccttctcgatgataaaggccgagatgcccttggtccccttggtgcggtcggtcatggcgaagatgacgaaggtgtcggcgacgccgccgttggtgatgaagattttcgagccgttgatcacgtagtggtcgccctccagcacggccacggtctgctgggcgcccgaatcggtgccggcgttcggctcggtcaggccgtaggcgccgatcttctcccccttggccagcggcaccaggtacttctgcttctgctcctcggtgccgtgctcgttaatcagcgaggcgcacagcgaggtgtgggccgacaggatcacgccggtggtgccgcacaccttcgacagctcctccacggcgatgatgtacgacagcacgtcgccgccggccccgccgtactccttcgagaacgggatgcccatcatgccgtactggcccatcttcttcacgttctccatcgggaaccgctccgtctcgtcgatctccgcggcaatcggcttcacctcgttctcggcgaactcccggaccatctggcgcaccagctcttgctcgcgcgtcaggttgaagtccatataaacttacctcctagcggttcttgaacccttcgatcttgcgcttctcaatgaaggcggtcatggcgtctttctggtcctcggtcgagaagcattccccgaacgcttccgactcgaaggcgagggccgtgtcaatgtcgcactgcatgccgcggttaatggcttgcttcgacagtttgacggccaccggggcattgctcacgattttgttggcgatctccttggcggtgttcatcagttccgagggctccaccaccttgttcaccaggccaatgcgcagggcctcatcggccttgatgttctgcgcggtgaaaatcagctgcttggccatgcccatgccgacgaggcgcgacagccgctgggtgccgccaaagcccggggtgatgcccaggcccacttccggctggccgaagcgggcgttcgaggaggcgatgcggatatcgcaggacatcgcaatttcgcacccgccgcccagggcgaacccattcacggccgcaatcaccggtttttccagcagctccagccgccgaaacactttgttccccagaatgccgaacttgcgcccctcgatggtattcatttctttcatctccgagatgtccgccccggcgacgaagctcttctcgcccgcgcccgtcaggatcacggcgagcacctccgagtcgttctcgatttcgccgatcacgtagtccatctctttcagggtgtccgagttcagggcgttgagggccttcgggcggttgatggtcaccaccgcgaccttgccttctttctccaggatgacattgttgagttccatgactaatcctcctaaaatgtgaaattgttatccgctcacaattccacacaacatacgagccggaagcataaagtgtaaagcctggggaagcttggggagaacaatcagcccggcaggggccgggctgattgtgcctgcgtgccgccggtgcttacggcagcttgaccacggcttccccggtgacggccagggccccgccttgggtaaagatccgggtcgtcagcgtggcgatcggtttgtcctcgcgcagcgcggtgacttccacctcggcggtcacttcgtcccccacgaacaccggcagtttgaacgagagcgattggcccaggtagatgctccccttgccggggagctgctggccgaggaggcccgagaacagcgaggcgagcagcatgccatgcacgatggggcgctcgaacgcggtggtcgcggcaaacgcggggtccaggtgcagcgggttgaagtcctcggagagcgccgcaaaggccgccacctccgccgcgccaaaccgcttggacagccgggctttttgcccgacctccagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 64 - DNA, 13749 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccagcaggctgcctcgataagcaaaaagggcggccccgcggggccgccctttttctttccggcgactgtcaggccactcagttgttggcggccttcacctgcgcgatcagctccgggaccactttgttcacgtcgcccacgatcgccaggtcggccaccttcataatcggggcctccacgtccttattgatggcgatgatgtagtccgagtcctgcatgccggccaggtgctggatcgcccccgagatgccgcaggcgatgtacagggtcgggcgcacggtcttgccggtctggcccacctggaggtctttgtccacccactctttctcgatggccgcgcgcgaggcggcgatggtgccgcccaggagcgaggccagctcctccagtttttcgaagttctccttcgagcccacgccgcggcccccggccacgagcaccttggcctcgccgatgtccgcgatgtctttggccagcttcaccactttcgacactttggtgcggatgtcggacgcggtcagcttgatggcgactttctcgatcttatcgtcggacacgttggcgtcgttcaccggcagcttctcgaagaagacgccggggcggaccgtggccatctgcgggcggtggtccgagcagacgatggtggcgatcaggttgccgccgaacgccgggcgggtcgccaggaggtcccggttctccacgtcgatgtccagcgaggtgcagtcggcggtcaggccggtcgacaggcgcgccgcgatccgcgggcccaggtcccgcccgatgaacgtggcgccaatgaacaggatctccggcttgcgctcgttcacgaggtcgcagatcaccttcgcgtagccgtcggtcgagaagtgggcgaggagttcattgtcggccgcgagcaccttatccgcgccgtggctcagcaggtccttcgacatcttctcggtgttgtggcccagcagcacggcggtcagttccacccccagcttctcggccatctccttgcccttgcccagcagctccagcgacaccttctgcagctcgccgtcgcgctgctccgcaaacacccacacgcccttgtagtcggccttgttcattgaaaaatccctcctaacttaaatatgtgttcttcttttagttctgcgagagcatatcggccgcttccttcaccggtttatcaatgacctcgccctgccctttcacttctttcgtgctcgacttcttcactttggtcggcgaccccttcaggcccagattggctttatccacgtcgatatcatccgcggtccacattttcacctccttgtcgaacgcgccgaagattttctccaccgacatgtaccgcgggacgttcagttccttaatggccgtcaggagcaccggggtcttgacttccacgacctcgtacccatcctcccaggccttccggatcttgagcgtgtccccatccacctccactttttccacgtaggtgacttggggaatcccgagatgttccgcaatctccggcccgacctgcgccgtatcgccgtcgatggcctggcggccggcaaagacaatgtcatatttcagcttcttaatgccggcggcaatggtatgcgaggtggcgagggtgtccgcgcccccaaacgcccggtccgtgagcagcaccgcctcatcggcccccatggccagcgcttcgaccagggcgttcttggcttggggggggcccatcgagatgaccgtgacgtgcgccccatagttatctttcagcacgagggcttcctcgagcgcgtttttgtcgtcggggttaatgatggacgggacgccctcccgaatcagggtgcctttgacgggatcaatgcggacctcggcggtgtcggggacttgtttgaggcagaccacaatattcatcctcttaacctccttaaattagcggaagatcttgcccgagatcaccagcttctgcacctccgaggtgccctcgtagatctccgtgatcttggcgtcgcgcatcatgcgctccacggggtagtccttggtgtagccatagccgccgaacagctgcacggccttggtggtcacgtccatggccacgttggcggcatgcagcttggcgcgggcggcatccacggtgtacggcaggcccgcctgcttcaggtaggcggccttgtacaccaggtagcgggccgactcgatggccacgtccatgtcggccatcatccaggccaggccctggaacttgtccagcgagcggccgaactgcttgcgttccttcatgtacgcgcgggcctcgttaaaggcgccttcggcgatgcccagggcctgggccgcgatgccgatgcggcccccgtccagggtcttcatggcgatcgggaagcccttgccctccttgccaatcatgttctccaccggcacgatcatgtcctcgaacaccagctcggtggtcgacgacgcgcggatgcccagcttttgctccaccttgccgatcgagaagcccttgaagcccttctcgatgataaaggccgagatgcccttggtccccttggtgcggtcggtcatggcgaagatgacgaaggtgtcggcgacgccgccgttggtgatgaagattttcgagccgttgatcacgtagtggtcgccctccagcacggccacggtctgctgggcgcccgaatcggtgccggcgttcggctcggtcaggccgtaggcgccgatcttctcccccttggccagcggcaccaggtacttctgcttctgctcctcggtgccgtgctcgttaatcagcgaggcgcacagcgaggtgtgggccgacaggatcacgccggtggtgccgcacaccttcgacagctcctccacggcgatgatgtacgacagcacgtcgccgccggccccgccgtactccttcgagaacgggatgcccatcatgccgtactggcccatcttcttcacgttctccatcgggaaccgctccgtctcgtcgatctccgcggcaatcggcttcacctcgttctcggcgaactcccggaccatctggcgcaccagctcttgctcgcgcgtcaggttgaagtccatataaacttacctcctagcggttcttgaacccttcgatcttgcgcttctcaatgaaggcggtcatggcgtctttctggtcctcggtcgagaagcattccccgaacgcttccgactcgaaggcgagggccgtgtcaatgtcgcactgcatgccgcggttaatggcttgcttcgacagtttgacggccaccggggcattgctcacgattttgttggcgatctccttggcggtgttcatcagttccgagggctccaccaccttgttcaccaggccaatgcgcagggcctcatcggccttgatgttctgcgcggtgaaaatcagctgcttggccatgcccatgccgacgaggcgcgacagccgctgggtgccgccaaagcccggggtgatgcccaggcccacttccggctggccgaagcgggcgttcgaggaggcgatgcggatatcgcaggacatcgcaatttcgcacccgccgcccagggcgaacccattcacggccgcaatcaccggtttttccagcagctccagccgccgaaacactttgttccccagaatgccgaacttgcgcccctcgatggtattcatttctttcatctccgagatgtccgccccggcgacgaagctcttctcgcccgcgcccgtcaggatcacggcgagcacctccgagtcgttctcgatttcgccgatcacgtagtccatctctttcagggtgtccgagttcagggcgttgagggccttcgggcggttgatggtcaccaccgcgaccttgccttctttctccaggatgacattgttgagttccatgactaatcctcctaaaatgtgaaattgttatccgctcacaattccacacaacatacgagccggaagcataaagtgtaaagcctggggaagcttcttaagtaataaaaataagagttaccttaaatggtaactcttatttttttaatattgtttcatagtttagaacgacttgatgtagatgtccttcagctccgagatcagcgggtagcgcgggttggcggtggtgcactggtcgtcgaaggccagctccgacatcttgtccagggtgttgtagaaatccttcttgttgatgccggcggccgagatgttctgcgggatcgacaggtcgatcttcagcttcgagatggcctcgatcagggcggtcaccttctccgtgtccgaggtgcccttcaggttcaggtactcggcgatctcggcgtacttgcgcttggcgttcggcgacttgtactgcgggaaggcggtctgcttggtcgggcagtcggtggcgttgtacttgatcacctcctcgatcagcacggcgcaggcgatgccgtgcggcacgtggtgcatggcgcccagcttgtgggccatcgagtggcacacgcccaggaaggcgttggcgaaggccatgccggcgatgttcgaggcgtgggccatcttctcgcgggcctcgatgtcgttggtgccgttcttgtaggcgcgcggcaggtacttgaagatcatcttgatggcgcgcagggccagctcgtcggtgtagtccgtggccatcaccgacacgtaggcctcgatggcgtgcaccagggcatcaatgccggtggcggcggtcagcttgcgcggcatgttcagcatcagctcggtgtcaatgatggccatgttcggggtcagttcgtacgaggtcagcgggtacttcatgccggtctcgtcgttggtgatcacggcgaacggggtggcctccgagccggtgccggcggtggtcgggatggccaccgagatggccttggtgcccagcttcgggaagttgcagatgcgcttgcggatgtccatgaaattgatggcgaggttctcgatctccgcctccgggtactcgtacagcaggtgcatcaccttggcggcgtccatcggcgagccgccgccgatcgagatgatggtgtccggctcgaagttcagcatctccttggcgcccttcttcaccgagtcaatggtcgggtccgacttgatatcggtgaagatcgagtacttgatgtcgatctcgtccagcaccttggtgatcttgttcacgtagcccagcttgaacaggtccttgtcggtcacgatgaaggcgcgcttcttgttcatgtccttcagctccttcagggcgaagcgcaggcagccgtacttgaagtagatcttctgcggcaccttgaaccacagcatattctcgcggcgctcggccaccgacttgatgttcagcaggtgcttcggctccacgttctgcgacaccgagttgccgccccaggtcccgcagcccagggtgaacgacggggcgatggcgaagttgtacaggtcgcccgaggcgccctgcgacgacggcatgttgatgaaggtgcgcgaggtcttcatggccaggccgaactccttcaccttgtccttgttgttctgcgagtcgatgtacagcgacgaggtgtggcccgagccgcccagctcgatcaggcgctgggccttcttgagggcctcatcgaagtccttcaccttgtacatggccagcaccggcgacagcttctcgtgcgagaacagctccgacttctccaccgactgcacctcgccgatgaggatcttggtggtctgcggcacctcgatgccggccatcttggcgatgatgtaggccgacttgcccacgatgtcggcgttgatggcgccgttcttgaacatcgtctccttgatcttggcgatctcgttctggttcaggatgtacgagccgcgcttcacgaactcctccttcaccttctcgtagatggagttcatcaccaggatcgactgctccgaggcgcagatcacgccgttgtcgtaggtcttcgacaggatgatcgacgacacggccatatcgatgtcggcggactcgtcgatgatggccggggtgttgcccgcgcccacgccgatggccggcttgcccgacgagtaggcggccttcaccatcgacgggccgccggtggccaggatgatatcggcctccgacatcaggtcctgcgacagctcgatcgacggctcgtcaatccagccgatgatgttcttcggggccccggccttcacggcggcgtccaggatcagcttggcggcggcgatggtcgacttcttggcgcgcgggtgcggcgagaagaagatggcgttgcgggtcttcagcgagatcagcgacttgaagatggcggtcgaggtcgggttcgtggtcggcacgatggcggccacgatgccgatcggctcggccaccttggtgatgcccagcgagtcgtcgtggtcgataatgccgcaggtcttctcgttcttgtacttgttgtagatgtactcggcggcgaagtggttcttgatgatcttgtcctccaccaggccgatgccggtctcctccacggccagcttggccaggttgatgcgctccttggcggcggcaatggcgcactgcttgaagatcttgtccacctgctcctgggtgtaggtggcgaacttcttctgggcctcgcgcagctcgttcagcttctgcttcagctccttctggttggtcaccttcattcttgaatctcctgaaaatttctttttatttcgagtagtcgtaaaagcccttgccgctcttgcggcccagccagcccgcccggacatactttttcagcagcgtgtgcgggcgatacttcgaatcgccggtttccgagtacagcacgtccatgatggccaggcaaatgtcgaggccgataaagtcccccagttccagcgggcccatcgggtggttcgcccccagcttcatggccttatcaatatcctcgaccgaggcaatcccttcggcgaggatgcccaccgcttcattgatcatcgggatcagaatgcggttgacgacgaaccccggcgcctcggcgacttccacggggtccttgccaatggcaatcgacgtctctttcaccgcgtcgaaggtttcctgcgacgtggcaatgccgcgaatgacctccacgagtttcatgacgggggcgggattaaagaaatgcatgccgatcactttgtcgttggtcttggtcgcgctcgccacctccgtgatcgagagcgagctggtgttggaggcgagaatcgtctccggtttgcagatgttgtcgagatccgcaaagatctgtttcttaatgtccatgcgctcgacggcggcttcaatcacgaggtcgcagtccgcggccatgttcaggtccacggtcccgctgatgcgggtgagaatctccaccttggtggcctcttcgatcttgccttttttgaccagtttcgacaggttcttgttaatgaaatccaggccgcgatcgacgaattcatccttgatgtcccggaggaccacctcgaagcctttggcggcgaaggcctgcgcgatgccggaccccatcgtccccgcgccaatgacgcacaccttcttcattcttgaatctcctgaaactagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 65 - DNA, 11155 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccagcaggctgcctcgataagcaaaaagggcggccccgcggggccgccctttttctttccggcgactgtcaggccactcagttgttggcggccttcacctgcgcgatcagctccgggaccactttgttcacgtcgcccacgatcgccaggtcggccaccttcataatcggggcctccacgtccttattgatggcgatgatgtagtccgagtcctgcatgccggccaggtgctggatcgcccccgagatgccgcaggcgatgtacagggtcgggcgcacggtcttgccggtctggcccacctggaggtctttgtccacccactctttctcgatggccgcgcgcgaggcggcgatggtgccgcccaggagcgaggccagctcctccagtttttcgaagttctccttcgagcccacgccgcggcccccggccacgagcaccttggcctcgccgatgtccgcgatgtctttggccagcttcaccactttcgacactttggtgcggatgtcggacgcggtcagcttgatggcgactttctcgatcttatcgtcggacacgttggcgtcgttcaccggcagcttctcgaagaagacgccggggcggaccgtggccatctgcgggcggtggtccgagcagacgatggtggcgatcaggttgccgccgaacgccgggcgggtcgccaggaggtcccggttctccacgtcgatgtccagcgaggtgcagtcggcggtcaggccggtcgacaggcgcgccgcgatccgcgggcccaggtcccgcccgatgaacgtggcgccaatgaacaggatctccggcttgcgctcgttcacgaggtcgcagatcaccttcgcgtagccgtcggtcgagaagtgggcgaggagttcattgtcggccgcgagcaccttatccgcgccgtggctcagcaggtccttcgacatcttctcggtgttgtggcccagcagcacggcggtcagttccacccccagcttctcggccatctccttgcccttgcccagcagctccagcgacaccttctgcagctcgccgtcgcgctgctccgcaaacacccacacgcccttgtagtcggccttgttcattgaaaaatccctcctaacttaaatatgtgttcttcttttagttctgcgagagcatatcggccgcttccttcaccggtttatcaatgacctcgccctgccctttcacttctttcgtgctcgacttcttcactttggtcggcgaccccttcaggcccagattggctttatccacgtcgatatcatccgcggtccacattttcacctccttgtcgaacgcgccgaagattttctccaccgacatgtaccgcgggacgttcagttccttaatggccgtcaggagcaccggggtcttgacttccacgacctcgtacccatcctcccaggccttccggatcttgagcgtgtccccatccacctccactttttccacgtaggtgacttggggaatcccgagatgttccgcaatctccggcccgacctgcgccgtatcgccgtcgatggcctggcggccggcaaagacaatgtcatatttcagcttcttaatgccggcggcaatggtatgcgaggtggcgagggtgtccgcgcccccaaacgcccggtccgtgagcagcaccgcctcatcggcccccatggccagcgcttcgaccagggcgttcttggcttggggggggcccatcgagatgaccgtgacgtgcgccccatagttatctttcagcacgagggcttcctcgagcgcgtttttgtcgtcggggttaatgatggacgggacgccctcccgaatcagggtgcctttgacgggatcaatgcggacctcggcggtgtcggggacttgtttgaggcagaccacaatattcatcctcttaacctccttaaattagcggaagatcttgcccgagatcaccagcttctgcacctccgaggtgccctcgtagatctccgtgatcttggcgtcgcgcatcatgcgctccacggggtagtccttggtgtagccatagccgccgaacagctgcacggccttggtggtcacgtccatggccacgttggcggcatgcagcttggcgcgggcggcatccacggtgtacggcaggcccgcctgcttcaggtaggcggccttgtacaccaggtagcgggccgactcgatggccacgtccatgtcggccatcatccaggccaggccctggaacttgtccagcgagcggccgaactgcttgcgttccttcatgtacgcgcgggcctcgttaaaggcgccttcggcgatgcccagggcctgggccgcgatgccgatgcggcccccgtccagggtcttcatggcgatcgggaagcccttgccctccttgccaatcatgttctccaccggcacgatcatgtcctcgaacaccagctcggtggtcgacgacgcgcggatgcccagcttttgctccaccttgccgatcgagaagcccttgaagcccttctcgatgataaaggccgagatgcccttggtccccttggtgcggtcggtcatggcgaagatgacgaaggtgtcggcgacgccgccgttggtgatgaagattttcgagccgttgatcacgtagtggtcgccctccagcacggccacggtctgctgggcgcccgaatcggtgccggcgttcggctcggtcaggccgtaggcgccgatcttctcccccttggccagcggcaccaggtacttctgcttctgctcctcggtgccgtgctcgttaatcagcgaggcgcacagcgaggtgtgggccgacaggatcacgccggtggtgccgcacaccttcgacagctcctccacggcgatgatgtacgacagcacgtcgccgccggccccgccgtactccttcgagaacgggatgcccatcatgccgtactggcccatcttcttcacgttctccatcgggaaccgctccgtctcgtcgatctccgcggcaatcggcttcacctcgttctcggcgaactcccggaccatctggcgcaccagctcttgctcgcgcgtcaggttgaagtccatataaacttacctcctagcggttcttgaacccttcgatcttgcgcttctcaatgaaggcggtcatggcgtctttctggtcctcggtcgagaagcattccccgaacgcttccgactcgaaggcgagggccgtgtcaatgtcgcactgcatgccgcggttaatggcttgcttcgacagtttgacggccaccggggcattgctcacgattttgttggcgatctccttggcggtgttcatcagttccgagggctccaccaccttgttcaccaggccaatgcgcagggcctcatcggccttgatgttctgcgcggtgaaaatcagctgcttggccatgcccatgccgacgaggcgcgacagccgctgggtgccgccaaagcccggggtgatgcccaggcccacttccggctggccgaagcgggcgttcgaggaggcgatgcggatatcgcaggacatcgcaatttcgcacccgccgcccagggcgaacccattcacggccgcaatcaccggtttttccagcagctccagccgccgaaacactttgttccccagaatgccgaacttgcgcccctcgatggtattcatttctttcatctccgagatgtccgccccggcgacgaagctcttctcgcccgcgcccgtcaggatcacggcgagcacctccgagtcgttctcgatttcgccgatcacgtagtccatctctttcagggtgtccgagttcagggcgttgagggccttcgggcggttgatggtcaccaccgcgaccttgccttctttctccaggatgacattgttgagttccatgactaatcctcctaaaatgtgaaattgttatccgctcacaattccacacaacatacgagccggaagcataaagtgtaaagcctggggaagcttcttaagtaataaaaataagagttaccttaaatggtaactcttatttttttaatattgtttcatagtatttctttttatttcgagtagtcgtaaaagcccttgccgctcttgcggcccagccagcccgcccggacatactttttcagcagcgtgtgcgggcgatacttcgaatcgccggtttccgagtacagcacgtccatgatggccaggcaaatgtcgaggccgataaagtcccccagttccagcgggcccatcgggtggttcgcccccagcttcatggccttatcaatatcctcgaccgaggcaatcccttcggcgaggatgcccaccgcttcattgatcatcgggatcagaatgcggttgacgacgaaccccggcgcctcggcgacttccacggggtccttgccaatggcaatcgacgtctctttcaccgcgtcgaaggtttcctgcgacgtggcaatgccgcgaatgacctccacgagtttcatgacgggggcgggattaaagaaatgcatgccgatcactttgtcgttggtcttggtcgcgctcgccacctccgtgatcgagagcgagctggtgttggaggcgagaatcgtctccggtttgcagatgttgtcgagatccgcaaagatctgtttcttaatgtccatgcgctcgacggcggcttcaatcacgaggtcgcagtccgcggccatgttcaggtccacggtcccgctgatgcgggtgagaatctccaccttggtggcctcttcgatcttgccttttttgaccagtttcgacaggttcttgttaatgaaatccaggccgcgatcgacgaattcatccttgatgtcccggaggaccacctcgaagcctttggcggcgaaggcctgcgcgatgccggaccccatcgtccccgcgccaatgacgcacaccttcttcattcttgaatctcctgaaactagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 66 - DNA, 13317 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccagcaggctgcctcgataagcaaaaagggcggccccgcggggccgccctttttctttccggcgactgtcaggccactcagttgttggcggccttgacttgggcgatcagctcgggcaccaccttgttcacatcgcccacaatggccaggtcggccaccttcatgatcggggcttccacgtccttgttgatcgcgatgatgtagtccgagtcctgcatgccggccaggtgctggatggcgcccgagatgccgcaggcgatgtacagggtcgggcgcacggtcttgccggtctgccccacttggaggtccttatccacccactccttttcgatggcggcgcgcgaggcggcgatggtgccgccgaggagggaggccagctcctccagcttctcaaagttctccttcgagcccacgccgcggcccccggccacgagcaccttggcctcgccgatatcggcaatatccttggcgagcttcaccactttcgagaccttggtgcggatgtccgaggcggtcagcttgatggccactttctcgatcttgtcgtccgacacgttggcgtcgttcaccggcagcttctcgaagaacacgccgggccgcaccgtggccatctgcgggcggtggtccgagcacacgatggtggcgatcaggttgccgccgaaggccgggcgggtcgcgagcagatcgcggttctccacgtcgatgtccagcgaggtgcagtcggcggtcaggccggtcgacagccgggcggcgatccgcgggccgaggtcgcggccgatgaacgtggcgccgatgaacaggatctccggcttgcgctcgttcaccaggtcacagatcaccttggcgtagccgtcggtgctgaaatgggccagcagctcgttgtcggcggccaggaccttgtccgcgccgtgcgacagcaggtccttcgacatcttctcggtgttgtgccccagcagcacggccgtcagctccacgcccagtttctcggccatttccttgcccttccccagcagttccagcgacaccttctgcagctcgccgtcgcgttgctcggcgaacacccacacgcccttgtagtcggctttgttcattgaaaaatccctcctaacttaaatatgtgttcttcttttagttctgcgacagcatatcggccgcctccttcacgggcttatcgatcacttcgccttggcccttgacttctttggtcgacgactttttcaccttggtcggcgagcccttcagccccaggttggccttgtcgacgtcgatgtcatccgcggtccacatcttgacctccttgtcgaacgcgccgaaaatcttttccaccgacatgtagcgcggcacgttcagctccttgatggccgtcagcaggaccggggtcttcacctccacgacctcgtagccgtcctcccaggccttgcggattttcagcgtatcgccatccacctcgactttctccacgtaggtcacctgggggatgcccagatgctcggcgatctccggcccgacctgggccgtgtcgccgtcaatggcctggcgcccggcaaacacgatatcatatttcagcttcttgatgcccgcggcgatggtgtgcgaggtcgccagggtatccgcccccccgaaggcgcggtcggtcagcagcaccgcctcgtccgcccccatggccagggcctccaccagggcgttcttggcctgcggggggcccatcgagatcacggtcacatgggccccgtagttgtctttcaggaccagcgcttcctccagcgcattcttgtcgtccgggttgatgatcgacggcacgccctcgcggatcagggtccccttcaccggatcgatgcgcacctcggcggtgtccggcacctgcttcagacacaccacgatgttcatcctcttaacctccttaaattagcggaaaatcttgccgctaatcaccagcttctgcacttccgacgtgccctcgtagatctcggtaatcttggcgtcgcgcatcatgcgctcgaccgggtagtccttcgtgtagccgtagccgccgaacagttgcaccgctttggtggtcacgtccatggcgacgttggcggcatgcagcttggcgcgcgcggcgtccacggtgtacggcaggcccgcctgcttcaggtaggcggccttatagaccagataccgggcggactcaatggccacgtccatatcggccatcatccaggccaggccctgaaacttgtccagcgagcggccgaactgcttgcgctccttcatgtaggcccgggcttcgttgaaggcgccctccgcgatccccagggcttgggccgcaatgccgatccgccccccatccagggttttcatcgcaatcgggaaccccttcccctctttgccaatcatgttttccaccggcacgatcatgtcctcgaacaccagttcggtggtcgacgacgcgcggatgcccagcttttgctccaccttgccgatcgagaagcccttaaaccctttctcaatgataaaggccgagatgcctttggtgcccttggtgcggtcggtcatcgcgaagatcacgaacgtgtccgccacgccgccgttggtaataaagattttggagccgttgatcacgtagtggtcgccttccaggacggccacggtctgctgcgcgcccgagtcggtgccggcgttcggttccgtcaggccgtacgcgccgatcttctcccctttggccagcgggaccaggtacttctgtttctgctcctcggtcccgtgctcgttgatcagcgaggcgcagagggaggtgtgggccgacaggatcaccccggtggtgccgcacaccttggacagctcctccacggcgatgatgtacgacagcacgtccccccccgccccgccgtactccttcgagaacggaatccccatcatgccatactggcccatcttcttcacgttctccatcgggaagcgctcggtctcgtcgatctccgcggcaatgggcttcacctcgttttccgcgaactcgcgcaccatctggcgcaccagctcctgctcgcgggtcaggttgaagtccatataaacttacctcctatctatgtgaaattgttatccgctcacaattccacacaacatacgagccggaagcataaagtgtaaagcctggggaagcttggggagaacaatcagcccggcaggggccgggctgattgtgcctgcgtgccttagaacgacttgatgtagatgtccttcagctccgagatcagcgggtagcgcgggttggcggtggtgcactggtcgtcgaaggccagctccgacatcttgtccagggtgttgtagaaatccttcttgttgatgccggcggccgagatgttctgcgggatcgacaggtcgatcttcagcttcgagatggcctcgatcagggcggtcaccttctccgtgtccgaggtgcccttcaggttcaggtactcggcgatctcggcgtacttgcgcttggcgttcggcgacttgtactgcgggaaggcggtctgcttggtcgggcagtcggtggcgttgtacttgatcacctcctcgatcagcacggcgcaggcgatgccgtgcggcacgtggtgcatggcgcccagcttgtgggccatcgagtggcacacgcccaggaaggcgttggcgaaggccatgccggcgatgttcgaggcgtgggccatcttctcgcgggcctcgatgtcgttggtgccgttcttgtaggcgcgcggcaggtacttgaagatcatcttgatggcgcgcagggccagctcgtcggtgtagtccgtggccatcaccgacacgtaggcctcgatggcgtgcaccagggcatcaatgccggtggcggcggtcagcttgcgcggcatgttcagcatcagctcggtgtcaatgatggccatgttcggggtcagttcgtacgaggtcagcgggtacttcatgccggtctcgtcgttggtgatcacggcgaacggggtggcctccgagccggtgccggcggtggtcgggatggccaccgagatggccttggtgcccagcttcgggaagttgcagatgcgcttgcggatgtccatgaaattgatggcgaggttctcgatctccgcctccgggtactcgtacagcaggtgcatcaccttggcggcgtccatcggcgagccgccgccgatcgagatgatggtgtccggctcgaagttcagcatctccttggcgcccttcttcaccgagtcaatggtcgggtccgacttgatatcggtgaagatcgagtacttgatgtcgatctcgtccagcaccttggtgatcttgttcacgtagcccagcttgaacaggtccttgtcggtcacgatgaaggcgcgcttcttgttcatgtccttcagctccttcagggcgaagcgcaggcagccgtacttgaagtagatcttctgcggcaccttgaaccacagcatattctcgcggcgctcggccaccgacttgatgttcagcaggtgcttcggctccacgttctgcgacaccgagttgccgccccaggtcccgcagcccagggtgaacgacggggcgatggcgaagttgtacaggtcgcccgaggcgccctgcgacgacggcatgttgatgaaggtgcgcgaggtcttcatggccaggccgaactccttcaccttgtccttgttgttctgcgagtcgatgtacagcgacgaggtgtggcccgagccgcccagctcgatcaggcgctgggccttcttgagggcctcatcgaagtccttcaccttgtacatggccagcaccggcgacagcttctcgtgcgagaacagctccgacttctccaccgactgcacctcgccgatgaggatcttggtggtctgcggcacctcgatgccggccatcttggcgatgatgtaggccgacttgcccacgatgtcggcgttgatggcgccgttcttgaacatcgtctccttgatcttggcgatctcgttctggttcaggatgtacgagccgcgcttcacgaactcctccttcaccttctcgtagatggagttcatcaccaggatcgactgctccgaggcgcagatcacgccgttgtcgtaggtcttcgacaggatgatcgacgacacggccatatcgatgtcggcggactcgtcgatgatggccggggtgttgcccgcgcccacgccgatggccggcttgcccgacgagtaggcggccttcaccatcgacgggccgccggtggccaggatgatatcggcctccgacatcaggtcctgcgacagctcgatcgacggctcgtcaatccagccgatgatgttcttcggggccccggccttcacggcggcgtccaggatcagcttggcggcggcgatggtcgacttcttggcgcgcgggtgcggcgagaagaagatggcgttgcgggtcttcagcgagatcagcgacttgaagatggcggtcgaggtcgggttcgtggtcggcacgatggcggccacgatgccgatcggctcggccaccttggtgatgcccagcgagtcgtcgtggtcgataatgccgcaggtcttctcgttcttgtacttgttgtagatgtactcggcggcgaagtggttcttgatgatcttgtcctccaccaggccgatgccggtctcctccacggccagcttggccaggttgatgcgctccttggcggcggcaatggcgcactgcttgaagatcttgtccacctgctcctgggtgtaggtggcgaacttcttctgggcctcgcgcagctcgttcagcttctgcttcagctccttctggttggtcaccttcattcttgaatctcctgaaagccggtgcttacggcagcttgaccacggcttccccggtgacggccagggccccgccttgggtaaagatccgggtcgtcagcgtggcgatcggtttgtcctcgcgcagcgcggtgacttccacctcggcggtcacttcgtcccccacgaacaccggcagtttgaacgagagcgattggcccaggtagatgctccccttgccggggagctgctggccgaggaggcccgagaacagcgaggcgagcagcatgccatgcacgatggggcgctcgaacgcggtggtcgcggcaaacgcggggtccaggtgcagcgggttgaagtcctcggagagcgccgcaaaggccgccacctccgccgcgccaaaccgcttggacagccgggctttttgcccgacctccagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 67 - DNA, 10723 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatccagcaggctgcctcgataagcaaaaagggcggccccgcggggccgccctttttctttccggcgactgtcaggccactcagttgttggcggccttgacttgggcgatcagctcgggcaccaccttgttcacatcgcccacaatggccaggtcggccaccttcatgatcggggcttccacgtccttgttgatcgcgatgatgtagtccgagtcctgcatgccggccaggtgctggatggcgcccgagatgccgcaggcgatgtacagggtcgggcgcacggtcttgccggtctgccccacttggaggtccttatccacccactccttttcgatggcggcgcgcgaggcggcgatggtgccgccgaggagggaggccagctcctccagcttctcaaagttctccttcgagcccacgccgcggcccccggccacgagcaccttggcctcgccgatatcggcaatatccttggcgagcttcaccactttcgagaccttggtgcggatgtccgaggcggtcagcttgatggccactttctcgatcttgtcgtccgacacgttggcgtcgttcaccggcagcttctcgaagaacacgccgggccgcaccgtggccatctgcgggcggtggtccgagcacacgatggtggcgatcaggttgccgccgaaggccgggcgggtcgcgagcagatcgcggttctccacgtcgatgtccagcgaggtgcagtcggcggtcaggccggtcgacagccgggcggcgatccgcgggccgaggtcgcggccgatgaacgtggcgccgatgaacaggatctccggcttgcgctcgttcaccaggtcacagatcaccttggcgtagccgtcggtgctgaaatgggccagcagctcgttgtcggcggccaggaccttgtccgcgccgtgcgacagcaggtccttcgacatcttctcggtgttgtgccccagcagcacggccgtcagctccacgcccagtttctcggccatttccttgcccttccccagcagttccagcgacaccttctgcagctcgccgtcgcgttgctcggcgaacacccacacgcccttgtagtcggctttgttcattgaaaaatccctcctaacttaaatatgtgttcttcttttagttctgcgacagcatatcggccgcctccttcacgggcttatcgatcacttcgccttggcccttgacttctttggtcgacgactttttcaccttggtcggcgagcccttcagccccaggttggccttgtcgacgtcgatgtcatccgcggtccacatcttgacctccttgtcgaacgcgccgaaaatcttttccaccgacatgtagcgcggcacgttcagctccttgatggccgtcagcaggaccggggtcttcacctccacgacctcgtagccgtcctcccaggccttgcggattttcagcgtatcgccatccacctcgactttctccacgtaggtcacctgggggatgcccagatgctcggcgatctccggcccgacctgggccgtgtcgccgtcaatggcctggcgcccggcaaacacgatatcatatttcagcttcttgatgcccgcggcgatggtgtgcgaggtcgccagggtatccgcccccccgaaggcgcggtcggtcagcagcaccgcctcgtccgcccccatggccagggcctccaccagggcgttcttggcctgcggggggcccatcgagatcacggtcacatgggccccgtagttgtctttcaggaccagcgcttcctccagcgcattcttgtcgtccgggttgatgatcgacggcacgccctcgcggatcagggtccccttcaccggatcgatgcgcacctcggcggtgtccggcacctgcttcagacacaccacgatgttcatcctcttaacctccttaaattagcggaaaatcttgccgctaatcaccagcttctgcacttccgacgtgccctcgtagatctcggtaatcttggcgtcgcgcatcatgcgctcgaccgggtagtccttcgtgtagccgtagccgccgaacagttgcaccgctttggtggtcacgtccatggcgacgttggcggcatgcagcttggcgcgcgcggcgtccacggtgtacggcaggcccgcctgcttcaggtaggcggccttatagaccagataccgggcggactcaatggccacgtccatatcggccatcatccaggccaggccctgaaacttgtccagcgagcggccgaactgcttgcgctccttcatgtaggcccgggcttcgttgaaggcgccctccgcgatccccagggcttgggccgcaatgccgatccgccccccatccagggttttcatcgcaatcgggaaccccttcccctctttgccaatcatgttttccaccggcacgatcatgtcctcgaacaccagttcggtggtcgacgacgcgcggatgcccagcttttgctccaccttgccgatcgagaagcccttaaaccctttctcaatgataaaggccgagatgcctttggtgcccttggtgcggtcggtcatcgcgaagatcacgaacgtgtccgccacgccgccgttggtaataaagattttggagccgttgatcacgtagtggtcgccttccaggacggccacggtctgctgcgcgcccgagtcggtgccggcgttcggttccgtcaggccgtacgcgccgatcttctcccctttggccagcgggaccaggtacttctgtttctgctcctcggtcccgtgctcgttgatcagcgaggcgcagagggaggtgtgggccgacaggatcaccccggtggtgccgcacaccttggacagctcctccacggcgatgatgtacgacagcacgtccccccccgccccgccgtactccttcgagaacggaatccccatcatgccatactggcccatcttcttcacgttctccatcgggaagcgctcggtctcgtcgatctccgcggcaatgggcttcacctcgttttccgcgaactcgcgcaccatctggcgcaccagctcctgctcgcgggtcaggttgaagtccatataaacttacctcctatctatgtgaaattgttatccgctcacaattccacacaacatacgagccggaagcataaagtgtaaagcctggggaagcttggggagaacaatcagcccggcaggggccgggctgattgtgcctgcgtgccgccggtgcttacggcagcttgaccacggcttccccggtgacggccagggccccgccttgggtaaagatccgggtcgtcagcgtggcgatcggtttgtcctcgcgcagcgcggtgacttccacctcggcggtcacttcgtcccccacgaacaccggcagtttgaacgagagcgattggcccaggtagatgctccccttgccggggagctgctggccgaggaggcccgagaacagcgaggcgagcagcatgccatgcacgatggggcgctcgaacgcggtggtcgcggcaaacgcggggtccaggtgcagcgggttgaagtcctcggagagcgccgcaaaggccgccacctccgccgcgccaaaccgcttggacagccgggctttttgcccgacctccagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 68 - DNA, 153 bp, Ralstonia eutropha
aacccgcatcacacccgcgtcttgaaatgcccctaccccgtccctataattagcactcgtcaggggtgagtgctaacagcctcctgcgacacctgaacatttctgacggccgtcgcctcggtagccggccgatttgtgatcaaaaactcactt

SEQ ID NO: 69 - protein, 422 aa, Jeotgalicoccus sp. ATCC 8456
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 70 - protein, 301 aa, Clostridium acetobutylicum
MIKSFNEIIMKVKSKEMKKVAVAVAQDEPVLEAVRDAKKNGIADAILVGDHDEIVSIALKIGMDVNDFEIVNEPNVKKAALKAVELVSTGKADMVMKGLVNTATFLRSVLNKEVGLRTGKTMSHVAVFETEKFDRLLFLTDVAFNTYPELKEKIDIVNNSVKVAHAIGIENPKVAPICAVEVINPKMPSTLDAAMLSKMSDRGQIKGCVVDGPLALDIALSEEAAHHKGVTGEVAGKADIFLMPNIETGNVMYKTLTYTTDSKNGGILVGTSAPVVLTSRADSHETKMNSIALAALVAGNK

SEQ ID NO: 71 - protein, 355 aa, Clostridium acetobutylicum
MYRLLIINPGSTSTKIGIYDDEKEIFEKTLRHSAEEIEKYNTIFDQFQFRKNVILDALKEANIEVSSLNAVVGRGGLLKPIVSGTYAVNQKMLEDLKVGVQGQHASNLGGIIANEIAKEINVPAYIVDPVVVDELDEVSRISGMADIPRKSIFHALNQKAVARRYAKEVGKKYEDLNLIVVHMGGGTSVGTHKDGRVIEVNNTLDGEGPFSPERSGGVPIGDLVRLCFSNKYTYEEVMKKINGKGGVVSYLNTIDFKAVVDKALEGDKKCALIYEAFTFQVAKEIGKCSTVLKGNVDAIILTGGIAYNEHVCNAIEDRVKFIAPVVRYGGEDELLALAEGGLRVLRGEEKAKEYK

SEQ ID NO: 72 - DNA, 94 bp, Clostridium acetobutylicum
taaagtcataaataatataatataaccagtacccatgtttataaaacttttgccctataaacatgggtattgttttttttttatttttttctga

SEQ ID NO: 73 - DNA, 3563 bp, Artificial Sequence
  synthetic gen
ggatccaacccgcatcacacccgcgtcttgaaatgcccctaccccgtccctataattagcactcgtcaggggtgagtgctaacagcctcctgcgacacctgaacatttctgacggccgtcgcctcggtagccggccgatttgtgatcaaaaactcactttttcaggagattcaagaatggccaccctgaagcgcgataagggcctcgataacacgctgaaggtgctgaagcagggctatctgtacacgaccaatcagcgcaaccgcctcaacacctcggtgttccagaccaaagcgctcggggggaagcccttcgtcgtggtgaccgggaaggagggcgccgagatgttctacaacaacgacgtggtgcaacgcgaaggcatgctgcccaagcgcattgtgaataccctgttcggcaaaggcgccatccataccgtggacggcaagaagcacgtggaccgcaaggcgctcttcatgtcgctgatgaccgaaggcaacctcaactacgtgcgggagctgacccgcaccctgtggcacgccaacacgcagcgcatggagtcgatggatgaggtgaacatctaccgcgaatcgatcgtgctcctgaccaaggtgggcacccgctgggccggggtccaggcgcccccggaggacattgagcgcatcgcgaccgacatggatattatgatcgacagctttcgcgccctggggggggcgtttaagggctacaaagcgtcgaaggaggcgcggcggcgcgtggaggattggctcgaggagcagatcatcgagacgcgcaagggcaacatccacccgcccgagggcaccgccctgtatgagtttgcccactgggaggactacctggggaacccgatggactcgcggacgtgcgcgattgacctgatgaacacctttcggccgctgatcgccatcaaccgcttcgtgtccttcgggctgcacgcgatgaacgagaatcccatcacccgcgagaagatcaagtcggagccggactacgcctacaagttcgcgcaggaggtccgccgctactacccgttcgtgccgttcctcccggggaaggccaaagtggatatcgacttccaaggcgtgaccattcccgccggcgtggggctggccctcgacgtgtatggcaccacccatgacgagagcctgtgggacgacccgaacgaatttcgcccggagcggttcgagacctgggacggctcgccgttcgacctcatcccgcagggcgggggcgactattggaccaaccaccgctgcgccggggagtggatcaccgtgattatcatggaggagaccatgaagtattttgcggaaaagatcacgtacgacgtcccggagcaggacctggaggtggatctgaattcgattccgggctatgtgaagtcggggttcgtgatcaaaaacgtgcgcgaggtggtcgatcgcacctaatttcaggagattcaagaatgatcaagtcgttcaacgaaatcatcatgaaagtgaaatcgaaggagatgaagaaagtcgccgtcgcggtcgcccaggacgaaccggtgctggaggccgtgcgggacgcgaagaagaacgggatcgccgacgccatcctggtgggcgatcacgatgaaatcgtgtccatcgccctgaagattggcatggatgtgaacgattttgaaattgtgaatgagcccaatgtgaaaaaggcggcgctgaaggcggtggaactggtgtcgaccggcaaggccgacatggtgatgaaggggctggtgaataccgccacgtttctgcgctcggtgctcaacaaggaagtgggcctgcgcacggggaaaacgatgtcgcatgtggccgtctttgagaccgagaagtttgaccgcctcctgtttctcacggacgtcgcctttaacacctacccggagctgaaagagaagatcgacattgtcaacaattcggtgaaggtggcccacgcgatcggcatcgaaaacccgaaagtggccccgatctgcgccgtggaggtgatcaaccccaaaatgccgtcgaccctggatgccgccatgctgagcaagatgtcggatcgcggccagatcaagggctgcgtggtggatggccccctggccctggacattgccctgtccgaggaagccgcgcaccataagggcgtgacgggggaggtggcgggcaaagcggatatcttcctcatgcccaacatcgagacgggcaacgtgatgtataaaaccctcacctatacgacggacagcaagaatgggggcatcctcgtgggcaccagcgccccggtcgtgctgacctcgcgggcggattcgcacgagaccaagatgaattcgattgcgctggcggccctcgtcgccgggaacaagtaaattaaagttaagtggaggaatgttaacatgtaccgcctcctcatcatcaaccccgggagcacctccacgaagatcggcatctatgacgacgagaaagagatctttgagaaaacgctgcgccactcggcggaggagatcgagaagtataataccattttcgaccagttccaattccgcaagaacgtcatcctggacgcgctcaaggaggccaacatcgaagtgtcgtcgctgaacgccgtcgtggggcgcgggggcctgctgaagcccatcgtgtcgggcacgtacgccgtgaaccaaaaaatgctggaagacctgaaggtgggggtgcagggccagcatgcctcgaacctgggcgggattatcgcgaacgagattgccaaggaaattaacgtgccggcctacatcgtggacccggtggtcgtcgacgaactggacgaggtgtcgcgcatctcgggcatggcggacatcccgcgcaagtccatttttcacgccctcaaccagaaggccgtggcccggcgctacgccaaagaagtggggaaaaagtacgaggatctgaacctgatcgtggtgcacatgggcggcgggacctcggtggggacccacaaggacggccgcgtgatcgaggtgaacaacaccctggatggcgagggcccgttttcgccggaacgctccggcggcgtgcccatcggcgacctcgtgcgcctgtgcttttcgaacaagtacacctacgaagaggtgatgaagaagatcaatgggaagggcggggtggtgtcgtacctgaataccatcgacttcaaggcggtcgtggacaaagccctggagggcgataaaaagtgcgccctgatttatgaggccttcaccttccaggtggcgaaagaaattggcaagtgctcgaccgtgctgaaggggaacgtggacgccatcatcctcaccggcggcatcgcctacaatgagcacgtgtgcaacgccattgaagaccgggtgaagttcatcgcgcccgtggtgcggtacggcggggaagacgagctgctcgccctggccgaaggcggcctgcgcgtgctccggggcgaggaaaaggcgaaggagtacaagtaataaagtcataaataatataatataaccagtacccatgtttataaaacttttgccctataaacatgggtattgttttttttttatttttttctgagagctc

SEQ ID NO: 74 - DNA, 14861 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctctcagaaaaaaataaaaaaaaaacaatacccatgtttatagggcaaaagttttataaacatgggtactggttatattatattatttatgactttattacttgtactccttcgccttttcctcgccccggagcacgcgcaggccgccttcggccagggcgagcagctcgtcttccccgccgtaccgcaccacgggcgcgatgaacttcacccggtcttcaatggcgttgcacacgtgctcattgtaggcgatgccgccggtgaggatgatggcgtccacgttccccttcagcacggtcgagcacttgccaatttctttcgccacctggaaggtgaaggcctcataaatcagggcgcactttttatcgccctccagggctttgtccacgaccgccttgaagtcgatggtattcaggtacgacaccaccccgcccttcccattgatcttcttcatcacctcttcgtaggtgtacttgttcgaaaagcacaggcgcacgaggtcgccgatgggcacgccgccggagcgttccggcgaaaacgggccctcgccatccagggtgttgttcacctcgatcacgcggccgtccttgtgggtccccaccgaggtcccgccgcccatgtgcaccacgatcaggttcagatcctcgtactttttccccacttctttggcgtagcgccgggccacggccttctggttgagggcgtgaaaaatggacttgcgcgggatgtccgccatgcccgagatgcgcgacacctcgtccagttcgtcgacgaccaccgggtccacgatgtaggccggcacgttaatttccttggcaatctcgttcgcgataatcccgcccaggttcgaggcatgctggccctgcacccccaccttcaggtcttccagcattttttggttcacggcgtacgtgcccgacacgatgggcttcagcaggcccccgcgccccacgacggcgttcagcgacgacacttcgatgttggcctccttgagcgcgtccaggatgacgttcttgcggaattggaactggtcgaaaatggtattatacttctcgatctcctccgccgagtggcgcagcgttttctcaaagatctctttctcgtcgtcatagatgccgatcttcgtggaggtgctcccggggttgatgatgaggaggcggtacatgttaacattcctccacttaactttaatttacttgttcccggcgacgagggccgccagcgcaatcgaattcatcttggtctcgtgcgaatccgcccgcgaggtcagcacgaccggggcgctggtgcccacgaggatgcccccattcttgctgtccgtcgtataggtgagggttttatacatcacgttgcccgtctcgatgttgggcatgaggaagatatccgctttgcccgccacctcccccgtcacgcccttatggtgcgcggcttcctcggacagggcaatgtccagggccagggggccatccaccacgcagcccttgatctggccgcgatccgacatcttgctcagcatggcggcatccagggtcgacggcattttggggttgatcacctccacggcgcagatcggggccactttcgggttttcgatgccgatcgcgtgggccaccttcaccgaattgttgacaatgtcgatcttctctttcagctccgggtaggtgttaaaggcgacgtccgtgagaaacaggaggcggtcaaacttctcggtctcaaagacggccacatgcgacatcgttttccccgtgcgcaggcccacttccttgttgagcaccgagcgcagaaacgtggcggtattcaccagccccttcatcaccatgtcggccttgccggtcgacaccagttccaccgccttcagcgccgcctttttcacattgggctcattcacaatttcaaaatcgttcacatccatgccaatcttcagggcgatggacacgatttcatcgtgatcgcccaccaggatggcgtcggcgatcccgttcttcttcgcgtcccgcacggcctccagcaccggttcgtcctgggcgaccgcgacggcgactttcttcatctccttcgatttcactttcatgatgatttcgttgaacgacttgatcattcttgaatctcctgaaattaggtgcgatcgaccacctcgcgcacgtttttgatcacgaaccccgacttcacatagcccggaatcgaattcagatccacctccaggtcctgctccgggacgtcgtacgtgatcttttccgcaaaatacttcatggtctcctccatgataatcacggtgatccactccccggcgcagcggtggttggtccaatagtcgcccccgccctgcgggatgaggtcgaacggcgagccgtcccaggtctcgaaccgctccgggcgaaattcgttcgggtcgtcccacaggctctcgtcatgggtggtgccatacacgtcgagggccagccccacgccggcgggaatggtcacgccttggaagtcgatatccactttggccttccccgggaggaacggcacgaacgggtagtagcggcggacctcctgcgcgaacttgtaggcgtagtccggctccgacttgatcttctcgcgggtgatgggattctcgttcatcgcgtgcagcccgaaggacacgaagcggttgatggcgatcagcggccgaaaggtgttcatcaggtcaatcgcgcacgtccgcgagtccatcgggttccccaggtagtcctcccagtgggcaaactcatacagggcggtgccctcgggcgggtggatgttgcccttgcgcgtctcgatgatctgctcctcgagccaatcctccacgcgccgccgcgcctccttcgacgctttgtagcccttaaacgccccccccagggcgcgaaagctgtcgatcataatatccatgtcggtcgcgatgcgctcaatgtcctccgggggcgcctggaccccggcccagcgggtgcccaccttggtcaggagcacgatcgattcgcggtagatgttcacctcatccatcgactccatgcgctgcgtgttggcgtgccacagggtgcgggtcagctcccgcacgtagttgaggttgccttcggtcatcagcgacatgaagagcgccttgcggtccacgtgcttcttgccgtccacggtatggatggcgcctttgccgaacagggtattcacaatgcgcttgggcagcatgccttcgcgttgcaccacgtcgttgttgtagaacatctcggcgccctccttcccggtcaccacgacgaagggcttccccccgagcgctttggtctggaacaccgaggtgttgaggcggttgcgctgattggtcgtgtacagatagccctgcttcagcaccttcagcgtgttatcgaggcccttatcgcgcttcagggtggccattcttgaatctcctgaaaaagtgagtttttgatcacaaatcggccggctaccgaggcgacggccgtcagaaatgttcaggtgtcgcaggaggctgttagcactcacccctgacgagtgctaattatagggacggggtaggggcatttcaagacgcgggtgtgatgcgggttggatccagcaggctgcctcgataagcaaaaagggcggccccgcggggccgccctttttctttccggcgactgtcaggccactcagaactgctcggcggtcatggcgttcaccggggcggcgcccgacacgatggcgtcgcgcagggccgaggcctgcgacaggatgtgctcggcgaagaaatgggcggtggcgatcttggcgtcgtagaagcccgggtcgccggcgcgcttggcgtcggccgccagcatggcgcggccgaactgccagcccgagaacacgatgccgcacagcttcaggtacggcaccgagccggcgaacacggcgttcgggtccgacttggcgttggccaccacgaaggcgaccacgtcctccagggcggcgcggcccttggccagctgggcctgcacggcggtgaagccggcgcacgagtgattgcccagggcggcctcggtctcggcgatctgggcgcagatggcgcgggccacggcgccgccgtcgcgcacggtcttgcggccgatcaggtcgttggcctggatcgcggtggtgccctcgtagatcggcaggatgcgggcgtcgcggtagtgctgggcggcgccggtctcctcgatgaagcccatgccgccgtgcacctgcacgcccagcgaggtcacgtcgatcgacagctcggtcgaccagcccttcaccaccggcaccaggaactcgtagaaggcctggttctgctggcgcacggcctcgtccgggtgctggtgggcggcgtccgaggcggcggcggccacgtaggccacggcgcgggcgccctcggtcagggcgcgcatggtcatcagcatgcgcttcacgtccgggtggtggatgatggtcacggcctcgcgggccgagccgtccaccgggcgcgactgcacgcgctcgcgggcgaaggccacggcctgctggtaggcgcgctccgacacggcgatcccctgcatgcccaccgagaagcgggccgagttcatcatgatgaacatgtactccaggccgcggttctcctcgcccaccagggtgccgatggcgccgccgtggtcgccgaactgcagcacggcggtcggcgaggccttgatgcccagcttgtgctcgatcgacacgcagtgcacgtcgttgcgctcgccggtcgagccgtcggcgttcaccatgaacttcggcacgatgaacagcgagatgcccttcacgccctccggggcggtcggggtgcgggccagcaccaggtgcacgatgttcttggccatgtcgtgctcgccgtaggtgatgaagatcttcgtgccgaacaccttgtaggtgccgtcgccctgcggctcggcgcgggtgcgcacggcggccaggtcggagccggcctgcggctccgtcaggttcatggtgccggtccactcgcccgagatcagcttcggcaggaaggtggccttctgctcgtcggtgccggcggtcagcagggcctcgatggcgccgtcggtcagcagcgggcacagggcgaacgacaggttggcggtgttcagcatctcgttgcaggcggtggccaccagcttcggcaggccctggccgccgaactcctgcgggtgcagcacgccctgccagccgccctcgccgaactggcggaaggcctccttgaagcccggggtggtggtcaccacgccgtccttccacgacgacgggtccaggtcgccggcgcggttcagcggggccaccacctgctcgttgaacttggcggcctcgtccagcacggcctcggcggtctccggggtggcctcctcgaagcccggcagcttcgacacggcctccaggccggccagctcgttcatcacaaacagcatgtccttgatcggggcgcggtaggtcatcgatgtctcccaacatgtatgaaaaagccgttcgcgggtcacccccggaacggctcgtacttgctaccagtgccggccgcctggcggccggcaaggcatcagcccagcgcggccaccagctccggcaccacggtgttcagatcgcccaccaggccgtagtcggccaccgagaagatcggggcctccgcgtccttgttgatggccacgatcaccttgctgtccttcatgccggccaggtgttggatcgcgccgctgatgcccacggcgatgtacagctgcggggccacaatcttgccggtctggcccacctggtagtcgttcggcacgaagcccgcgtccacggcggcgcgcgaggcgcccagggccgcccccagcttgtcggccagcggggtcagcaccttggtgtagttctcgcccgagcccacgccgcggccgcccgacacgatgatcttggcggcggtcagctccgggcggtccgacttggccacctcgcggctcacgaactgcgacacgccggcatcggccacggccggcagggtctcgaccgcggccgagccgccctcggcggcggccgcgtcgaaggcggtgccgcgcacggtgatcactttgatcttgtcctccgacttcacggtggcgatggcgttgccggcgtagatcgggcgctcgaaggtgtccggggcgtccaccttcgagatctccgagatctgggccacgtccagcttcgcggccacgcgcggcaggatgttcttcccgtacggggtggccggggccagaatgtgcgagtagtcgttggcaatggccagggcctgctccgccacgttctccgcgaggccgtcgccgaagtacggggcatcggccagcaggaccttggtcacccccgcgatcttggcggcggcgtccgcggcggctttggcgttggcgccggccaccagcacgtgcacgtcgcccccgcactgggcggcggccgtcacggtgttcagggtggcggccttgatcgactggttgtcgtgctcggcgatcaccagggcggtcatgttatttctcccccgcgctcaaatcaccttggcctcgttcttgagcttctgcaccagggtcgccacgtccggcaccatcaccccggccgagcgcttggccggctccacgactttcagcgtcgacaggcgcggtttgacgtccacgccgaggtcctccggcttgacgatgtccagctgtttcttcttggctttcatgatgttcggcagcgtcacgtagcgcggctcgttcaggcgcaggtcggtggtcaccaccgcggggagcttcagcgagagggtttccaggcccccatccacctcgcgcgtcacggaggccttgccgtcggcgaccacgactttcgaggcgaaggtggcctgcggcaggccggcgagggcggccaccatctggccggtctggttcgagtcgtcgtcgatcgcctgcttgccgaggatcaccagttgcggttgctccttatcgatgagggccttcaggagtttcgccaccgccagcggctgcaggtcttcgttcgattccacgaggatgccccggtccgccccgatggccatcgccgtgcgcagggtctcctggcattgggtcacgccgcacgacaccgcgatgacttccgtcacgacgcccgcttctttgaggcggacggcctcctccacggcaatctcgtcgaacgggttcatgctcatcttcacgttggccaggtcgacgccgctgccatccgccttcacgcgcaccttgacgttgtagtccaccacgcgcttcaccgccaccaggaccttcatgcgctcactccactgatatgtgaaattgttatccgctcacaattccacacaacatacgagccggaagcataaagtgtaaagcctggggaagcttggggagaacaatcagcccggcaggggccgggctgattgtgcctgcgtgccgccggtgcttacggcagcttgaccacggcctcgcccgtcacggcgagcgcgccgccttgcgtgaagatccgggtggtcagggtggcgatcggcttgtcctcgcgcagcgcggtgacctccacttccgccgtcacctcgtcgcccacgaacaccgggagcttgaacgacagcgattggcccaggtagatcgagcccttgcccggcagttgttggccgagcaggcccgagaacagcgacgccagcagcatgccgtggacgatcgggcgctcaaaggcggtcgtcgcggcgaaggccgggtccaggtgcagcgggttgaagtcctccgacagggcggcaaacgcggccacctccgccgcgccaaaccgcttcgacaggcgggctttctgccccacctcgagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 75 - DNA, 14244 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctctcagaaaaaaataaaaaaaaaacaatacccatgtttatagggcaaaagttttataaacatgggtactggttatattatattatttatgactttattacttgtactccttcgccttttcctcgccccggagcacgcgcaggccgccttcggccagggcgagcagctcgtcttccccgccgtaccgcaccacgggcgcgatgaacttcacccggtcttcaatggcgttgcacacgtgctcattgtaggcgatgccgccggtgaggatgatggcgtccacgttccccttcagcacggtcgagcacttgccaatttctttcgccacctggaaggtgaaggcctcataaatcagggcgcactttttatcgccctccagggctttgtccacgaccgccttgaagtcgatggtattcaggtacgacaccaccccgcccttcccattgatcttcttcatcacctcttcgtaggtgtacttgttcgaaaagcacaggcgcacgaggtcgccgatgggcacgccgccggagcgttccggcgaaaacgggccctcgccatccagggtgttgttcacctcgatcacgcggccgtccttgtgggtccccaccgaggtcccgccgcccatgtgcaccacgatcaggttcagatcctcgtactttttccccacttctttggcgtagcgccgggccacggccttctggttgagggcgtgaaaaatggacttgcgcgggatgtccgccatgcccgagatgcgcgacacctcgtccagttcgtcgacgaccaccgggtccacgatgtaggccggcacgttaatttccttggcaatctcgttcgcgataatcccgcccaggttcgaggcatgctggccctgcacccccaccttcaggtcttccagcattttttggttcacggcgtacgtgcccgacacgatgggcttcagcaggcccccgcgccccacgacggcgttcagcgacgacacttcgatgttggcctccttgagcgcgtccaggatgacgttcttgcggaattggaactggtcgaaaatggtattatacttctcgatctcctccgccgagtggcgcagcgttttctcaaagatctctttctcgtcgtcatagatgccgatcttcgtggaggtgctcccggggttgatgatgaggaggcggtacatgttaacattcctccacttaactttaatttacttgttcccggcgacgagggccgccagcgcaatcgaattcatcttggtctcgtgcgaatccgcccgcgaggtcagcacgaccggggcgctggtgcccacgaggatgcccccattcttgctgtccgtcgtataggtgagggttttatacatcacgttgcccgtctcgatgttgggcatgaggaagatatccgctttgcccgccacctcccccgtcacgcccttatggtgcgcggcttcctcggacagggcaatgtccagggccagggggccatccaccacgcagcccttgatctggccgcgatccgacatcttgctcagcatggcggcatccagggtcgacggcattttggggttgatcacctccacggcgcagatcggggccactttcgggttttcgatgccgatcgcgtgggccaccttcaccgaattgttgacaatgtcgatcttctctttcagctccgggtaggtgttaaaggcgacgtccgtgagaaacaggaggcggtcaaacttctcggtctcaaagacggccacatgcgacatcgttttccccgtgcgcaggcccacttccttgttgagcaccgagcgcagaaacgtggcggtattcaccagccccttcatcaccatgtcggccttgccggtcgacaccagttccaccgccttcagcgccgcctttttcacattgggctcattcacaatttcaaaatcgttcacatccatgccaatcttcagggcgatggacacgatttcatcgtgatcgcccaccaggatggcgtcggcgatcccgttcttcttcgcgtcccgcacggcctccagcaccggttcgtcctgggcgaccgcgacggcgactttcttcatctccttcgatttcactttcatgatgatttcgttgaacgacttgatcattcttgaatctcctgaaattaggtgcgatcgaccacctcgcgcacgtttttgatcacgaaccccgacttcacatagcccggaatcgaattcagatccacctccaggtcctgctccgggacgtcgtacgtgatcttttccgcaaaatacttcatggtctcctccatgataatcacggtgatccactccccggcgcagcggtggttggtccaatagtcgcccccgccctgcgggatgaggtcgaacggcgagccgtcccaggtctcgaaccgctccgggcgaaattcgttcgggtcgtcccacaggctctcgtcatgggtggtgccatacacgtcgagggccagccccacgccggcgggaatggtcacgccttggaagtcgatatccactttggccttccccgggaggaacggcacgaacgggtagtagcggcggacctcctgcgcgaacttgtaggcgtagtccggctccgacttgatcttctcgcgggtgatgggattctcgttcatcgcgtgcagcccgaaggacacgaagcggttgatggcgatcagcggccgaaaggtgttcatcaggtcaatcgcgcacgtccgcgagtccatcgggttccccaggtagtcctcccagtgggcaaactcatacagggcggtgccctcgggcgggtggatgttgcccttgcgcgtctcgatgatctgctcctcgagccaatcctccacgcgccgccgcgcctccttcgacgctttgtagcccttaaacgccccccccagggcgcgaaagctgtcgatcataatatccatgtcggtcgcgatgcgctcaatgtcctccgggggcgcctggaccccggcccagcgggtgcccaccttggtcaggagcacgatcgattcgcggtagatgttcacctcatccatcgactccatgcgctgcgtgttggcgtgccacagggtgcgggtcagctcccgcacgtagttgaggttgccttcggtcatcagcgacatgaagagcgccttgcggtccacgtgcttcttgccgtccacggtatggatggcgcctttgccgaacagggtattcacaatgcgcttgggcagcatgccttcgcgttgcaccacgtcgttgttgtagaacatctcggcgccctccttcccggtcaccacgacgaagggcttccccccgagcgctttggtctggaacaccgaggtgttgaggcggttgcgctgattggtcgtgtacagatagccctgcttcagcaccttcagcgtgttatcgaggcccttatcgcgcttcagggtggccattcttgaatctcctgaaaaagtgagtttttgatcacaaatcggccggctaccgaggcgacggccgtcagaaatgttcaggtgtcgcaggaggctgttagcactcacccctgacgagtgctaattatagggacggggtaggggcatttcaagacgcgggtgtgatgcgggttggatccagcaggctgcctcgataagcaaaaagggcggccccgcggggccgccctttttctttccggcgactgtcaggccactcagttgttggcggccttgacttgggcgatcagctcgggcaccaccttgttcacatcgcccacaatggccaggtcggccaccttcatgatcggggcttccacgtccttgttgatcgcgatgatgtagtccgagtcctgcatgccggccaggtgctggatggcgcccgagatgccgcaggcgatgtacagggtcgggcgcacggtcttgccggtctgccccacttggaggtccttatccacccactccttttcgatggcggcgcgcgaggcggcgatggtgccgccgaggagggaggccagctcctccagcttctcaaagttctccttcgagcccacgccgcggcccccggccacgagcaccttggcctcgccgatatcggcaatatccttggcgagcttcaccactttcgagaccttggtgcggatgtccgaggcggtcagcttgatggccactttctcgatcttgtcgtccgacacgttggcgtcgttcaccggcagcttctcgaagaacacgccgggccgcaccgtggccatctgcgggcggtggtccgagcacacgatggtggcgatcaggttgccgccgaaggccgggcgggtcgcgagcagatcgcggttctccacgtcgatgtccagcgaggtgcagtcggcggtcaggccggtcgacagccgggcggcgatccgcgggccgaggtcgcggccgatgaacgtggcgccgatgaacaggatctccggcttgcgctcgttcaccaggtcacagatcaccttggcgtagccgtcggtgctgaaatgggccagcagctcgttgtcggcggccaggaccttgtccgcgccgtgcgacagcaggtccttcgacatcttctcggtgttgtgccccagcagcacggccgtcagctccacgcccagtttctcggccatttccttgcccttccccagcagttccagcgacaccttctgcagctcgccgtcgcgttgctcggcgaacacccacacgcccttgtagtcggctttgttcattgaaaaatccctcctaacttaaatatgtgttcttcttttagttctgcgacagcatatcggccgcctccttcacgggcttatcgatcacttcgccttggcccttgacttctttggtcgacgactttttcaccttggtcggcgagcccttcagccccaggttggccttgtcgacgtcgatgtcatccgcggtccacatcttgacctccttgtcgaacgcgccgaaaatcttttccaccgacatgtagcgcggcacgttcagctccttgatggccgtcagcaggaccggggtcttcacctccacgacctcgtagccgtcctcccaggccttgcggattttcagcgtatcgccatccacctcgactttctccacgtaggtcacctgggggatgcccagatgctcggcgatctccggcccgacctgggccgtgtcgccgtcaatggcctggcgcccggcaaacacgatatcatatttcagcttcttgatgcccgcggcgatggtgtgcgaggtcgccagggtatccgcccccccgaaggcgcggtcggtcagcagcaccgcctcgtccgcccccatggccagggcctccaccagggcgttcttggcctgcggggggcccatcgagatcacggtcacatgggccccgtagttgtctttcaggaccagcgcttcctccagcgcattcttgtcgtccgggttgatgatcgacggcacgccctcgcggatcagggtccccttcaccggatcgatgcgcacctcggcggtgtccggcacctgcttcagacacaccacgatgttcatcctcttaacctccttaaattagcggaaaatcttgccgctaatcaccagcttctgcacttccgacgtgccctcgtagatctcggtaatcttggcgtcgcgcatcatgcgctcgaccgggtagtccttcgtgtagccgtagccgccgaacagttgcaccgctttggtggtcacgtccatggcgacgttggcggcatgcagcttggcgcgcgcggcgtccacggtgtacggcaggcccgcctgcttcaggtaggcggccttatagaccagataccgggcggactcaatggccacgtccatatcggccatcatccaggccaggccctgaaacttgtccagcgagcggccgaactgcttgcgctccttcatgtaggcccgggcttcgttgaaggcgccctccgcgatccccagggcttgggccgcaatgccgatccgccccccatccagggttttcatcgcaatcgggaaccccttcccctctttgccaatcatgttttccaccggcacgatcatgtcctcgaacaccagttcggtggtcgacgacgcgcggatgcccagcttttgctccaccttgccgatcgagaagcccttaaaccctttctcaatgataaaggccgagatgcctttggtgcccttggtgcggtcggtcatcgcgaagatcacgaacgtgtccgccacgccgccgttggtaataaagattttggagccgttgatcacgtagtggtcgccttccaggacggccacggtctgctgcgcgcccgagtcggtgccggcgttcggttccgtcaggccgtacgcgccgatcttctcccctttggccagcgggaccaggtacttctgtttctgctcctcggtcccgtgctcgttgatcagcgaggcgcagagggaggtgtgggccgacaggatcaccccggtggtgccgcacaccttggacagctcctccacggcgatgatgtacgacagcacgtccccccccgccccgccgtactccttcgagaacggaatccccatcatgccatactggcccatcttcttcacgttctccatcgggaagcgctcggtctcgtcgatctccgcggcaatgggcttcacctcgttttccgcgaactcgcgcaccatctggcgcaccagctcctgctcgcgggtcaggttgaagtccatataaacttacctcctatctatgtgaaattgttatccgctcacaattccacacaacatacgagccggaagcataaagtgtaaagcctggggaagcttggggagaacaatcagcccggcaggggccgggctgattgtgcctgcgtgccgccggtgcttacggcagcttgaccacggcttccccggtgacggccagggccccgccttgggtaaagatccgggtcgtcagcgtggcgatcggtttgtcctcgcgcagcgcggtgacttccacctcggcggtcacttcgtcccccacgaacaccggcagtttgaacgagagcgattggcccaggtagatgctccccttgccggggagctgctggccgaggaggcccgagaacagcgaggcgagcagcatgccatgcacgatggggcgctcgaacgcggtggtcgcggcaaacgcggggtccaggtgcagcgggttgaagtcctcggagagcgccgcaaaggccgccacctccgccgcgccaaaccgcttggacagccgggctttttgcccgacctccagcgactgggccgacatgcggcgtcctcctctgtttcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 76 - DNA, 14676 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctctcagaaaaaaataaaaaaaaaacaatacccatgtttatagggcaaaagttttataaacatgggtactggttatattatattatttatgactttattacttgtactccttcgccttttcctcgccccggagcacgcgcaggccgccttcggccagggcgagcagctcgtcttccccgccgtaccgcaccacgggcgcgatgaacttcacccggtcttcaatggcgttgcacacgtgctcattgtaggcgatgccgccggtgaggatgatggcgtccacgttccccttcagcacggtcgagcacttgccaatttctttcgccacctggaaggtgaaggcctcataaatcagggcgcactttttatcgccctccagggctttgtccacgaccgccttgaagtcgatggtattcaggtacgacaccaccccgcccttcccattgatcttcttcatcacctcttcgtaggtgtacttgttcgaaaagcacaggcgcacgaggtcgccgatgggcacgccgccggagcgttccggcgaaaacgggccctcgccatccagggtgttgttcacctcgatcacgcggccgtccttgtgggtccccaccgaggtcccgccgcccatgtgcaccacgatcaggttcagatcctcgtactttttccccacttctttggcgtagcgccgggccacggccttctggttgagggcgtgaaaaatggacttgcgcgggatgtccgccatgcccgagatgcgcgacacctcgtccagttcgtcgacgaccaccgggtccacgatgtaggccggcacgttaatttccttggcaatctcgttcgcgataatcccgcccaggttcgaggcatgctggccctgcacccccaccttcaggtcttccagcattttttggttcacggcgtacgtgcccgacacgatgggcttcagcaggcccccgcgccccacgacggcgttcagcgacgacacttcgatgttggcctccttgagcgcgtccaggatgacgttcttgcggaattggaactggtcgaaaatggtattatacttctcgatctcctccgccgagtggcgcagcgttttctcaaagatctctttctcgtcgtcatagatgccgatcttcgtggaggtgctcccggggttgatgatgaggaggcggtacatgttaacattcctccacttaactttaatttacttgttcccggcgacgagggccgccagcgcaatcgaattcatcttggtctcgtgcgaatccgcccgcgaggtcagcacgaccggggcgctggtgcccacgaggatgcccccattcttgctgtccgtcgtataggtgagggttttatacatcacgttgcccgtctcgatgttgggcatgaggaagatatccgctttgcccgccacctcccccgtcacgcccttatggtgcgcggcttcctcggacagggcaatgtccagggccagggggccatccaccacgcagcccttgatctggccgcgatccgacatcttgctcagcatggcggcatccagggtcgacggcattttggggttgatcacctccacggcgcagatcggggccactttcgggttttcgatgccgatcgcgtgggccaccttcaccgaattgttgacaatgtcgatcttctctttcagctccgggtaggtgttaaaggcgacgtccgtgagaaacaggaggcggtcaaacttctcggtctcaaagacggccacatgcgacatcgttttccccgtgcgcaggcccacttccttgttgagcaccgagcgcagaaacgtggcggtattcaccagccccttcatcaccatgtcggccttgccggtcgacaccagttccaccgccttcagcgccgcctttttcacattgggctcattcacaatttcaaaatcgttcacatccatgccaatcttcagggcgatggacacgatttcatcgtgatcgcccaccaggatggcgtcggcgatcccgttcttcttcgcgtcccgcacggcctccagcaccggttcgtcctgggcgaccgcgacggcgactttcttcatctccttcgatttcactttcatgatgatttcgttgaacgacttgatcattcttgaatctcctgaaattaggtgcgatcgaccacctcgcgcacgtttttgatcacgaaccccgacttcacatagcccggaatcgaattcagatccacctccaggtcctgctccgggacgtcgtacgtgatcttttccgcaaaatacttcatggtctcctccatgataatcacggtgatccactccccggcgcagcggtggttggtccaatagtcgcccccgccctgcgggatgaggtcgaacggcgagccgtcccaggtctcgaaccgctccgggcgaaattcgttcgggtcgtcccacaggctctcgtcatgggtggtgccatacacgtcgagggccagccccacgccggcgggaatggtcacgccttggaagtcgatatccactttggccttccccgggaggaacggcacgaacgggtagtagcggcggacctcctgcgcgaacttgtaggcgtagtccggctccgacttgatcttctcgcgggtgatgggattctcgttcatcgcgtgcagcccgaaggacacgaagcggttgatggcgatcagcggccgaaaggtgttcatcaggtcaatcgcgcacgtccgcgagtccatcgggttccccaggtagtcctcccagtgggcaaactcatacagggcggtgccctcgggcgggtggatgttgcccttgcgcgtctcgatgatctgctcctcgagccaatcctccacgcgccgccgcgcctccttcgacgctttgtagcccttaaacgccccccccagggcgcgaaagctgtcgatcataatatccatgtcggtcgcgatgcgctcaatgtcctccgggggcgcctggaccccggcccagcgggtgcccaccttggtcaggagcacgatcgattcgcggtagatgttcacctcatccatcgactccatgcgctgcgtgttggcgtgccacagggtgcgggtcagctcccgcacgtagttgaggttgccttcggtcatcagcgacatgaagagcgccttgcggtccacgtgcttcttgccgtccacggtatggatggcgcctttgccgaacagggtattcacaatgcgcttgggcagcatgccttcgcgttgcaccacgtcgttgttgtagaacatctcggcgccctccttcccggtcaccacgacgaagggcttccccccgagcgctttggtctggaacaccgaggtgttgaggcggttgcgctgattggtcgtgtacagatagccctgcttcagcaccttcagcgtgttatcgaggcccttatcgcgcttcagggtggccattcttgaatctcctgaaaaagtgagtttttgatcacaaatcggccggctaccgaggcgacggccgtcagaaatgttcaggtgtcgcaggaggctgttagcactcacccctgacgagtgctaattatagggacggggtaggggcatttcaagacgcgggtgtgatgcgggttggatccagcaggctgcctcgataagcaaaaagggcggccccgcggggccgccctttttctttccggcgactgtcaggccactcagttgttggcggccttcacctgcgcgatcagctccgggaccactttgttcacgtcgcccacgatcgccaggtcggccaccttcataatcggggcctccacgtccttattgatggcgatgatgtagtccgagtcctgcatgccggccaggtgctggatcgcccccgagatgccgcaggcgatgtacagggtcgggcgcacggtcttgccggtctggcccacctggaggtctttgtccacccactctttctcgatggccgcgcgcgaggcggcgatggtgccgcccaggagcgaggccagctcctccagtttttcgaagttctccttcgagcccacgccgcggcccccggccacgagcaccttggcctcgccgatgtccgcgatgtctttggccagcttcaccactttcgacactttggtgcggatgtcggacgcggtcagcttgatggcgactttctcgatcttatcgtcggacacgttggcgtcgttcaccggcagcttctcgaagaagacgccggggcggaccgtggccatctgcgggcggtggtccgagcagacgatggtggcgatcaggttgccgccgaacgccgggcgggtcgccaggaggtcccggttctccacgtcgatgtccagcgaggtgcagtcggcggtcaggccggtcgacaggcgcgccgcgatccgcgggcccaggtcccgcccgatgaacgtggcgccaatgaacaggatctccggcttgcgctcgttcacgaggtcgcagatcaccttcgcgtagccgtcggtcgagaagtgggcgaggagttcattgtcggccgcgagcaccttatccgcgccgtggctcagcaggtccttcgacatcttctcggtgttgtggcccagcagcacggcggtcagttccacccccagcttctcggccatctccttgcccttgcccagcagctccagcgacaccttctgcagctcgccgtcgcgctgctccgcaaacacccacacgcccttgtagtcggccttgttcattgaaaaatccctcctaacttaaatatgtgttcttcttttagttctgcgagagcatatcggccgcttccttcaccggtttatcaatgacctcgccctgccctttcacttctttcgtgctcgacttcttcactttggtcggcgaccccttcaggcccagattggctttatccacgtcgatatcatccgcggtccacattttcacctccttgtcgaacgcgccgaagattttctccaccgacatgtaccgcgggacgttcagttccttaatggccgtcaggagcaccggggtcttgacttccacgacctcgtacccatcctcccaggccttccggatcttgagcgtgtccccatccacctccactttttccacgtaggtgacttggggaatcccgagatgttccgcaatctccggcccgacctgcgccgtatcgccgtcgatggcctggcggccggcaaagacaatgtcatatttcagcttcttaatgccggcggcaatggtatgcgaggtggcgagggtgtccgcgcccccaaacgcccggtccgtgagcagcaccgcctcatcggcccccatggccagcgcttcgaccagggcgttcttggcttggggggggcccatcgagatgaccgtgacgtgcgccccatagttatctttcagcacgagggcttcctcgagcgcgtttttgtcgtcggggttaatgatggacgggacgccctcccgaatcagggtgcctttgacgggatcaatgcggacctcggcggtgtcggggacttgtttgaggcagaccacaatattcatcctcttaacctccttaaattagcggaagatcttgcccgagatcaccagcttctgcacctccgaggtgccctcgtagatctccgtgatcttggcgtcgcgcatcatgcgctccacggggtagtccttggtgtagccatagccgccgaacagctgcacggccttggtggtcacgtccatggccacgttggcggcatgcagcttggcgcgggcggcatccacggtgtacggcaggcccgcctgcttcaggtaggcggccttgtacaccaggtagcgggccgactcgatggccacgtccatgtcggccatcatccaggccaggccctggaacttgtccagcgagcggccgaactgcttgcgttccttcatgtacgcgcgggcctcgttaaaggcgccttcggcgatgcccagggcctgggccgcgatgccgatgcggcccccgtccagggtcttcatggcgatcgggaagcccttgccctccttgccaatcatgttctccaccggcacgatcatgtcctcgaacaccagctcggtggtcgacgacgcgcggatgcccagcttttgctccaccttgccgatcgagaagcccttgaagcccttctcgatgataaaggccgagatgcccttggtccccttggtgcggtcggtcatggcgaagatgacgaaggtgtcggcgacgccgccgttggtgatgaagattttcgagccgttgatcacgtagtggtcgccctccagcacggccacggtctgctgggcgcccgaatcggtgccggcgttcggctcggtcaggccgtaggcgccgatcttctcccccttggccagcggcaccaggtacttctgcttctgctcctcggtgccgtgctcgttaatcagcgaggcgcacagcgaggtgtgggccgacaggatcacgccggtggtgccgcacaccttcgacagctcctccacggcgatgatgtacgacagcacgtcgccgccggccccgccgtactccttcgagaacgggatgcccatcatgccgtactggcccatcttcttcacgttctccatcgggaaccgctccgtctcgtcgatctccgcggcaatcggcttcacctcgttctcggcgaactcccggaccatctggcgcaccagctcttgctcgcgcgtcaggttgaagtccatataaacttacctcctagcggttcttgaacccttcgatcttgcgcttctcaatgaaggcggtcatggcgtctttctggtcctcggtcgagaagcattccccgaacgcttccgactcgaaggcgagggccgtgtcaatgtcgcactgcatgccgcggttaatggcttgcttcgacagtttgacggccaccggggcattgctcacgattttgttggcgatctccttggcggtgttcatcagttccgagggctccaccaccttgttcaccaggccaatgcgcagggcctcatcggccttgatgttctgcgcggtgaaaatcagctgcttggccatgcccatgccgacgaggcgcgacagccgctgggtgccgccaaagcccggggtgatgcccaggcccacttccggctggccgaagcgggcgttcgaggaggcgatgcggatatcgcaggacatcgcaatttcgcacccgccgcccagggcgaacccattcacggccgcaatcaccggtttttccagcagctccagccgccgaaacactttgttccccagaatgccgaacttgcgcccctcgatggtattcatttctttcatctccgagatgtccgccccggcgacgaagctcttctcgcccgcgcccgtcaggatcacggcgagcacctccgagtcgttctcgatttcgccgatcacgtagtccatctctttcagggtgtccgagttcagggcgttgagggccttcgggcggttgatggtcaccaccgcgaccttgccttctttctccaggatgacattgttgagttccatgactaatcctcctaaaatgtgaaattgttatccgctcacaattccacacaacatacgagccggaagcataaagtgtaaagcctggggaagcttcttaagtaataaaaataagagttaccttaaatggtaactcttatttttttaatattgtttcatagtatttctttttatttcgagtagtcgtaaaagcccttgccgctcttgcggcccagccagcccgcccggacatactttttcagcagcgtgtgcgggcgatacttcgaatcgccggtttccgagtacagcacgtccatgatggccaggcaaatgtcgaggccgataaagtcccccagttccagcgggcccatcgggtggttcgcccccagcttcatggccttatcaatatcctcgaccgaggcaatcccttcggcgaggatgcccaccgcttcattgatcatcgggatcagaatgcggttgacgacgaaccccggcgcctcggcgacttccacggggtccttgccaatggcaatcgacgtctctttcaccgcgtcgaaggtttcctgcgacgtggcaatgccgcgaatgacctccacgagtttcatgacgggggcgggattaaagaaatgcatgccgatcactttgtcgttggtcttggtcgcgctcgccacctccgtgatcgagagcgagctggtgttggaggcgagaatcgtctccggtttgcagatgttgtcgagatccgcaaagatctgtttcttaatgtccatgcgctcgacggcggcttcaatcacgaggtcgcagtccgcggccatgttcaggtccacggtcccgctgatgcgggtgagaatctccaccttggtggcctcttcgatcttgccttttttgaccagtttcgacaggttcttgttaatgaaatccaggccgcgatcgacgaattcatccttgatgtcccggaggaccacctcgaagcctttggcggcgaaggcctgcgcgatgccggaccccatcgtccccgcgccaatgacgcacaccttcttcattcttgaatctcctgaaactagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 77 - DNA, 1689 bp, Aquincola tertiaricarbonis L108
  CDS (1)..(1689)
atgacctggcttgagccgcagataaagtcccaactccaatcggagcgcaaggactgggaagcgaacgaagtcggcgccttcttgaagaaggcgcccgagcgcaaggagcagttccacacgatcggggacttcccggtccagcgcacctacaccgctgccgacatcgccgacacgccgctggaggacatcggtcttccggggcgctacccgttcacgcgcgggccctacccgacgatgtaccgcagccgcacctggacgatgcgccagatcgccggcttcggcaccggcgaggacaccaacaagcgcttcaagtatctgatcgcgcagggccagaccggcatctccaccgacttcgacatgcccacgctgatgggctacgactccgaccacccgatgagcgacggcgaggtcggccgcgagggcgtggcgatcgacacgctggccgacatggaggcgctgctggccgacatcgacctcgagaagatctcggtctcgttcacgatcaacccgagcgcctggatcctgctcgcgatgtacgtggcgctcggcgagaagcgcggctacgacctgaacaagctgtcgggcacggtgcaggccgacatcctgaaggagtacatggcgcagaaggagtacatctacccgatcgcgccgtcggtgcgcatcgtgcgcgacatcatcacctacagcgcgaagaacctgaagcgctacaacccgatcaacatctcgggctaccacatcagcgaggccggctcctcgccgctccaggaggcggccttcacgctggccaacctgatcacctacgtgaacgaggtgacgaagaccggtatgcacgtcgacgaattcgcgccgcggttggccttcttcttcgtgtcgcaaggtgacttcttcgaggaggtcgcgaagttccgcgccctgcgccgctgctacgcgaagatcatgaaggagcgcttcggtgcaagaaatccggaatcgatgcggttgcgcttccactgtcagaccgcggcggcgacgctgaccaagccgcagtacatggtcaacgtcgtgcgtacgtcgctgcaggcgctgtcggccgtgctcggcggcgcgcagtcgctgcacaccaacggctacgacgaagccttcgcgatcccgaccgaggatgcgatgaagatggcgctgcgcacgcagcagatcattgccgaggagagtggtgtcgccgacgtgatcgacccgctgggtggcagctactacgtcgaggcgctgaccaccgagtacgagaagaagatcttcgagatcctcgaggaagtcgagaagcgcggtggcaccatcaagctgatcgagcagggctggttccagaagcagattgcggacttcgcttacgagaccgcgctgcgcaagcagtccggccagaagccggtgatcggggtgaaccgcttcgtcgagaacgaagaggacgtcaagatcgagatccacccgtacgacaacacgacggccgaacgccagatttcccgcacgcgccgcgttcgcgccgagcgcgacgaggccaaggtgcaagcgatgctcgaccaactggtggctgtcgccaaggacgagtcccagaacctgatgccgctgaccatcgaactggtgaaggccggcgcaacgatgggggacatcgtcgagaagctgaaggggatctggggtacctaccgcgagacgccggtcttctga

SEQ ID NO: 78 - protein, 562 aa, Aquincola tertiaricarbonis L108
MTWLEPQIKSQLQSERKDWEANEVGAFLKKAPERKEQFHTIGDFPVQRTYTAADIADTPLEDIGLPGRYPFTRGPYPTMYRSRTWTMRQIAGFGTGEDTNKRFKYLIAQGQTGISTDFDMPTLMGYDSDHPMSDGEVGREGVAIDTLADMEALLADIDLEKISVSFTINPSAWILLAMYVALGEKRGYDLNKLSGTVQADILKEYMAQKEYIYPIAPSVRIVRDIITYSAKNLKRYNPINISGYHISEAGSSPLQEAAFTLANLITYVNEVTKTGMHVDEFAPRLAFFFVSQGDFFEEVAKFRALRRCYAKIMKERFGARNPESMRLRFHCQTAAATLTKPQYMVNVVRTSLQALSAVLGGAQSLHTNGYDEAFAIPTEDAMKMALRTQQIIAEESGVADVIDPLGGSYYVEALTTEYEKKIFEILEEVEKRGGTIKLIEQGWFQKQIADFAYETALRKQSGQKPVIGVNRFVENEEDVKIEIHPYDNTTAERQISRTRRVRAERDEAKVQAMLDQLVAVAKDESQNLMPLTIELVKAGATMGDIVEKLKGIWGTYRETPVF

SEQ ID NO: 79 - DNA, 411 bp, Aquincola tertiaricarbonis L108
  CDS (1)..(411)
atggaccaaatcccgatccgcgttcttctcgccaaagtcggcctcgacggccatgaccgaggcgtcaaggtcgtcgctcgcgcgctgcgcgacgccggcatggacgtcatctactccggccttcatcgcacgcccgaagaggtggtcaacaccgccatccaggaagacgtggacgtgctgggtgtaagcctcctgtccggcgtgcagctcacggtcttccccaagatcttcaagctcctggacgagagaggcgctggcgacttgatcgtgatcgccggtggcgtgatgccggacgaggacgccgcggccatccgcaagctcggcgtgcgcgaggtgctcctgcaggacacgcccccgcaggccatcatcgactcgatccgctccttggtcgccgcgcgcggcgcccgctga

SEQ ID NO: 80 - protein, 136 aa, Aquincola tertiaricarbonis L108
MDQIPIRVLLAKVGLDGHDRGVKVVARALRDAGMDVIYSGLHRTPEEVVNTAIQEDVDVLGVSLLSGVQLTVFPKIFKLLDERGAGDLIVIAGGVMPDEDAAAIRKLGVREVLLQDTPPQAIIDSIRSLVAARGAR

SEQ ID NO: 81 - DNA, 984 bp, Aquincola tertiaricarbonis L108
  CDS (1)..(984)
atgacttacgttccctcatccgccttgctcgagcaactccgagccggcaatacctgggcgcttggccgcctgatctcgcgcgccgaggccggtgtggccgaggcgcggccagcattggccgaggtctatcggcacgccggctcggcgcatgtgatcggtctcaccggggtgccggggagtggcaagtcgactctcgtggcgaagctcacggccgccctgcgcaagcgtggtgaaaaggtcggcatcgtcgcaatcgatccgtcgagcccgtactcgggcggtgcgatcctcggcgaccgtatccgaatgaccgaactcgccaacgattccggcgtattcatccgcagcatggccacgcgcggcgcgacggggggcatggcgcgtgccgccctcgacgccgtggacctgctggatgtcgccggctatcacaccatcatcctggagactgtcggagtcggtcaggacgaggtggaggtggcgcacgcatcggacacgacagtcgtcgtatcggcgccaggccttggagacgagatccaggccatcaaagccggcgtcctggaaatcgccgacatccatgttgtcagcaagtgtgaccgcgacgacgcgaatcgcacgctcaccgatctcaagcagatgctgacgctcggcaccatggtcgggcccaagcgcgcatgggcgatcccggtcgtcggtgtcagttcgtacacaggcgaaggcgtcgacgacctgctcggtcgcatcgccgcccaccgccaggcgacggccgacaccgaactcggccgcgaacggcgccgtcgcgtagccgaattccgccttcagaagaccgccgagacgctgctcctggagcgattcaccaccggagcgcagcccttctcgcctgcgctcgcagacagcctcagcaaccgtgcgtcggatccctacgccgcagcacgcgaactcatcgcccgaacgatccgcaaggagtactcgaatgacctggcttga

SEQ ID NO: 82 - protein, 327 aa, Aquincola tertiaricarbonis L108
MTYVPSSALLEQLRAGNTWALGRLISRAEAGVAEARPALAEVYRHAGSAHVIGLTGVPGSGKSTLVAKLTAALRKRGEKVGIVAIDPSSPYSGGAILGDRIRMTELANDSGVFIRSMATRGATGGMARAALDAVDLLDVAGYHTIILETVGVGQDEVEVAHASDTTVVVSAPGLGDEIQAIKAGVLEIADIHVVSKCDRDDANRTLTDLKQMLTLGTMVGPKRAWAIPVVGVSSYTGEGVDDLLGRIAAHRQATADTELGRERRRRVAEFRLQKTAETLLLERFTTGAQPFSPALADSLSNRASDPYAAARELIARTIRKEYSNDLA

SEQ ID NO: 83 - DNA, 9837 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcagcaggctgcctcgataagcaaaaagggcggccccgcggggccgccctttttctttccggcgactgtcaggccacttacaggatgacgacggccttgatcaggtccttcggcttgtctttcatcagcaggagggcctcctcgatgtggtcgaagccatggtacacgtgcgtcaccagtttcgacaggtcgacgcgattatacaccaccatgtcgcgcagcatctccgcgcgcagccgcccgccggggcacaggccgcccttaatggtcttatgggccatgccgcagccccattccacgcgggggatgagcagcgcgtcgcccgagccgtggtagttgatgttcgaaatgatcccgcccggcttcaccatcgagacggcctgcgacagcgtttcggacccccccccggccatgatgacgcggtccacgcccttgccgttggtcagcttcatgacctggtccacaatatggccgttcttgtagttcaggatgtcggtggcgccgtagaacttggccgcctccacgcagatcggccgcgagcccacgccgatgatgcggcccgccccccgcagcttcgcgcccgcgatgcccatgagccccaccgccccgatgccgatcacgacgaccgagctgcccatctggatatccgccagctcggccccgtggaaccccgtggtcatcatatcggtgatcatcaccgcgttttccagcggcatatctttcggcaggatggccaggttcatgtcggcgtcgttcacatgaaagtactccccgaacacgccgtccttgaagttcgagaacttccacccggcgagcatgccgttcgaatgctgctggaacccggcctggacctccagcgagcgccagtcgggggtggtgcacggcacgatcacccggtcgccgggcttaaagtccttcacctccgaccccacctccaccacctcgcccacggcctcgtgccccaggatcatattcttgcggtcccccagcgcgccctcaaacacggtgtggatgtccgacgtacacggcgacacggcgagcgggcgcacgatggcgtcatacgagccggccaccgggcgctccttctcgatccagcccagtttgttgatgcccagcatggcgaagcccttcattcttgaatctcctgaaaaagtgagtttttgatcacaaatcggccggctaccgaggcgacggccgtcagaaatgttcaggtgtcgcaggaggctgttagcactcacccctgacgagtgctaattatagggacggggtaggggcatttcaagacgcgggtgtgatgcgggttactagtggatcccaatattaaaaaaataagagttaccatttaaggtaactcttatttttattacttcaggtagtcgtagatgacctccgcccggggcaggataatatcggcgaggatatgcgaggacgacacgatttctttcaccggcaggtcattcaggggcgccatcgcatggtcgaagagctgcaggcgcgtggggcccgtccaggcttcatgcacggtcacgtcggtgatcttcgcgttgatcagttcgcaaatgcgcggggagccgtcgtaattggggatgattttcagcatgtagttggggcggcaaatctggtccttcgcttcattcgcgtccagcgccttgtgcttgtagcccatggtggcggtggcgacgcgcagtttgccgtagtcgagggtgccgaccagcgtgtccgaatccacaaacagcttcggatagcccagcttcttggggtaggcggacagctcgcggcccacggcaatggccggttcgttgtccaggtacatcatgtgcaggtaatcccccttcacgccattaaacgagaccggaatcgcctggccgctttcggtatagcagcccaggccggaggtgtcatgcatcgccataatctcgaagcgcaccaggggctcatcgatctccagcggttccgggacgaccttgcggagggcatccatgtcggtgcggtacacgatgttgaagtactcgcggttgtggaacttgtagggcccccgcggaaaggcgggggacgtcagcggggtgctgatttgtttgatgacttcgtccttgagcattcttgaatctcctgaaatgtgaaattgttatccgctcacaattccacacaacatacgagccggaagcataaagtgtaaagcctggggaagctttcgatgttcaagaaaacacccgataactttcgctatcgggtgtttttattgattagttgaggcgttcgatcaccatggcgatgccctggcccccgccgatgcacagggtggccaggcccagcgtcttatcccgggcctgcatggcgtgcagcagggtcaccaggatgcgcgcgcccgaggcgccgatcggatggcccagggcgatcgcgccgccgttcacattcaccttctccgagtcgaagcccaggttcttgcccaccgccaggaactgggcggcgaaggcctcgttggcctcgatcaggtcgatgtccgccagttgcaggcccgccagctgcagggccttctgggtggccggcaccgggcccatgcccatcagggccggcggcaccccgcccgaggcgtacgacttgatgcgggccagcggggtcagccccgcggccagcgcggccgactcctccatgatcaccagcgccgcggcgccgtcgttgatgcccgaggcgttgcccgcggtcacggtgccggctttgtcgaaggccgggcgcagggcccccagggcctcggcggtcgagttggccttcgggaactcgtcctgcgagaacacgaaggtcttcttgcgggtcaccacgttcaccggcacgatctcggccgtgaaggcgcccgactcgatggcggcggcggccttgcgctgcgagtgcagggccagctcgtcctgcatctcgcgggtgatgccgtactccttggccacgttctcggcggtgatgcccatgtggtagccgtgggtggcgcacatgaggccgtcgcggaggatcacgtcgtacacctggccgtcccccagccgatagcccgagcgggccttggcgtccagcaggtacggggccagcgacatgttctccatgccgccggccacgatcgattgggcttgcccggcttggatggcttgggcggcgagggcgaccgacttcaggccggagccgcacaccttgttcacggtgaagccgcacacggtctcggcgaggcccgacttcaggagggcctgccgggccgggttctggcccagcccggcttgcagcacgttgcccatgatcacctcgtccacgtgttgcgagtcgatcttggcgcgctcgatggccgccttgatcacggtggcccccaggtcgatggccgaggtcgaggcgagcgagccgttgaacgagccgatcgcggtgcggacggccgacacgatcacgcagttcttcattttatattccttctgtttgtaggggtgccgtcacaggtcgccgcgctgggtgttcaggtcggcggccacctcgaagcgggcctcggtcttggcgcgcacggtcgcgaggtcgcagccgtcggcgatctcggtcagccacatcttgccgtcgatgaagcggaacacggccagttcggtcaccagcatgtgcacggcgtgctgggccgtcagcggcatggtgcagcggcgcaggatcttggccgagccatccttggcgcagtgctccatcgcaatgatcaccttgcgcgagccggtcacgaggtccatggccccccccatgcccgggaccattttgcccggcacgacccagttggccaggttggcctcctcgtccacctggagcccgccgaggacgcaggcgtcgatgtgccccccccggatcagggcgaacgacatggccgagtcgaacatcgcggcgcccggcagcaccccgcacggctggccccccgcgttcaccaggtccgggtgggcggtggtcaccgggcccaggcccaggaagccgttctccgactgcagggtgatgtggatgccctccggcagatagttggccaccatggtcggcaggccgatgcccaggttcacgatgtcgccgtcgcgcagttcctgcgcgacgcggcgggcgatgcgctgcttggcgtccattacttcgactcttgggagacgatgatatggtcgatcaccgcgccgggggtcacgatatgatccggttgcagctccccggtttcgacgagttcgtccggttccacgagggtgatatccgcggccagcgcaatcagcgggttgaagttccgggcggacagctggtacgtcaggttgcccagggtgtcgcagcgatgggcgcgaatcagggccagatcggcgcgcagcgggcgttccagcagccaggtcttcccgtccagggtcagcgtctgcttgccttcctcgaccacggtgcccacgccggtcggcgtcaggaacccgccgaggcccgccccgccgcaccggatttgctcaatgagggtcccctgcgggaccagcaccacatccatctcgcccgagatcatccggcggccggtctccgggttcgtgccgatgtggctggcgatcactttgcggacgcggccgttgacaatcagggggccaatgccggtgtccacgaaggcggtgtcgttggcaatgagcgtcagatcgcgcacccccgactccaggagggcttcgaccagccgcgacggcgtcccgatgcccatgaagccccccaccatgatggtcatgccatcgcggaaaaagccggtggcgtcctgcagcgtcatgagcttggtcttcattttttatccctcttgcatacggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 84 - DNA, 9867 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcagcaggctgcctcgataagcaaaaagggcggccccgcggggccgccctttttctttccggcgactgtcaggccacttacaggatgacgacggccttgatcaggtccttcggcttgtctttcatcagcaggagggcctcctcgatgtggtcgaagccatggtacacgtgcgtcaccagtttcgacaggtcgacgcgattatacaccaccatgtcgcgcagcatctccgcgcgcagccgcccgccggggcacaggccgcccttaatggtcttatgggccatgccgcagccccattccacgcgggggatgagcagcgcgtcgcccgagccgtggtagttgatgttcgaaatgatcccgcccggcttcaccatcgagacggcctgcgacagcgtttcggacccccccccggccatgatgacgcggtccacgcccttgccgttggtcagcttcatgacctggtccacaatatggccgttcttgtagttcaggatgtcggtggcgccgtagaacttggccgcctccacgcagatcggccgcgagcccacgccgatgatgcggcccgccccccgcagcttcgcgcccgcgatgcccatgagccccaccgccccgatgccgatcacgacgaccgagctgcccatctggatatccgccagctcggccccgtggaaccccgtggtcatcatatcggtgatcatcaccgcgttttccagcggcatatctttcggcaggatggccaggttcatgtcggcgtcgttcacatgaaagtactccccgaacacgccgtccttgaagttcgagaacttccacccggcgagcatgccgttcgaatgctgctggaacccggcctggacctccagcgagcgccagtcgggggtggtgcacggcacgatcacccggtcgccgggcttaaagtccttcacctccgaccccacctccaccacctcgcccacggcctcgtgccccaggatcatattcttgcggtcccccagcgcgccctcaaacacggtgtggatgtccgacgtacacggcgacacggcgagcgggcgcacgatggcgtcatacgagccggccaccgggcgctccttctcgatccagcccagtttgttgatgcccagcatggcgaagcccttcattcttgaatctcctgaaaaagtgagtttttgatcacaaatcggccggctaccgaggcgacggccgtcagaaatgttcaggtgtcgcaggaggctgttagcactcacccctgacgagtgctaattatagggacggggtaggggcatttcaagacgcgggtgtgatgcgggttactagtggatcccaatattaaaaaaataagagttaccatttaaggtaactcttatttttattacttcaggtagtcgtagatgacctccgcccggggcaggataatatcggcgaggatatgcgaggacgacacgatttctttcaccggcaggtcattcaggggcgccatcgcatggtcgaagagctgcaggcgcgtggggcccgtccaggcttcatgcacggtcacgtcggtgatcttcgcgttgatcagttcgcaaatgcgcggggagccgtcgtaattggggatgattttcagcatgtagttggggcggcaaatctggtccttcgcttcattcgcgtccagcgccttgtgcttgtagcccatggtggcggtggcgacgcgcagtttgccgtagtcgagggtgccgaccagcgtgtccgaatccacaaacagcttcggatagcccagcttcttggggtaggcggacagctcgcggcccacggcaatggccggttcgttgtccaggtacatcatgtgcaggtaatcccccttcacgccattaaacgagaccggaatcgcctggccgctttcggtatagcagcccaggccggaggtgtcatgcatcgccataatctcgaagcgcaccaggggctcatcgatctccagcggttccgggacgaccttgcggagggcatccatgtcggtgcggtacacgatgttgaagtactcgcggttgtggaacttgtagggcccccgcggaaaggcgggggacgtcagcggggtgctgatttgtttgatgacttcgtccttgagcattcttgaatctcctgaaatgtgaaattgttatccgctcacaattccacacaacatacgagccggaagcataaagtgtaaagcctggggaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaattccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 85 - DNA, 9859 bp, Artificial Sequence
  vector
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcagcaggctgcctcgataagcaaaaagggcggccccgcggggccgccctttttctttccggcgactgtcaggccacttacaggatgacgacggccttgatcaggtccttcggcttgtctttcatcagcaggagggcctcctcgatgtggtcgaagccatggtacacgtgcgtcaccagtttcgacaggtcgacgcgattatacaccaccatgtcgcgcagcatctccgcgcgcagccgcccgccggggcacaggccgcccttaatggtcttatgggccatgccgcagccccattccacgcgggggatgagcagcgcgtcgcccgagccgtggtagttgatgttcgaaatgatcccgcccggcttcaccatcgagacggcctgcgacagcgtttcggacccccccccggccatgatgacgcggtccacgcccttgccgttggtcagcttcatgacctggtccacaatatggccgttcttgtagttcaggatgtcggtggcgccgtagaacttggccgcctccacgcagatcggccgcgagcccacgccgatgatgcggcccgccccccgcagcttcgcgcccgcgatgcccatgagccccaccgccccgatgccgatcacgacgaccgagctgcccatctggatatccgccagctcggccccgtggaaccccgtggtcatcatatcggtgatcatcaccgcgttttccagcggcatatctttcggcaggatggccaggttcatgtcggcgtcgttcacatgaaagtactccccgaacacgccgtccttgaagttcgagaacttccacccggcgagcatgccgttcgaatgctgctggaacccggcctggacctccagcgagcgccagtcgggggtggtgcacggcacgatcacccggtcgccgggcttaaagtccttcacctccgaccccacctccaccacctcgcccacggcctcgtgccccaggatcatattcttgcggtcccccagcgcgccctcaaacacggtgtggatgtccgacgtacacggcgacacggcgagcgggcgcacgatggcgtcatacgagccggccaccgggcgctccttctcgatccagcccagtttgttgatgcccagcatggcgaagcccttcattcttgaatctcctgaaaaagtgagtttttgatcacaaatcggccggctaccgaggcgacggccgtcagaaatgttcaggtgtcgcaggaggctgttagcactcacccctgacgagtgctaattatagggacggggtaggggcatttcaagacgcgggtgtgatgcgggttactagtggatcccaatattaaaaaaataagagttaccatttaaggtaactcttatttttattacttcaggtagtcgtagatgacctccgcccggggcaggataatatcggcgaggatatgcgaggacgacacgatttctttcaccggcaggtcattcaggggcgccatcgcatggtcgaagagctgcaggcgcgtggggcccgtccaggcttcatgcacggtcacgtcggtgatcttcgcgttgatcagttcgcaaatgcgcggggagccgtcgtaattggggatgattttcagcatgtagttggggcggcaaatctggtccttcgcttcattcgcgtccagcgccttgtgcttgtagcccatggtggcggtggcgacgcgcagtttgccgtagtcgagggtgccgaccagcgtgtccgaatccacaaacagcttcggatagcccagcttcttggggtaggcggacagctcgcggcccacggcaatggccggttcgttgtccaggtacatcatgtgcaggtaatcccccttcacgccattaaacgagaccggaatcgcctggccgctttcggtatagcagcccaggccggaggtgtcatgcatcgccataatctcgaagcgcaccaggggctcatcgatctccagcggttccgggacgaccttgcggagggcatccatgtcggtgcggtacacgatgttgaagtactcgcggttgtggaacttgtagggcccccgcggaaaggcgggggacgtcagcggggtgctgatttgtttgatgacttcgtccttgagcattcttgaatctcctgaaatgtgaaattgttatccgctcacaattccacacaacatacgagccggaagcataaagtgtaaagcctggggaagcttattatcttaagtaataaaaataagagttaccttaaatggtaactcttatttttttaatattgtttcatagtatttctttctacacggccatcgggcgcagctcattgctgatgagcaggtcggcggccgtcagcgagcgaatttcgtcaatggtggtgttcttgttgatttcggtgaggaggaggccgtcgttaatcacttcgatcacccccagttcggtcacaatcaggttcgcctgcgatttggcggtcagcggcagggtgcacttcttcaggatcttcggttggcccttgttggtgtggcgcatggcaatgatgaccttcttggccccgttcaccagatccatggcgccgcccatccccgacagcatcttgcccggcacgatccagttggcgatattgcccttctcgtccacctgcagcgcgcccagcaccgtgacatccacatgcccgccccgaatgagcgagaacgacaccgacgagtcgaaaaaggtgccgtccggcagcacggtggtatagtcgccccccgcattcaccacgtccttgtccgcctcattgattttcgggctggcccccatgcccacaatgccattttccgactggaaggtgatcttgaaattcttcgggatgtagtcggccaccatcgtgggcaggcccacgcccagattgaccagctgcccgtttttcagttcgcgggccacgcgcttcgcaataatctccttggccaggtttttgtcattaatcattttaggcgggctccttcacgatgtagttgatgagcacgcccggcgtcatggccttttccttctccagcttctcgcaggagaccaggttttcggcttccacgatcacggttttggcggccatcgccatgtacgggttgaagttcttggtggtgcccttgtagaaggtgttgccggcttcgtcgacaatcgagcctttaatcagggccacgtcggcggtcagcggcagctccaggaggtattcggtcccgttgatggagatcttctttttgcctttttcgatgagggtccccaggccggtcttggtcaggacccccccgaggcccgagccccccgcgcgaatgcgctccaccagcgtgccctgcggcgacagttccacttccagctcattgttgaacagttttttccccgtgtccggattcgagccaatgtacgaggcgatcagtttcttcacctggttgttcgagatgagcttgccgatgccggtgttcgggtagcaggtatcgttggagataatggtgagattcttgatgttcaggttcaccagaaaatcaatcagcttggtcggggtgccgcagttcagaaagcccccaatcataatcgtcatgccgtccttgaagaacgagcggaggttctcaaagcggatgatcttcgagttcattttaatccctccttttaaactttctagcacttttccagcaggatcgcggtgccctggccgccgccgatgcacagggtcgccagccccttcttggcgtcgcgcttctgcatcgcgtgcaccagggtcaccaggatgcgggcgcccgaggcgccgatcgggtgccccagggcgatggcgccgccattcacgttcactttgttcatgtcgaacttcaggtccttggcgacggccagcgactgggcggcaaaggcctcgttcgactcgatcaggtccagctcgtcgaccgtccagccggctttctcgatcgccgccttggtggcgtagaacgggccgtagcccatgatggccgggtccacgccggccgagccatacgacacgatcttcgccagcggtttcacgcccagctccttggccttttcggccgacatgatcaccagcacggccgcgcagtcgttcaggcccgaggcgttgcccgcggtcacggtgccgtccttcttgaaggccggcttcagcttggccaggccctcgatcgtcgacccgaagcgcgggtgctcgtccgtgtccaccacggtctcgcccttgcggcccttaatcaccaccggcacgatctcgtccttgaactggcccgacttgatggcttcctccgccttcttttgcgaggccagggcgaactcgtcctgctcctcgcgcgagatgttccagcgctcggcgatgttctcggcggtgatgcccatgtggtagtcgttgaaggcgtcccacaggccgtcggtgatcatctcgtccacgaacttggcgttccccatgcggtagccccagcgggcgttgttggccaggtacggggcgcgcgacatgttttccatgccgccggcaatgatcacgtcggcgtcgccggccttgatgatctgggccgccagcgacacggtgcgcaggcccgagccgcacaccttgttgatggtcatggcggggatctccaccgggaggccggctttgaagctcgcctggcgggccgggttctggccgagcccggcctgcagcacgttgcccaggatcacctcgttcacgtcctccggcttgatgccggccttcttcacggcctccttaatggcggtggcgcccaggtccacggcgggcacgtctttcaggctcttgccgtacgagccgatcgcggtgcgcacggccgaggcaatcaccacctccttcattcttgaatctcctgaaaggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 86 - DNA, 510 bp, Ralstonia eutropha
atgtcaacctataccaatgaacaggtcgcccacctcgtcgatggttccctcgattgggagaccaccttccgcatgctctcgatgcccaaggacgagagccggttcgcgcagtacctgtcagccttgcaggccaaggtgaactttcctgaccgcatcgtgctaccgctcagcccgcacatgtttatcgtgcagtcggtcaagagcaagaagtgggtagtcaagtgcgactgcggtcacgagttctgtgattaccgcgagaactggaagctgcatgccgcgatctacgtgcgagacaccgaggaggcgatgacagaggtgtatccgaagctgatggcccccgacacccagtggcaggtgtatcgcgaatactactgcccatcgtgcggcaccatgcacgacgtcgaagcgcctaccccgtggtatcccgtgatccacgacttcgagccggacatcgaggccttctacaaggagtgggtgaatctcccggtgccggagcgcgcgtccgactaa

SEQ ID NO: 87 - DNA, 1222 bp, Escherichia coli
tctagatttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaaggtctaga

SEQ ID NO: 88 - DNA, 8512 bp, Artificial Sequence
  vector
accttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacccccatgggcaaatattatacgcaaggcgacaaggtgctgatgccgctggcgattcaggttcatcatgccgtttgtgatggcttccatgtcggcagaatgcttaatgaattacaacagtttttatgcatgcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcgaattcctgcagcccgggggatccactagttctagatttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaaggtctagaaataattttgtttaactttaagaaggaattcaggagcccttcaccatgacctggcttgagccgcagataaagtcccaactccaatcggagcgcaaggactgggaagcgaacgaagtcggcgccttcttgaagaaggcccccgagcgcaaggagcagttccacacgatcggggacttcccggtccagcgcacctacaccgctgccgacatcgccgacacgccgctggaggacatcggtcttccggggcgctacccgttcacgcgcgggccctacccgacgatgtaccgcagccgcacctggacgatgcgccagatcgccggcttcggcaccggcgaggacaccaacaagcgcttcaagtatctgatcgcgcagggccagaccggcatctccaccgacttcgacatgcccacgctgatgggctacgactccgaccacccgatgagcgacggcgaggtcggccgcgagggcgtggcgatcgacacgctggccgacatggaggcgctgctggccgacatcgacctcgagaagatctcggtctcgttcacgatcaacccgagcgcctggatcctgctcgcgatgtacgtggcgctcggcgagaagcgcggctacgacctgaacaagctgtcgggcacggtgcaggccgacatcctgaaggagtacatggcgcagaaggagtacatctacccgatcgcgccgtcggtgcgcatcgtgcgcgacatcatcacctacagcgcgaagaacctgaagcgctacaacccgatcaacatctcgggctaccacatcagcgaggccggctcctcgccgctccaggaggcggccttcacgctggccaacctgatcacctacgtgaacgaggtgacgaagaccggtatgcacgtcgacgaattcgcgccgcggttggccttcttcttcgtgtcgcaaggtgacttcttcgaggaggtcgcgaagttccgcgccctgcgccgctgctacgcgaagatcatgaaggagcgcttcggtgcaagaaatccggaatcgatgcggttgcgcttccactgtcagaccgcggcggcgacgctgaccaagccgcagtacatggtcaacgtcgtgcgtacgtcgctgcaggcgctgtcggccgtgctcggcggcgcgcagtcgctgcacaccaacggctacgacgaagccttcgcgatcccgaccgaggatgcgatgaagatggcgctgcgcacgcagcagatcattgccgaggagagtggtgtcgccgacgtgatcgacccgctgggtggcagctactacgtcgaggcgctgaccaccgagtacgagaagaagatcttcgagatcctcgaggaagtcgagaagcgcggtggcaccatcaagctgatcgagcagggctggttccagaagcagattgcggacttcgcttacgagaccgcgctgcgcaagcagtccggccagaagccggtgatcggggtgaaccgcttcgtcgagaacgaagaggacgtcaagatcgagatccacccgtacgacaacacgacggccgaacgccagatttcccgcacgcgccgcgttcgcgccgagcgcgacgaggccaaggtgcaagcgatgctcgaccaactggtggctgtcgccaaggacgagtcccagaacctgatgccgctgaccatcgaactggtgaaggccggcgcaacgatgggggacatcgtcgagaagctgaaggggatctggggtacctaccgcgagacgccggtcttctgagcaggaagcttcccaccatggaccaaatcccgatccgcgttcttctcgccaaagtcggcctcgacggccatgaccgaggcgtcaaggtcgtcgctcgcgcgctgcgcgacgccggcatggacgtcatctactccggccttcatcgcacgcccgaagaggtggtcaacaccgccatccaggaagacgtggacgtgctgggtgtaagcctcctgtccggcgtgcagctcacggtcttccccaagatcttcaagctcctggacgagagaggcgctggcgacttgatcgtgatcgccggtggcgtgatgccggacgaggacgccgcggccatccgcaagctcggcgtgcgcgaggtgctcctgcaggacacgcccccgcaggccatcatcgactcgatccgctccttggtcgccgcgcgcggcgcccgctgaaagggcgagctctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgag

SEQ ID NO: 89 - DNA, 9451 bp, Artificial Sequence
  Vector
accttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacccccatgggcaaatattatacgcaaggcgacaaggtgctgatgccgctggcgattcaggttcatcatgccgtttgtgatggcttccatgtcggcagaatgcttaatgaattacaacagtttttatgcatgcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgataaatttagatctggagaccggaatgacttacgttccctcatccgccttgctcgagcaactccgagccggcaatacctgggcgcttggccgcctgatctcgcgcgccgaggccggtgtggccgaggcgcggccagcattggccgaggtctatcggcacgccggctcggcgcatgtgatcggtctcaccggggtgccggggagtggcaagtcgactctcgtggcgaagctcacggccgccctgcgcaagcgtggtgaaaaggtcggcatcgtcgcaatcgatccgtcgagcccgtactcgggcggtgcgatcctcggcgaccgtatccgaatgaccgaactcgccaacgattccggcgtattcatccgcagcatggccacgcgcggcgcgacggggggcatggcgcgtgccgccctcgacgccgtggacctgctggatgtcgccggctatcacaccatcatcctggagactgtcggagtcggtcaggacgaggtggaggtggcgcacgcatcggacacgacagtcgtcgtatcggcgccaggccttggagacgagatccaggccatcaaagccggcgtcctggaaatcgccgacatccatgttgtcagcaagtgcgaccgcgacgacgcgaatcgcacgctcaccgatctcaagcagatgctgacgctcggcaccatggtcgggcccaagcgcgcatgggcgatcccggtcgtcggtgtcagttcgtacacaggcgaaggcgtcgacgacctgctcggtcgcatcgccgcccaccgccaggcgacggccgacaccgaactcggccgcgaacggcgccgtcgcgtagccgaattccgccttcagaagaccgccgagacgctgctcctggagcgattcaccaccggagcgcagcccttctcgcctgcgctcgcagacagcctcagcaaccgtgcgtcggatccctacgccgcagcacgcgaactcatcgcccgaacgatccgcaaggagtactcgaatgacctggcttgcgccaagcttaccataacctggcttgagccgcagataaagtcccaactccaatcggagcgcaaggactgggaagcgaacgaagtcggcgccttcttgaagaaggcgcccgagcgcaaggagcagttccacacgatcggggacttcccggtccagcgcacctacaccgctgccgacatcgccgacacgccgctggaggacatcggtcttccggggcgctacccgttcacgcgcgggccctacccgacgatgtaccgcagccgcacctggacgatgcgccagatcgccggcttcggcaccggcgaggacaccaacaagcgcttcaagtatctgatcgcgcagggccagaccggcatctccaccgacttcgacatgcccacgctgatgggctacgactccgaccacccgatgagcgacggcgaggtcggccgcgagggcgtggcgatcgacacgctggccgacatggaggcgctgctggccgacatcgacctcgagaagatctcggtctcgttcacgatcaacccgagcgcctggatcctgctcgcgatgtacgtggcgctcggcgagaagcgcggctacgacctgaacaagctgtcgggcacggtgcaggccgacatcctgaaggagtacatggcgcagaaggagtacatctacccgatcgcgccgtcggtgcgcatcgtgcgcgacatcatcacctacagcgcgaagaacctgaagcgctacaacccgatcaacatctcgggctaccacatcagcgaggccggctcctcgccgctccaggaggcggccttcacgctggccaacctgatcacctacgtgaacgaggtgacgaagaccggtatgcacgtcgacgaattcgcgccgcggttggccttcttcttcgtgtcgcaaggtgacttcttcgaggaggtcgcgaagttccgcgccctgcgccgctgctacgcgaagatcatgaaggagcgcttcggtgcaagaaatccggaatcgatgcggttgcgcttccactgtcagaccgcggcggcgacgctgaccaagccgcagtacatggtcaacgtcgtgcgtacgtcgctgcaggcgctgtcggccgtgctcggcggcgcgcagtcgctgcacaccaacggctacgacgaagccttcgcgatcccgaccgaggatgcgatgaagatggcgctgcgcacgcagcagatcattgccgaggagagtggtgtagccgacgtgatcgacccgctgggtggcagctactacgtcgaggcgctgaccaccgagtacgagaagaagatcttcgagatcctcgaggaagtcgagaagcgcggtggcaccatcaagctgatcgagcagggctggttccagaagcagattgcggacttcgcttacgagaccgcgctgcgcaagcagtccggccagaagccggtgatcggggtgaaccgcttcgtcgagaacgaagaggacgtcaagatcgagatccacccgtacgacaacacgacggccgaacgccagatttcccgcacgcgccgcgttcgcgccgagcgcgacgaggccaaggtgcaagcgatgctcgaccaactggtggctgtcgccaaggacgagtcccagaacctgatgccgctgaccatcgaactggtgaaggccggcgcaacgatgggggacatcgtcgagaagctgaaggggatctggggtacctaccgcgagacgccggtcttctgagcactagttggagagcttcccaccatggaccaaatcccgatccgcgttcttctcgccaaagtcggcctcgacggccatgaccgaggcgtcaaggtcgtcgctcgcgcgctgcgcgacgccggcatggacgtcatctactccggccttcatcgcacgcccgaagaggtggtcaacaccgccatccaggaagacgtggacgtgctgggtgtaagcctcctgtccggcgtgcagctcacggtcttccccaagatcttcaagctcctggacgagagaggcgctggcgacttgatcgtgatcgccggtggcgtgatgccggacgaggacgccgcggccatccgcaagctcggcgtgcgcgaggtgctcctgcaggacacgcccccgcaggccatcatcgactcgatccgctccttggtcgccgcgcgcggcgcccgctgaaagggcgagctctttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaataaggaagggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgag

(19) United States
(12) Patent Application Publication
(10) Pub. No.: US 2016/0138058 A1
(43) Pub. Date: May 19, 2016

(54) METHOD FOR PRODUCING ORGANIC COMPOSITIONS FROM OXYHYDROGEN AND CO2 VIA ACETOACETYL-COA AS INTERMEDIATE PRODUCT

(71) Applicants: Eva Maria WITTMANN, Traunreut (DE); Thomas HAAS, Muenster (DE); Steffen SCHAFFER, Herten (DE); Markus POETTER, Shanghai (CN); Yvonne SCHIEMANN, Essen (DE); Nigole KIRCHNER, Recklinghausen (DE)

(72) Inventors: Eva Maria WITTMANN, Traunreut (DE); Thomas HAAS, Muenster (DE); Steffen SCHAFFER, Herten (DE); Markus POETTER, Shanghai (CN); Yvonne SCHIEMANN, Essen (DE); Nigole KIRCHNER, Recklinghausen (DE)

(73) Assignee: Evonik Degussa GmbH, Essen (DE)

(21) Appl. No.: 14/898,417

(22) PCT Filed: May 30, 2014
(86) PCT No.: PCT/EP2014/061245

§ 371 Date: Dec. 14, 2015

(30) Foreign Application Priority Data
Jun. 14, 2013 (EP) 13172030.2

(51) Int. Cl. C12P 7/52; C12N 15/52; C12P 7/42; C12P 7/16; C12P 5/02

(52) U.S. Cl. CPC C12P 7/52; C12P 7/16; C12P 5/026; C12P 7/42; C12N 15/52

(57) ABSTRACT

The invention relates to a method for producing organic compositions comprising the method steps: A) providing an oxyhydrogen bacterium having an activity of an enzyme E₁, which is increased by comparison with the wild type thereof and which can catalyse the conversion of 2 acetyl-CoA to acetoacetyl-CoA and CoA, in an aqueous medium; B) bringing the aqueous medium into contact with a gas containing H₂, CO₂ and O₂ in a weight ratio from 20-70 to 10-45 to 5-35 and optionally C) purifying the organic composition.

### FIELD OF THE INVENTION

The present invention relates to a method for preparing organic compounds, comprising the method steps of

A) providing in an aqueous medium a hydrogen-oxidizing bacterium having an increased activity, compared to the wild type thereof, of an enzyme E₁ which is capable of catalyzing the conversion of
2 acetyl-CoA to acetoacetyl-CoA and CoA
B) contacting the aqueous medium with a gas comprising
H₂, CO₂ and O₂
in a weight ratio of from 20 to 70, to from 10 to 45, to from 5 to 35, and optionally
C) purifying the organic compound.

### PRIOR ART

Bio-based fuels and chemicals are nowadays typically prepared from carbohydrates, such as glucose, dextrose or glycerol. This has a multiplicity of disadvantages:

i) Prices for carbohydrates, such as glucose, dextrose or glycerol, will possibly develop analogously to raw fossil materials, since they can be converted into products having high calorific value.

ii) Carbohydrates, such as glucose, dextrose or glycerol, are subject to huge price fluctuations.

iii) Carbohydrates, such as glucose, dextrose or glycerol, are not available as raw materials in all regions in sufficient quantity.

iv) The use of carbohydrates, such as glucose or dextrose, as raw fermentation materials competes with use as foodstuffs.

Technologies for the highly selective preparation of organic compounds by means of biocatalysts and using H₂ and CO₂ as raw materials under energetically favorable, aerobic conditions are currently not yet available, but would make it possible to prepare, on the basis of raw materials and high regional flexibility, these chemicals from cost-effective raw materials or waste streams (natural gas, communal waste, biomass, converter gas, etc.).

### DESCRIPTION OF THE INVENTION

It has been found that, surprisingly, the method described hereinafter is capable of overcoming at least one of the disadvantages of the prior art.

The present invention therefore provides the method described in claim 1 and in the claims dependent thereon.

The invention further provides for the use of the hydrogen-oxidizing bacteria disclosed within the context of the invention for preparing organic compounds.

The present invention therefore provides a method for preparing an organic compound, comprising the method steps of

A) providing in an aqueous medium a hydrogen-oxidizing bacterium having an increased activity, compared to the wild type thereof, of an enzyme E₁ which is capable of catalyzing the conversion
of 2 acetyl-CoA to acetoacetyl-CoA and CoA
B) contacting the aqueous medium with a gas comprising
H₂, CO₂ and O₂
in a weight ratio of from 20 to 70, to from 10 to 45, to from 5 to 35, more particularly of from 30 to 55, to from 15 to 40, to from 10 to 30, and optionally
C) purifying the organic compound.

The term “hydrogen-oxidizing bacterium” is to be understood to mean a bacterium which is capable of chemolithoautotrophic growth and able to construct carbon skeletons having more than one carbon atom from H₂ and CO₂ in the presence of oxygen, in which the hydrogen is oxidized and the oxygen is used as terminal electron acceptor. According to the invention, it is possible to use either those bacteria which are naturally hydrogen-oxidizing bacteria or else bacteria which have become hydrogen-oxidizing bacteria by genetic modification, such as, for example, an E. coli cell which, as a result of recombinant insertion of the necessary enzymes, has been enabled to construct carbon skeletons having more than one carbon atom from H₂ and CO₂ in the presence of oxygen, in which the hydrogen is oxidized and the oxygen is used as terminal electron acceptor. Preferably, the hydrogen-oxidizing bacteria used in the method according to the invention are those which are already hydrogen-oxidizing bacteria as the wild type.

Hydrogen-oxidizing bacteria preferably used according to the invention are selected from the genera Achromobacter, Acidithiobacillus, Acidovorax, Alcaligenes, Anabena, Aquifex, Arthrobacter, Azospirillum, Bacillus, Bradyrhizobium, Cupriavidus, Derxia, Helicobacter, Herbaspirillum, Hydrogenobacter, Hydrogenobaculum, Hydrogenophaga, Hydrogenophilus, Hydrogenothermus, Hydrogenovibrio, Ideonella sp. O1, Kyrpidia, Metallosphaera, Methanobrevibacter, Myobacterium, Nocardia, Oligotropha, Paracoccus, Pelomonas, Polaromonas, Pseudomonas, Pseudonocardia, Rhizobium, Rhodococcus, Rhodopseudomonas, Rhodospirillum, Streptomyces, Thiocapsa, Treponema, Variovorax, Xanthobacter, Wautersia, wherein Cupriavidus is particularly preferred, particularly from the species Cupriavidus necator (alias Ralstonia eutropha, Wautersia eutropha, Alcaligenes eutrophus, Hydrogenomonas eutropha), Achromobacter ruhlandii, Acidithiobacillus ferrooxidans, Acidovorax facilis, Alcaligenes hydrogenophilus, Alcaligenes latus, Anabena cylindrica, Anabena oscillaroides, Anabena sp., Anabena spiroides, Aquifex aeolicus, Aquifex pyrophilus, Arthrobacter strain 11X, Bacillus schlegelii, Bradyrhizobium japonicum, Cupriavidus necator, Derxia gummosa, Escherichia coli, Heliobacter pylori, Herbaspirillum autotrophicum, Hydrogenobacter hydrogenophilus, Hydrogenobacter thermophilus, Hydrogenobaculum acidophilum, Hydrogenophaga flava, Hydrogenophaga palleronii, Hydrogenophaga pseudoflava, Hydrogenophaga taeniospiralis, Hydrogeneophilus thermoluteolus, Hydrogenothermus marinus, Hydrogenovibrio marinus, Ideonella sp. O-1, Kyrpidia tusciae, Metallosphaera sedula, Methanobrevibactercuticularis, Mycobacterium gordonae, Nocardia autotrophica, Oligotropha carboxidivorans, Paracoccus denitrificans, Pelomonas saccharophila, Polaromonas hydrogenivorans, Pseudomonas hydrogenovora, Pseudomonas thermophila, Rhizobium japonicum, Rhodococcus opacus, Rhodopseudomonas palustris, Seliberia carboxydohydrogena, Streptomyces thermoautotrophicus, Thiocapsa roseopersicina, Treponema primitia, Variovorax paradoxus, Xanthobacter autrophicus, Xanthobacter flavus,

particularly from the strains Cupriavidus necator H16, Cupriavidus necator H1 or Cupriavidus necator Z-1.

The hydrogen-oxidizing bacterium of the method according to the invention has an increased activity, compared to the wild type thereof, of an enzyme E₁.

The term “wild type” of a cell denotes here a cell whose genome is present in a state as has arisen naturally by evolution. The term is used both for the whole cell and for individual genes. The term “wild type”, therefore, particularly does not include those cells or genes whose gene sequences have been at least partially modified by man by means of recombinant techniques. The term “increased activity of an enzyme”, as used above and in the following comments in connection with the present invention, is preferably to be understood to mean that the wild type has been genetically modified in such a way that the relevant increase in activity occurs. Preferably, this term is to be understood to mean increased intracellular activity.

The following comments regarding the increase in enzyme activity in cells apply both to the increase in activity of enzyme E₁ and to all enzymes mentioned below whose activity may possibly be increased.

In principle, an increase in the enzymatic activity can be achieved by increasing the copy number of the gene sequence(s) coding for the enzyme, by using a strong promoter, by altering the codon usage of the gene, by increasing in various ways the half-life of the mRNA or of the enzyme, by modifying the regulation of expression of the gene or by using a gene or allele coding for a corresponding enzyme with increased activity and by combining these measures as appropriate. Genetically modified microorganisms used according to the invention are generated, for example, by transformation, transduction, conjugation, or a combination of these methods, with a vector containing the desired gene, an allele of this gene or parts thereof and a promoter enabling the gene to be expressed. Heterologous expression is achieved in particular by integrating the gene or alleles into the chromosome of the cell or an extrachromosomally replicating vector.

An overview of the options for increasing enzyme activity in cells is given for pyruvate carboxylase by way of example in DE-A-100 31 999, which is hereby incorporated by way of reference and whose disclosure forms part of the disclosure of the present invention regarding the options for increasing enzyme activity in cells.

Expression of the enzymes or genes specified above and all enzymes or genes specified below is detectable with the aid of 1- and 2-dimensional protein gel separation and subsequent optical identification of the protein concentration in the gel using appropriate evaluation software. If the increase in an enzyme activity is based exclusively on an increase in expression of the corresponding gene, the increase in said enzyme activity can be quantified in a simple manner by comparing the 1- or 2-dimensional protein separations between wild type and genetically modified cells. A customary method of preparing protein gels in the case of bacteria and of identifying said proteins is the procedure described by Hermann et al. (Electrophoresis, 22: 1712-23 (2001)). The protein concentration may likewise be analyzed by Western blot hybridization using an antibody specific for the protein to be detected (Sambrook et al., Molecular Cloning: a laboratory manual, 2nd Ed. Cold Spring Harbor Laboratory Press, Cold Spring Harbor, N.Y. USA, 1989) and subsequent optical evaluation using appropriate software for determination of concentration (Lohaus and Meyer (1989) Biospektrum, 5: 32-39; Lottspeich (1999), Angewandte Chemie 111: 2630-2647).

This method is also always used when possible products of the reaction catalyzed by the enzyme activity to be determined may be rapidly metabolized in the microorganism or else the activity in the wild type itself is too low to be able to adequately determine, on the basis of product formation, the enzyme activity to be determined.

Enzyme E₁, the activity of which is increased in the hydrogen-oxidizing bacterium compared to the wild type thereof, is preferably selected from acetyl-CoA:acetyl-CoA C-acetyltransferases from enzyme class EC 2.3.1.9.

The accession numbers cited in connection with the present invention correspond to the NCBI protein bank database entries dated Jun. 26, 2012; generally, in the present case, the version number of the entry is identified by “number” such as, for example, “1”.

Acetyl-CoA:acetyl-CoA C-acetyltransferases preferred according to the invention are selected from the list

### AAC26023.1, ABR35750.1 and ABR25255.1

and also
proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection is understood to mean the conversion of two acetyl-CoA to acetoacetyl-CoA and CoA.

A method for determining the activity is described in Middleton et al. The Biochemical journal (1972), 126(1), 27-34.

The hydrogen-oxidizing bacterium is provided in an aqueous medium in method step A). The aqueous medium used must appropriately fulfill the needs of the particular strains. Descriptions of culture media for various microorganisms are contained in the handbook “Manual of Methods for General Bacteriology” of the American Society for Bacteriology (Washington D.C., USA, 1981).

The medium may contain nitrogen sources; the nitrogen sources used may be organic nitrogen-containing compounds such as peptones, yeast extract, meat extract, malt extract, corn steep liquor, soybean meal and urea or inorganic compounds such as ammonium sulfate, ammonium chloride, ammonium phosphate, ammonium carbonate and ammonium nitrate, ammonia, ammonium hydroxide or aqueous ammonia. The nitrogen sources may be used individually or as a mixture.

The medium may contain phosphorus sources; the phosphorus sources used may be phosphoric acid, potassium dihydrogen phosphate or dipotassium hydrogen phosphate or the corresponding sodium-containing salts.

The medium may also contain carbon sources, though they should be limited in that the hydrogen-oxidizing bacterium is substantially dependent on the utilization of the carbon-containing gases present in the gas comprising H₂, CO₂ and O₂.

Furthermore, the medium must contain salts of metals such as, for example, magnesium sulfate or iron sulfate that are necessary for growth. Finally, essential growth substances such as amino acids and vitamins may be used in addition to the substances mentioned above. Moreover, suitable precursors may be added to the medium. The aforementioned starting materials may be added to the culture in the form of a single batch or be appropriately fed in during cultivation.

To control the pH of the culture, appropriate use is made of basic compounds such as sodium hydroxide, potassium hydroxide, ammonia or aqueous ammonia or acidic compounds such as phosphoric acid or sulfuric acid. To control the evolution of foam, it is possible to use antifoams such as, for example, fatty acid polyglycol esters. To maintain the stability of plasmids, it is possible to add to the medium suitable selective substances such as, for example, antibiotics.

In method step B), the aqueous medium is contacted with a gas comprising H₂, CO₂ and O₂. As a result of this, the hydrogen-oxidizing bacterium is provided with the gas comprising H₂, CO₂ and O₂ as carbon source. The hydrogen-oxidizing bacterium synthesizes acetoacetyl-CoA at least partly from this carbon source, which acetoacetyl-CoA is metabolized to other organic compounds. Unlike in an acetogenic process, which takes place under strictly anaerobic conditions, method step B) of the method according to the invention is carried out under aerobic conditions.

The partial pressure of hydrogen in the gas comprising H₂, CO₂ and O₂ is preferably 0.1 to 100 bar, preferably 0.2 to 10 bar, particularly preferably 0.5 to 4 bar.

The partial pressure of carbon dioxide in the gas comprising H₂, CO₂ and O₂ is preferably 0.03 to 100 bar, particularly preferably 0.05 to 1 bar, particularly 0.05 to 0.3 bar. The partial pressure of oxygen in the gas comprising H₂, CO₂ and O₂ is preferably 0.001 to 100 bar, particularly preferably 0.04 to 1 bar, particularly 0.04 to 0.5 bar.

Synthesis gas is particularly suitable as source of the H₂ and CO₂ in method step B), which is used admixed with oxygen; therefore, according to the invention, preference is given to the gas in method step B) comprising synthesis gas.

Synthesis gas can be provided e.g. from the by-product of carbon gasification. The hydrogen-oxidizing bacterium consequently converts a substance that is a waste product into a valuable raw material.

Alternatively, synthesis gas can be provided by the gasification of widely available, cost-effective agricultural raw materials for the method according to the invention. There are numerous examples of raw materials which can be converted into synthesis gas since almost all forms of vegetation can be utilized for this purpose. Preferred raw materials are selected from the group comprising perennial grasses such as Miscanthus sinensis, cereal residues, processing waste such as sawdust. In general, synthesis gas is obtained in a gasification apparatus from dried biomass, primarily by pyrolysis, partial oxidation and steam reformation, the primary products being CO, H₂ and CO₂.

Normally, some of the product gas is processed in order to optimize product yields and to avoid tar formation. The cracking of the undesired tar into synthesis gas and CO can be carried out with the use of lime and/or dolomite. These processes are described in detail in e.g. Reed, 1981 (Reed, T. B., 1981, Biomass gasification: principles and technology, Noves Data Corporation, Park Ridge, N.J.). It is also possible to use mixtures of different sources for generating synthesis gas.

With particular preference, the carbon contained in the synthesis gas in method step B) accounts for at least 50% by weight, preferably at least 70% by weight, particularly preferably at least 90% by weight, of the carbon of all carbon sources available to the hydrogen-oxidizing bacterium in method step B), the percentages by weight of carbon being based on the carbon atoms. The remaining carbon sources may be present, for example, in the form of carbohydrates in aqueous medium or also CO₂ from a source other than synthesis gas. Other CO₂ sources are waste gases such as, for example, flue gas, petroleum refinery waste gases, gases formed as a result of yeast fermentation or clostridial fermentation, waste gases from the gasification of cellulose-containing materials or of carbon gasification. These waste gases do not necessarily have to be formed as secondary phenomena of different processes, but may be produced specially for use in the method according to the invention.

The organic substance to be prepared by the method according to the invention is preferably selected from substances comprising three or four carbon atoms, particularly butanol, butene, propene, butyric acid, acetone, 2-hydroxyisobutyric acid and 2-propanol, and 2-hydroxyisobutyric acid.

### First Embodiment

### Butanol

A first embodiment of the method according to the invention is characterized in that the organic substance is butanol, E₁ is preferably selected from acetyl-CoA:acetyl-CoA C-acetyltransferases, and the hydrogen-oxidizing bacterium preferably has an increased activity, compared to the wild type thereof, of an enzyme E₂ which is capable of catalyzing the conversion

of acetoacetyl-CoA and NADH or NADPH to 3-hydroxybutyryl-CoA and NAD+ or NADP+. Enzyme E₂ is preferably selected from 3-hydroxybutyryl-CoA dehydrogenases from enzyme class EC:1.1.1.157.

3-Hydroxybutyryl-CoA dehydrogenases preferred according to the invention are selected from the list

NP_349314.1, YP_001307469.1 and CAQ53138.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₂ is generally understood to mean in particular the conversion of acetoacetyl-CoA and NADH to 3-hydroxybutyryl-CoA and NAD+.

A method for determining the activity is described in Senior et al. Biochemical Journal (1973), 134(1), 225-38.

A preferred first embodiment of the method according to the invention is characterized in that the hydrogen-oxidizing bacterium has, besides the increased activity of enzyme E₁ and optionally E₂, an increased activity, compared to the wild type thereof, of an enzyme E₃ which is capable of catalyzing the conversion

of 3-hydroxybutyryl-CoA to crotonyl-CoA and water.

Enzyme E₃ is preferably selected from 3-hydroxybutyryl-CoA dehydratases from enzyme class EC:4.2.1.55.

3-Hydroxybutyryl-CoA dehydratases preferred according to the invention are selected from the list

NP_349318.1, YP_001307465.1 and CAQ53134.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₃ is generally understood to mean in particular the conversion of 3-hydroxybutyryl-CoA to crotonyl-CoA and water.

A method for determining the activity is described in Boynton et al. Journal of bacteriology (1996), 178(11), 3015-24.

A further preferred first embodiment of the method according to the invention is characterized in that the hydrogen-oxidizing bacterium has, besides the increased activity of enzyme E₁ and optionally E₂ and/or E₃, an increased activity, compared to the wild type thereof, of an enzyme E₄ which is capable of catalyzing the conversion

of crotonyl-CoA and NADH or NADPH to butyryl-CoA and NAD+ or NADP+.

Enzyme E₄ is preferably selected from butyryl-CoA dehydrogenases from enzyme class EC:1.3.99.2.

Butyryl-CoA dehydrogenases preferred according to the invention are selected from the list NP_349317.1, YP_001307466.1 and CAQ53135.1

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₄ is generally understood to mean in particular the conversion of crotonyl-CoA and NADH to butyryl-CoA and NAD+.

A method for determining the activity is described in R. Graf, Ulm University dissertation 2002 and in Rhead et al., Proc. Natl. Acad. Sci. USA Vol. 77, No. 1, pp. 580-583, January 1980, Medical Sciences.

A further preferred first embodiment of the method according to the invention is characterized in that the hydrogen-oxidizing bacterium has, besides the increased activity of enzyme E₁ and optionally E₂, E₃ and/or E₄, an increased activity, compared to the wild type thereof, of an enzyme E₅ which is capable of catalyzing the conversion

of butyryl-CoA and NADH or NADPH to butyraldehyde, NAD+ or NADP+ and HS-CoA or the conversions
of butyryl-CoA and NADH or NADPH to butyraldehyde, NAD+ or NADP+ and HS-CoA and of butyraldehyde and NADH or NADPH to n-butanol and NAD+ or NADPH+.

Preferably, enzyme E₅ is selected from bifunctional aldehyde/alcohol dehydrogenases from enzyme class EC:1.2.1.10 or EC:1.1.1.1 or from butyraldehyde dehydrogenases from enzyme class EC:1.2.1.10.

The first-mentioned enzymes catalyze both of the aforementioned reactions, whereas butyraldehyde dehydrogenases only convert butyryl-CoA.

Bifunctional aldehyde/alcohol dehydrogenases preferred according to the invention are selected from the list

NP_149199.1, NP_563447.1 and YP_002861217.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₅ is, in the case of a bifunctional aldehyde/alcohol dehydrogenase, understood to mean in particular the conversion of at least one of the two reactions
of butyryl-CoA and NADH or NADPH to butyraldehyde, NAD+ or NADP+ and HS-CoA and of butyraldehyde and NADH or NADPH to n-butanol and NAD+ or NADPH+.

A method for determining the two activities is described in Yan et al. Appl. Environ. Microbiol. 1990 56 (9), 2591-9.

Butyraldehyde dehydrogenases preferred according to the invention are selected from the list YP_001310903.1 and CAQ57983.1

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₅ is, in the case of a butyraldehyde dehydrogenase, understood to mean in particular the conversion of butyryl-CoA and NADH to butyraldehyde, NAD+ and HS-CoA.

A further preferred first embodiment of the method according to the invention is characterized in that the hydrogen-oxidizing bacterium has, besides the increased activity of enzyme E₁ and optionally E₂, E₃, E₄ and/or E₅, an increased activity, compared to the wild type thereof, of an enzyme E₆ which is capable of catalyzing the conversion

of butyraldehyde and NAD(P)H to n-butanol and NAD(P)+.

Preferably, enzyme E₆ is selected from butanol dehydrogenases from enzyme class EC:1.1.1.-. Butanol dehydrogenases preferred according to the invention are selected from the list YP_001310904.1, YP_001310904 and CAQ53139.1 and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₆ is generally understood to mean in particular the conversion of butyraldehyde and NAD(P)H to n-butanol and NAD(P)+.

A method for determining the activity is described in Duerre et al., Applied Microbiology and Biotechnology (1987), 26(3), 268-72.

Particular preference is given to the hydrogen-oxidizing bacterium having an increased activity of E₆ when it has an increased activity of an enzyme E₅ which is capable of catalyzing only the conversion of butyryl-CoA and NADH or NADPH to butyraldehyde, NAD+ or NADP+ and HS-CoA and is therefore preferably a butyraldehyde dehydrogenase.

A further preferred first embodiment of the method according to the invention is characterized in that the hydrogen-oxidizing bacterium has, besides the increased activity of enzyme E₁ and optionally E₂, E₃, E₄, E₅ and/or E₆, an increased activity, compared to the wild type thereof, of an enzyme E₇ selected from electron transfer flavoproteins from enzyme class EC:2.8.3.9.

Particularly suitable here is an enzyme E₇ which is a heterodimeric enzyme constructed from two subunits, wherein the

alpha-subunit is selected from NP_349315.1, YP_001307468.1 and CAQ53137.1 and the beta-subunit is selected from NP_349316.1, YP_001307467.1 and CAQ53136.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence.

Particular preference is given here to combining alpha- and beta-subunits from the same organism.

According to the invention, it is preferred that the hydrogen-oxidizing bacterium which is used has an increased activity of E₇ when there is already an increased activity of E₄.

In particularly preferred first embodiments of the method according to the invention, the hydrogen-oxidizing bacterium has increased activities in the combinations

E₁E₂, E₁E₃, E₁E₄, E₁E₅, E₁E₅, E₁E₆, E₁E₇,
E₁E₂E₃, E₁E₃E₄, E₁E₅E₆, E₁E₂E₄, E₁E₂E₅, E₁E₂E₆, E₁E₃E₄, E₁E₃E₅, E₁E₃E₆, E₁E₄E₅, E₁E₄E₆, E₁E₆E₇,
E₁E₃E₄E₅E₆E₇, E₁E₂E₄E₅E₆E₇, E₁E₂E₃E₅E₆E₇, E₁E₂E₃E₄E₆E₇, E₁E₂E₃E₄E₅E₆ and E₁E₂E₃E₄E₅E₆E₇, wherein E₁E₂E₃E₄E₅E₆E₇, E₁E₃E₄E₅E₆E₇, E₁E₃E₄E₅E₇, E₁E₂E₃E₄E₅E₇
is particularly preferred.

### Second Embodiment

### Butene

A second embodiment of the method according to the invention is characterized in that the organic substance is butene, E₁ is preferably selected from acetyl-CoA:acetyl-CoA C-acetyltransferases, and the hydrogen-oxidizing bacterium preferably has an increased activity, compared to the wild type thereof, of an enzyme E₂ which is capable of catalyzing the conversion

of acetoacetyl-CoA and NADH or NADPH to 3-hydroxybutyryl-CoA and NAD+ or NADP+. Enzyme E₂ is preferably selected from 3-hydroxybutyryl-CoA dehydrogenases from enzyme class EC:1.1.1.157.

3-Hydroxybutyryl-CoA dehydrogenases preferred according to the invention are selected from the list

NP_349314.1, YP_001307469.1 and CAQ53138.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₂ is generally understood to mean in particular the conversion of acetoacetyl-CoA and NADH to 3-hydroxybutyryl-CoA and NAD+.

A preferred second embodiment of the method according to the invention is characterized in that the hydrogen-oxidizing bacterium has, besides the increased activity of enzyme E₁ and optionally E₂, an increased activity, compared to the wild type thereof, of an enzyme E₃ which is capable of catalyzing the conversion

of 3-hydroxybutyryl-CoA to crotonyl-CoA and water.

Enzyme E₃ is preferably selected from 3-hydroxybutyryl-CoA dehydratases from enzyme class EC:4.2.1.55.

3-Hydroxybutyryl-CoA dehydratases preferred according to the invention are selected from the list

NP_349318.1, YP_001307465.1 and CAQ53134.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₃ is generally understood to mean in particular the conversion of 3-hydroxybutyryl-CoA to crotonyl-CoA and water.

A further preferred second embodiment of the method according to the invention is characterized in that the hydrogen-oxidizing bacterium has, besides the increased activity of enzyme E₁ and optionally E₂ and/or E₃, an increased activity, compared to the wild type thereof, of an enzyme E₄ which is capable of catalyzing the conversion of crotonyl-CoA and NADH or NADPH to butyryl-CoA and NAD+ or NADP+.

Enzyme E₄ is preferably selected from butyryl-CoA dehydrogenases from enzyme class EC:1.3.99.2.

Butyryl-CoA dehydrogenases preferred according to the invention are selected from the list NP_349317.1, YP_001307466.1 and CAQ53135.1

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₄ is generally understood to mean in particular the conversion of crotonyl-CoA and NADH to butyryl-CoA and NAD+.

A further preferred second embodiment of the method according to the invention is characterized in that the hydrogen-oxidizing bacterium has, besides the increased activity of enzyme E₁ and optionally E₂, E₃ and/or E₄, an increased activity, compared to the wild type thereof, of an enzyme E₅ which is capable of catalyzing the conversion

of butyryl-CoA and NADH or NADPH to butyraldehyde, NAD+ or NADP+ and HS-CoA or the conversions
of butyryl-CoA and NADH or NADPH to butyraldehyde, NAD+ or NADP+ and HS-CoA and of butyraldehyde and NADH or NADPH to n-butanol and NAD+ or NADPH+.

Preferably, enzyme E₅ is selected from bifunctional aldehyde/alcohol dehydrogenases from enzyme class EC:1.2.1.10 or EC:1.1.1.1 or from butyraldehyde dehydrogenases from enzyme class EC:1.2.1.10.

The first-mentioned enzymes catalyze both of the aforementioned reactions, whereas butyraldehyde dehydrogenases only convert butyryl-CoA.

Bifunctional aldehyde/alcohol dehydrogenases preferred according to the invention are selected from the list

NP_149199.1, NP_563447.1 and YP_002861217.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₅ is, in the case of a bifunctional aldehyde/alcohol dehydrogenase, understood to mean in particular the conversion of at least one of the two reactions
of butyryl-CoA and NADH or NADPH to butyraldehyde, NAD+ or NADP+ and HS-CoA and of butyraldehyde and NADH or NADPH to n-butanol and NAD+ or NADPH+.

Butyraldehyde dehydrogenases preferred according to the invention are selected from the list YP_001310903.1 and CAQ57983.1

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₅ is, in the case of a butyraldehyde dehydrogenase, understood to mean in particular the conversion of butyryl-CoA and NADH to butyraldehyde, NAD+ and HS-CoA.

A further preferred second embodiment of the method according to the invention is characterized in that the hydrogen-oxidizing bacterium has, besides the increased activity of enzyme E₁ and optionally E₂, E₃, E₄ and/or E₆, an increased activity, compared to the wild type thereof, of an enzyme E₆ which is capable of catalyzing the conversion of butyraldehyde and NAD(P)H to n-butanol and NAD(P)+.

Preferably, enzyme E₆ is selected from butanol dehydrogenases from enzyme class EC:1.1.1.-. Butanol dehydrogenases preferred according to the invention are selected from the list YP_001310904.1, YP_001310904 and CAQ53139.1 and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₆ is generally understood to mean in particular the conversion of butyraldehyde and NAD(P)H to n-butanol and NAD(P)+.

Particular preference is given to the hydrogen-oxidizing bacterium having an increased activity of E₆ when it has an increased activity of an enzyme E₅ which is capable of catalyzing only the conversion of butyryl-CoA and NADH or NADPH to butyraldehyde, NAD+ or NADP+ and HS-CoA and is therefore preferably a butyraldehyde dehydrogenase.

A further preferred second embodiment of the method according to the invention is characterized in that the hydrogen-oxidizing bacterium has, besides the increased activity of enzyme E₁ and optionally E₂, E₃, E₄, E₅ and/or E₆, an increased activity, compared to the wild type thereof, of an enzyme E₇ selected from electron transfer flavoproteins from enzyme class EC:2.8.3.9.

Particularly suitable here is an enzyme E₇ which is a heterodimeric enzyme constructed from two subunits, wherein the

alpha-subunit is selected from NP_349315.1, YP_001307468.1 and CAQ53137.1 and the beta-subunit is selected from NP_349316.1, YP_001307467.1 and CAQ53136.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence.

Particular preference is given here to combining alpha- and beta-subunits from the same organism.

A further preferred second embodiment of the method according to the invention is characterized in that the hydrogen-oxidizing bacterium has, besides the increased activity of enzyme E₁ and optionally E₂, E₃, E₄, E₅, E₆ and/or E₇, an increased activity, compared to the wild type thereof, of an enzyme E₈ which is capable of catalyzing the conversion of n-butanol to 1-butene and water.

Preferably, enzyme E₈ is selected from oleate hydratases from enzyme class EC:4.2.1.53, kievitone hydratases from enzyme class EC:4.2.1.95 and phaseollidin hydratases from enzyme class EC:4.2.1.97.

Oleate hydratases preferred according to the invention are selected from the list ACT54545, OLHYD_STRPZ and OLHYD_FLAME, preferred kievitone hydratase is AAA87627.1, and also, for both enzyme classes, proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₈ is generally understood to mean in particular the conversion of n-butanol to 1-butene and water.

A method for determining the activity is described in Cleveland et al. Physio. Plant. Pathol. 22 (1983) 129-142; only kievitone needs to be replaced by butanol as substrate.

In particularly preferred second embodiments of the method according to the invention, the hydrogen-oxidizing bacterium has increased activities in the combinations E₁E₂E₈, E₁E₃E₈, E₁E₄E₈, E₁E₅E₈, E₁E₆E₈, E₁E₃E₄E₅E₆E₇E₈, E₁E₂E₄E₅E₆E₇E₈, E₁E₂E₃E₅E₆E₇E₈, E₁E₂E₃E₄E₆E₇E₈, E₁E₂E₃E₄E₅E₆E₈ and E₁E₂E₃E₄E₅E₆E₁E₈,

wherein E₁E₂E₃E₄E₅E₆E₁E₈, E₁E₃E₄E₅E₆E₁E₈, E₁E₃E₄E₅E₇E₈, E₁E₂E₃E₄E₅E₁E₈ is particularly preferred.

### Third Embodiment

### Propene and Butyric Acid

A third embodiment of the method according to the invention is characterized in that the organic substance is propene or butyric acid, E₁ is preferably selected from acetyl-CoA:acetyl-CoA C-acetyltransferases, and the hydrogen-oxidizing bacterium preferably has an increased activity, compared to the wild type thereof, of an enzyme E₂ which is capable of catalyzing the conversion

of acetoacetyl-CoA and NADH or NADPH to 3-hydroxybutyryl-CoA and NAD+ or NADP+.

Enzyme E₂ is preferably selected from 3-hydroxybutyryl-CoA dehydrogenases from enzyme class EC:1.1.1.157.

3-Hydroxybutyryl-CoA dehydrogenases preferred according to the invention are selected from the list

NP_349314.1, YP_001307469.1 and CAQ53138.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₂ is generally understood to mean in particular the conversion of acetoacetyl-CoA and NADH to 3-hydroxybutyryl-CoA and NAD+.

A preferred third embodiment of the method according to the invention is characterized in that the hydrogen-oxidizing bacterium has, besides the increased activity of enzyme E₁ and optionally E₂, an increased activity, compared to the wild type thereof, of an enzyme E₃ which is capable of catalyzing the conversion of 3-hydroxybutyryl-CoA to crotonyl-CoA and water.

Enzyme E₃ is preferably selected from 3-hydroxybutyryl-CoA dehydratases from enzyme class EC:4.2.1.55.

3-Hydroxybutyryl-CoA dehydratases preferred according to the invention are selected from the list

NP_349318.1, YP_001307465.1 and CAQ53134.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₃ is generally understood to mean in particular the conversion of 3-hydroxybutyryl-CoA to crotonyl-CoA and water.

A further preferred third embodiment of the method according to the invention is characterized in that the hydrogen-oxidizing bacterium has, besides the increased activity of enzyme E₁ and optionally E₂ and/or E₃, an increased activity, compared to the wild type thereof, of an enzyme E₄ which is capable of catalyzing the conversion

of crotonyl-CoA and NADH or NADPH to butyryl-CoA and NAD+ or NADP+.

Enzyme E₄ is preferably selected from butyryl-CoA dehydrogenases from enzyme class EC:1.3.99.2.

Butyryl-CoA dehydrogenases preferred according to the invention are selected from the list

NP_349317.1, YP_001307466.1 and CAQ53135.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₄ is generally understood to mean in particular the conversion of crotonyl-CoA and NADH to butyryl-CoA and NAD+.

A further preferred third embodiment of the method according to the invention is characterized in that the hydrogen-oxidizing bacterium has, besides the increased activity of enzyme E₁ and optionally E₂, E₃ and/or E₄, an increased activity, compared to the wild type thereof, of an enzyme E₉ which is capable of catalyzing the conversion

of n-butyryl-CoA and Pᵢ to butyryl phosphate and HS-CoA.

In this connection, Pᵢ is an inorganic phosphate.

Preferably, enzyme E₉ is selected from phosphate butyryltransferases from enzyme class EC:2.3.1.19.

Phosphate butyryltransferases preferred according to the invention are selected from the list ABR32393.1 and ZP_05394269.1

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₉ is generally understood to mean in particular the conversion of n-butyryl-CoA and Pᵢ to butyryl phosphate and HS-CoA.

A method for determining the activity is described in Wiesenborn et al. Applied and Environmental Microbiology (1989), 55(2), 317-22.

A further preferred third embodiment of the method according to the invention is characterized in that the hydrogen-oxidizing bacterium has, besides the increased activity of enzyme E₁ and optionally E₂, E₃, E₄ and/or E₉, an increased activity, compared to the wild type thereof, of an enzyme E₁₀ which is capable of catalyzing the conversion

of butyryl phosphate and ADP to butyrate and ATP.

Preferably, enzyme E₁₀ is selected from butyrate kinases from enzyme class EC:2.7.2.7. Butyrate kinases preferred according to the invention are selected from the list ABR32394.1 and ZP_05392467.1

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₀ is generally understood to mean in particular the conversion of butyryl phosphate and ADP to butyrate and ATP.

A method for determining the activity is described in Hartmanis J Biol Chem. 1987 Jan. 15; 262(2):617-21.

A further preferred third embodiment of the method according to the invention is characterized in that the hydrogen-oxidizing bacterium has, besides the increased activity of enzyme E₁ and optionally E₂, E₃, E₄, E₉ and/or E₁₀, an increased activity, compared to the wild type thereof, of an enzyme E₁ₗ which is capable of catalyzing the conversion

of butyrate and H₂O₂ to propene and H₂O.

Preferably, enzyme E₁ₗ is selected from cytochrome P450 of the CYP152 family.

Cytochrome P450 of the CYP152 family that are preferred according to the invention are selected from the list

HQ709266.1, NP_388092.1 and NP_739069.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁ₗ is generally understood to mean in particular the conversion of butyrate and H₂O₂ to propene and H₂O.

A method for determining the activity is described in Mathew Applied and Environmental Microbiology, Mar. 2011, pp. 1718-1727.

In particularly preferred third embodiments of the method according to the invention, the hydrogen-oxidizing bacterium has increased activities in the combinations E₁E₂E₃E₄E₉, E₁E₂E₃E₄E₁₀, E₁E₂E₃E₄E₁₁, E₁E₃E₄E₉E₁₀E₁₁, E₁E₂E₄E₉E₁₀E₁₁, E₁E₂E₃E₉E₁₀E₁ₗ, E₁E₂E₃E₄E₁₀E₁₁, E₁E₂E₃E₄E₉E₁ₗ, E₁E₂E₃E₄E₉E₁₀ and E₁E₂E₃E₄E₉E₁₀E₁₁,

wherein E₁E₂E₃E₄E₉E₁₀E₁₁, E₁E₃E₄E₉E₁₀E₁₁ is particularly preferred.

In the third embodiment of the method according to the invention, the combination of enzymes E₉E₁₀, even in combination with the aforementioned other enzymes as described above, which catalyzes in total the reaction from n-butyryl-CoA to butyrate, can be replaced by at least one enzyme, in which the n-butyryl-CoA is converted to butyrate by a thioesterase or acyl-CoA synthetase or acyl-CoA/acylate:CoA-transferase.

### Fourth Embodiment

### Acetone

A fourth embodiment of the method according to the invention is characterized in that the organic substance is acetone, E₁ is preferably selected from acetyl-CoA:acetyl-CoA C-acetyltransferases, and the hydrogen-oxidizing bacterium has an increased activity, compared to the wild type thereof, of an enzyme E₁₂ which is capable of catalyzing the conversion of acetoacetyl-CoA to acetoacetate and CoA.

Preferably, enzyme E₁₂ is selected from acetoacetyl-CoA:acetate/acyl:CoA transferases from enzyme class EC:3.1.2.11, from butyrate-acetoacetate CoA-transferases from enzyme class EC 2.8.3.9 or from acyl-CoA hydrolases from enzyme class EC 3.1.2.20.

Acetoacetyl-CoA:acetate/acyl:CoA transferases preferred according to the invention are selected from the list

transferases constructed from two subunits, wherein
the alpha-subunit is selected from NP_149326.1, YP_001310904.1 and CAQ57984.1 and the beta-subunit is selected from NP_149327.1, YP_001310905.1 and CAQ57985.1 and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₂ is generally understood to mean in particular the conversion of acetoacetyl-CoA to acetoacetate and CoA.

Butyrate-acetoacetate CoA-transferases preferred according to the invention are selected from ctfA and ctfB from Clostridium acetobutylicum and atoD and atoA from Escherichia coli and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₂ is generally understood to mean in particular the conversion of acetoacetyl-CoA to acetoacetate and CoA.

Acyl-CoA hydrolases preferred according to the invention are selected from tell from B. subtilis and ybgC from Heamophilus influenzae

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₂ is generally understood to mean in particular the conversion of acetoacetyl-CoA to acetoacetate and CoA.

A method for determining the transferase activity of enzyme E₁₂ is described in Jeffrey et al., Applied and Environmental Microbiology, the one for the hydrolase activity of enzyme E₁₂ in Aragon et al. Journal of Biological Chemistry (1983), 258(8), 4725-33.

A preferred fourth embodiment of the method according to the invention is characterized in that the hydrogen-oxidizing bacterium has, besides the increased activity of enzyme E₁ and optionally E₁₂, an increased activity, compared to the wild type thereof, of an enzyme E₁₃ which is capable of catalyzing the conversion

of acetoacetate to acetone and CO₂.

Enzyme E₁₃ is preferably selected from acetoacetate decarboxylases from enzyme class EC:4.1.1.4 or from acetone:CO2 ligases from enzyme class EC 6.4.1.6.

Acetoacetate decarboxylases preferred according to the invention are selected from the list NP_149328.1, YP_001310906.1 and CAQ57986.1

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₃ is generally understood to mean in particular the conversion of acetoacetate to acetone and CO₂.

A method for determining the activity is described in Daniel et al. Appl. Environ. Microbiol. 1990, pp. 3491-3498 Vol. 56, No. 11.

Acetone:CO2 ligases preferred according to the invention are selected from the list of oligomeric proteins having sequences.

A method for determining the activity of acetone:CO2 ligases is described by Miriam K. Sluis et al. in Proc Natl Acad Sci USA. 1997 August 5; 94(16): 8456-8461, the substrates used here being acetoacetate, AMP and orthophosphate.

In particularly preferred fourth embodiments of the method according to the invention, the hydrogen-oxidizing bacterium has increased activities in the combinations E₁E₁₂, E₁E₁₃ and E₁E₁₂E₁₃,

wherein E₁E₁₂E₁₃ is particularly preferred.

### Fifth Embodiment

### Propan-2-ol; Optionally Followed by Dehydration to Propene

A fifth embodiment of the method according to the invention is characterized in that the organic substance is propan-2-ol, E₁ is preferably selected from acetyl-CoA:acetyl-CoA C-acetyltransferases, and the hydrogen-oxidizing bacterium has an increased activity, compared to the wild type thereof, of an enzyme E₁₂ which is capable of catalyzing the conversion of acetoacetyl-CoA to acetoacetate and CoA.

Preferably, enzyme E₁₂ is selected from acetoacetyl-CoA:acetate/acyl:CoA transferases from enzyme class EC:3.1.2.11, from butyrate-acetoacetate CoA-transferases from enzyme class EC 2.8.3.9 or from acyl-CoA hydrolases from enzyme class EC 3.1.2.20.

Acetoacetyl-CoA:acetate/acyl:CoA transferases preferred according to the invention are selected from the list

transferases constructed from two subunits, wherein
the alpha-subunit is selected from NP_149326.1, YP_001310904.1 and CAQ57984.1 and the beta-subunit is selected from NP_149327.1, YP_001310905.1 and CAQ57985.1 and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₂ is generally understood to mean in particular the conversion of acetoacetyl-CoA to acetoacetate and CoA.

Butyrate-acetoacetate CoA-transferases preferred according to the invention are selected from ctfA and ctfB from Clostridium acetobutylicum and atoD and atoA from Escherichia coli and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₂ is generally understood to mean in particular the conversion of acetoacetyl-CoA to acetoacetate and CoA.

Acyl-CoA hydrolases preferred according to the invention are selected from tell from B. subtilis and ybgC from Heamophilus influenzae

and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₂ is generally understood to mean in particular the conversion of acetoacetyl-CoA to acetoacetate and CoA.

A preferred fifth embodiment of the method according to the invention is characterized in that the hydrogen-oxidizing bacterium has, besides the increased activity of enzyme E₁ and optionally E₁₂, an increased activity, compared to the wild type thereof, of an enzyme E₁₃ which is capable of catalyzing the conversion

of acetoacetate to acetone and CO₂.

Enzyme E₁₃ is preferably selected from acetoacetate decarboxylases from enzyme class EC:4.1.1.4 or from acetone:CO2 ligases from enzyme class EC 6.4.1.6.

Acetoacetate decarboxylases preferred according to the invention are selected from the list

NP_149328.1, YP_001310906.1 and CAQ57986.1
and also proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₃ is generally understood to mean in particular the conversion of acetoacetate to acetone and CO₂.

A method for determining the activity is described in Daniel et al. Appl. Environ. Microbiol. 1990, pp. 3491-3498 Vol. 56, No. 11.

A method for determining the activity of acetone:CO2 ligases is described by Miriam K. Sluis et al. in Proc Natl Acad Sci USA. 1997 August 5; 94(16): 8456-8461, the substrates used here being acetoacetate, AMP and orthophosphate.

A further preferred fifth embodiment of the method according to the invention is characterized in that the hydrogen-oxidizing bacterium has, besides the increased activity of enzyme E₁ and optionally E₁₂, and/or E₁₃, an increased activity, compared to the wild type thereof, of an enzyme E₁₄ which is capable of catalyzing the conversion

of acetone, NADPH and H⁺ to propan-2-ol+NADP⁺.

Preferably, enzyme E₁₄ is selected from propan-2-ol:NADP⁺ oxidoreductase from enzyme class EC:1.1.1.80.

A method for determining the activity of propan-2-ol:NADP⁺ oxidoreductase is described in Antonio D. Uttaro et al. in Molecular and Biochemical Parasitology 85 (1997) 213-219. In particularly preferred fifth embodiments of the method according to the invention, the hydrogen-oxidizing bacterium has increased activities in the combinations E₁E₁₄, E₁E₁₂ E₁₄, E₁E₁₂E₁₃ E₁₄ and E₁E₁₂E₁₃E₁₄,

wherein E₁E₁₂E₁₃E₁₄ is particularly preferred.

In a particularly preferred fifth embodiment of the method according to the invention, in method step C), the propan-2-ol is isolated from the aqueous solution by distillation as an azeotrope. Methods for isolating propan-2-ol are described in, inter alia, Lei Zhigang, Zhang Jinchang, Chen Biaohua Separation of aqueous isopropanol by reactive extractive distillation in Journal of Chemical Technology and Biotechnology Volume 77, Issue 11 pages 1251-1254, November 2002, Lloyd Berg et al. Separation of the propyl alcohols from water by azeotropic or extractive distillation U.S. Pat. No. 5,085,739 and Berg, Lloyd Separation of ethanol, isopropanol and water mixtures by azeotropic distillation U.S. Pat. No. 5,762,765.

A particularly preferred fifth embodiment of the method according to the invention comprises a method step D).

Dehydration of the propan-2-ol isolated in method step C) to give propene.

Preferably, the isolated propan-2-ol is converted in method step D) by chemical dehydration over an acidic catalyst to give propene. Relevant methods are described in Maria L. Martinez et al. Synthesis, characterization and catalytic activity of AISBA-3 mesoporous catalyst having variable silicon-to-aluminum ratios Microporous and Mesoporous Materials 144 (2011) 183-190 and A. S. Araujo et al. Kinetic study of isopropanol dehydration over silicoaluminophosphate catalyst Reaction Kinetics and Catalysis Letters January 1999, Volume 66, Issue 1, pp. 141-146.

### Sixth Embodiment

### 2-Hydroxyisobutyric Acid

A sixth embodiment of the method according to the invention is characterized in that the organic substance is 2-hydroxyisobutyric acid or a salt of 2-hydroxyisobutyric acid, E₁ is preferably selected from acetyl-CoA:acetyl-CoA C-acetyltransferases, the hydrogen-oxidizing bacterium optionally has an increased activity, compared to the wild type thereof, of an enzyme E₂ which is capable of catalyzing the conversion

of acetoacetyl-CoA and NADH or NADPH to 3-hydroxybutyryl-CoA and NAD+ or NADP+ and the hydrogen-oxidizing bacterium has an increased activity, compared to the wild type thereof, of an enzyme E₁₅ which is capable of catalyzing the conversion
of 3-hydroxybutyryl-coenzyme A to 2-hydroxyisobutyryl-coenzyme A.

Enzymes E₂ preferred in this connection are those specified as preferred ones in the first embodiment.

Enzyme E₁₅ is preferably a hydroxyisobutyryl-CoA mutase, an isobutyryl-CoA mutase (EC 5.4.99.13) or a methylmalonyl-CoA mutase (EC 5.4.99.2), preferably in each case a coenzyme B12-dependent mutase.

Enzyme E₁₅ is preferably those enzymes which can be isolated from the microorganisms Aquincola tertiaricarbonis L108, DSM18028, DSM18512, Methylibium petroleiphilum PM1, Methylibium sp. R8, Xanthobacter autotrophicus Py2, Rhodobacter sphaeroides (ATCC 17029), Nocardioides sp. JS614, Marinobacter algicola DG893, Sinorhizobium medicae WSM419, Roseovarius sp. 217, Pyrococcus furiosus DSM 3638.

In a preferred configuration of the sixth embodiment, enzyme E₁₅ is a heterodimeric enzyme comprising sequences selected from Seq ID Nos. 78 and 80 and also

proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the respective aforementioned reference sequences by deletion, insertion, substitution or a combination thereof and which still have at least 50%, preferably 65%, particularly preferably 80%, in particular more than 90%, of the activity of the protein having the corresponding aforementioned reference sequence, wherein 100% activity of the reference protein is understood to mean the increase in activity of the cells used as biocatalyst, i.e., the substance amount converted per unit time based on the cell amount used (units per gram of cell dry weight [U/g CDW]), in comparison with the activity of the biocatalyst without the presence of the reference protein, wherein the activity in this connection and in connection with the determination of the activity of enzyme E₁₅ is generally understood to mean in particular the conversion of 3-hydroxybutyryl-coenzyme A to 2-hydroxyisobutyryl-coenzyme A.

In particularly preferred sixth embodiments of the method according to the invention, the hydrogen-oxidizing bacterium has increased activities in the combinations E₁E₂E₁₅.

In the sixth embodiment of the method according to the invention, it is preferred that the hydrogen-oxidizing bacterium has, besides enzyme E₁₅, additionally an increased amount, compared to the wild type thereof, of a MeaB protein, particularly one having Seq ID No. 82. The MeaB protein is preferably those selected from sequence ID No. 82, YP_001023545.1 (Methylibium petroleiphilum PM1), YP_001409454.1 (Xanthobacter autotrophicus Py2), YP_001045518.1 (Rhodobacter sphaeroides ATCC 17029), YP_002520048.1 (Rhodobacter sphaeroides), AAL86727.1 (Methylobacterium extorquens AMI), CAX21841.1 (Methylobacterium extorquens DM4), YP_001637793.1 (Methylobacterium extorquens PA1), AAT28130.1 (Aeromicrobium erythreum), CAJ91091 (Polyangium cellulosum), AAM77046.1 (Saccharopolyspora erythraea) and NP_417393.1 (Escherichia coli str. K-12 substr. MG1655) and also

proteins having a polypeptide sequence in which up to 60%, preferably up to 25%, particularly preferably up to 15%, in particular up to 10, 9, 8, 7, 6, 5, 4, 3, 2, 1%, of the amino acid residues are modified with respect to the respective aforementioned reference sequences by deletion, insertion, substitution or a combination thereof.

In particular, enzymes E₁₅ and the MeaB protein can be expressed as fusion proteins, as disclosed for example in PCT/EP2010/065151 and which are used with particular preference. A method for determining the activity is described in Murthy V V et al. in Biochim Biophys Acta. 1977 Aug. 11; 483(2): 487-91.

The following figures form part of the examples:

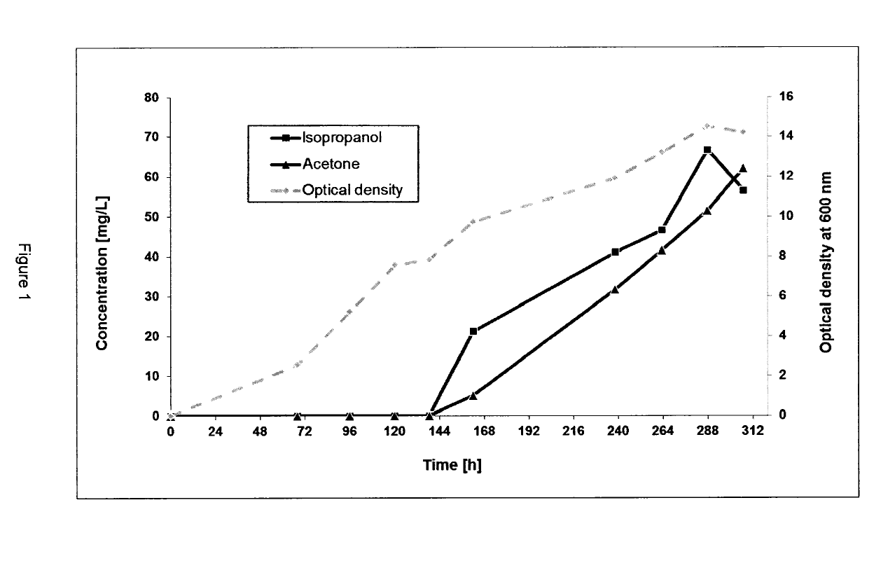

FIG. 1: Exemplary acetone and isopropanol production, C. necator H16 pBBR-EcatoDAB|Caadc

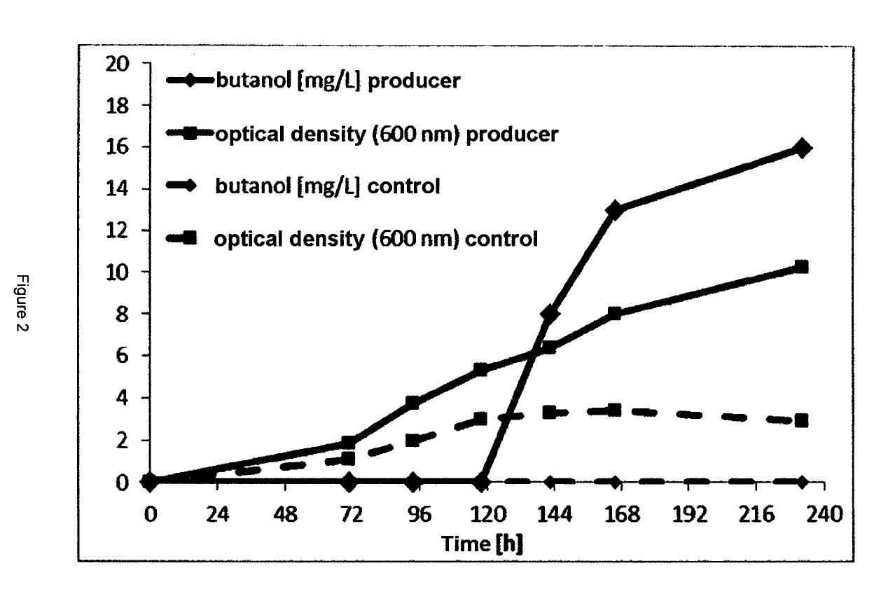

FIG. 2: Exemplary butanol production with C. necator H16 pBBR-RephaABJ-CaadhE₂

### EXAMPLES

### Working Example 1

### Construction of Plasmids for the Preparation of Acetone Using Cupriavidus necator

Plasmids for the preparation of acetone using C. necator were constructed by synthesizing five synthetic expression cassettes consisting of the following components:
1. the E. coli atoDAB operon, encoding the β-subunit of the acetyl-CoA:acetoacetyl-CoA transferase AtoD (Seq ID No. 13), the α-subunit of the acetyl-CoA:acetoacetyl-CoA transferase AtoA (Seq ID No. 14) and the thiolase AtoB (Seq ID No. 15), including the atoD, atoA and atoB ribosomal binding sites and the atoB terminator, wherein the regions encoding AtoD, AtoA and AtoB have been codon-optimized for translation in C. necator (nRBS-EcatoD-nRBS-EcatoA-nRBS-EcatoB-T-EcatoB; Seq ID No. 16)
2. the C. necator phaA gene, encoding the thiolase PhaA (Seq ID No. 17), including the phaA ribosomal binding site, and the C. acetobutylicum ctfAB operon, encoding the α- and β-subunits of the acetyl/butyryl-CoA-acetoacetate:CoA transferase CtfA (Seq ID No. 18) and CtfB (Seq ID No. 19), including the ctfA and ctfB ribosomal binding sites and the ctfAB terminator, wherein the regions encoding CtfA and CtfB have been codon-optimized for translation in C. necator (nRBS-RephaA-nRBS-CactfA-nRBS-CactfB-T-CactfAB; Seq ID No. 20)
3. the ribosomal binding site of the C. necator groEL gene (Seq ID No. 1), the C. acetobutylicum thIA gene, encoding the thiolase ThIA (Seq ID No. 21) and also the C. acetobutylicum ctfAB operon, encoding the α- and β-subunits of the acetyl/butyryl-CoA-acetoacetate:CoA transferase CtfA (Seq ID No. 18) and CtfB (Seq ID No. 19), including the native ctfA and ctfB ribosomal binding sites and the native ctfAB terminator, wherein the regions encoding ThIA, CtfA and CtfB have been codon-optimized for translation in C. necator (RBS-RegroEL-CathIA-nRBS-CactfA-nRBS-CactfB-T-CactfAB; Seq ID No. 22)
4. the ribosomal binding site of the C. necator groEL gene (Seq ID No. 1), followed by the C. acetobutylicum thIA gene, encoding the thiolase ThIA (Seq ID No. 21), the ribosomal binding site of the C. necator groEL gene (Seq ID No. 1), followed by the H. influenzae ybgC gene, encoding the thioesterase YbgC (Seq ID No. 23) and lastly the ribosomal binding site of the C. necator groEL gene (Seq ID No. 1), followed by the C. acetobutylicum adc gene, encoding the acetoacetate decarboxylase Adc (Seq ID No. 24), including the adc terminator, wherein the regions encoding ThIA, YbgC and Adc have been codon-optimized for translation in C. necator (RBS-RegroEL-CathIA-RBS-RegroEL-HiybgC-RBS-RegroEL-Caadc-T-Caadc; Seq ID No. 25)
5. the E. coli lacZ promoter (Seq ID No. 26), the ribosomal binding site of the C. necator groEL gene (Seq ID No. 1) and also the C. acetobutylicum adc gene, encoding the acetoacetate decarboxylase Adc (Seq ID No. 24), including the adc terminator, wherein the region encoding Adc has been codon-optimized for translation in C. necator (Plac-RBS-RegroEL-Caadc-T-Caadc; Seq ID No. 27)

The expression cassettes nRBS-EcatoD-nRBS-EcatoA-nRBS-EcatoB-T-EcatoB, nRBS-RephaA-nRBS-CactfA-nRBS-CactfB-T-CactfAB, RBS-RegroEL-CactfA-nRBS-CactfA-nRBS-CactfB-T-CactfAB and RBS-RegroEL-CathIA-RBS-RegroEL-HiybgC-RBS-RegroEL-Caadc-T-Caadc were then cloned via KpnI/HindIII into the broad-host-range expression vector pBBR1MCS-2 (Seq ID No. 10), and so the expression of the genes is under the control of the E. coli lacZ promoter. The resulting expression plasmids were designated pBBR-EcatoDAB, pBBR-RephaA-CactfAB, pBBR-CathIA-ctfAB and pBBR-CathIA-HiybgC-Caadc and correspond to the Seq ID Nos. 28, 29, 30 and 31.

The expression cassette Plac-RBS-RegroEL-Caadc-T-Caadc was then cloned via HindIII/BamHI into the vectors pBBR-EcatoDAB, pBBR-RephaA-CactfAB and pBBR-CathIA-ctfAB (Seq ID Nos. 28, 29 and 30). The resulting expression plasmids were designated pBBR-EcatoDAB|Caadc, pBBR-RephaA-CactfAB|adc and pBBR-CathIA-ctfAB|adc and correspond to the Seq ID Nos. 32, 33 and 34.

### Working Example 2

### Construction of Plasmids for the Preparation of Acetone Using Cupriavidus necator

Plasmids for the Preparation of Acetone Using C. necator were Constructed by Synthesizing Five Synthetic Expression Cassettes Consisting of the Following Components:
1. the E. coli atoDAB operon, encoding the β-subunit of the acetyl-CoA:acetoacetyl-CoA transferase AtoD (Seq ID No. 13), the α-subunit of the acetyl-CoA:acetoacetyl-CoA transferase AtoA (Seq ID No. 14) and the thiolase AtoB (Seq ID No. 15), including the atoD, atoA and atoB ribosomal binding sites and the atoB terminator, wherein the regions encoding AtoD, AtoA and AtoB have been codon-optimized for translation in C. necator (nRBS-EcatoD-nRBS-EcatoA-nRBS-EcatoB-T-EcatoB; Seq ID No. 16)
2. the C. necator phaA gene, encoding the thiolase PhaA (Seq ID No. 17), including the phaA ribosomal binding site, and the C. acetobutylicum ctfAB operon, encoding the α- and β-subunits of the acetyl/butyryl-CoA-acetoacetate:CoA transferase CtfA (Seq ID No. 18) and CtfB (Seq ID No. 19), including the ctfA and ctfB ribosomal binding sites and the ctfAB terminator, wherein the regions encoding CtfA and CtfB have been codon-optimized for translation in C. necator (nRBS-RephaA-nRBS-CactfA-nRBS-CactfB-T-CactfAB; Seq ID No. 20)
3. the ribosomal binding site of the C. necator groEL gene (Seq ID No. 1), the C. acetobutylicum thIA gene, encoding the thiolase ThIA (Seq ID No. 21) and also the C. acetobutylicum ctfAB operon, encoding the α- and β-subunits of the acetyl/butyryl-CoA-acetoacetate:CoA transferase CtfA (Seq ID No. 18) and CtfB (Seq ID No. 19), including the native ctfA and ctfB ribosomal binding sites and the native ctfAB terminator, wherein the regions encoding ThIA, CtfA and CtfB have been codon-optimized for translation in C. necator (RBS-RegroEL-CathIA-nRBS-CactfA-nRBS-CactfB-T-CactfAB; Seq ID No. 22)
4. the ribosomal binding site of the C. necator groEL gene (Seq ID No. 1), followed by the C. acetobutylicum thIA gene, encoding the thiolase ThIA (Seq ID No. 21), the ribosomal binding site of the C. necator groEL gene (Seq ID No. 1), followed by the H. influenzae ybgC gene, encoding the thioesterase YbgC (Seq ID No. 23) and lastly the ribosomal binding site of the C. necator groEL gene (Seq ID No. 1), followed by the Cupriavidus necator JMP134 acbB gene, encoding the acetone carboxylase beta-subunit AcbB (Seq ID No. 9), the ribosomal binding site of the C. necator groEL gene (Seq ID No. 1), followed by the Cupriavidus necator JMP134 acbA gene, encoding the acetone carboxylase alpha-subunit AcbA (Seq ID No. 8) and the ribosomal binding site of the C. necator groEL gene (Seq ID No. 1), followed by the Cupriavidus necator JMP134 acbC gene, encoding the acetone carboxylase gamma-subunit AcbC (Seq ID No. 86) including the adc terminator, wherein the regions encoding ThIA and YbgC have been codon-optimized for translation in C. necator (RBS-RegroEL-CathIA-RBS-RegroEL-HiybgC-RBS-RegroEL-AcbB-RBS-RegroEL-AcbA-RBS-RegroEL-AcBC-T-Caadc; Seq ID No. 7)
5. the E. coli lacZ promoter (Seq ID No. 26), the ribosomal binding site of the C. necator groEL gene (Seq ID No. 1) followed by the Cupriavidus necator JMP134 acbB gene, encoding the acetone carboxylase beta-subunit AcbB (Seq ID No. 9), the ribosomal binding site of the C. necator groEL gene (Seq ID No. 1), followed by the Cupriavidus necator JMP134 acbA gene, encoding the acetone carboxylase alpha-subunit AcbA (Seq ID No. 8) and the ribosomal binding site of the C. necator groEL gene (Seq ID No. 1), followed by the Cupriavidus necator JMP134 acbC gene, encoding the acetone carboxylase gamma-subunit AcbC (Seq ID No. 86) including the adc terminator (Plac-RBS-RegroEL-AcbB-RBS-RegroEL-AcbA-RBS-RegroEL-AcBC-T-Caadc; Seq ID No. 6)

The expression cassettes nRBS-EcatoD-nRBS-EcatoA-nRBS-EcatoB-T-EcatoB, nRBS-RephaA-nRBS-CactfA-nRBS-CactfB-T-CactfAB, RBS-RegroEL-CathIA-nRBS-CactfA-nRBS-CactfB-T-CactfAB and RBS-RegroEL-CathIA-RBS-RegroEL-HiybgC-RBS-RegroEL-AcbB-RBS-RegroEL-AcbA-RBS-RegroEL-AcBC-T-Caadc were then cloned via KpnI/HindIII into the broad-host-range expression vector pBBR1MCS-2 (Seq ID No. 10), and so the expression of the genes is under the control of the E. coli lacZ promoter. The resulting expression plasmids were designated pBBR-EcatoDAB, pBBR-RephaA-CactfAB, pBBR-CathIA-ctfAB and pBBR-CathIA-HiybgC-ReacbBAC and correspond to the Seq ID Nos. 28, 29, 30 and 5.

The expression cassette Plac-RBS-RegroEL-AcbB-RBS-RegroEL-AcbA-RBS-RegroEL-AcBC-T-Caadc was then cloned via HindIII/BamHI into the vectors pBBR-EcatoDAB, pBBR-RephaA-CactfAB and pBBR-CathIA-ctfAB (Seq ID Nos. 28, 29 and 30). The resulting expression plasmids were designated pBBR-EcatoDAB|ReacbBAC, pBBR-RephaA-CactfAB|ReacbBAC and pBBR-CathIA-ctfAB|ReacbBAC and correspond to the Seq ID Nos. 4, 3 and 2.

### Working Example 3

### Construction of Plasmids for the Preparation of 1-Butanol Using Cupriavidus necator

Seven synthetic expression cassettes were synthesized, consisting of the following components:
1. the C. necator phaA gene, encoding the thiolase PhaA (Seq ID No. 17), including the phaA ribosomal binding site, the C. necator phaB1 gene, encoding the (R)-3-hydroxybutyryl-CoA dehydrogenase PhaB1 (Seq ID No. 35), including the phaB1 ribosomal binding site, the Aeromonas caviae phaJ gene, encoding the (R)-3-hydroxybutyryl-CoA dehydratase PhaJ (Seq ID No. 36), including the phaJ ribosomal binding site, the ribosomal binding site of the C. necator groEL gene (Seq ID No. 1), the C. acetobutylicum adhE₂ gene, encoding the bifunctional butyryl-CoA/butyraldehyde dehydrogenase AdhE₂ (Seq ID No. 37), and also the A. caviae phaJ terminator (Seq ID No. 38), wherein the regions encoding PhaJ and AdhE₂ have been codon-optimized for translation in C. necator (nRBS-RephaA-nRBS-RephaB1-nRBS-AcphaJ-RBS-RegroEL-CaadhE₂-T-AcphaJ; Seq ID No. 39)
2. the C. necator phaA gene, encoding the thiolase PhaA (Seq ID No. 17), including the phaA ribosomal binding site, the C. necator phaB1 gene, encoding the (R)-3-hydroxybutyryl-CoA dehydrogenase PhaB1 (Seq ID No. 35), including the phaB1 ribosomal binding site and the Aeromonas caviae phaJ gene, encoding the (R)-3-hydroxybutyryl-CoA dehydratase PhaJ (Seq ID No. 36), including the phaJ ribosomal binding site and also the Aeromonas caviae phaJ terminator (Seq ID No. 38), wherein the region encoding PhaJ has been codon-optimized for translation in C. necator (nRBS-RephaA-nRBS-RephaB1-nRBS-AcphaJ-T-AcphaJ; Seq ID No. 40)
3. the ribosomal binding site of the C. necator groEL gene (Seq ID No. 1), followed by the C. acetobutylicum thIA gene, encoding the thiolase ThIA (Seq ID No. 21), the ribosomal binding site of the C. necator groEL gene (Seq ID No. 1), followed by the C. acetobutylicum hbd gene, encoding the (S)-3-hydroxybutyryl-CoA dehydrogenase Hbd (Seq ID No. 41), the ribosomal binding site of the C. necator groEL gene (Seq ID No. 1), followed by the C. acetobutylicum adhE₂ gene, encoding the bifunctional butyryl-CoA/butyraldehyde dehydrogenase AdhE₂ (Seq ID No. 37) and the C. acetobutylicum ctfAB terminator (Seq ID No. 42), wherein the regions encoding ThIA, Hbd and AdhE₂ have been codon-optimized for translation in C. necator (RBS-RegroEL-CathIA-RBS-RegroEL-Cahbd-RegroEL-CaadhE₂-T-CactfAB; Seq ID No. 43)
4. the ribosomal binding site of the C. necator groEL gene (Seq ID No. 1), followed by the C. acetobutylicum thIA gene, encoding the thiolase ThIA (Seq ID No. 21), the ribosomal binding site of the C. necator groEL gene (Seq ID No. 1), followed by the C. acetobutylicum hbd gene, encoding the (S)-3-hydroxybutyryl-CoA dehydrogenase Hbd (Seq ID No. 41) and the C. acetobutylicum ctfAB terminator (Seq ID No. 42), wherein the regions encoding ThIA and Hbd have been codon-optimized for translation in C. necator (RBS-RegroEL-CathIA-RBS-RegroEL-Cahbd-T-CactfAB; Seq ID No. 44)
5. the E. coli lacZ promoter (Seq ID No. 26), the C. necator etfBA-bcd operon, encoding the β-subunit of the electron transfer protein EtfB (Seq ID No. 45), the α-subunit of the electron transfer protein EtfA (Seq ID No. 46) and the butyryl-CoA dehydrogenase Bcd (Seq ID No. 46), including the etfB, etfA and bcd ribosomal binding sites and the C. necator etfBA-bcd terminator (Seq ID No. 48) (Plac-nRBS-ReetfB-nRBS-ReetfA-nRBS-Rebcd-nT; Seq ID No. 49)
6. the E. coli lacZ promoter (Seq ID No. 26), the C. acetobutylicum crt gene, encoding the crotonase Crt (Seq ID No. 50), including the crt ribosomal binding site, the C. acetobutylicum etfBA-bcd operon, encoding the β-subunit of the electron transfer protein EtfB (Seq ID No. 51), the α-subunit of the electron transfer protein EtfA (Seq ID No. 52) and the butyryl-CoA dehydrogenase Bcd (Seq ID No. 53), including the etfB, etfA and bcd ribosomal binding sites and the C. necator etfBA-bcd terminator (Seq ID No. 48) (Plac-nRBS-Cacrt-nRBS-CaeffB-nRBS-CaetfA-nRB-Cabcd-T-Rebcd; Seq ID No. 54)
7. the E. coli lacZ promoter (Seq ID No. 26), the C. acetobutylicum etfBA-bcd operon, encoding the β-subunit of the electron transfer protein EtfB (Seq ID No. 51), the α-subunit of the electron transfer protein EtfA (Seq ID No. 52) and the butyryl-CoA dehydrogenase Bcd (Seq ID No. 53), including the etfB, etfA and bcd ribosomal binding sites and the C. necator etfBA-bcd terminator (Seq ID No. 48) (Plac-nRBS-CaetfB-nRBS-CaetfA-nRB-Cabcd-T-Rebcd; Seq ID No. 55)

The expression cassettes nRBS-RephaA-nRBS-RephaB1-nRBS-AcphaJ-RBS-RegroEL-CaadhE₂-T-AcphaJ, nRBS-RephaA-nRBS-RephaB1-nRBS-AcphaJ-T-AcphaJ, RBS-RegroEL-CathIA-RBS-RegroEL-Cahbd-RegroEL-CaadhE₂-T-CactfAB and RBS-RegroEL-CathIA-RBS-RegroEL-Cahbd-T-CactfAB were then cloned via KpnI/HindIII into the broad-host-range expression vector pBBR1MCS-2 (Seq ID No. 10), and so the expression of the genes is under the control of the E. coli lacZ promoter. The resulting expression plasmids were designated pBBR-RephaABJ-CaadhE₂, pBBR-RephaABJ, pBBR-CathIA-hbd-adhE₂-ctfAB and pBBR-CathIA-hbd-ctfAB and correspond to the Seq ID Nos. 56, 57, 58 and 59.

The expression cassette Plac-nRBS-ReeffB-nRBS-ReetfA-nRBS-Rebcd-nT was then cloned via HindIII/BamHI into the vectors pBBR-RephaABJ-CaadhE₂ and pBBR-RephaABJ (Seq ID Nos. 56 and 57). The resulting expression plasmids were designated pBBR-RephaABJ-CaadhE₂|ReetfBA-bcd and pBBR-RephaABJ|ReetfBA-bcd and correspond to the Seq ID Nos. 60 and 61.

The expression cassette Plac-nRBS-Cacrt-nRBS-CaetfB-nRBS-CaetfA-nRB-Cabcd-T-Rebcd was then cloned via HindIII/BamHI into the vectors pBBR-RephaABJ-CaadhE₂, pBBR-RephaABJ, pBBR-CathIA-hbd-adhE₂-cffAB and pBBR-CathIA-hbd-ctfAB (Seq ID Nos. 56, 57, 58 and 59). The resulting expression plasmids were designated pBBR-RephaABJ-CaadhE₂|Cacrt-etfBA-Cabcd, pBBR-RephaABJ|Cacrt-etfBA-Cabcd, pBBR-CathIA-hbd-adhE₂-ctfAB|crt-effBA-bcd and pBBR-CathIA-hbd-ctfAB|crt-etfBA-bcd and correspond to the Seq ID Nos. 62, 63, 64 and 65.

The expression cassette Plac-nRBS-CaetfB-nRBS-CaetfA-nRB-Cabcd-T-Rebcd was then cloned via HindIII/BamHI into the vectors pBBR-RephaABJ-CaadhE₂ and pBBR-RephaABJ (Seq ID Nos. 56 and 57). The resulting expression plasmids were designated pBBR-RephaABJ-CaadhE₂|etfBA-bcd and pBBR-RephaABJ|CaetfBA-bcd and correspond to the Seq ID Nos. 66 and 67.

### Working Example 4

### Construction of Plasmids for the Preparation of Butyrate and 1-Propene Using Cupriavidus necator

Plasmids for the preparation of butyrate and 1-propene using C. necator were constructed by synthesizing a synthetic expression cassette consisting of the following components:
1. the C. necator groEL promoter (Seq ID No. 68), the ribosomal binding site of the C. necator groEL gene (Seq ID No. 1), followed by the Jeotgalicoccus sp. ATCC 8456 oleT gene, encoding the terminal olefin-forming fatty acid decarboxylase OleT (Seq ID No. 69), the ribosomal binding site of the C. necator groEL gene (Seq ID No. 1) and lastly the C. acetobutylicum ptb-buk operon, encoding the phosphotransbutyrylase Ptb (Seq ID No. 70) and the butyrate kinase Buk (Seq ID No. 71), including the buk ribosomal binding site and the ptb-buk terminator (Seq ID No. 72), wherein the regions encoding OleT, Ptb and Buk have been codon-optimized for translation in C. necator (PRegroESL-RBS-RegroEL-JeoleT-RBS-RegroEL-Captb-nRBS-Cabuk-T-Captb-buk; Seq ID No. 73)

The expression cassette PRegroESL-RBS-RegroEL-JeoleT-RBS-RegroEL-Captb-nRBS-Cabuk-T-Captb-buk was then cloned via BamHI/SacI into the vectors pBBR-RephaABJ|ReetfBA-bcd, pBBR-RephaABJ|CaetfBA-bcd and pBBR-CathIA-hbd-ctfAB|crt-etfBA-bcd (Seq ID Nos. 58, 60 and 62). The resulting expression plasmids were designated pBBR-RephaABJ|etfBA-bcd|JeoleT-Captb-buk, pBBR-RephaABJ|CaetfBA-bcd|JeoleT-Captb-buk and pBBR-CathIA-hbd-ctfAB|crt-etfBA-bcd|JeoleT-Captb-buk and correspond to the Seq ID Nos. 74, 75 and 76.

### Working Example 5

### Construction of Plasmids for the Preparation of 2-Propanol Using Cupriavidus necator

Plasmids for the preparation of 2-propanol using C. necator were constructed by synthesizing a synthetic expression cassette consisting of the following components:
1. the C. necator groEL promoter (Seq ID No. 68), the ribosomal binding site of the C. necator groEL gene (Seq ID No. 1), followed by the Clostridium beijerinckii NRRL B593 adh gene, encoding a primary and secondary alcohol-oxidizing alcohol dehydrogenase (Seq ID No. 11), wherein the region encoding the primary and secondary alcohol-oxidizing alcohol dehydrogenase has been codon-optimized for translation in C. necator (PRegroESL-RBS-Cbadh-T-Rebcd; Seq ID No. 12)

The expression cassette PRegroESL-RBS-Cbadh-T-Rebcd was then cloned via SacI/SpeI into the vectors pBBR-EcatoDAB|Caadc, pBBR-RephaA-CactfAB|adc and pBBR-CathIA-ctfAB|adc (Seq ID Nos. 32, 33 and 34). The resulting expression plasmids were designated pBBR-EcatoDAB|Caadc|Cbadh, pBBR-RephaA-CactfAB|adc|Cbadh and pBBR-CathIA-ctfAB|adc|Cbadh and correspond to the Seq ID Nos. 83, 84 and 85.

### Working Example 6

### Generation of the Vectors pBBR1MCS-2:HCM-phaA and pBBR1MCS-2:meaBhcmA-hcmB-phaA

The vector pBBR1MCS-2:HCM-phaA was generated starting from the plasmid pBBR1MCS-2:HCM (generation and properties described in patent application EP12173010). Construction was carried out by synthesizing a synthetic expression cassette consisting of the following components:
1. the C. necator phaA gene, encoding the thiolase PhaA (Seq ID No. 17), including the phaA ribosomal binding site; XbaI restriction sites are attached upstream and downstream of the sequence (nRBS-RephaA; Seq ID No. 87).

The plasmid pBBR1MCS-2:HCM was linearized using the restriction endonuclease XbaI and subsequently ligated with the expression cassette nRBS-RephaA, prepared by likewise carrying out restriction digestion using XbaI. All molecular biology tasks are carried out in a manner known to a person skilled in the art.

The genes are expressed after successful cloning under the control of the E. coli lacZ promoter. The resulting expression plasmid is designated pBBR1MCS-2:HCM-phaA and corresponds to the Seq ID No. 88.

The vector pBBR1MCS-2:meaBhcmA-hcmB-phaA was generated starting from the plasmid pBBR1MCS-2:meaBhcmA-hcmB (generation and properties described in patent application WO2011/057871). Construction was carried out by synthesizing a synthetic expression cassette consisting of the following components:
1. the C. necator phaA gene, encoding the thiolase PhaA (Seq ID No. 17), including the phaA ribosomal binding site; XbaI restriction sites are attached upstream and downstream of the sequence (nRBS-RephaA; Seq ID No. 87).

The plasmid pBBR1MCS-2:meaBhcmA-hcmB was linearized using the restriction endonuclease SacI and subsequently ligated with the expression cassette nRBS-RephaA, prepared by likewise carrying out restriction digestion using SacI. All molecular biology tasks are carried out in a manner known to a person skilled in the art.

The genes are expressed after successful cloning under the control of the E. coli lacZ promoter. The resulting expression plasmid is designated pBBR1MCS-2:meaBhcmA-hcmB-phaA and corresponds to the Seq ID No. 89.

### Working Example 7

### Introduction of Plasmids for the Preparation of Acetone, 2-Propanol, 1-Butanol, Butyrate, 2-Hydroxyisobutyric Acid and 1-Propene into Cupriavidus necator

The plasmids are transferred into competent E. coli S17-1 cells, a strain which makes possible the conjugative transfer of plasmids into, inter alia, Cupriavidus necator strains. To this end, a spot mating conjugation (as described in FRIEDRICH et al., 1981, Naturally occurring genetic transfer of hydrogen-oxidizing ability between strains of Alcaligenes eutrophus. J Bacteriol 147:198-205) is carried out with, as donors, the E. coli S17-1 strains bearing the respective plasmids and, as recipients, R. eutopha H16 (reclassified as Cupriavidus necator, DSMZ 428) and also R. eutropha PHB-4 (reclassified as Cupriavidus necator, DSMZ 541).

In all cases, transconjugants bearing the respective plasmids are obtained and the corresponding strains are designated as follows:
C. necator H16 pBBR-EcatoDAB|Caadc
C. necator H16 pBBR-RephaA-CactfAB|adc
C. necator H16 pBBR-CathIA-ctfAB|adc
C. necator H16 pBBR-RephaABJ-CaadhE₂
C. necator H16 pBBR-RephaABJ
C. necator H16 pBBR-CathIA-hbd-adhE₂-ctfAB
C. necator H16 pBBR-CathIA-hbd-ctfAB
C. necator H16 pBBR-RephaABJ-CaadhE₂|ReetfBA-bcd
C. necator H16 pBBR-RephaABJ|ReetfBA-bcd
C. necator H16 pBBR-RephaABJ-CaadhE₂|crt-etfBA-bcd
C. necator H16 pBBR-RephaABJ|crt-etfBA-bcd
C. necator H16 pBBR-RephaABJ-CaadhE₂|etfBA-bcd
C. necator H16 pBBR-RephaABJ|CaetfBA-bcd
C. necator H16 pBBR-CathIA-hbd-adhE₂-ctfAB|crt-etfBA-bcd
C. necator H16 pBBR-CathIA-hbd-ctfAB|crt-etfBA-bcd
C. necator H16 pBBR-RephaABJ|etfBA-bcd|JeoleT-Captb-buk
C. necator H16 pBBR-RephaABJ|CaetfBA-bcd|JeoleT-Captb-buk
C. necator H16 pBBR-CathIA-hbd-ctfAB|crt-etfBA-bcd|JeoleT-Captb-buk
C. necator H16 pBBR-EcatoDAB|Caadc|Cbadh
C. necator H16 pBBR-RephaA-CactfAB|adc|Cbadh
C. necator H16 pBBR-CathIA-ctfAB|adc|Cbadh
C. necator H16 pBBR-EcatoDAB|ReacbBAC
C. necator H16 pBBR-RephaA-CactfAB|ReacbBAC
C. necator H16 pBBR-CathIA-ctfAB|ReacbBAC
C. necator H16 pBBR1MCS-2:HCM-phaA
C. necator PHB-4 pBBR-EcatoDAB|Caadc
C. necator PHB-4 pBBR-RephaA-CactfAB|adc
C. necator PHB-4 pBBR-CathIA-ctfAB|adc
C. necator PHB-4 pBBR-RephaABJ-CaadhE₂
C. necator PHB-4 pBBR-RephaABJ
C. necator PHB-4 pBBR-CathIA-hbd-adhE₂-ctfAB
C. necator PHB-4 pBBR-CathIA-hbd-ctfAB
C. necator PHB-4 pBBR-RephaABJ-CaadhE₂|ReetfBA-bcd
C. necator PHB-4 pBBR-RephaABJ|ReetfBA-bcd
C. necator PHB-4 pBBR-RephaABJ-CaadhE₂|crt-etfBA-bcd
C. necator PHB-4 pBBR-RephaABJ|crt-etfBA-bcd
C. necator PHB-4 pBBR-RephaABJ-CaadhE₂|etfBA-bcd
C. necator PHB-4 pBBR-RephaABJ|CaetfBA-bcd
C. necator PHB-4 pBBR-CathIA-hbd-adhE₂-ctfAB|crt-etfBA-bcd
C. necator PHB-4 pBBR-CathIA-hbd-ctfAB|crt-etfBA-bcd
C. necator PHB-4 pBBR-RephaABJ|etfBA-bcd|JeoleT-Captb-buk
C. necator PHB-4 pBBR-RephaABJ|CaetfBA-bcd|JeoleT-Captb-buk
C. necator PHB-4 pBBR-CathIA-hbd-ctfAB|crt-etfBA-bcd|JeoleT-Captb-buk
C. necator PHB-4 pBBR-EcatoDAB|Caadc|Cbadh
C. necator PHB-4 pBBR-RephaA-CactfAB|adc|Cbadh
C. necator PHB-4 pBBR-CathIA-ctfAB|adc|Cbadh
C. necator PHB-4 pBBR-EcatoDAB|ReacbBAC
C. necator PHB-4 pBBR-RephaA-CactfAB|ReacbBAC
C. necator PHB-4 pBBR-CathIA-ctfAB|ReacbBAC
C. necator PHB-4 pBBR1MCS-2:HCM-phaA
C. necator PHB-4 pBBR1MCS-2:meaBhcmA-hcmB-phaA

### Working Example 8

### Quantification of Acetone, 2-Propanol, 1-Butanol, 2-Hydroxyisobutyric Acid, Butyrate and 1-Propene

### Butyrate and Butanol

Butanol and butyrate from a fermentation broth are quantified by means of HPLC/RID and DAD. 2 mL of each fermentation sample are centrifuged in a 2 mL Eppendorf reaction tube for 5 min at 13 000 rpm. Afterwards, the supernatant is sterile-filtered into an HPLC vial via a 0.2 μm syringe filter. If necessary, the samples must be diluted in line with the measurement range.

Measurement is carried out under the following conditions:

| | |
|---|---|
| HPLC | Agilent 1200, Agilent Technologies, Waldbronn |
| Mobile phase: | Mobile phase A1: H₂O (Millipore) + 5 mM |
| | aqueous H₂SO₄ |
| Gradient: | Isocratic |
| Column: | Supelcogel C-610H (9 μm particle size, |
| | L × I.D. 30 cm × 7.8 mm) |
| | Cat. No.: 59320-U (from Aldrich) |
| Precolumn: | Supelcogel H guard column (9 μm particle size, |
| | L × I.D. 5 cm × 4.6 mm) Cat. No.: 59319 (from Aldrich) |
| Column | 80° C. |
| temperature: | |
| Flow rate: | 1.0 mL/min |
| Detector: | RID |
| | DAD (210 nm) |
| Injection | 20 μL, “flush solvent” injector needle:isopropanol/ |
| | water (1:1) |
| volume: | |
| Run time: | 27 min |
| Measurement | For all analytes, about 0.100 g/L-12.0 g/L |
| range: | |

### Calibration and Evaluation:

Butanol and butyrate standard substances (˜20 mg per analyte) are weighed together into a 10 mL volumetric flask. The volumetric flask is filled with solvent (Millipore water) up to the calibration mark (solution S1: stock solution). From the stock solution, dilution with Millipore water is carried out to prepare a 5-point calibration. 1 mL of S1 is pipetted into a 10 mL volumetric flask and filled with solvent up to the calibration mark. In each measurement series, the calibration standards are measured in HPLC vials before and after the samples. For the evaluation, both calibration series are averaged. Butyrate is evaluated via the DAD signal at 210 nm. Butanol is evaluated in the RID chromatogram.

### 2-Hydroxyisobutyric Acid

Determination is carried out by quantitative ¹H-NMR spectroscopy. Trimethylpropionic acid is used as internal standard for quantification.

### Acetone, 2-Propanol and 1-Propene

The analytes acetone, isopropanol and 1-propene in the aqueous phase are determined by headspace GC/FID measurement and standard addition. The most important chromatographic parameters are summarized in the following table.

| | |
|---|---|
| Column | DB-Wax, 30 m × 250 μm, film thickness: 0.25 μm |
| GC system | Agilent 7890 |
| Gas flow rate | He, 0.9 mL/min |
| Column oven | Equilibration time: 0.5 min, |
| | temperature gradient: 0-4 min: 40° C., |
| | 5° C./min from 50 to 130° C., |
| | 30° C./min from 130 to 250° C., |
| | 250° C. for 12 min |
| Detector | FID, 250° C. |
| Injection | Headspace, temperature: 90° C., |
| | septum purge flow 2 mL/min, split 1:2 |
| Calibration | Calibration is carried out via standard addition with |
| | the corresponding analytes (internal 1-point calibration), |
| | linear measurement range 1-100 mg/L |

### Working Example 9

### Preparation of Acetone, 2-Propanol, 1-Butanol, Butyrate and 1-Propene Using Recombinant Ralstonia eutropha Cells

To investigate the formation of acetone, 2-propanol, 1-butanol, butyrate and 1-propene, the plasmid-bearing C. necator strains described in Example 7 are cultivated for the purposes of the preculture in 2×250 ml shake flasks with baffles in 25 ml of medium according to Vollbrecht et al., 1978. The medium consists of (NH₄)₂HPO₄ (2.0 g/l); KH₂PO₄ (2.1 g/l); MgSO₄×7H₂O (0.2 g/l); FeCl₃×6H₂O (6 mg/l); CaCl₂×2H₂O (10 mg); trace element solution (Pfennig and Lippert, 1966) 0.1 ml. The trace element solution is composed of Titriplex III (10 g/l), FeSO₄×7H₂O (4 g/l), ZnSO₄×7H₂O (0.2 g/l), MnCl₂×4H₂O (60 mg/l), H₃BO₃ (0.6 g/l), CoCl₂×6H₂O (0.4 g/l), CuCl₂×2H₂O (20 mg/l), NiCl₂×6H₂O (40 mg/l), Na₂Mo₄×2H₂O (60 mg/l). The preculture medium was additionally supplemented with fructose (5 g/l), kanamycin (300 μg/ml). The flasks were inoculated 1% (v/v) from a cryogenic culture. The cultures are incubated on a shaker at 30° C. and 150 rpm for 24 h. Thereafter, the cultures are combined and an OD₆₀₀ of about 3.5 is determined.

The main culture is carried out chemolithoautotrophically in a 2 l stainless steel reactor, Biostat B from Sartorius, filled with 1-2 l of medium having the composition (NH₄)₂HPO₄ (2.0 g/l), KH₂PO₄ (2.1 g/l), MgSO₄×7H₂O (3 g/l), FeCl₃×6H₂O (6 mg/l), CaCl₂×2H₂O (10 mg), biotin (1 mg/l), thiamine HCl (1 mg/l), Ca pantothenate (1 mg/l), nicotinic acid (20 mg/l), trace element solution (0.1 ml) and polypropylene glycol (PPG 1000 diluted 1:5 with water).

Cultivation is carried out at 30° C., 500-1500 rpm, pH 7. The pH is controlled unilaterally using 1 M NaOH. Gas is supplied using a gas mixture composed of H₂ (90%), CO₂ (6%), O₂ (4%) at a positive pressure of 0-2 bar with a gas flow rate of 0.19 vvm. The reactor is inoculated with 0.1% of the preculture. To this end, the required volume of preculture is centrifuged at 20° C. and 4500 rpm in 50 ml Falcon tubes for 10 min and resuspended in 10 ml of phosphate-buffered salt solution. Washing is repeated 3×; the last resuspension is carried out in 10 ml of main culture medium. The cultivation time is 76-150 h. Sampling is carried out by removing each time a 10 ml sample via a septum using a syringe having a sterile cannula. The following are determined: OD₆₀₀, dry biomass, and the product to be expected depending on the strain in accordance with Example 7.

The formation of acetone can be detected after cultivation of the following strains:
C. necator H16 pBBR-EcatoDAB|Caadc (see FIG. 1)
C. necator H16 pBBR-RephaA-CactfAB|adc
C. necator H16 pBBR-CathIA-ctfAB|adc
C. necator PHB-4 pBBR-EcatoDAB|Caadc
C. necator PHB-4 pBBR-RephaA-CactfAB|adc
C. necator PHB-4 pBBR-CathIA-ctfAB|adc

The formation of 2-propanol can be detected after cultivation of the following strains:
C. necator H16 pBBR-EcatoDAB|Caadc (see FIG. 1)
C. necator H16 pBBR-EcatoDAB|Caadc|Cbadh
C. necator H16 pBBR-RephaA-CactfAB|adc|Cbadh
C. necator H16 pBBR-CathIA-ctfAB|adc|Cbadh
C. necator PHB-4 pBBR-EcatoDAB|Caadc|Cbadh
C. necator PHB-4 pBBR-RephaA-CactfAB|adc|Cbadh
C. necator PHB-4 pBBR-CathIA-ctfAB|adc|Cbadh

The formation of 1-butanol can be detected after cultivation of the following strains:
C. necator H16 pBBR-RephaABJ-CaadhE₂ (see producer in FIG. 2)
C. necator H16 pBBR-RephaABJ
C. necator H16 pBBR-CathIA-hbd-adhE₂-ctfAB
C. necator H16 pBBR-CathIA-hbd-ctfAB
C. necator H16 pBBR-RephaABJ-CaadhE₂|ReetfBA-bcd
C. necator H16 pBBR-RephaABJ|ReetfBA-bcd
C. necator H16 pBBR-RephaABJ-CaadhE₂|crt-etfBA-bcd
C. necator H16 pBBR-RephaABJ|crt-effBA-bcd
C. necator H16 pBBR-RephaABJ-CaadhE₂|etfBA-bcd
C. necator H16 pBBR-RephaABJ|CaetfBA-bcd
C. necator H16 pBBR-CathIA-hbd-adhE₂-ctfAB|crt-etfBA-bcd
C. necator H16 pBBR-CathIA-hbd-ctfAB|crt-etfBA-bcd
C. necator PHB-4 pBBR-RephaABJ-CaadhE₂
C. necator PHB-4 pBBR-RephaABJ
C. necator PHB-4 pBBR-CathIA-hbd-adhE₂-ctfAB
C. necator PHB-4 pBBR-CathIA-hbd-ctfAB
C. necator PHB-4 pBBR-RephaABJ-CaadhE₂|ReetfBA-bcd
C. necator PHB-4 pBBR-RephaABJ|ReetfBA-bcd
C. necator PHB-4 pBBR-RephaABJ-CaadhE₂|crt-etfBA-bcd
C. necator PHB-4 pBBR-RephaABJ|crt-effBA-bcd
C. necator PHB-4 pBBR-RephaABJ-CaadhE₂|etfBA-bcd
C. necator PHB-4 pBBR-RephaABJ|CaetfBA-bcd
C. necator PHB-4 pBBR-CathIA-hbd-adhE₂-cffAB|crt-etfBA-bcd
C. necator PHB-4 pBBR-CathIA-hbd-ctfAB|crt-etfBA-bcd

The formation of butyrate can be detected after cultivation of the following strains:
C. necator H16 pBBR-RephaABJ|etfBA-bcd|JeoleT-Captb-buk
C. necator H16 pBBR-RephaABJ|CaetfBA-bcd|JeoleT-Captb-buk
C. necator H16 pBBR-CathIA-hbd-ctfAB|crt-etfBA-bcd|JeoleT-Captb-buk
C. necator PHB-4 pBBR-RephaABJ|etfBA-bcd|JeoleT-Captb-buk
C. necator PHB-4 pBBR-RephaABJ|CaetfBA-bcd|JeoleT-Captb-buk
C. necator PHB-4 pBBR-CathIA-hbd-ctfAB|crt-etfBA-bcd|JeoleT-Captb-buk

The formation of 1-propene can be detected after cultivation of the following strains:
C. necator H16 pBBR-RephaABJ|etfBA-bcd|JeoleT-Captb-buk
C. necator H16 pBBR-RephaABJ|CaetfBA-bcd|JeoleT-Captb-buk
C. necator H16 pBBR-CathIA-hbd-ctfAB|crt-etfBA-bcd|JeoleT-Captb-buk
C. necator PHB-4 pBBR-RephaABJ|etfBA-bcd|JeoleT-Captb-buk
C. necator PHB-4 pBBR-RephaABJ|CaetfBA-bcd|JeoleT-Captb-buk
C. necator PHB-4 pBBR-CathIA-hbd-ctfAB|crt-etfBA-bcd|JeoleT-Captb-buk

### Working Example 9

### Preparation of 2-Hydroxyisobutyric Acid Using Recombinant Cupriavidus necator Cells

A production phase of a plasmid-bearing Cupriavidus necator was used for the biotransformation of oxyhydrogen to 2-hydroxyisobutyric acid (2HIB). In this approach, the bacterium takes up H₂ and CO₂ from the conducted gas phase and forms 2HIB. For the cultivation, pressure-resistant glass bottles which can be sealed in an air-tight manner using a butyl rubber stopper were used. The plasmid-bearing C. necator strains used were the strains C. necator pBBR1 MCS2:HCM and C. necator pBBR1 MCS2:HCM-phaA.

To investigate the formation of 2-hydroxyisobutyric acid, the plasmid-bearing C. necator strains were firstly spread out on LB-R agar plates containing antibiotic and incubated at 30° C. for 3 days.

For the purposes of the preculture, the strains were cultivated in 200 ml of H16 mineral medium (modified according to Schlegel et al., 1961) in pressure-resistant 500 ml glass bottles. The medium consisted of Na₂HPO4×12 H₂O (9.0 g/l); KH₂PO₄ (1.5 g/l); NH₄Cl (1.0 g/l); MgSO4×7H₂O (0.2 g/l); FeCl₃×6H₂O (10 mg/l); CaCl₂×2H₂O (0.02 g/l); trace element solution SL-6 (Pfennig, 1974) (1 ml/l).

The trace element solution was composed of ZnSO₄×7H₂O (100 mg/l), MnCl₂×4H₂O (30 mg/l), H₃BO₃ (300 mg/l), CoCl₂×6H₂O (200 mg/l), CuCl₂×2H₂O (10 mg/l), NiCl₂×6H₂O (20 mg/l), Na₂Mo₄×2H₂O (30 mg/l). The pH of the medium was adjusted to 6.8 by addition of 1 M NaOH.

The bottles were inoculated with a single colony from the incubated agar plates and the cultivation was carried out chemolithoautotrophically on an N₂/H₂/O₂/CO₂ mixture (ratio 80%/10%/4%/6%). The cultures were incubated in an open water bath shaker at 28° C., 150 rpm and a gas flow rate of 1 l/h for 137 h, up to an OD>1.0. Gas was introduced into the medium via a gas supply frit which had a pore size of 10 μm and which was attached to a gas supply tube in the center of the reactor. The cells were subsequently centrifuged, washed with 10 ml of wash buffer (0.769 g/L NaOH, gassed through for at least 1 h at 28° C. and 150 rpm with a gas containing 6% CO₂) and recentrifuged.

For the production phase, sufficient washed cells were transferred from the growth culture to 200 mL of production buffer (NaOH (0.769 g/l), gassed through for at least 1 h at 28° C. and 150 rpm with a gas containing 6% CO₂, setting a pH of about 7.4) to set an OD₆₀₀ₙₘ of 1.0. The main culture was carried out chemolithoautotrophically in pressure-resistant 500 ml glass bottles at 28° C. and 150 rpm in an open water bath shaker with a gas flow rate of 1 l/h with an N₂/H₂/O₂/CO₂ mixture (ratio 80%/10%/4%/6%) for 116 h. The gas was introduced into the medium via a gas supply frit which had a pore size of 10 μm and which was attached to a gas supply tube in the center of the reactors. Sampling entailed removal of 5 ml samples in each case for the determination of OD₆₀₀ₙₘ, pH and the product spectrum. The product concentration was determined by semiquantitative 1H-NMR spectroscopy. The internal quantification standard used was sodium trimethylsilylpropionate (T(M)SP).

Over the cultivation time in the production phase, the strain C. necator pBBR1 MCS2:HCM exhibited the formation of up to 0.3 mg/l 2HIB, whereas the strain C. necator pBBR1 MCS2:HCM-phaA exhibited the formation of up to 0.8 mg/l 2HIB.

A production phase of a plasmid-bearing Cupriavidus necator is used for the biotransformation of oxyhydrogen to 2-hydroxyisobutyric acid (2HIB). In this approach, the bacterium takes up H₂ and CO₂ from the conducted gas phase and forms 2HIB. For the cultivation, pressure-resistant glass bottles which can be sealed in an air-tight manner using a butyl rubber stopper are used.

The plasmid-bearing C. necator strains used are the strains C. necator pBBR1 MCS2:meaBhcmA-hcmB and C. necator pBBR1 MCS2:meaBhcmA-hcmB-phaA.

To investigate the formation of 2-hydroxyisobutyric acid, the plasmid-bearing C. necator strains are firstly spread out on LB-R agar plates containing antibiotic and incubated at 30° C. for 3 days. For the purposes of the preculture, the strains are cultivated in 200 ml of H16 mineral medium (modified according to Schlegel et al., 1961) in pressure-resistant 500 ml glass bottles. The medium consists of Na₂HPO₄×12 H₂O (9.0 g/l); KH₂PO₄ (1.5 g/l); NH₄Cl (1.0 g/l); MgSO₄×7 H₂O (0.2 g/l); FeCl₃×6H₂O (10 mg/l); CaCl₂×2H₂O (0.02 g/l); trace element solution SL-6 (Pfennig, 1974) (1 ml/l).

The trace element solution is composed of ZnSO₄×7H₂O (100 mg/l), MnCl₂×4H₂O (30 mg/l), H3BO3 (300 mg/l), CoCl2×6H2O (200 mg/l), CuCl₂×2H₂O (10 mg/l), NiCl₂×6H₂O (20 mg/l), Na₂Mo₄×2H₂O (30 mg/l). The pH of the medium is adjusted to 6.8 by addition of 1 M NaOH. All media used are supplied with 300 μg/ml kanamycin and 76 nM CoB₁₂.

The bottles are inoculated with a single colony from the incubated agar plates and the cultivation is carried out chemolithoautotrophically on an N₂/H₂/O₂/CO₂ mixture (ratio 80%/10%/4%/6%). The cultures are incubated in an open water bath shaker at 28° C., 150 rpm and a gas flow rate of 1 l/h for 137 h, up to an OD>1.0. Gas is introduced into the medium via a gas supply frit which has a pore size of 10 μm and which is attached to a gas supply tube in the center of the reactor. The cells are subsequently centrifuged, washed with 10 ml of wash buffer (0.769 g/L NaOH, gassed through for at least 1 h at 28° C. and 150 rpm with a gas containing 6% CO₂) and recentrifuged.

For the production phase, sufficient washed cells are transferred from the growth culture to 200 mL of production buffer (NaOH (0.769 g/l), gassed through for at least 1 h at 28° C. and 150 rpm with a gas containing 6% CO₂, setting a pH of about 7.4) to set an OD₆₀₀ₙₘ of 1.0. The main culture is carried out chemolithoautotrophically in pressure-resistant 500 ml glass bottles at 28° C. and 150 rpm in an open water bath shaker with a gas flow rate of 1 l/h with an N₂/H₂/O₂/CO₂ mixture (ratio 80%/10%/4%/6%) for 116 h. The gas is introduced into the medium via a gas supply frit which has a pore size of 10 μm and which is attached to a gas supply tube in the center of the reactors. Sampling entails removal of 5 ml samples in each case for the determination of OD₆₀₀ₙₘ, pH and the product spectrum. The product concentration is determined by semiquantitative 1H-NMR spectroscopy. The internal quantification standard used is sodium trimethylsilylpropionate (T(M)SP).

Over the cultivation time in the production phase, the strain C. necator pBBR1 MCS2:meaBhcmA-hcmB exhibits the formation of up to 113 mg/l 2HIB, whereas the strain C. necator pBBR1 MCS2:meaBhcmA-hcmB-phaA exhibits the formation of up to 156 mg/l 2HIB.

The formation of 2-hydroxyisobutyric acid could be detected after cultivation of the following strains:
C. necator PHB-4 pBBR1MCS-2:HCM-phaA
C. necator H16 pBBR1MCS-2:HCM-phaA
C. necator PHB-4 pBBR1MCS-2:meaBhcmA-hcmB-phaA
C. necator H16 pBBR1MCS-2:meaBhcmA-hcmB-phaA

1. A method for preparing an organic compound, comprising
A) contacting (i) an aqueous medium comprising a hydrogen-oxidizing bacterium having an increased activity, compared to a wild type thereof, of an enzyme E₁ which is capable of catalyzing the conversion of 2 acetyl-CoA to acetoacetyl-CoA and CoA, with (ii) a gas comprising H₂, CO₂ and O₂ in a weight ratio of from 20 to 70, to from 10 to 45, to from 5 to 35, to obtain an organic compound, and optionally
(B) purifying the organic compound.

2. The method of claim 1, wherein the hydrogen-oxidizing bacterium is selected from the group of genera consisting of Achromobacter, Acidithiobacillus, Acidovorax, Alcaligenes, Anabena, Aquifex, Arthrobacter, Azospirillum, Bacillus, Bradyrhizobium, Cupriavidus, Derxia, Helicobacter, Herbaspirillum, Hydrogenobacter, Hydrogenobaculum, Hydrogenophaga, Hydrogenophilus, Hydrogenothermus, Hydrogenovibrio, Ideonella sp. O1, Kyrpidia, Metallosphaera, Methanobrevibacter, Myobacterium, Nocardia, Oligotropha, Paracoccus, Pelomonas, Polaromonas, Pseudomonas, Pseudonocardia, Rhizobium, Rhodococcus, Rhodopseudomonas, Rhodospirillum, Streptomyces, Treponema, Thiocapsa, Variovorax, Xanthobacter, and Wautersia.

3. The method of claim 1, wherein the enzyme E₁ is an acetyl-CoA:acetyl-CoA C-acetyltransferase from enzyme class EC 2.3.1.9.

4. The method of claim 1, wherein the enzyme E₁ is AAC26023.1, ABR35750.1 or ABR25255.1, or is a protein having a polypeptide sequence in which up to 60% of the amino acid residues are modified with respect to AAC26023.1, ABR35750.1 or ABR25255.1 by deletion, insertion, substitution or a combination thereof.

5. The method of claim 1, wherein the gas comprises synthesis gas.

6. The method of claim 5, wherein the synthesis gas accounts for at least 50% by weight, of all carbon sources available to the hydrogen-oxidizing bacterium.

7. The method of claim 1, wherein the CO₂ accounts for at least 50% by weight, of all carbon sources available to the hydrogen-oxidizing bacterium.

8. The method of claim 1, wherein:
(a) the organic compound is butanol, butene, propene or 2-hydroxyisobutyric acid, and the hydrogen-oxidizing bacterium has an increased activity, compared to the wild type thereof, of an enzyme E₂ which is capable of catalyzing the conversion of acetoacetyl-CoA and NADH or NADPH to 3-hydroxybutyryl-CoA and NAD+ or NADP+;
(b) the organic compound is butanol, butene, butyric acid or propene, and the hydrogen-oxidizing bacterium has an increased activity, compared to the wild type thereof, of an enzyme E₃ which is capable of catalyzing the conversion of 3-hydroxybutyryl-CoA to crotonyl-CoA and water; and/or
(c) the organic compound is butanol, butene, butyric acid or propene, and the hydrogen-oxidizing bacterium has an increased activity, compared to the wild type thereof, of an enzyme E₄ which is capable of catalyzing the conversion of crotonyl-CoA and NADH or NADPH to butyryl-CoA and NAD+ or NADP+.

9. The method of claim 8, wherein:
the enzyme E₂ is a 3-hydroxybutyryl-CoA dehydrogenase from enzyme class EC:1.1.1.157;
the enzyme E₃ is a 3-hydroxybutyryl-CoA dehydratase from enzyme class EC:4.2.1.55; and
the enzyme E₄ is a butyryl-CoA dehydrogenase from enzyme class EC:1.3.99.2.

10. The method of claim 9, wherein:
the enzyme E₂ is NP_349314.1, YP_001307469.1 or CAQ53138.1, or is a protein having a polypeptide sequence in which up to 60% of the amino acid residues are modified with respect to NP_349314.1, YP_001307469.1 or CAQ53138.1 by deletion, insertion, substitution or a combination thereof;
the enzyme E₃ is NP_349318.1, YP_001307465.1 or CAQ53134.1, or is a protein having a polypeptide sequence in which up to 60% of the amino acid residues are modified with respect to NP_349318.1, YP_001307465.1 or CAQ53134.1 by deletion, insertion, substitution or a combination thereof; and
the enzyme E₄ is NP_349317.1, YP_001307466.1 or CAQ53135.1, or is a protein having a polypeptide sequence in which up to 60% of the amino acid residues are modified with respect to NP_349317.1, YP_001307466.1 or CAQ53135.1 by deletion, insertion, substitution or a combination thereof.

11-16. (canceled)

17. The method of claim 1, wherein:
the organic compound is butanol or butene, and the hydrogen-oxidizing bacterium has an increased activity, compared to the wild type thereof, of an enzyme E₅ which is capable of catalyzing the conversion of butyryl-CoA and NADH or NADPH to butyraldehyde, NAD+ or NADP+ and HS-CoA or the conversions of butyryl-CoA and NADH or NADPH to butyraldehyde, NAD+ or NADP+ and HS-CoA and of butyraldehyde and NADH or NADPH to n-butanol and NAD+ or NADP+;
the organic compound is butanol or butene, and the hydrogen-oxidizing bacterium has an increased activity, compared to the wild type thereof, of an enzyme E₆ which is capable of catalyzing the conversion of butyraldehyde and NAD(P)H to n-butanol and NAD(P)+; and/or
the organic compound is butene, and the hydrogen-oxidizing bacterium has an increased activity, compared to the wild type thereof, of an enzyme E₈ which is capable of catalyzing the conversion of n-butanol to 1-butene and water.

18. The method of claim 17, wherein:
the enzyme E₅ is a bifunctional aldehyde/alcohol dehydrogenase from enzyme class EC:1.2.1.10 or EC:1.1.1.1 or a butyraldehyde dehydrogenase from enzyme class EC:1.2.1.10;
the enzyme E₆ is a butanol dehydrogenase from enzyme class EC:1.1.1; and
the enzyme E₈ is an oleate hydratase from enzyme class EC:4.2.1.53, a kievitone hydratase from enzyme class EC:4.2.1.95, or a phaseollidin hydratase from enzyme class EC:4.2.1.97.

19. The method of claim 18, wherein:
the enzyme E₅ is NP_149199.1, NP_563447.1, YP_002861217.1, YP_001310903.1 or CAQ57983.1, or is a protein having a polypeptide sequence in which up to 60% of the amino acid residues are modified with respect to NP_149199.1, NP_563447.1, YP_002861217.1, YP_001310903.1 or CAQ57983.1 by deletion, insertion, substitution or a combination thereof;
the enzyme E₆ is YP_001310904.1, YP_001310904 or CAQ53139.1, or is a protein having a polypeptide sequence in which up to 60% of the amino acid residues are modified with respect to YP_001310904.1, YP_001310904 or CAQ53139.1 by deletion, insertion, substitution or a combination thereof; and
the enzyme E₈ is ACT54545, OLHYD_STRPZ, OLHYD_FLAME or AAA87627.1, or is a protein having a polypeptide sequence in which up to 60% of the amino acid residues are modified with respect to ACT54545, OLHYD_STRPZ, OLHYD_FLAME or AAA87627.1 by deletion, insertion, substitution or a combination thereof.

20-22. (canceled)

23. The method of claim 1, wherein the hydrogen-oxidizing bacterium has an increased activity, compared to the wild type thereof, of an enzyme E₇ that is an electron transfer flavoprotein from enzyme class EC:2.8.3.9, and of an enzyme E₆ which is capable of catalyzing the conversion of butyraldehyde and NAD(P)H to n-butanol and NAD(P)+.

24. The method of claim 23, wherein:
the enzyme E₇ is a heterodimeric enzyme constructed from two subunits, wherein the alpha-subunit is NP_349315.1, YP_001307468.1 or CAQ53137.1, or is a protein having a polypeptide sequence in which up to 60% of the amino acid residues are modified with respect to NP_349315.1, YP_001307468.1 or CAQ53137.1 by deletion, insertion, substitution or a combination thereof, and the beta-subunit is NP_349316.1, YP_001307467.1 or CAQ53136.1, or is a protein having a polypeptide sequence in which up to 60% of the amino acid residues are modified with respect to NP_349316.1, YP_001307467.1 or CAQ53136.1 by deletion, insertion,
substitution or a combination thereof; and
the enzyme E₆ is a butanol dehydrogenase from enzyme class EC:1.1.1.

25-27. (canceled)

28. The method of claim 1, wherein:
the organic compound is propene or butyric acid, and the hydrogen-oxidizing bacterium has an increased activity, compared to the wild type thereof, of an enzyme E₉ which is capable of catalyzing the conversion of butyryl-CoA and Pᵢ to butyryl phosphate and HS-CoA;
the organic compound is propene or butyric acid, and the hydrogen-oxidizing bacterium has an increased activity, compared to the wild type thereof, of an enzyme E₁₀ which is capable of catalyzing the conversion of butyryl phosphate and ADP to butyrate and ATP; and/or
the organic compound is propene, and the hydrogen-oxidizing bacterium has an increased activity, compared to the wild type thereof, of an enzyme E₁₁ which is capable of catalyzing the conversion of butyrate and H₂O₂ to propene and H₂O.

29. The method of claim 28, wherein:
the enzyme E₉ is a phosphate butyryltransferase from enzyme class EC:2.3.1.19;
the enzyme E₁₀ is a butyrate kinase from enzyme class EC:2.7.2.7; and
the enzyme E₁₁ is a cytochrome P450 of the CYP152 family.

30. The method of claim 29, wherein:
the enzyme E₉ is ABR32393.1 or ZP_05394269.1, or a protein having a polypeptide sequence in which up to 60% of the amino acid residues are modified with respect to ABR32393.1 or ZP_05394269.1 by deletion, insertion, substitution or a combination thereof;
the enzyme E₁₀ is ABR32394.1 or ZP_05392467.1, or a protein having a polypeptide sequence in which up to 60% of the amino acid residues are modified with respect to ABR32394.1 or ZP_05392467.1 by deletion, insertion, substitution or a combination thereof; and
the enzyme E₁₁ is HQ709266.1, NP_388092.1 or NP_739069.1, or a protein having a polypeptide sequence in which up to 60% of the amino acid residues are modified with respect to HQ709266.1, NP_388092.1 or NP_739069.1 by deletion, insertion, substitution or a combination thereof.

31-36. (canceled)

37. The method of claim 1, wherein:
the organic compound is acetone, 2-propanol or propene, and the hydrogen-oxidizing bacterium has an increased activity, compared to the wild type thereof, of an enzyme E₁₂ which is capable of catalyzing the conversion of acetoacetyl-CoA to acetoacetate and CoA;
the organic compound is acetone, 2-propanol or propene, and the hydrogen-oxidizing bacterium has an increased activity, compared to the wild type thereof, of an enzyme E₁₃ which is capable of catalyzing the conversion of acetoacetate to acetone and CO₂;
the organic compound is 2-propanol or propene, and the hydrogen-oxidizing bacterium has an increased activity, compared to the wild type thereof, of an enzyme E₁₄ which is capable of catalyzing the conversion of acetone, NADPH and H+ to propan-2-ol+ NADP+; and/or
the organic compound is 2-hydroxyisobutyric acid and the hydrogen-oxidizing bacterium has an increased activity, compared to the wild type thereof, of an enzyme E₁₅ which is capable of catalyzing the conversion of 3-hydroxybutyryl-coenzyme A to 2-hydroxyisobutyryl-coenzyme A.

38. The method of claim 37, wherein:
the enzyme E₁₂ is an acetoacetyl-CoA:acetate/acyl:CoA transferase from enzyme class EC:3.1.2.11, a butyrate-acetoacetate CoA-transferase from enzyme class EC:2.8.3.9 or an acyl-CoA hydrolase from enzyme class EC:3.1.2.20;
the enzyme E₁₃ is an acetoacetate decarboxylase from enzyme class EC:4.1.1.4 or an acetone:CO2 ligase from enzyme class EC 6.4.1.6;
the enzyme E₁₄ is a propan-2-ol:NADP+ oxidoreductase from enzyme class EC:1.1.1.80; and
the enzyme E₁₅ is a hydroxyisobutyryl-CoA mutase, an isobutyryl-CoA mutase from enzyme class EC 5.4.99.13, or a methylmalonyl-CoA mutase from enzyme class EC 5.4.99.2.

39. The method of claim 38, wherein:
the enzyme E₁₂ is selected from the group consisting of (i), (ii) and (iii):
(i) a heterodimeric acetoacetyl-CoA:acetate/acyl:CoA transferase constructed from two subunits, wherein
an alpha-subunit is selected from the group consisting of NP_149326.1, YP_001310904.1 and CAQ57984.1
and a beta-subunit is selected from the group consisting of NP_149327.1, YP_001310905.1 and CAQ57985.1, or
a protein having a polypeptide sequence in which up to 60% of the amino acid residues are modified with respect to NP_149326.1, YP_001310904.1, CAQ57984.1, NP_149327.1, YP_001310905.1 or CAQ57985.1 by deletion, insertion, substitution or a combination thereof,
(ii) the butyrate-acetoacetate CoA-transferases ctfA and ctfB from Clostridium acetobutylicum and atoD and atoA from Escherichia coli, or a protein having a polypeptide sequence in which up to 60% of the amino acid residues are modified with respect to ctfA, ctfB, atoD or atoA by deletion, insertion, substitution or a combination thereof, and
(iii) the acyl-CoA hydrolases tell from B. subtilis and ybgC from Heamophilus influenza, or a protein having a polypeptide sequence in which up to 60% of the amino acid residues are modified with respect to teII or ybgC by deletion, insertion, substitution or a combination thereof;
the enzyme E₁₃ is NP_149328.1, YP_001310906.1 or CAQ57986.1, or a protein having a polypeptide sequence in which up to 60% of the amino acid residues are modified with respect to NP_149328.1, YP_001310906.1 or CAQ57986.1 by deletion, insertion, substitution or a combination thereof;
the enzyme E₁₄ is P14941.1, P35630.1, P75214.1 or P25984.1, or a protein having a polypeptide sequence in which up to 60% of the amino acid residues are modified with respect to P14941.1, P35630.1, P75214.1 or P25984.1 by deletion, insertion, substitution or a combination thereof; and/or
the enzyme E₁₅ is an enzyme isolated from a microorganism selected from the group consisting of Aquincola tertiaricarbonis L108, DSM18028, DSM18512, Methylibium petroleiphilum PM1, Methylibium sp. R8, Xanthobacter autotrophicus Py2, Rhodobacter sphaeroides (ATCC 17029), Nocardioides sp. JS614, Marinobacter algicola DG893, Sinorhizobium medicae WSM419, Roseovarius sp. 217, and Pyrococcus furiosus DSM 3638.

40-49. (canceled)

50. The method of claim 8, wherein the hydrogen-oxidizing bacterium with increased expression of the enzyme E₁₅ has an increased amount, compared to the wild type thereof, of a MeaB protein.
